(19) United States
(12) Patent Application Publication
(10) Pub. No.: US 2025/0002489 A1
(43) Pub. Date: Jan. 2, 2025

(54) BIFUNCTIONAL COMPOUNDS FOR DEGRADING ITK VIA UBIQUITIN PROTEOSOME PATHWAY

(71) Applicant: NURIX THERAPEUTICS, INC., San Francisco, CA (US)

(72) Inventors: Kerem OZBOYA, San Francisco, CA (US); Yan MENG, San Francisco, CA (US); Daisuke KATO, San Francisco, CA (US); Katherine KEENAN, San Francisco, CA (US); Ge PENG, San Francisco, CA (US); Daniel W. ROBBINS, San Francisco, CA (US)

(21) Appl. No.: 18/702,666

(22) PCT Filed: Oct. 19, 2022
(86) PCT No.: PCT/US2022/047129

§ 371 Date: Apr. 18, 2024

Related U.S. Application Data
(60) Provisional application No. 63/257,557, filed on Oct. 19, 2021.

(51) Int. Cl. C07D 417/14; A61K 31/4155; A61K 31/422; A61K 31/427; A61K 31/4439; A61K 31/454; A61K 31/4545; A61K 31/496; A61K 31/506; C07D 403/14; C07D 413/14

(52) U.S. Cl. CPC C07D 417/14; A61K 31/4155; A61K 31/422; A61K 31/427; A61K 31/4439; A61K 31/454; A61K 31/4545; A61K 31/496; A61K 31/506; C07D 403/14; C07D 413/14

(57) ABSTRACT

This disclosure relates to compounds of formulae I, II and III useful for degrading ITK via a ubiquitin proteolytic pathway. This disclosure also provides pharmaceutically acceptable compositions comprising said compounds, and methods of using the compositions in the treatment of various diseases, conditions, and/or disorders.

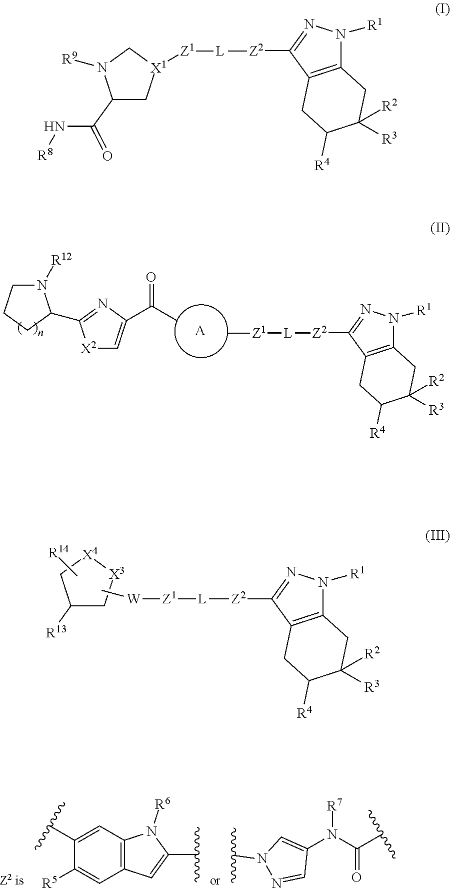

### CROSS REFERENCE

The present application claims the benefit of U.S. provisional patent application No. 63/257,557, filed Oct. 19, 2021, the contents of which are hereby incorporated by reference in its entirety.

### FIELD

This disclosure provides novel bifunctional compounds for proteolytically degrading targeted IL-2 inducible T-cell kinase (ITK) and methods for treating diseases modulated by ITK.

### BACKGROUND

IL-2 inducible T-cell kinase (ITK) is a tyrosine protein kinase and a member of the TEC family of kinases. Gibson et al., 1993, Blood 82(5):1561-1572. ITK is highly expressed in T-cells. Gomez-Rodriguez et al., 2011, FEBS J. 278(12):1980-1989. ITK is reported to be activated downstream of the T-cell receptor (TCR) via phosphorylation from Lck kinase. Gomez-Rodriguez et al., 2011. ITK is believed to activate phospholipase Cγ1 (PLCγ1) to drive T-cell function and immune responses. Gomez-Rodriguez et al., 2011; Kosaka et al., 2006, Trends Immunol. 27(10):453-60. ITK has been shown to be involved in numerous inflammatory, autoimmune, and proliferative diseases including allergic asthma, atopic dermatitis, aplastic anemia, inflammatory bowel disease, neuroinflammation, and T cell lymphomas. Lechner et al., 2020, J. Mol. Medicine 98:1385-1395. ITK provides a promising target for therapies for treating several inflammatory, autoimmune, and proliferative diseases and disorders.

### SUMMARY

Provided herein are compounds capable of binding, inhibiting, and/or degrading ITK. The compounds are useful for the treatment or prevention of inflammatory, autoimmune, and proliferative diseases and disorders in a subject in need thereof.

In a first aspect, provided herein are compounds according to Formula (A):

ITK Hook-Linker-Ubiquitin Ligase Harness (A)

The compounds of Formula (A) comprise an ITK hook. The ITK hook is a moiety capable of binding ITK in vitro, in vivo, and/or in a cell. Useful ITK hooks are described herein. The Ubiquitin Ligase Harness is a moiety capable of harnessing a ubiquitin ligase in vitro, in vivo, and/or in a cell. In certain embodiments, the ubiquitin ligase is an E3 ligase. In certain embodiments, the ubiquitin ligase is cereblon. Useful ubiquitin ligase harnesses are described herein. The compounds of Formula (A) further comprise a Linker. The Linker is any moiety capable of covalently binding the Harness and the Hook while permitting each to bind or harness its target. By harnessing a ubiquitin ligase and binding ITK, the compounds of Formula (A) are capable of targeting ITK for degradation under the appropriate conditions, for instance in a cell.

As shown in the Examples herein, the compounds of Formula (A) degrade ITK in splenocytes and in vivo. Degrading ITK provides a mechanism useful for treating inflammatory, autoimmune, and proliferative diseases and disorders in subjects in need thereof.

In one aspect, provided herein are compounds of Formula (I), or stereoisomers and pharmaceutically acceptable salts thereof:

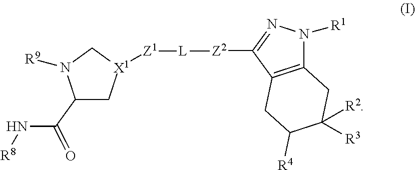

The left side of the molecule is a ubiquitin ligase harness. The right side of the molecule is an ITK hook. The middle portion of the molecule is a linker. The compounds of Formula (I) are described in detail herein.

In one more aspect, provided herein are compounds of Formula (II), or stereoisomers and pharmaceutically acceptable salts thereof:

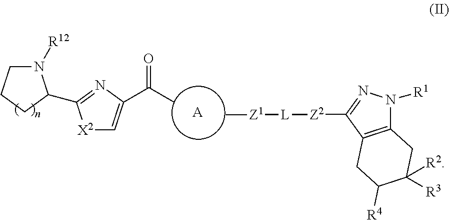

The left side of the molecule is a ubiquitin ligase harness. The right side of the molecule is an ITK hook. The middle portion of the molecule is a linker. The compounds are described in detail herein.

In another aspect, provided herein are compounds of Formula (III), or stereoisomers and pharmaceutically acceptable salts thereof:

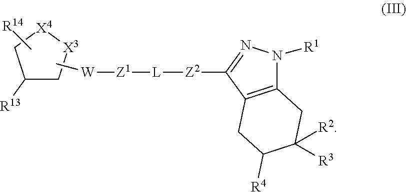

The left side of the molecule is a ubiquitin ligase harness. The right side of the molecule is an ITK hook. The middle portion of the molecule is a linker. The compounds of Formula (III) are described in detail herein.

In another aspect, provided herein are pharmaceutical compositions. The pharmaceutical compositions comprise the compounds of Formulae (A), (I), (II) and (III) along with one or more pharmaceutically acceptable carriers, diluents, and/or excipients.

In another aspect, provided herein are methods of treating a disease or disorder in a subject in need thereof comprising the step of administering a therapeutically effective amount of the compound of Formulae (A), (I), (II) and (III), or compositions thereof to the subject. In certain embodiments, provided herein are the compounds and compositions of Formulae (A), (I), (II) and (III) for use in therapy. In certain embodiments, provided herein are the compounds and compositions of Formulae (A), (I), (II) and (III) for use in the treatment or prevention of inflammatory, autoimmune, or proliferative diseases and disorders. In certain embodiments, provided herein are the uses of the compounds and compositions of Formulae (A), (I), (II) and (III) for the manufacture of medicaments. In certain embodiments, provided herein are the uses of the compounds and compositions of Formulae (A), (I), (II) and (III) for the manufacture of medicaments for the treatment or prevention of inflammatory, autoimmune, or proliferative diseases and disorders. In certain embodiments, the disease or disorder is an autoimmune disease or disorder. In certain embodiments, the disease or disorder is a proliferative disease or disorder, for instance a T-cell lymphoma. In certain embodiments, the disease or disorder is cancer.

Also provided herein are the compounds and compositions of Formulae (A), (I), (II) and (III) for use in the treatment of cancer. Also provided herein are uses of compounds of Formulae (A), (I), (II) and (III) described herein, and compositions thereof, for the treatment of autoimmune diseases and inflammatory diseases.

### DETAILED DESCRIPTION

### Definitions

For purposes herein, the chemical elements are identified in accordance with the Periodic Table of the Elements, CAS version, Handbook of Chemistry and Physics, 75th Ed.

Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated herein by reference.

As described herein, “protecting group” refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis,” 4th Ed, Wuts, P. G. M. and Greene, T. W., Wiley-Interscience, New York: 2006.

As described herein, compounds herein optionally may be substituted with one or more substituents, such as those illustrated generally herein, or as exemplified by particular classes, subclasses, and species of this description.

As used herein, the term “hydroxyl” or “hydroxy” refers to an —OH moiety.

As used herein, the term “aliphatic” encompasses the terms alkyl, alkenyl, and alkynyl, each of which are optionally substituted as set forth below.

As used herein, an “alkyl” group refers to a saturated aliphatic hydrocarbon group containing one to twelve (e.g., one to eight, one to six, or one to four) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl.

An alkyl group can be substituted (i.e., optionally substituted) with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, or heterocycloaliphaticamino), sulfonyl (e.g., aliphatic-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-4 or 2-6) carbon atoms and at least one double bond. Like an alkyl group, an alkenyl group can be straight or branched. Examples of an alkenyl group include, but are not limited to, allyl, 1- or 2-isopropenyl, 2-butenyl, and 2-hexenyl. An alkenyl group can be optionally substituted with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, heterocycloaliphaticamino, or aliphaticsulfonylamino), sulfonyl (e.g., alkyl-SO₂—, cycloaliphatic-SO₂—, or aryl-S₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy.

Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-4 or 2-6) carbon atoms and has at least one triple bond. An alkynyl group can be straight or branched. Examples of an alkynyl group include, but are not limited to, propargyl and butynyl.

An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl (e.g., aliphaticsulfanyl or cycloaliphaticsulfanyl), sulfinyl (e.g., aliphaticsulfinyl or cycloaliphaticsulfinyl), sulfonyl (e.g., aliphatic-SO₂—, aliphaticamino-SO₂—, or cycloaliphatic-SO₂—), amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino, or heteroarylaminocarbonyl), urea, thiourea, sulfamoyl, sulfamide, alkoxycarbonyl, alkylcarbonyloxy, cycloaliphatic, heterocycloaliphatic, aryl, heteroaryl, acyl (e.g., (cycloaliphatic)carbonyl or (heterocycloaliphatic)carbonyl), amino (e.g., aliphaticamino), sulfoxy, oxo, carboxy, carbamoyl, (cycloaliphatic)oxy, (heterocycloaliphatic)oxy, or (heteroaryl)alkoxy.

As used herein, an “amido” group encompasses both “aminocarbonyl” and “carbonylamino.” These terms when used alone or in connection with another group refer to an amido group such as —N(R^{X})—C(O)—R^{Y} or —C(O)—N(R^{X})₂ when used terminally, and —C(O)—N(R^{X})— or —N(R^{X})—C(O)— when used internally, wherein R^{X} and R^{Y} can be aliphatic, cycloaliphatic, aryl, araliphatic, heterocycloaliphatic, heteroaryl, or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen (H or —H), aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined elsewhere herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, or tetrahydroindenyl); and tricyclic (e.g., fluorenyl tetrahydrofluorenyl, tetrahydroanthracenyl, or anthracenyl) ring systems in which the monocyclic ring system is aromatic or at least one of the rings in a bicyclic or tricyclic ring system is aromatic.

The bicyclic and tricyclic groups include benzofused (e.g., 2-3 membered, or bi- or tricyclic carbocyclic) rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (i.e., on a non-aromatic carbon within a carbocyclic ring of a benzofused bicyclic or tricyclic aryl); nitro; carboxy; amido; acyl (e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-SO₂— or amino-SO₂—); sulfinyl (e.g., aliphatic-S(O)— or cycloaliphatic-S(O)—); sulfanyl (e.g., aliphatic-S—); cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl (e.g., mono-, di- (such as p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl or ((dialkyl)amino)aryl); (cyanoalkyl)aryl; (alkoxy)aryl; (sulfamoyl)aryl (e.g., (aminosulfonyl)aryl); (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined elsewhere herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl, including carboxyalkyl, hydroxyalkyl, or haloalkyl such as trifluoromethyl), cycloaliphatic (e.g., cycloalkyl or cycloalkenyl), (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino), cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 membered (e.g., 8-12 or 9-, 10-, or 11-membered) structures that form two rings, wherein the two rings have at least one atom in common (e.g., two atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which are optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A “cycloalkyl” or “cycloalkenyl” group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino), nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl (e.g., alkyl-SO₂— and aryl-SO₂—), sulfinyl (e.g., alkyl-S(O)—), sulfanyl (e.g., alkyl-S—), sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3- to 10-membered mono- or bicylic (e.g., 5- to 10-membered mono- or bicyclic) saturated ring structure (e.g., fused, bridged, or spiro), in which one or more of the ring atoms is a heteroatom (e.g., nitrogen (N), oxygen (O), sulfur (S), or combinations thereof). Non-limiting examples of a heterocycloalkyl group include piperidyl or piperidinyl, piperazyl or piperazinyl, tetrahydropyranyl, tetrahydrofuryl or tetrahydrofuranyl, 1,4-dioxolanyl, 1,4-dithianyl, 1,3-dioxolanyl, oxazolidyl or oxazolidinyl, isoxazolidyl or isoxazolidinyl, morpholinyl, thiomorpholinyl, octahydrobenzofuryl or octahydrobenzofuranyl, octahydrochromenyl, octahydrothiochromenyl, octahydroindolyl or octahydroindolinyl, octahydropyrindinyl or octahydro-1H-cyclopenta[x]pyridine where x is b or c, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, decahydro-2,7-naphthyridine, 2,8-diazaspiro[4.5]decane, 2,7-diazaspiro[3.5]nonane, octahydropyrrolo[3,4-c]pyrrole, octahydro-1H-pyrrolo[3,4-b]pyridine, and 2,6-dioxa-tricyclo[3.3.1.0^{3,7}]nonyl. A monocyclic heterocycloalkyl group can be fused with a phenyl moiety to form, for example, tetrahydroisoquinoline, that could be categorized as a heteroaryl as defined elsewhere herein.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloalkenyls are numbered according to standard chemical nomenclature.

A heterocycloalkyl or heterocycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino, nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), nitro, cyano, halo, hydroxy, mercapto, sulfonyl (e.g., alkylsulfonyl or arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

A “heteroaryl” group, as used herein, refers to a monocyclic, bicyclic, or tricyclic ring system having four to fifteen ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having two to three rings. For example, a benzofused group includes one or two 4- to 8-membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are pyridyl, 1H-indazolyl, furyl or furanyl, pyrrolyl, thienyl, thiazolyl, oxazolyl, imidazolyl, tetrazolyl, benzofuryl or benzofuranyl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl or purinyl, cinnolyl, quinolyl, quinazolyl, phthalazyl, quinazolyl, quinoxalyl, isoquinolyl, 4H-quinolizyl, benzo-1,2,5-thiadiazolyl, or 1,8-naphthyridyl. Other examples of heteroaryls include 1,2,3,4-tetrahydroisoquinoline and 4,5,6,7-tetrahydropyrazolo[1,5-a]pyrazine.

Without limitation, monocyclic heteroaryls include furyl, thiophene-yl, 2H-pyrrolyl, oxazolyl, thiazolyl, imidazolyl, pyrazolyl, isoxazolyl, isothiazolyl, 1,3,4-thiadiazolyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphaticsulfonyl or aminosulfonyl); sulfinyl (e.g., aliphaticsulfinyl); sulfanyl (e.g., aliphaticsulfanyl); nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkylsulfonyl)amino)heteroaryl and ((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfamoyl)heteroaryl (e.g., (aminosulfonyl)heteroaryl); (sulfonyl)heteroaryl (e.g., (alkylsulfonyl)heteroaryl); (hydroxyalkyl)heteroaryl; (alkoxyalkyl)heteroaryl; (hydroxy)heteroaryl; ((carboxy)alkyl)heteroaryl; (((dialkyl)amino)alkyl)heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl (e.g., (alkylcarbonyl)heteroaryl); (alkyl)heteroaryl; or (haloalkyl)heteroaryl (e.g., trihaloalkylheteroaryl).

As used herein, a “heteroaraliphatic” (such as a heteroaralkyl group) refers to an aliphatic group (e.g., a C₁₋₄ alkyl group) that is substituted with a heteroaryl group. “Aliphatic,” “alkyl,” and “heteroaryl” have been defined above.

As used herein, a “heteroaralkyl” group refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic (or heterocycloaliphatic) ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined herein. For example, aroyl includes benzoyl.

As used herein, an “alkoxy” group refers to an alkyl-O— group where “alkyl” has been defined previously herein.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, when used as a terminal group; or —OC(O)—, or —C(O)O— when used as an internal group.

As used herein, an “ester” refers to —COOR^{X} when used as a terminal group; or —COOR^{X}— when used as an internal group, wherein R^{X} has been defined above. As used herein, an “alkoxycarbonyl,” which is encompassed by the term ester, used alone or in connection with another group refers to a group such as alkyl-O—C(O)—.

As used herein, a “formate” refers to —OC(O)H.

As used herein, an “acetate” refers to —OC(O)R^{X}, wherein R^{X} has been defined above. In one embodiment, acetate is —OC(O)Me

As used herein, a “haloaliphatic” group refers to an aliphatic group substituted with one to three halogen atoms. For instance, haloalkyl includes —CF₃.

As used herein, a “mercapto” or “sulfhydryl” group refers to —SH.

As used herein, a “sulfo” group refers to —SO₃H, or —SO₃R^{X} when used terminally or —S(O)₃— when used internally. In one embodiment, —SO₃H is a sulfonic acid. In one embodiment, SO₃R^{X} is a sulfonate.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —O—S(O)₂—NR^{Y}R^{Z} wherein R^{Y}, and R^{Z} have been defined above. In one embodiment, —O—S(O)₂—NR^{Y}R^{Z} is a sulfamate.

As used herein, a “sulfonamide” group refers to the structure —S(O)₂—NR^{X}R^{Y} or —NR^{X}—S(O)₂—R^{Z} when used terminally; or —S(O)₂—NR^{X}—, or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like. In certain embodiments, —S—R^{X} is a sulfide.

As used herein a “sulfinyl” group refers to —S(O)—R^{X} when used terminally and —S(O)— when used internally, wherein R^{X} has been defined above. Examples of sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, and/or the like.

As used herein, a “sulfonyl” group refers to —S(O)₂—R^{X} when used terminally and —S(O)₂— when used internally, wherein R^{X} has been defined above. Examples of sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂—, and/or the like.

As used herein, a “sulfoxy” group refers to —O—S(O)—R^{X}, or —S(O)—O—R^{X}, when used terminally and —O—S(O)— or —S(O)—O— when used internally, where R^{X} has been defined above. In certain embodiments, —O—S(O)— or —S(O)—O— are sulphinates.

As used herein, a “halogen” or “halo” group refers to fluorine (F), chlorine (Cl), bromine (Br), or iodine (I).

As used herein, an “alkoxyalkyl” refers to an alkyl group modified with an alkoxy group, such as alkyl-O-alkyl-, wherein alkyl has been defined above.

As used herein, a “carbonyl” or “oxo” refers to —C(O)— or ═O.

As used herein, the term “phospho” refers to phosphinates, phosphonates, phosphine oxides, phosphoramidates, phosphinic amides, and phosphonamidates. Examples of phosphinates, phosphonates, phosphine oxides, phosphoramidates, phosphinic amides, and phosphonamidates include —P(O)(R^{P})₂, (R^{P})₂P(O)OR^{P}, and R^{P}—PO(OR^{P})₂, wherein R^{P} is aliphatic, alkoxy, aryloxy, heteroaryloxy, (cycloaliphatic)oxy, (heterocycloaliphatic)oxy, aryl, heteroaryl, cycloaliphatic, or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} each when used terminally and —NR^{X}—CO—NR^{Y}— or —NR^{X}—CS—NR^{Y}— each when used internally, wherein R^{X}, R^{Y}, and R^{Z} have been defined above.

As used herein, a “guanidine” group refers to the structure —N═C(N(R^{X}R^{Y})(N(R^{X}R^{Y}) or —NR^{X}—C(═NR^{X})NR^{X}R^{Y} wherein R^{X} and R^{Y} have been defined above.

As used herein, the term “amidino” group refers to the structure —C(NR^{X})NR^{X}R^{Y} wherein R^{X} and R have been defined above.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on the same carbon atom.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is one to twelve. A branched aliphatic chain is a straight aliphatic chain that is substituted with one or more aliphatic groups. A branched aliphatic chain has the structure —[CQQ]ᵥ-, where each Q is independently a hydrogen (H or —H) or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used herein interchangeably with the phrase “substituted or unsubstituted.” As described herein, compounds herein can optionally be substituted with one or more substituents, as illustrated generally above, or as exemplified by particular classes, subclasses, and species of the description. As described herein, the variables R, R¹, R², L, and Z, and other variables contained in Formula (I), (Ia), (Ib), (II), (IIa), (IIb), (III), (III-I), (III-Ia), (III-Ib), (III-II), (III-IIa), or (III-IIb), described herein encompass specific groups, such as alkyl and aryl. Unless otherwise noted, each of the specific groups for the variables R, R¹⁰, R¹, R², L, L¹, A, W, and Z, and other variables contained therein can be optionally substituted with one or more substituents described herein. Each substituent of a specific group is further optionally substituted with one to three halo, cyano, oxo, alkoxy, hydroxy, amino, nitro, aryl, cycloaliphatic, heterocycloaliphatic, heteroaryl, haloalkyl, and/or alkyl. For instance, an alkyl group can be substituted with alkylsulfanyl and the alkylsulfanyl can be optionally substituted with one to three halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

As used herein, the term “substituted,” whether preceded by the term “optionally” or not, refers generally to the replacement of one or more hydrogen atoms in a given chemical structure with the radical of a specified substituent. Specific substituents are defined above and described below within the compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, for example, both rings share one common atom. Non-limiting examples of spiro heterocycloalkyls include

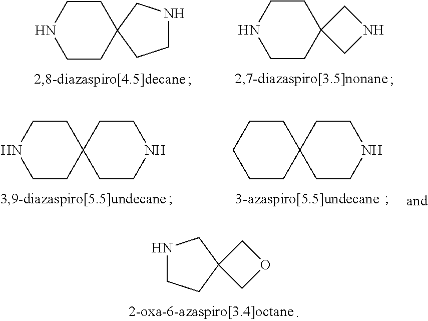 [SMILES: C1CC2(CCN1)CCNC2 ; C1CC2(CCN1)CNC2 ; C1CC2(CCN1)CCNCC2 ; C1CCC2(CC1)CCNCC2 ; C1CC2(CN1)COC2]

Spiro compounds depicted with overlapping rings indicate that the spirocyclic rings can bond at any vertex. For instance, in the spiro group

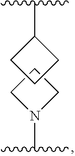

the two rings can bond at any of the three available vertex atoms in either ring.

As one of ordinary skill in the art will recognize, combinations of substituents envisioned by this description are those combinations that result in the formation of stable or chemically feasible compounds.

As used herein, the phrase “stable or chemically feasible” refers to compounds that are not substantially altered when subjected to conditions to allow for their production, detection, recovery, purification, and/or use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to an animal, alternatively a mammal, including a human.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions may be sterile.

The term “excipients” as used herein includes pharmaceutically acceptable excipients, carriers, vehicles, or stabilizers that are nontoxic to the cell or mammal being exposed thereto at the dosages and concentrations employed. In certain embodiments, the physiologically acceptable excipient is an aqueous pH buffered solution.

The terms “treating” or “treatment” of a disease refer to executing a protocol, which may include administering one or more therapeutic agent to an individual (human or otherwise), in an effort to obtain beneficial or desired results in the individual, including clinical results. In certain embodiments, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total).

In certain embodiments, “treatment” also can mean prolonging survival as compared to expected survival of an individual not receiving treatment. In certain embodiments, “treating” and “treatment” may occur by administration of one dose of a therapeutic agent or therapeutic agents, or may occur upon administration of a series of doses of a therapeutic agent or therapeutic agents.

In certain embodiments, “treating” or “treatment” does not require complete alleviation of signs or symptoms, and does not require a cure. In certain embodiments, “treatment” also can refer to clinical intervention, such as administering one or more therapeutic agents to an individual, designed to alter the natural course of the individual or cell being treated (i.e., to alter the course of the individual or cell that would occur in the absence of the clinical intervention). In certain embodiments, the term “therapeutic agent” can refer to a drug that induces the proteolytic degradation of IL-2 inducible T-cell kinase or compositions thereof.

The term an “individual,” a “patient,” or a “subject” refers to a mammal. In certain embodiments, a “mammal” for purposes of treatment includes humans; non-human primates; domestic and farm animals; and zoo, sports, or pet animals, such as dogs, horses, rabbits, cattle, pigs, hamsters, gerbils, mice, ferrets, rats, cats, etc. In some embodiments, the individual or subject is human.

As used herein, the term “about” means within +10% of a value. For example, a dose that is about 100 mg/kg provides that the dose can be 90 mg/kg to 110 mg/kg. By way of further example, an amount of an additional therapeutic agent ranging from about 50% to about 100% provides that the amount of additional therapeutic agent ranges from 45-55% to 90-110%. A person of skill in the art will appreciate the scope and application of the term “about” when used to describe other values disclosed herein.

Unless otherwise stated, structures depicted herein also are meant to include all isomeric (e.g., enantiomeric, diastereomeric, and geometric (or conformational)) forms of the structure; for example, the (R)- and (S)- configurations for each asymmetric center, (Z)- and (E)-double bond isomers, and syn-/cis- and anti-/trans-conformational isomers. Therefore, single stereochemical isomers as well as enantiomeric, diastereomeric, and geometric (or conformational) mixtures of the present compounds are within the scope of the description. Alternatively, as used herein, “enantiomeric excess (ee)” refers to a dimensionless mol ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]−[(S)-compound])/([(R)-compound]+[(S)-compound])×100, where the (R)-compound>(S)-compound; or % ee=([(S)-compound]−[(R)-compound])/([(S)-compound]+[(R)-compound])×100, where the (S)-compound>(R)-compound. Moreover, as used herein, “diastereomeric excess (de)” refers to a dimensionless mol ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

In certain embodiments, the compounds or inhibitors described herein have an ee, de, % ee, or % de greater than zero. For example, in certain embodiments, the compounds or inhibitors described herein have an ee, de, % ee, or % de of ten. In certain embodiments, the compounds or inhibitors described herein have an ee, de, % ee, or % de of twenty-five. In certain embodiments, the compounds or inhibitors described herein have an ee, de, % ee, or % de of fifty. In certain embodiments, the compounds or inhibitors described herein have an ee, de, % ee, or % de of seventy-five.

In certain embodiments, the compounds or inhibitors described herein have an ee, de, % ee, or % de range from ninety to one hundred. In certain embodiments, the compounds or inhibitors described herein have an ee, de, % ee, or % de range from ninety-five to one hundred. In certain embodiments, the compounds or inhibitors described herein have an ee, de, % ee, or % de range from ninety-seven to one hundred. In certain embodiments, the compounds or inhibitors described herein have an ee, de, % ee, or % de range from ninety-eight to one hundred. In certain embodiments, the compounds or inhibitors described herein have an ee, de, % ee, or % de range from ninety-nine to one hundred.

In one embodiment of a compound or inhibitor described herein, the ee, de, % ee, or % de is one. In one embodiment of a compound or inhibitor described herein, the ee, de, % ee, or % de is two. In one embodiment of a compound or inhibitor described herein, the ee, de, % ee, or % de is three. In one embodiment of a compound or inhibitor described herein, the ee, de, % ee, or % de is four. In one embodiment of a compound or inhibitor described herein, the ee, de, % ee, or % de is five. In one embodiment of a compound or inhibitor described herein, the ee, de, % ee, or % de is six. In one embodiment of a compound or inhibitor described herein, the ee, de, % ee, or % de is seven. In one embodiment of a compound or inhibitor described herein, the ee, de, % ee, or % de is eight. In one embodiment of a compound or inhibitor described herein, the ee, de, % ee, or % de is nine. In one embodiment of a compound or inhibitor described herein, the ee, de, % ee, or % de is ten. In one embodiment of a compound or inhibitor described herein, the ee, de, % ee, or % de is eleven. In one embodiment of a compound or inhibitor described herein, the ee, de, % ee, or % de is twelve. In one embodiment of a compound or inhibitor described herein, the ee, de, % ee, or % de is thirteen. In one embodiment of a compound or inhibitor described herein, the ee, de, % ee, or % de is fourteen. In one embodiment of a compound or inhibitor described herein, the ee, de, % ee, or % de is fifteen. In one embodiment of a compound or inhibitor described herein, the ee, de, % ee, or % de is sixteen. In one embodiment of a compound or inhibitor described herein, the ee, de, % ee, or % de is seventeen. In one embodiment of a compound or inhibitor described herein, the ee, de, % ee, or % de is eighteen. In one embodiment of a compound or inhibitor described herein, the ee, de, % ee, or % de is nineteen. In one embodiment of a compound or inhibitor described herein, the ee, de, % ee, or % de is twenty. In one embodiment of a compound or inhibitor described herein, the ee, de, % ee, or % de is twenty-one. In one embodiment of a compound or inhibitor described herein, the ee, de, % ee, or % de is twenty-two.

In one embodiment of a compound or inhibitor described herein, the ee, de, % ee, or % de is twenty-three. In one embodiment of a compound or inhibitor described herein, the ee, de, % ee, or % de is twenty-four. In one embodiment of a compound or inhibitor described herein, the ee, de, % ee, or % de is twenty-five. In one embodiment of a compound or inhibitor described herein, the ee, de, % ee, or % de is twenty-six. In one embodiment of a compound or inhibitor described herein, the ee, de, % ee, or % de is twenty-seven. In one embodiment of a compound or inhibitor described herein, the ee, de, % ee, or % de is twenty-eight. In one embodiment of a compound or inhibitor described herein, the ee, de, % ee, or % de is twenty-nine. In one embodiment of a compound or inhibitor described herein, the ee, de, % ee, or % de is thirty. In one embodiment of a compound or inhibitor described herein, the ee, de, % ee, or % de is thirty-one. In one embodiment of a compound or inhibitor described herein, the ee, de, % ee, or % de is thirty-two. In one embodiment of a compound or inhibitor described herein, the ee, de, % ee, or % de is thirty-three. In one embodiment of a compound or inhibitor described herein, the ee, de, % ee, or % de is thirty-four. In one embodiment of a compound or inhibitor described herein, the ee, de, % ee, or % de is thirty-five. In one embodiment of a compound or inhibitor described herein, the ee, de, % ee, or % de is thirty-six. In one embodiment of a compound or inhibitor described herein, the ee, de, % ee, or % de is thirty-seven. In one embodiment of a compound or inhibitor described herein, the ee, de, % ee, or % de is thirty-eight. In one embodiment of a compound or inhibitor described herein, the ee, de, % ee, or % de is thirty-nine. In one embodiment of a compound or inhibitor described herein, the ee, de, % ee, or % de is forty. In one embodiment of a compound or inhibitor described herein, the ee, de, % ee, or % de is forty-one. In one embodiment of a compound or inhibitor described herein, the ee, de, % ee, or % de is forty-two. In one embodiment of a compound or inhibitor described herein, the ee, de, % ee, or % de is forty-three. In one embodiment of a compound or inhibitor described herein, the ee, de, % ee, or % de is forty-four. In one embodiment of a compound or inhibitor described herein, the ee, de, % ee, or % de is forty-five. In one embodiment of a compound or inhibitor described herein, the ee, de, % ee, or % de is forty-six. In one embodiment of a compound or inhibitor described herein, the ee, de, % ee, or % de is forty-seven. In one embodiment of a compound or inhibitor described herein, the ee, de, % ee, or % de is forty-eight. In one embodiment of a compound or inhibitor described herein, the ee, de, % ee, or % de is forty-nine. In one embodiment of a compound or inhibitor described herein, the ee, de, % ee, or % de is fifty. In one embodiment of a compound or inhibitor described herein, the ee, de, % ee, or % de is fifty-one. In one embodiment of a compound or inhibitor described herein, the ee, de, % ee, or % de is fifty-two. In one embodiment of a compound or inhibitor described herein, the ee, de, % ee, or % de is fifty-three. In one embodiment of a compound or inhibitor described herein, the ee, de, % ee, or % de is fifty-four. In one embodiment of a compound or inhibitor described herein, the ee, de, % ee, or % de is fifty-five. In one embodiment of a compound or inhibitor described herein, the ee, de, % ee, or % de is fifty-six. In one embodiment of a compound or inhibitor described herein, the ee, de, % ee, or % de is fifty-seven. In one embodiment of a compound or inhibitor described herein, the ee, de, % ee, or % de is fifty-eight. In one embodiment of a compound or inhibitor described herein, the ee, de, % ee, or % de is fifty-nine. In one embodiment of a compound or inhibitor described herein, the ee, de, % ee, or % de is sixty. In one embodiment of a compound or inhibitor described herein, the ee, de, % ee, or % de is sixty-one. In one embodiment of a compound or inhibitor described herein, the ee, de, % ee, or % de is sixty-two. In one embodiment of a compound or inhibitor described herein, the ee, de, % ee, or % de is sixty-three. In one embodiment of a compound or inhibitor described herein, the ee, de, % ee, or % de is sixty-four. In one embodiment of a compound or inhibitor described herein, the ee, de, % ee, or % de is sixty-five. In one embodiment of a compound or inhibitor described herein, the ee, de, % ee, or % de is sixty-six. In one embodiment of a compound or inhibitor described herein, the ee, de, % ee, or % de is sixty-seven. In one embodiment of a compound or inhibitor described herein, the ee, de, % ee, or % de is sixty-eight. In one embodiment of a compound or inhibitor described herein, the ee, de, % ee, or % de is sixty-nine. In one embodiment of a compound or inhibitor described herein, the ee, de, % ee, or % de is seventy. In one embodiment of a compound or inhibitor described herein, the ee, de, % ee, or % de is seventy-one. In one embodiment of a compound or inhibitor described herein, the ee, de, % ee, or % de is seventy-two. In one embodiment of a compound or inhibitor described herein, the ee, de, % ee, or % de is seventy-three. In one embodiment of a compound or inhibitor described herein, the ee, de, % ee, or % de is seventy-four. In one embodiment of a compound or inhibitor described herein, the ee, de, % ee, or % de is seventy-five. In one embodiment of a compound or inhibitor described herein, the ee. de. % ee or % de is seventy-six. In one embodiment of a compound or inhibitor described herein, the ee, de, % ee, or % de is seventy-seven. In one embodiment of a compound or inhibitor described herein, the ee, de, % ee, or % de is seventy-eight. In one embodiment of a compound or inhibitor described herein, the ee, de, % ee, or % de is seventy-nine. In one embodiment of a compound or inhibitor described herein, the ee, de, % ee, or % de is eighty. In one embodiment of a compound or inhibitor described herein, the ee, de, % ee, or % de is eighty-one. In one embodiment of a compound or inhibitor described herein, the ee, de, % ee, or % de is eighty-two. In one embodiment of a compound or inhibitor described herein, the ee, de, % ee, or % de is eighty-three. In one embodiment of a compound or inhibitor described herein, the ee, de, % ee, or % de is eighty-four. In one embodiment of a compound or inhibitor described herein, the ee, de, % ee, or % de is eighty-five. In one embodiment of a compound or inhibitor described herein, the ee, de, % ee, or % de is eighty-six. In one embodiment of a compound or inhibitor described herein, the ee, de, % ee, or % de is eighty-seven. In one embodiment of a compound or inhibitor described herein, the ee, de, % ee, or % de is eighty-eight. In one embodiment of a compound or inhibitor described herein, the ee, de, % ee, or % de is eighty-nine. In one embodiment of a compound or inhibitor described herein, the ee, de, % ee, or % de is ninety. In one embodiment of a compound or inhibitor described herein, the ee, de, % ee, or % de is ninety-one. In one embodiment of a compound or inhibitor described herein, the ee, de, % ee, or % de is ninety-two. In one embodiment of a compound or inhibitor described herein, the ee, de, % ee, or % de is ninety-three. In one embodiment of a compound or inhibitor described herein, the ee, de, % ee, or % de is ninety-four. In one embodiment of a compound or inhibitor described herein, the ee, de, % ee, or % de is ninety-five. In one embodiment of a compound or inhibitor described herein, the ee, de, % ee, or % de is ninety-six. In one embodiment of a compound or inhibitor described herein, the ee, de, % ee, or % de is ninety-seven. In one embodiment of a compound or inhibitor described herein, the ee, de, % ee, or % de is ninety-eight. In one embodiment of a compound or inhibitor described herein, the ee, de, % ee, or % de is ninety-nine. In one embodiment of a compound or inhibitor described herein, the ee, de, % ee, or % de is one hundred. In certain embodiments, compounds or inhibitors described within Table 1 herein have an ee, de, % ee, or % de as described within this paragraph. In certain embodiments, compounds or inhibitors described in the Examples and/or Biological Examples have an ee, de, % ee, or % de as described within this paragraph. Unless otherwise stated, all tautomeric forms of the compounds of this description are within the scope of this description. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this description. Such compounds are useful, for example, as analytical tools or probes in biological assays, or as therapeutic agents.

As used herein, the term “&1” means that a compound including the “&1” notation at a particular chemical element or atom (e.g., carbon) within the compound was prepared as a mixture of two stereoisomers at the noted chemical element or atom (e.g., a diastereomeric mixture having a de or % de as described above).

Chemical structures and nomenclature are derived from ChemDraw, version 19.0, Cambridge, MA.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

### Compounds

In one embodiment, provided is a compound of Formula (I)

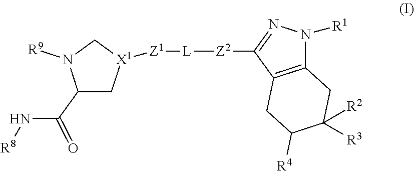

wherein X¹ is C—H or nitrogen; Z¹ is a bond, —CH₂, —C(O)—, —C(O)—N(R)—, or —N(R)—, wherein R is H or CH₃; L is a linker according to -L¹-L²-L³-L⁴-L⁵-L⁶-L⁷-, wherein -L¹- is absent, —N(R¹⁰)—, —C(R¹¹)—, —C(O)—, —C₁₋₈ alkylene-, —C₂₋₈ alkynylene-, —C₆-C₁₀ aryl-, —C₆-C₁₀ heteroaryl-, Q¹, or Q² each -L²-, -L³-, -L⁴-, and -L⁵- is independently, absent, —N(R¹⁰)—, —C(R¹¹)—, —C(O)—, —O—, —(CH₂—CH₂—O)₁₋₈—, —C₁₋₈ alkylene-, —C₂₋₈ alkynylene-, —C₆-C₁₀ aryl-, —C₆-C₁₀ heteroaryl-, Q¹, Q², or Q³; each -L⁶- and -L⁷-, is independently, absent, —N(R¹⁰)—, —C(R¹⁰)—, —C(O)—, —C(O)—N(R¹⁰)—, —N(R¹¹)—C(O)—, or —C(R¹¹)—C(O)—N(R¹⁰)—; each Q¹ is a three- to seven-membered heterocycloalkylene comprising at least one nitrogen; each Q² is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring; each Q³ is a three- to six-membered cycloalkylene; Z² is

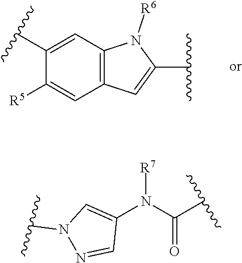

R¹ is hydrogen or methyl; R² is methyl; R³ is methyl or methylene bound to R⁴ to form a substituted cyclopropyl; R⁴ is hydrogen, or methylene bound to R³ to form the substituted cyclopropyl; wherein when R³ and R⁴ form substituted cyclopropyl, then the cyclopropyl is substituted with difluoro; R⁵ is hydrogen or halogen; each R⁶ is hydrogen or methyl; each R⁷ is hydrogen or methyl; R⁸ is alkyl, alkenyl, alkylene, alkynyl, aryl, cycloalkyl, heterocycloalkyl, heteroaryl, —S(O)(R^{q}), or —S(O)₂(R^{q}); wherein Ra is —H, —OH, alkyl, alkenyl, alkynyl, aryl, heterocycle, or heteroaryl, wherein each alkyl, alkenyl, alkylene, alkynyl, aryl, cycloalkyl, heterocycloalkyl, and heteroaryl is optionally substituted; R⁹ is hydrogen, optionally substituted C₁₋₈ alkyl, -AA¹-AA²-R¹⁵, wherein each AA¹ and AA² is an amino acid residue, and R¹⁵ is hydrogen or methyl; each R¹⁰ is hydrogen or methyl; and each R¹¹ is hydrogen, methyl, aryl, or heteroaryl; or a stereoisomer, and/or pharmaceutical salt thereof.

In one embodiment, provided is a compound of Formula (II)

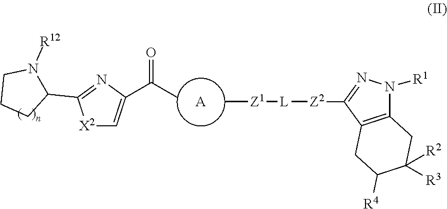

wherein X² is —CH₂, —N—R, oxygen, or sulfur, wherein R is H or CH₃; A is phenyl or C₆-heteroaryl; Z¹ is a bond, —CH₂—, —C(O)—, —C(O)—N(R)—, —N(R)—, or —O—, wherein R is H or CH₃; L is a linker according to -L¹-L²-L³-L⁴-L⁵-L⁶-L⁷-, wherein -L¹- is absent, —N(R¹⁰)—, C(R¹¹)—, —C(O)—, —C₁₋₈ alkylene-, —C₂₋₈ alkynylene-, —C₆-C₁₀ aryl-, —C₆-C₁₀ heteroaryl-, Q¹, or Q²; each -L²-, -L³-, -L⁴-, and -L⁵- is independently, absent, —N(R¹⁰)—, —C(R¹¹)—, —C(O)—, —O—, —(CH₂—CH₂—O)₁₋₈—, —C₁₋₈ alkylene-, —C₂₋₈ alkynylene-, —C₆-C₁₀ aryl-, —C₆-C₁₀ heteroaryl-, Q¹, Q², or Q³; each -L⁶- and -L⁷- is independently, absent, —N(R¹⁰)—, —C(R¹⁰)—, —C(O)—, —C(O)—N(R¹⁰)—, —N(R¹⁰)—C(O)—, or —C(R¹¹)—C(O)—N(R¹⁰)—; each Q¹ is a three- to seven-membered heterocycloalkylene comprising at least one nitrogen; each Q² is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring; each Q³ is a three- to six-membered cycloalkylene; Z² is

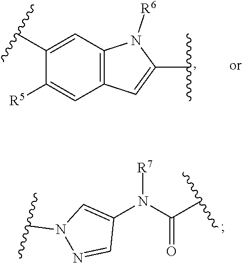

R¹ is hydrogen or methyl; R² is methyl; R³ is methyl or methylene bound to R⁴ to form a substituted cyclopropyl; R⁴ is hydrogen or methylene bound to R³ to form the substituted cyclopropyl; wherein when R³ and R⁴ form the substituted cyclopropyl, then the cyclopropyl is substituted with difluoro; R⁵ is hydrogen or halogen; each R⁶ is hydrogen or methyl; each R⁷ is hydrogen or methyl; R¹² is optionally substituted C₁₋₈ alkyl, -AA¹-AA²-R⁵, wherein each AA¹ and AA² is an amino acid residue, and R¹⁵ is H or methyl; and n is 0 or 1; or a stereoisomer, and/or pharmaceutical salt thereof.

In one embodiment, provided is a compound of Formula (III)

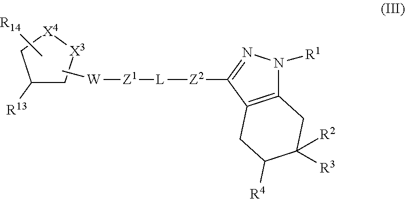

wherein X³ is nitrogen and X⁴ is C—H; Z¹ is a bond, —CH₂—, —C(O)—, —C(O)—N(R)—, or —N(R), wherein R is H or CH₃; L is a linker according to -L¹-L²-L³-L⁴-L⁵-L⁶-L⁷-, wherein -L¹- is absent, —N(R¹⁰)—, —C(R¹¹)—, —C(O)—, —C₁₋₈ alkylene-, —C₂₋₈ alkynylene-, —C₆-C₁₀ aryl-, —C₆-C₁₀ heteroaryl-, Q¹, or Q²; each -L²-, -L³-, -L⁴-, and -L⁵- is independently, absent, —N(R¹⁰)—, —C(R¹¹)—, —C(O)—, —O—, —(CH₂—CH₂—O)₁₋₈—, —C₁₋₈ alkylene-, —C₂₋₈ alkynylene-, —C₆-C₁₀ aryl-, —C₆-C₁₀ heteroaryl-, Q¹, Q², or Q³; each -L⁶- and -L⁷- is independently, absent, —N(R¹⁰)—, —C(R¹⁰)—, —C(O)—, —C(O)—N(R¹⁰)—, —N(R¹⁰)—C(O)—, or —C(R¹¹)—C(O)—N—(R¹⁰); each Q¹ is a three- to seven-membered heterocycloalkylene comprising at least one nitrogen; each Q² is a five- to thirteen-membered bicyclic heterocycloalkylene comprising at least one nitrogen, wherein the five- to thirteen-membered spiro bicyclic heterocycloalkylene is optionally a spiro bicyclic heterocycloalkylene ring; each Q³ is a three- to six-membered cycloalkylene; W is

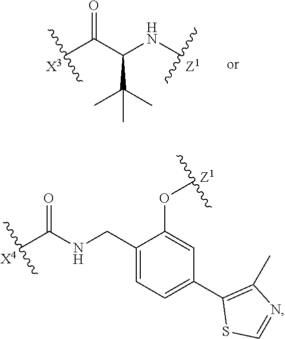

wherein,

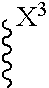

designates attachment to X³, wherein

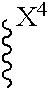

designates attachment to X⁴, wherein,

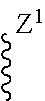

designates attachment to Z¹; Z² is

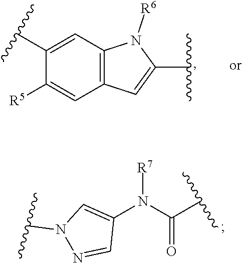

R¹ is hydrogen or methyl; R² is methyl; R³ is methyl or methylene bound to R⁴ to form a substituted cyclopropyl; R⁴ is hydrogen or methylene bound to R³ to form the substituted cyclopropyl; wherein when R³ and R⁴ form the substituted cyclopropyl, then the cyclopropyl is substituted with difluoro; R⁵ is hydrogen or halogen; each R⁶ is hydrogen or methyl; each R⁷ is hydrogen or methyl; R¹³ is —H, —OH, halogen, —NH, —C₁-C₃ alkyl, —C₂-C₆ alkenyl, —C₂-C₆ alkynyl, —C₁-C₃ alkoxy, —C₁-C₃ thioalkyl, —C₁-C₃ alkylamine, —C₆-C₁₀ aryl, cycloalkyl, heterocycloalkyl, or heteroaryl; R⁴ is —C(O)—N(H)—C₆-C₁₀ aralkyl, —C(O)—CH(tbutyl)-N(H)—C₃-C₆ cycloalkyl, or -AA¹-AA²-R¹⁵, wherein each AA¹ and AA² is an amino acid residue, and R¹⁵ is H or methyl; or a stereoisomer, and/or pharmaceutical salt thereof.

In one aspect, provided herein are compounds of Formula (I), and stereoisomers and pharmaceutically acceptable salts thereof

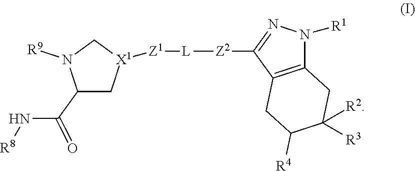

In Formula (I), X¹ is C—H or nitrogen; Z¹ is a bond, —CH₂, —C(O)—, —C(O)—N(R)—, or —N(R)—, wherein R is hydrogen or CH₃; R¹ is hydrogen or methyl; R² is methyl; R³ is methyl, or methylene bound to R⁴ to form a substituted cyclopropyl; R⁴ is hydrogen or methylene bound to R³ to form a substituted cyclopropyl; wherein when R³ and R⁴ form the substituted cyclopropyl, then the cyclopropyl is substituted with difluoro (e.g., in certain embodiments, geminal difluoro); R⁸ is alkyl, alkenyl, alkylene, alkynyl, aryl, cycloalkyl, heterocycloalkyl, heteroaryl, —S(O)(R^{q}), or —S(O)₂(R^{q}); wherein Ra is hydrogen, —OH, alkyl, alkenyl, alkynyl, aryl, heterocycle or heterocyclyl, or heteroaryl, wherein each alkyl, alkenyl, alkylene, alkynyl, aryl, cycloalkyl, heterocycloalkyl, and heteroaryl is optionally substituted; R⁹ is hydrogen, optionally substituted C₁₋₈ alkyl, or -AA¹-AA²-R⁵, wherein each AA¹ and AA² is, independently, an amino acid residue or a 2-amino-2-cyclohexylacetic acid residue, and R¹⁵ is hydrogen or methyl.

In certain embodiments, X¹ is CH. In certain embodiments, Z¹ is NH. In certain embodiments, Z¹ is a bond. In certain embodiments, R² is methyl. In certain embodiments, R³ is methyl. In certain embodiments, R⁴ is hydrogen. In certain embodiments, R² is methyl; R³ is methyl; and R⁴ is hydrogen. In certain embodiments, R² is hydrogen; and R³ and R⁴ form difluorocyclopropane. In certain embodiments, R³ and R⁴ form geminal difluorocyclopropane.

In certain embodiments, R⁸ is tetrahydronaphthyl. In certain embodiments, R⁸ has the following structure

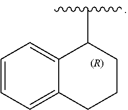

In certain embodiments, R⁹ is -AA¹-AA²-R¹⁵. In certain embodiments, AA¹ is a phenylalanine or 2-amino-2-cyclohexylacetic acid residue and AA² is an alanine residue. In certain embodiments, AA¹ is (S)-2-amino-2-cyclohexylacetic acid. In certain embodiments, AA¹ is phenylalanine. In certain embodiments, AA² is alanine. In certain embodiments, R⁹ has the following structure

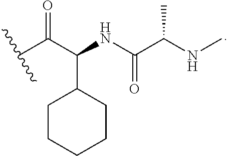 [SMILES: *C(=O)[C@@H](NC(=O)[C@H](C)NC)C1CCCCC1]

In certain embodiments, R⁹ has the following structure

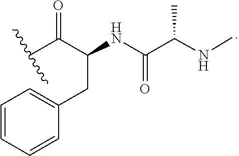 [SMILES: *C(=O)[C@H](Cc1ccccc1)NC(=O)[C@H](C)NC]

In one aspect, provided herein are compounds of Formula (II), and stereoisomers and pharmaceutically acceptable salts thereof

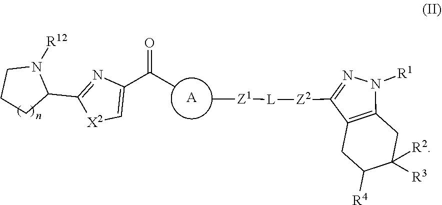

In Formula (II), X² is —CH₂—, —N—R, oxygen, or sulfur, wherein R is hydrogen or CH₃; A is phenyl or C₅₋₆ heteroaryl; Z¹ is a bond, —CH₂, —C(O)—, —C(O)—N(R)—, —N(R)—, or —O—, wherein R is hydrogen or CH₃; R¹ is hydrogen or methyl; R² is methyl; R³ is methyl or methylene bound to R⁴ to form a substituted cyclopropyl; R⁴ is hydrogen or methylene bound to R³ to form a substituted cyclopropyl; wherein when R³ and R⁴ form the substituted cyclopropyl, then the cyclopropyl is substituted with difluoro (e.g., in certain embodiments, geminal difluoro); R¹² is optionally substituted C₁₋₈ alkyl, -AA¹-AA²-R⁵, wherein each AA¹ and AA², independently, is an amino acid residue or a 2-amino-2-cyclohexylacetic acid residue, and R¹⁵ is hydrogen or methyl; and n is zero or one.

In certain embodiments, X² is sulfur. In certain embodiments, Z¹ is oxygen. In certain embodiments, R² is methyl; R³ is methyl; and R⁴ is hydrogen. In certain embodiments, R² is hydrogen; and R³ and R⁴ form difluorocyclopropane or geminal difluorocyclopropane. In certain embodiments, A is phenyl.

In certain embodiments, R¹² is -AA¹-AA²-R⁵. In certain embodiments, AA¹ is (S)-2-amino-2-cyclohexylacetic acid. In certain embodiments, AA¹ is phenylalanine. In certain embodiments, AA² is alanine. In certain embodiments, AA¹ is a phenylalanine or 2-amino-2-cyclohexylacetic acid residue and AA² is an alanine residue. In certain embodiments, R¹² has the following structure:

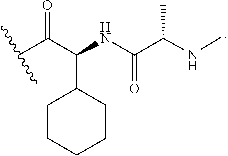 [SMILES: *C(=O)[C@@H](NC(=O)[C@H](C)NC)C1CCCCC1]

In certain embodiments, R⁹ has the following structure:

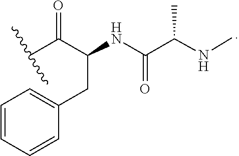 [SMILES: *C(=O)[C@H](Cc1ccccc1)NC(=O)[C@H](C)NC]

In one aspect, provided herein are compounds of Formula (III), and stereoisomers and pharmaceutically acceptable salts thereof

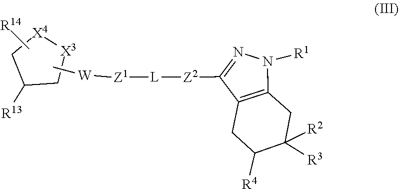

In Formula (III), X³ is nitrogen and X⁴ is C—H; Z¹ is a bond, —CH₂, —C(O)—, —C(O)—N(R)—, or —N—(R)—, wherein R is hydrogen or CH₃; W is

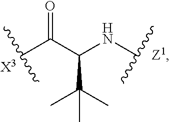

### —C(O)—, or

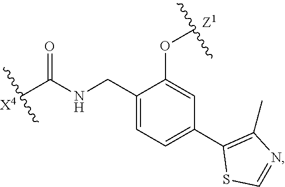

wherein,

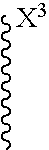

designates attachment to X³, wherein

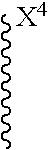

designates attachment to X⁴, and wherein,

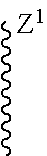

designates attachment to Z¹; R¹ is hydrogen or methyl; R² is methyl; R³ is methyl or methylene bound to R⁴ to form a substituted cyclopropyl; R⁴ is hydrogen or methylene bound to R³ to form a substituted cyclopropyl; wherein when R³ and R⁴ form the substituted cyclopropyl, then the cyclopropyl is substituted with difluoro (e.g., in certain embodiments, geminal difluoro); R¹² is halo; R¹³ is hydrogen, —OH, halogen, —NH₂, —C₁-C₃ alkyl, —C₂-C₆ alkenyl, —C₂-C₆ alkynyl, —C₁-C₃ alkoxy, —C₁-C₃ thioalkyl, —C₁-C₃ alkylamine, —C₆-C₁₀ aryl, cycloalkyl, heterocycloalkyl, or heteroaryl; R¹⁴ is —C(O)—N(H)—C₆-C₁₀ aralkyl,

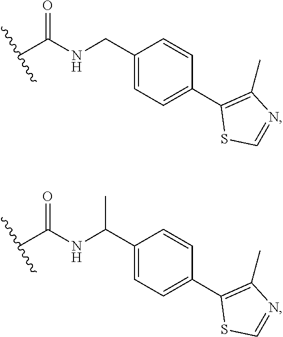 [SMILES: *C(=O)NCc1ccc(-c2scnc2C)cc1 ; *C(=O)NC(C)c1ccc(-c2scnc2C)cc1]

—C(O)—CH(t-butyl)-N(H)—C₃-C₆ cycloalkyl, —C(O)—CH(t-butyl)-N(H)C(O)—C₃-C₆ cycloalkyl,

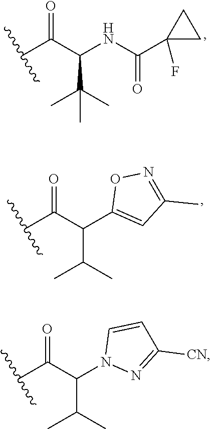 [SMILES: *C(=O)[C@@H](NC(=O)C1(F)CC1)C(C)(C)C ; *C(=O)C(c1cc(C)no1)C(C)C ; *C(=O)C(C(C)C)n1ccc(C#N)n1]

or -AA¹-AA²-R⁵, wherein each AA¹ and AA² is, independently, an amino acid residue or a 2-amino-2-cyclohexylacetic acid residue; R¹⁵ is hydrogen or methyl; X is oxygen or sulfur; and n is an integer from one to eight.

In certain embodiments, X³ is attached to W, and X⁴ is attached to R¹⁴. In certain embodiments, X⁴ is attached to W, and X³ is attached to R¹⁴. In certain embodiments, Z¹ is a bond.

In certain embodiments, W is

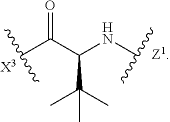

In certain embodiments, W is —C(O)—. In certain embodiments, W is

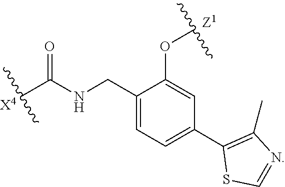

In certain embodiments, R² is methyl. In certain embodiments, R³ is methyl. In certain embodiments, R⁴ is hydrogen. In certain embodiments, R² is methyl; R³ is methyl; and R⁴ is hydrogen. In certain embodiments, R² is hydrogen. In certain embodiments, R³ and R⁴ form difluorocyclopropane. In certain embodiments, R² is hydrogen; and R³ and R⁴ form difluorocyclopropane. In certain embodiments, R³ and R⁴ form geminal difluorocyclopropane. In certain embodiments. R¹² is bromo. In certain embodiments. R¹² is chloro. In certain embodiments. R¹² is fluoro. In certain embodiments. R¹² is iodo. In certain embodiments, R¹³ is —OH.

In certain embodiments, R⁴ is —C(O)—CH(t-butyl)-N(H)—C₃-C₆ cycloalkyl. In certain embodiments, R⁴ is —C(O)—N(H)—C₆-C₁₀ aralkyl. In certain embodiments, R⁴ is —C(O)—CH(t-butyl)-N(H)C(O)—C₃-C₆ cycloalkyl. In certain embodiments, R⁴ is

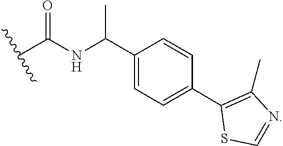 [SMILES: *C(=O)NC(C)c1ccc(-c2scnc2C)cc1]

In certain embodiments, R¹⁴ is

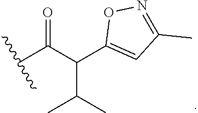 [SMILES: *C(=O)C(c1cc(C)no1)C(C)C]

In certain embodiments, R¹⁴ is

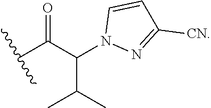 [SMILES: *C(=O)C(C(C)C)n1ccc(C#N)n1]

In certain embodiments, R¹⁴ is -AA¹-AA²-R⁵. In certain embodiments, each AA¹ and AA² is, independently, an amino acid residue or a 2-amino-2-cyclohexylacetic acid residue. In certain embodiments, AA¹ is (S)-2-amino-2-cyclohexylacetic acid. In certain embodiments, AA¹ is phenylalanine. In certain embodiments, AA² is alanine. In certain embodiments, R¹⁴ has the following structure

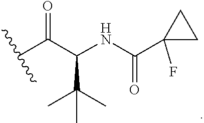 [SMILES: *C(=O)[C@@H](NC(=O)C1(F)CC1)C(C)(C)C]

In certain embodiments, R¹⁴ has the following structure

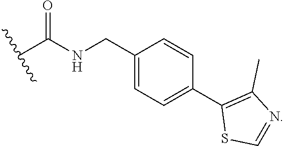 [SMILES: *C(=O)NCc1ccc(-c2scnc2C)cc1]

In certain embodiments, R¹⁴ has the following structure

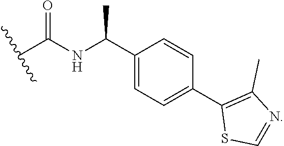 [SMILES: *C(=O)N[C@@H](C)c1ccc(-c2scnc2C)cc1]

In certain embodiments. R¹⁴ has the following structure

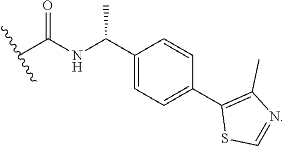 [SMILES: *C(=O)N[C@H](C)c1ccc(-c2scnc2C)cc1]

In certain embodiments, W is

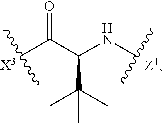

### and R¹⁴ is

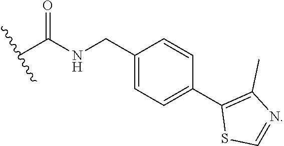 [SMILES: *C(=O)NCc1ccc(-c2scnc2C)cc1]

In certain embodiments, W is

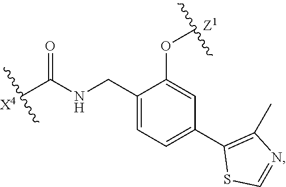

### and R¹⁴ is

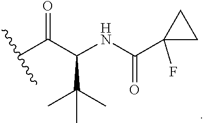 [SMILES: *C(=O)[C@@H](NC(=O)C1(F)CC1)C(C)(C)C]

In certain embodiments, provided herein is the compound of Formula (I), having the following Formula (Ia), and stereoisomers and pharmaceutically acceptable salts thereof

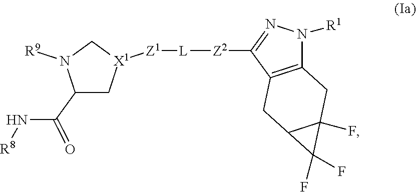

wherein the variables are described below.

In certain embodiments, provided herein is the compound of Formula (Ia), having the following Formula (Ib), and stereoisomers and pharmaceutically acceptable salts thereof

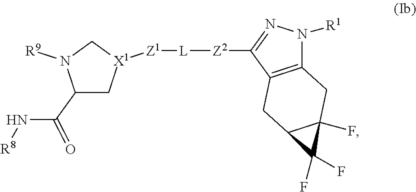

wherein the variables are described below.

In certain embodiments, provided herein is the compound of Formula (II), having the following Formula (Ia), and stereoisomers and pharmaceutically acceptable salts thereof

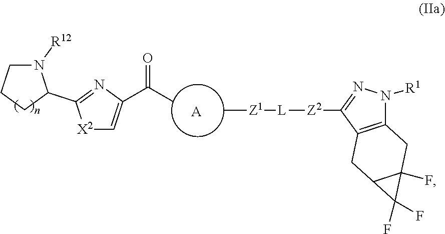

wherein the variables are described below.

In certain embodiments, provided herein is the compound of Formula (IIa), having the following Formula (IIb), and stereoisomers and pharmaceutically acceptable salts thereof

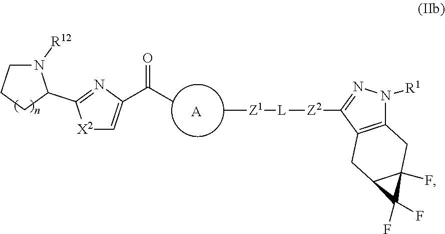

wherein the variables are described below.

In certain embodiments, provided herein is the compound of Formula (III), having the following Formula (III-I), and stereoisomers and pharmaceutically acceptable salts thereof

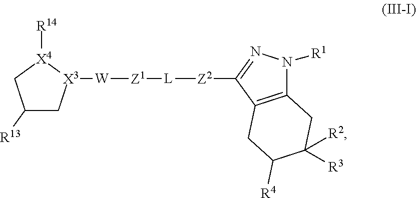

wherein the variables are described below.

In certain embodiments, provided herein is the compound of Formula (III-I), having the following Formula (III-Ia), and stereoisomers and pharmaceutically acceptable salts thereof

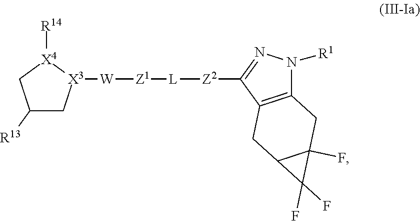

wherein the variables are described below.

In certain embodiments, provided herein is the compound of Formula (III-Ia), having the following Formula (III-Ib), and stereoisomers and pharmaceutically acceptable salts thereof

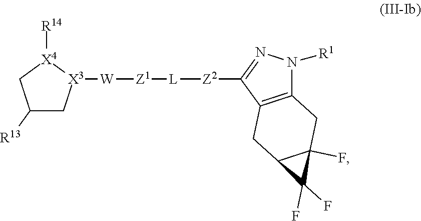

wherein the variables are described below.

In certain embodiments, provided herein is the compound of Formula (III), having the following Formula (III-II), and stereoisomers and pharmaceutically acceptable salts thereof

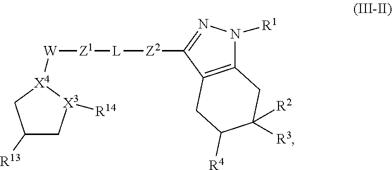

wherein the variables are described below.

In certain embodiments, provided herein is the compound of Formula (III-II), having the following Formula (III-IIa), and stereoisomers and pharmaceutically acceptable salts thereof

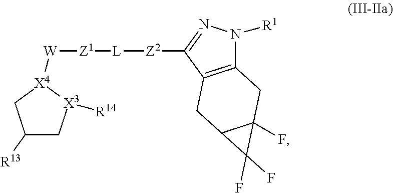

wherein the variables are described below.

In certain embodiments, provided herein is the compound of Formula (III-IIa), having the following Formula (III-IIb), and stereoisomers and pharmaceutically acceptable salts thereof:

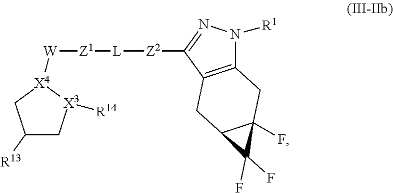

wherein the variables are described below.

In Formula (I), (Ia), (Ib), (II), (IIa), (IIb), (III), (III-I), (III-Ia), (III-Ib), (III-II), (III-IIa), or (III-IIb), Z² is

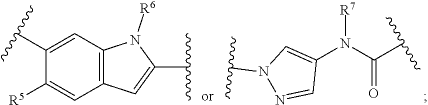

wherein R⁵ is hydrogen or halogen; R⁶ is hydrogen or methyl; and R⁷ is hydrogen or methyl. In certain embodiments, R⁵ is hydrogen. In certain embodiments, R⁵ is halogen. In certain embodiments, R⁵ is bromo. In certain embodiments, R⁵ is chloro. In certain embodiments, R⁵ is fluoro. In certain embodiments, R⁵ is iodo. In certain embodiments, R⁶ is hydrogen. In certain embodiments, R⁶ is methyl. In certain embodiments, R⁷ is hydrogen. In certain embodiments, R⁷ is methyl.

In certain embodiments, Z² is

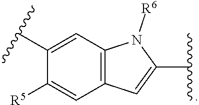

In certain embodiments, Z² is

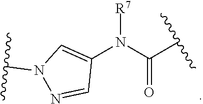

In Formula (I), (Ia), (Ib), (II), (IIa), (IIb), (III), (III-I), (III-Ia), (III-Ib), (III-II), (III-IIa), or (III-IIb), L is a linker. The linker can be any linker suitable for linking the right and left portions of the molecule or Formulae herein. In particular embodiments, the linker does not interfere with the harness or hook functions of the molecule or Formulae herein. In advantageous embodiments, the linker provides useful solubility, flexibility, and/or distance between the portions of the molecule or Formulae herein. In certain embodiments, L is a linker according to -L¹-L²-L³-L⁴-L⁵-L⁶-L⁷- or -L⁷-L⁶-L⁵-L⁴-L³-L²-L¹-. Each group L^{x} is described in detail below.

In certain embodiments, the linker L comprises at least one heterocyclic group. In certain embodiments, the linker L comprises one heterocyclic group. In certain embodiments, the linker L comprises two heterocyclic groups. In certain embodiments, the linker L comprises three heterocyclic groups. In certain embodiments, the linker L comprises at least one spiro bicyclic heterocycloalkylene groups. In certain embodiments, the linker L comprises one spiro bicyclic heterocycloalkylene group. In certain embodiments, the linker L comprises two spiro bicyclic heterocycloalkylene groups. In certain embodiments, the linker L comprises three spiro bicyclic heterocycloalkylene groups. In certain embodiments, the linker L comprises at least one heterocycloalkylene group and at least one spiro bicyclic heterocycloalkylene. The remaining groups of or within the linker are selected for chemical compatibility with adjacent groups, as will be recognized by those of skill in the art.

In certain embodiments, L is a linker according to -L-L²-L³-L⁴-L⁵-L⁶-L⁷-. In certain embodiments, L is a linker according to -L⁷-L⁶-L⁵-L⁴-L³-L²-L¹-. In certain embodiments, -L¹- is absent, —N(R¹⁰)—, —C(R¹¹)₂—, —C(O)—, —C₁₋₈ alkylene-, —C₂₋₈ alkynylene-, —C₆-C₁₀ aryl-, -heteroaryl-, —C₆-C₁₀ heteroaryl-, -Q¹-, or -Q²-; each -L²-, -L³-, -L⁴-, and -L⁵- is independently, absent, —N(R¹⁰)—, —C(R¹¹)₂—, —C(O)—, —O—, —(CH₂—CH₂—O)₁₋₈—, C₁₋₈ alkylene-, —C₂₋₈ alkynylene-, —C₆-C₁₀ aryl-, substituted —C₆-C₁₀ aryl-, -heteroaryl-, —C₄-C₁₀ heteroaryl-, -Q¹-, -Q²-, or -Q³-; each -L⁶- and -L⁷- is independently, absent, —N(R¹⁰)—, —C(R¹⁰)₂)—, —C(O)—, —C(O)—N(R¹⁰)—, —N(R¹⁰)—C(O)—, heteroaryl-, —C₄-C₁₀ heteroaryl-,

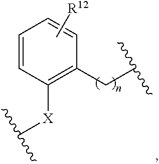

or —C(R¹¹)₂—C(O)—N(R¹⁰)—. In certain embodiments, L comprises at least one -Q¹-. In certain embodiments, L comprises one -Q¹-. In certain embodiments, L comprises two -Q¹-. In certain embodiments, L comprises three -Q¹-. In certain embodiments, L comprises at least one -Q²-. In certain embodiments, L comprises one -Q²-. In certain embodiments, L comprises two -Q²-. In certain embodiments, L comprises three -Q²-. In certain embodiments, L comprises at least one -Q¹- and at least one -Q²-. In certain embodiments, L comprises one -Q¹- and one -Q²-.

In certain embodiments, each -Q¹- is a three- to seven-membered heterocycloalkylene comprising at least one nitrogen; each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring; each -Q³- is a three- to six-membered cycloalkylene; each R¹⁰ is hydrogen or methyl; and each R¹¹ is hydrogen, methyl, aryl, substituted aryl, or heteroaryl. In certain embodiments, R¹⁰ is hydrogen. R¹⁰ is methyl. In certain embodiments, R¹¹ is hydrogen. In certain embodiments, R¹¹ is methyl. In certain embodiments, R¹¹ is aryl. In certain embodiments, R¹¹ is substituted aryl. In certain embodiments, R¹¹ is heteroaryl.

In certain embodiments of Formula (I), (Ia), (Ib), (II), (IIa), (IIb), (III), (III-I), (III-Ia), (III-Ib), (III-II), (III-IIa), or (III-IIb), L comprises at least one -Q¹- according to

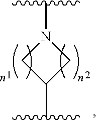

wherein n¹ is one or two, and n² is one or two.

In certain embodiments of Formula (I), (Ia), (Ib), (II), (IIa), (IIb), (III), (III-I), (III-Ia), (III-Ib), (III-II), (III-IIa), or (III-IIb), L comprises at least one -Q¹-.

In certain embodiments of Formula (I), (Ia), (Ib), (II), (IIa), (IIb), (III), (III-I), (III-Ia), (III-Ib), (III-II) (III-IIa), or ((III-IIb), L is selected from
-Q¹-N(Me)-CH₂-Q¹-C(O)—;
—N(Me)-Q¹-CH₂-Q¹-C(O)—;
-Q²-CH₂-Q¹-C(O)—;
-Q¹-CH₂-Q¹-C(O)—;
-Q¹-Q¹-C(O)—;
-Q¹-CH₂—N(Me)-Q¹-C(O)—;
-Q¹-CH₂-Q¹-CH₂—C(O)—N(Me)-;
-Q¹-CH₂-Q¹-;
-Q¹-CH₂-Q²-;
-Q¹-CH₂—CH₂-Q¹-;
-Q¹-CH₂—CH₂-Q²-;
-Q¹-C(O)-Q¹-;
-Q¹-C(O)-Q²-;
-Q¹-CH₂-Q¹-N(Me)-C(O)—;
—CH₂—CH₂—CH₂—CH₂-Q¹-C(O)—;
-Q¹-C(O)-;
-Q¹-C(O)-Q¹-CH(C₆H₅)—;
—C≡—CCH₂-Q¹-C(O)—;
-Q¹-CH₂-Q¹-NH—C(O)—;
—CH₂—CH₂—CH₂-Q¹-C(O)—;
-Q¹-CH₂-Q¹-C(Me)-C(O)—N(Me)-;
—CH₂-Q¹-;
-Q-C(O)-Q¹-CH₂—;
—N(H)—(CH₂)₅—C(O)-Q¹-CH(C₆H₅)—;
—N(H)—(CH₂)₂—O—(CH₂)₂—C(O)-Q¹-CH(C₆H₅)—;
-Q¹-(CH₂)₃—C(O)-Q¹-CH(C₆H₅)—;
-Q²-C(O)-Q¹-CH(C₆H₅)—;
-Q²-CH₂—C(O)-Q¹-CH(C₆H₅)—;
-Q²-(CH₂)₃—C(O)-Q¹-CH(C₆H₅)—;
-Q²-(CH₂)₂—C(O)-Q¹-CH(C₆H₅)—;
—(CH₂)₆-Q¹-CH(C₆H₅)—;
-Q¹-Q¹-C(O)-Q¹-CH(C₆H₅)—;
-Q¹-CH₂—C(O)-Q¹-CH(C₆H₅)—;
-Q¹-(CH₂)₂—C(O)-Q¹-CH(C₆H₅)—;
—(CH₂)₃—C(O)-Q¹-CH(C₆H₅)—;
—(CH₂)₄—C(O)-Q¹-CH(C₆H₅)—;
—(CH₂)₅—C(O)-Q¹-CH(C₆H₅)—;
—(CH₂)₆—C(O)-Q¹-CH(C₆H₅)—;
—(CH₂)₃-Q¹-CH₂—C(O)-Q¹-CH(C₆H₅)—;
—(CH₂)₃—O-Q³-C(O)-Q¹-CH(C₆H₅)—;
—(CH₂)₃—O—(CH₂)₂—C(O)-Q¹-CH(C₆H₅)—;
—(CH₂)₃—O—(CH₂)₂—C(O)-Q¹-CH(pyrid-2-yl)-;
—(CH₂)₄-Q¹-CH(C₆H₅)—;
—(CH₂)₅-Q¹-CH(C₆H₅)—;
—(CH₂)₆-Q¹-CH(pyrid-2-yl)-;
—(CH₂)₇-Q¹-CH(C₆H₅)—;
—(CH₂)₇-Q¹-CH(Me)-C(O)—N(Me)-;
—N(H)—(CH₂)₂—O—(CH₂)₂-Q¹-CH(Me)-C(O)—N(Me)-;
—(CH₂)₃—O—(CH₂)₂—C(O)-Q¹-CH(Me)-C(O)—N(Me)-;
—N(H)—(CH₂)₂—O—(CH₂)₂-Q¹-CH(C₆H₅)—;
—N(H)—(CH₂)₂—[O—(CH₂)₂]₂—C(O)-Q¹-CH(C₆H₅)—;
—N(H)—(CH₂)₂—[O—(CH₂)₂]₃—C(O)-Q¹-CH(C₆H₅)—;
—N(H)—(CH₂)₂—[O—(CH₂)₂]₄—C(O)-Q¹-CH(C₆H₅)—;
—N(H)—(CH₂)₂—[O—(CH₂)₂]₅—C(O)-Q¹-CH(C₆H₅)—;
—N(H)—(CH₂)₂—[O—(CH₂)₂]₆—C(O)-Q¹-CH(C₆H₅)—;
—N(H)—(CH₂)₂—[O—(CH₂)₂]₇—C(O)-Q¹-CH(C₆H₅)—;
—N(H)—(CH₂)₂—[O—(CH₂)₂]s-C(O)-Q¹-CH(C₆H₅)—;
—N(H)-Q³-O—(CH₂)₂—CH₂—;
—N(H)—(CH₂)₃-Q¹-(CH₂)₂—;
—C(O)—N(H)—[(CH₂)₃—O]₃—(CH₂)₂—NH—;
—C(O)—N(H)—[(CH₂)₃—O]₃—(CH₂)₂—;
Q¹-C(O)—[(CH₂)₂—O]₃—(CH₂)₂—NH—;
-Q¹-(CH₂)₃—O—CH₂—;
-Q¹-C(O)—(C₆H₆)—CH₂—;
-Q¹-(2-pyridyl)-O—CH₂—,

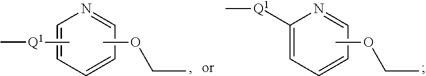

—N(H)-Q³-X-(2-pyridyl)-O—,

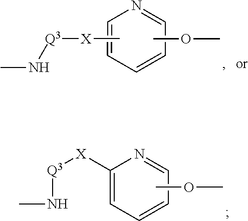

—N(H)-Q³-X-(4-pyridyl)-, or

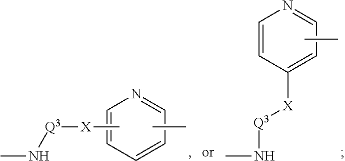

—N(H)—(CH₂)₂-Q³-X-(2-pyridyl)-O—CH₂—,

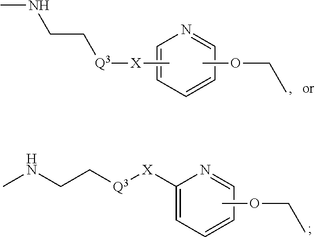

—CH═C—(CH₂)₂-Q¹-;
-Q¹-;
—C(O)—(CH₂—CH₂—O)—(CH₂)₂—C(O)-Q¹-CH(C₆H₅)—;
—N(H)—C(O)—(CH₂—CH₂—O)₄—(CH₂)₂—C(O)-Q¹-CH(C₆H₅)—;
—N(H)—C(O)—(CH₂—CH₂—O)₅—(CH₂)₂—C(O)-Q¹-CH(C₆H₅)—;
—(CH₂—CH₂—O)₅—(CH₂)₂—C(O)-Q¹-CH(C₆H₅)—;
—(CH₂—CH₂—O)—(CH₂)₂—C(O)-Q¹-CH(C₆H₅)—;
—(CH₂—CH₂—O)₄—(CH₂)₂—C(O)-Q¹-CH(C₆H₅)—;
—(CH₂)₄-Q¹-CH(C₆H₅)—;
—(CH₂)₃-Q¹-CH(C₆H₅)—;
—(CH₂)₆-Q¹-CH(C₆H₅)—;
—(CH₂)₅-Q¹-CH(C₆H₅)—;
—(CH₂—CH₂—O)—(CH₂)₂-Q¹-CH(C₆H₅)—;
—C(O)—(CH₂—CH₂—O)₄—(CH₂)₂-Q¹-CH(C₆H₅)—;
—C(O)—(CH₂—CH₂—O)₅—(CH₂)₂—C(O)-Q¹-CH(C₆H₅)—;
—C(O)—(CH₂—CH₂—O)₆—(CH₂)₂—C(O)-Q¹-CH(C₆H₅)—;
—C(O)—(CH₂—CH₂—O)₃—(CH₂)₂—C(O)-Q¹-CH(C₆H₅)—;
—(CH₂)₅—C(O)-Q¹-CH(C₆H₅)—;
—(CH₂—CH₂—O)₃—(CH₂)₂—C(O)-Q¹-CH(C₆H₅)—;
—(CH₂)₇—C(O)-Q¹-CH(C₆H₅)—;
—C(O)-pyrimidine-Q¹-C(O)-Q¹-CH(C₆H₅)— or

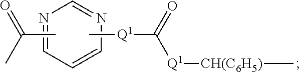

—C(O)—(CH₂)-Q¹-3-pyridyl-C(O)-Q¹-CH(C₆H₅)—,

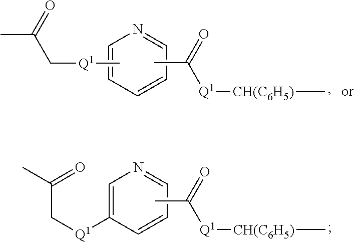

C(O)-2-pyridyl-Q¹-CH₂—C(O)-Q¹-CH(C₆H₅)—,

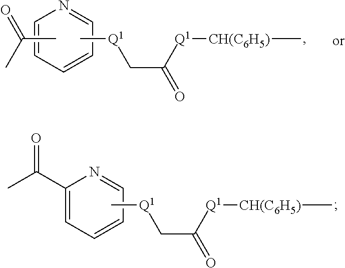

—C(O)—(CH₂)-Q¹-CH₂—C(O)-Q¹-CH(C₆H₅)—;
—(CH₂)₂—C(O)-Q¹-CH(C₆H₅)—;
—C(O)—(CH₂)₂-Q¹-(CH₂)₂—C(O)-Q¹-CH(C₆H₅)—;
—C(O)—(CH₂)₉—C(O)-Q¹-CH(C₆H₅)—;
—C(O)—(CH₂)₇—C(O)-Q¹-CH(C₆H₅)—;
—C(O)—(CH₂)₅—C(O)-Q¹-CH(C₆H₅)—;
—C(O)—(CH₂—CH₂—O)₂—(CH₂)₂—C(O)-Q¹-CH(C₆H₅)—;
—C(O)-2-pyridyl-Q¹-C(O)-Q¹-CH(C₆H₅)—,

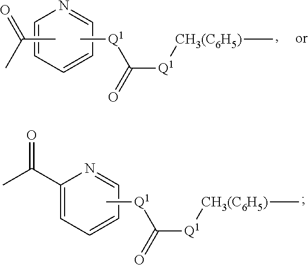

—CH₂—C(O)-Q¹-CH(C₆H₅)—;
—(CH₂)₃—C(O)-Q¹-CH(C₆H₅)—;
—(CH₂)₄—C(O)-Q¹-CH(C₆H₅)—; and
Q¹-4-pyridyl-Q¹-CH(C₆H₅)—,

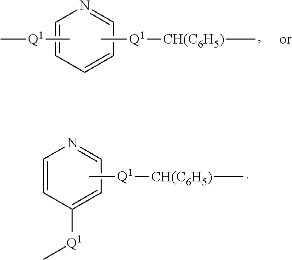

In some embodiments, X is oxygen or sulfur.

In certain embodiments of Formula (I), Formula (II), Formula (III), Formula (III-I), or Formula (III-II), L comprises at least one -Q¹- selected from the group consisting of

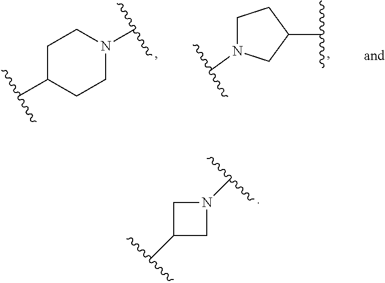 [SMILES: *C1CCN(*)CC1 ; *C1CCN(*)C1 ; *C1CN(*)C1]

In certain embodiments of Formula (I), Formula (II), Formula (III), Formula (III-I), or Formula (III-II), L comprises at least one -Q²- according to

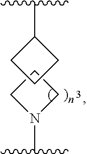

wherein n³ is one or two.

In certain embodiments of Formula (I), Formula (II), Formula (III), Formula (III-I), or Formula (III-II), L comprises at least one -Q²- according to

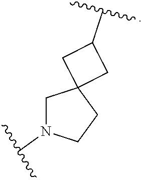 [SMILES: *C1CC2(CCN(*)C2)C1]

In certain embodiments of Formula (I), Formula (II), Formula (III), Formula (III-I), or Formula (III-II), L comprises at least one -Q²- according to

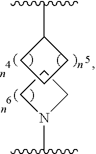

wherein n⁴ is one or two, n⁵ is one or two, and n⁶ is one or two.

In certain embodiments of Formula (I), Formula (II), Formula (III), Formula (III-I), or Formula (III-II), L comprises at least one -Q²- according to

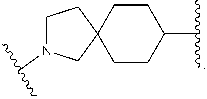 [SMILES: *C1CCC2(CC1)CCN(*)C2]

In certain embodiments of Formula (I), Formula (II), Formula (III), Formula (III-I), or Formula (III-II), L comprises at least one -Q²- according to

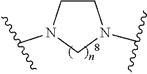

wherein n⁸ is one or two.

In certain embodiments of Formula (I), Formula (II), Formula (ITT), Formula (III-I), or Formula (III-II), L comprises at least one -Q²- according to

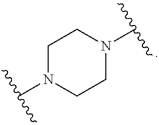 [SMILES: *N1CCN(*)CC1]

In certain embodiments of Formula (I), Formula (II), Formula (III), Formula (III-I), or Formula (III-II), L comprises at least one -Q²- according to

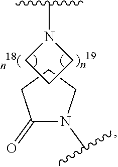

wherein n¹⁸ and n¹⁹ is two, or n¹⁸ is two and n¹⁹ is three, or n¹⁸ is three and n¹⁹ is two.

In certain embodiments of Formula (I), Formula (II), Formula (III), Formula (III-I), or Formula (III-II), L comprises at least one -Q²- according to

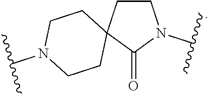 [SMILES: *N1CCC2(CC1)CCN(*)C2=O]

In certain embodiments of Formula (I), Formula (II), Formula (III), Formula (III-I), or Formula (III-II), L comprises at least one -Q²- according to

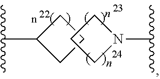

wherein n²² is zero to two; n²³ is zero to two, and n²⁴ is one or two, or wherein n²² is two and each n²³ and n²⁴ is one; or n²² is two and each n²³ and n²⁴ is two.

In certain embodiments of Formula (I), Formula (II), Formula (III), Formula (III-I), or Formula (III-II), L comprises at least one -Q²- according to

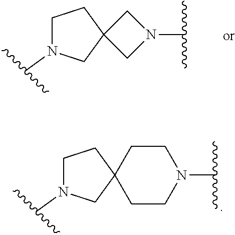 [SMILES: *N1CCC2(C1)CN(*)C2 ; *N1CCC2(CC1)CCN(*)C2]

In certain embodiments of Formula (I), Formula (II), Formula (III), Formula (III-I), or Formula (III-II), L comprises at least one -Q²- according to

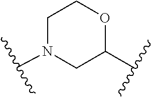 [SMILES: *C1CN(*)CCO1]

In certain embodiments of Formula (I), Formula (II), Formula (III), Formula (III-I), or Formula (III-II), L comprises at least one -Q³- according to

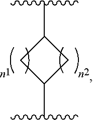

wherein n¹ is one or two, and n² is one or two.

In certain embodiments of Formula (I), Formula (II), Formula (III), Formula (III-I), or Formula (III-II), L comprises at least one -Q³- selected from the group consisting of

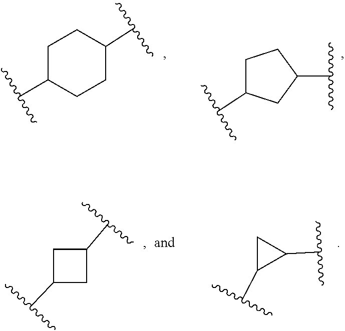 [SMILES: *C1CCC(*)CC1 ; *C1CCC(*)C1 ; *C1CC(*)C1 ; *C1CC1*]

In certain embodiments of Formula (I), (Ia), (Ib), (II), (IIa), (IIb), (III), (III-I), (III-Ia), (III-Ib), (III-II) (III-IIa), or ((III-IIb), the linker L is selected from:

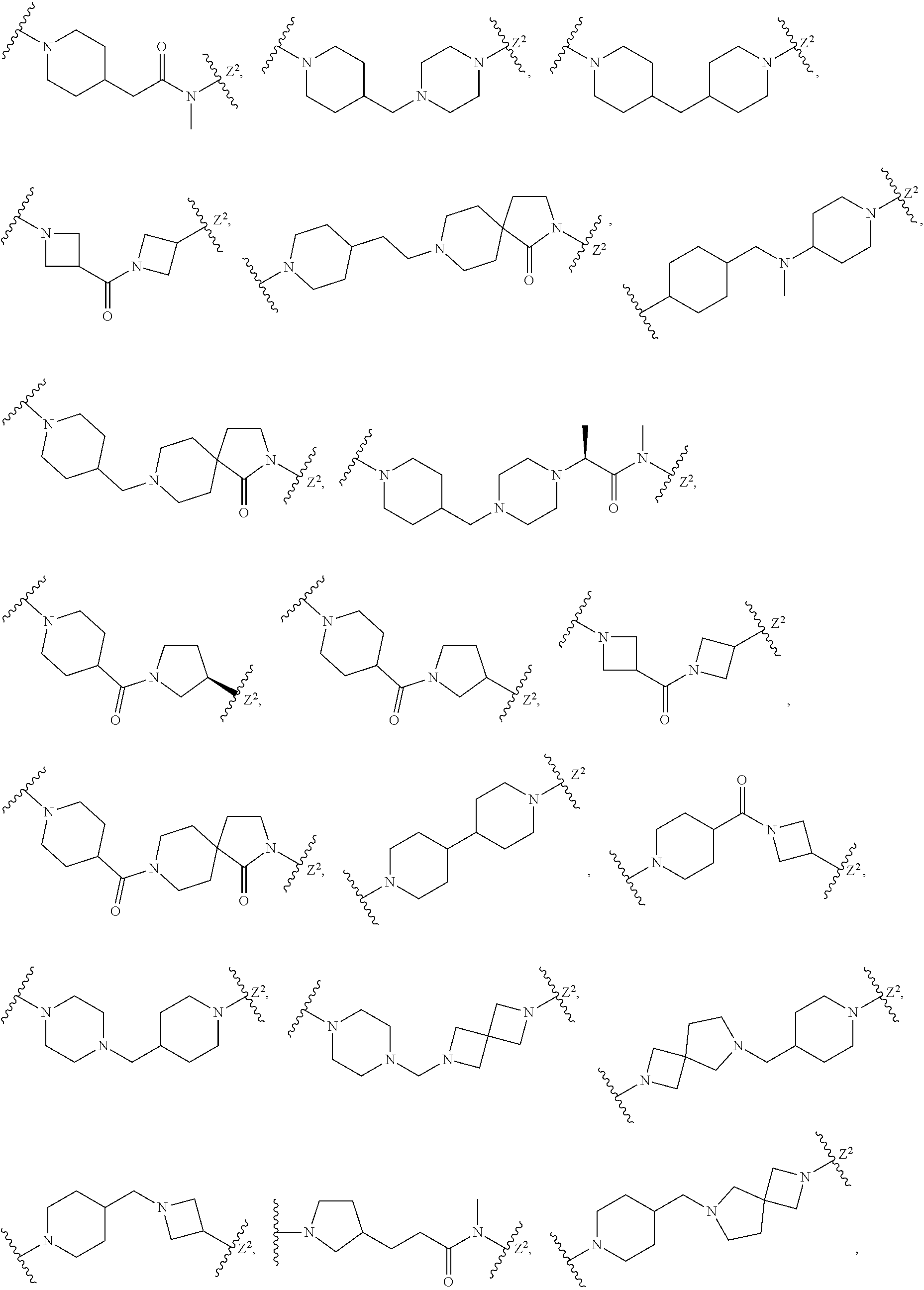

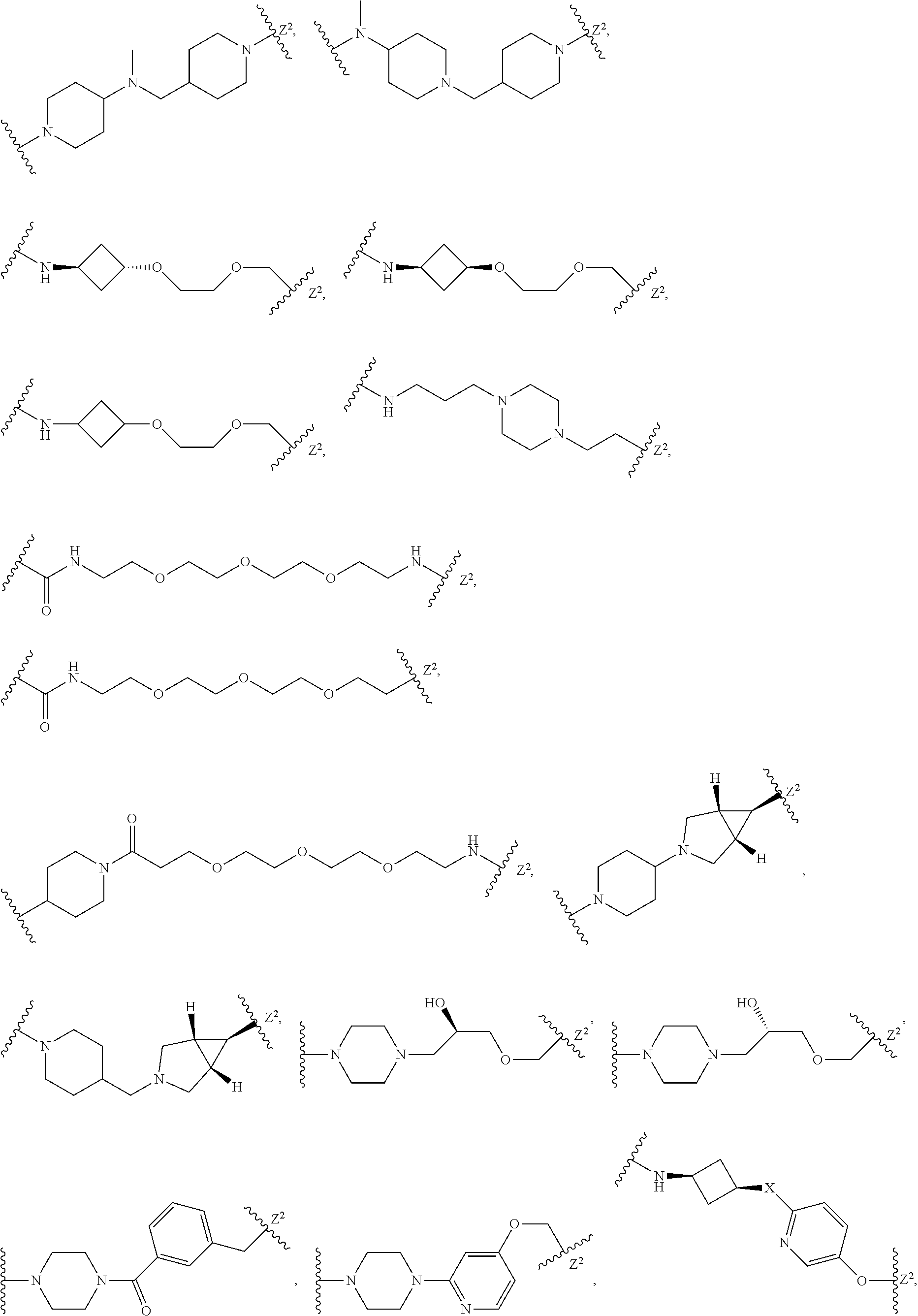

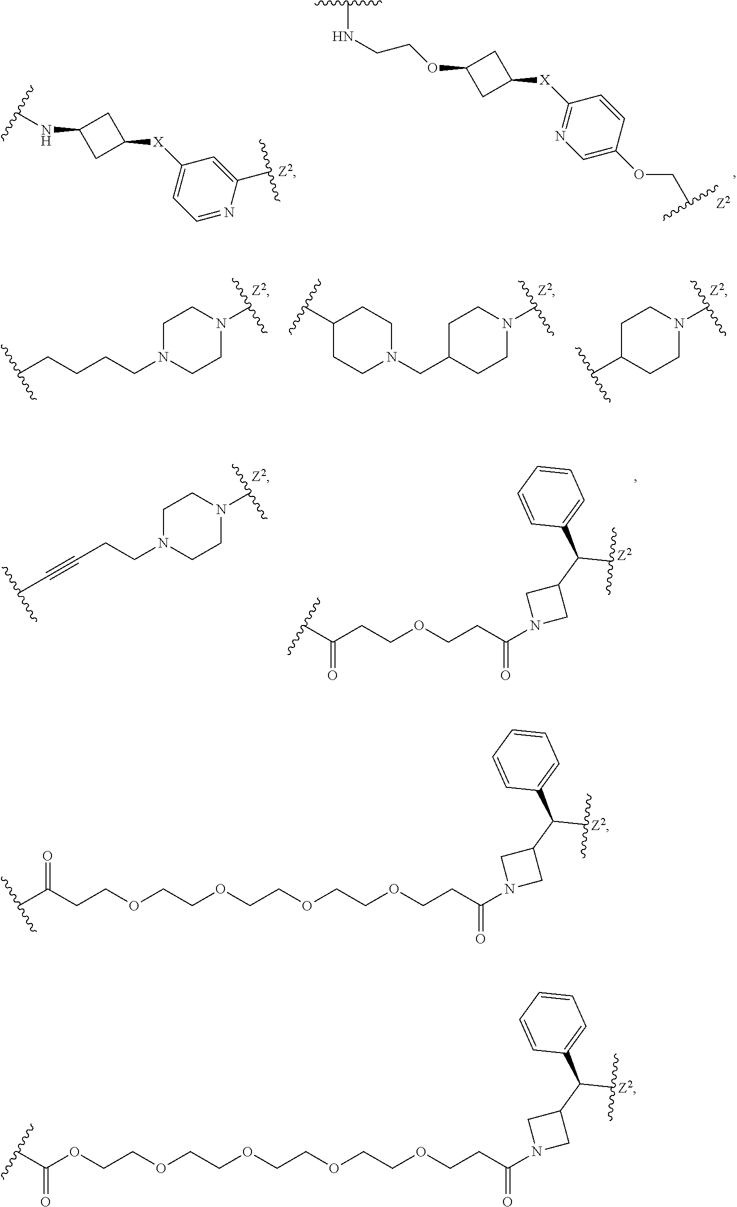

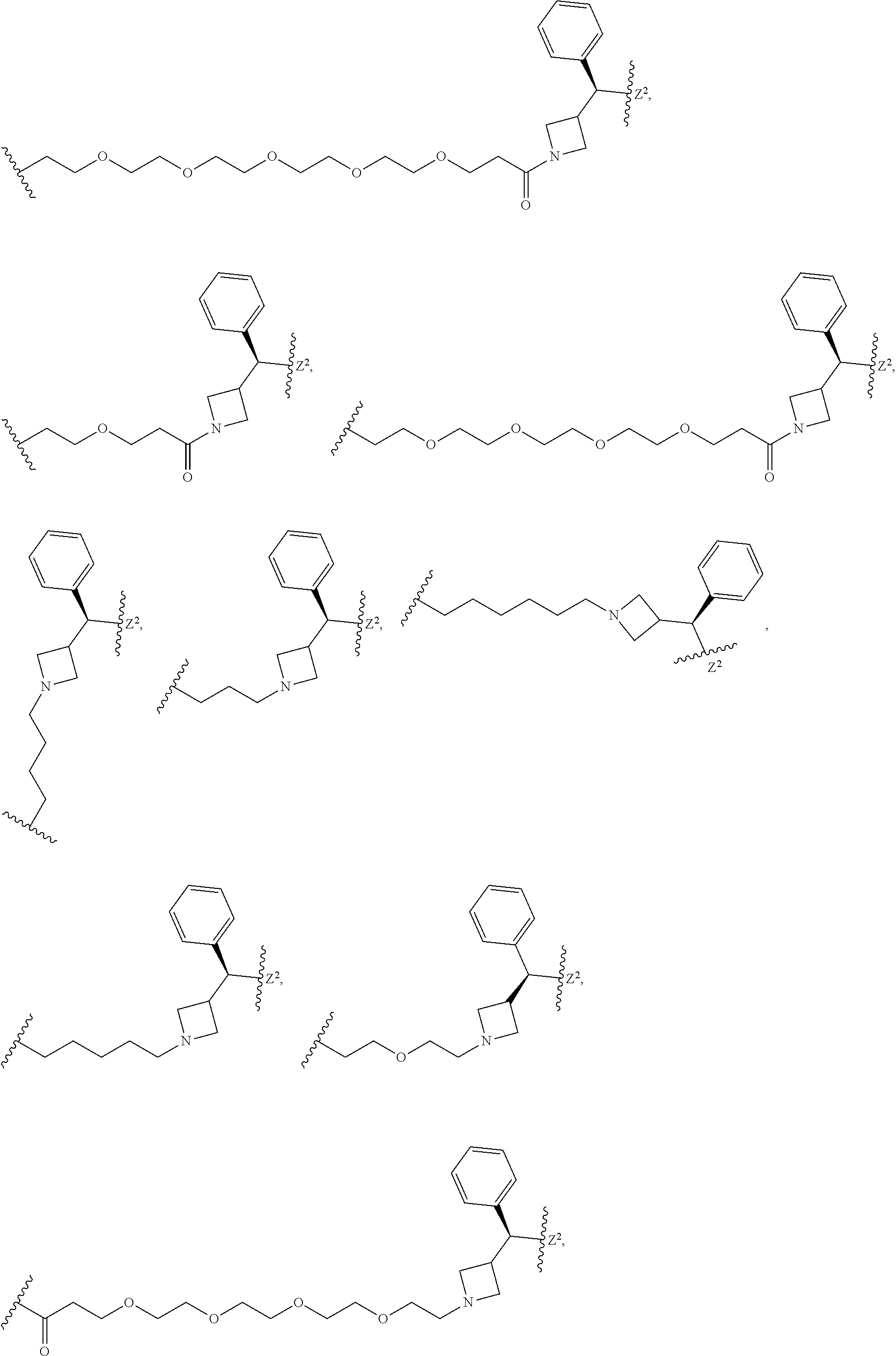

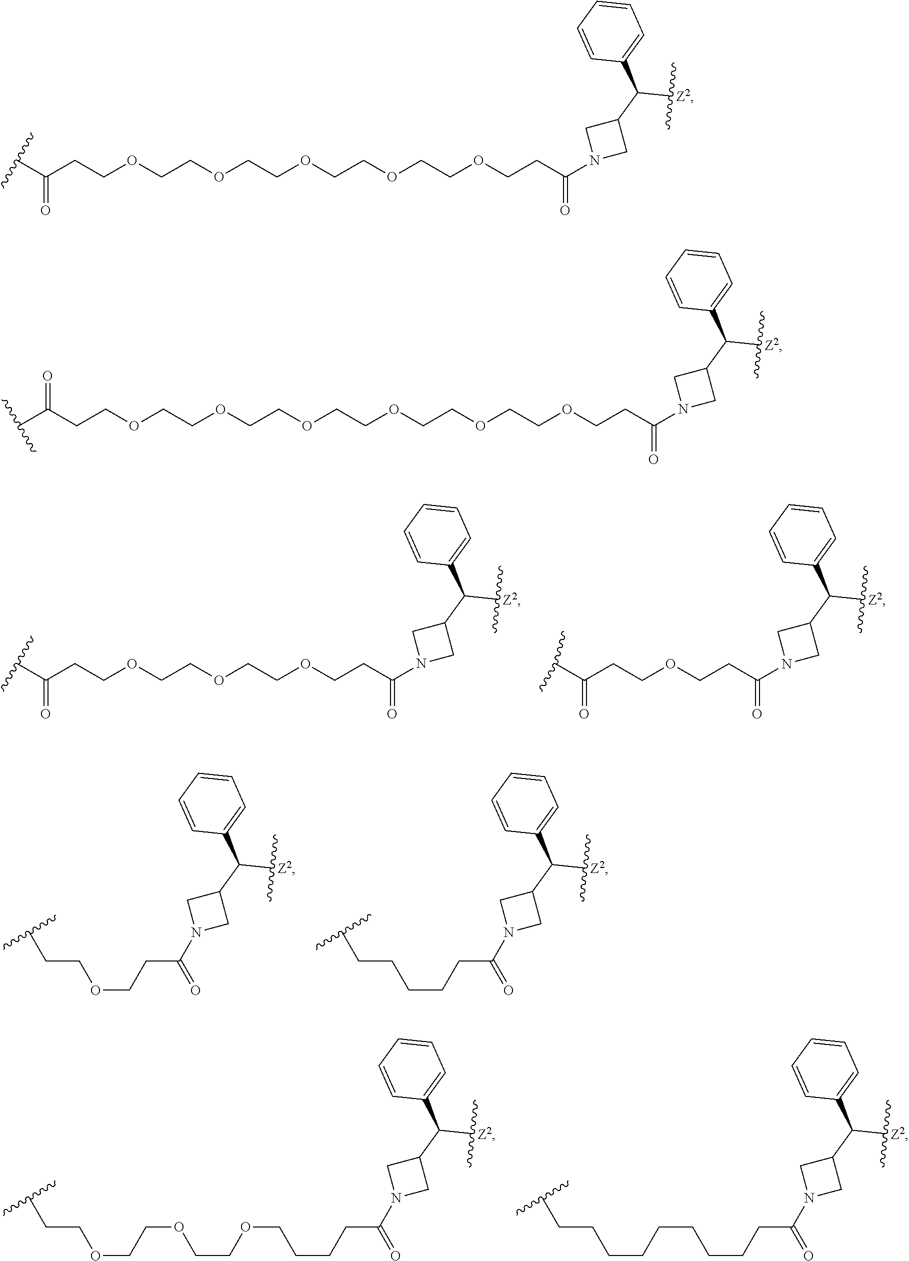

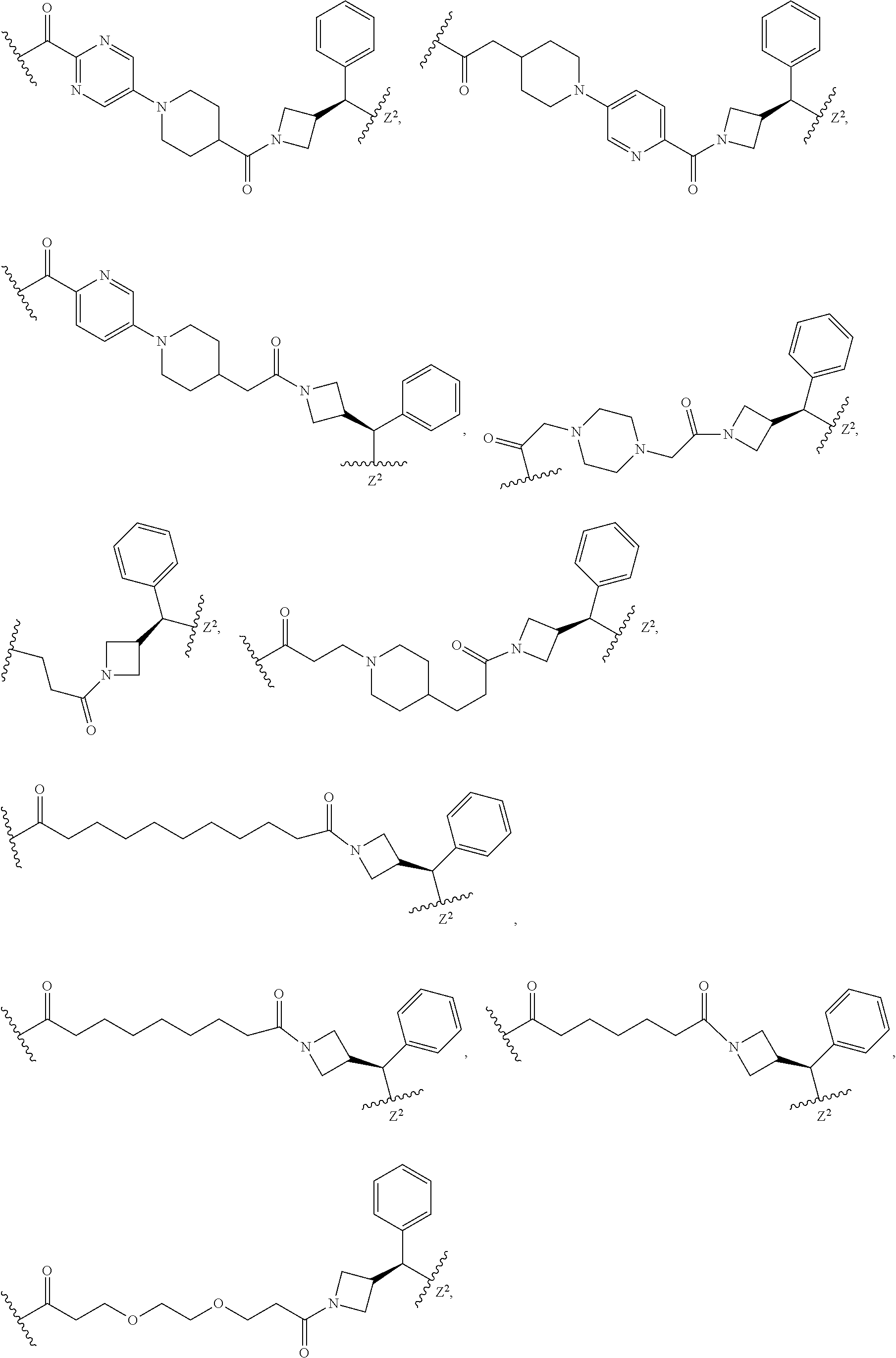

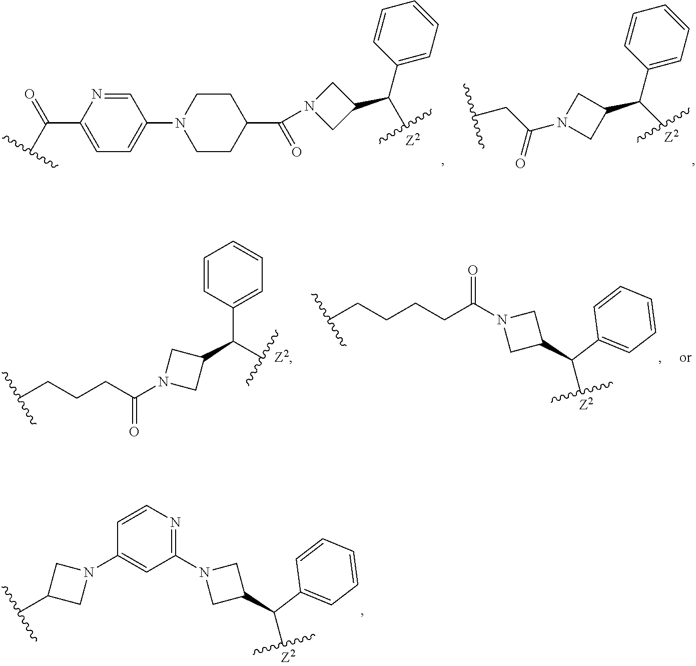 [SMILES: *C(=O)c1ccc(N2CCC(C(=O)N3CC([C@H](*)c4ccccc4)C3)CC2)cn1 ; *CC(=O)N1CC([C@H](*)c2ccccc2)C1 ; *CCCC(=O)N1CC([C@H](*)c2ccccc2)C1 ; *CCCCC(=O)N1CC([C@H](*)c2ccccc2)C1 ; *C1CN(c2ccnc(N3CC([C@H](*)c4ccccc4)C3)c2)C1]

wherein

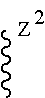

designates attachment to Z²; and wherein X is oxygen or sulfur.

In certain embodiments, provided herein are compounds of the following Table 1, and stereoisomers and pharmaceutically acceptable salts thereof.

| Compound | |
|---|---|
| No. | Structure |
| 1 | 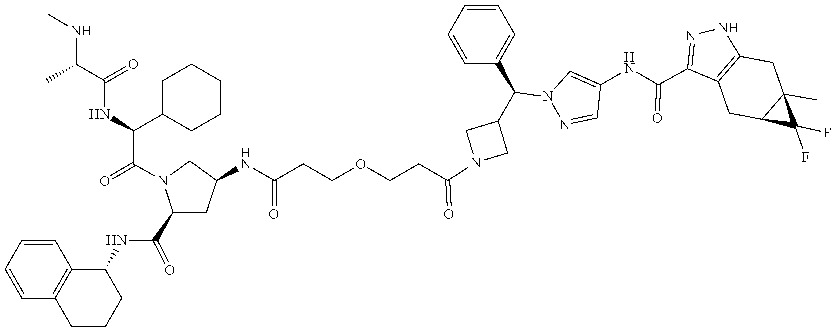 [SMILES: CN[C@@H](C)C(=O)N[C@H](C(=O)N1C[C@@H](NC(=O)CCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1] |
| 2 | 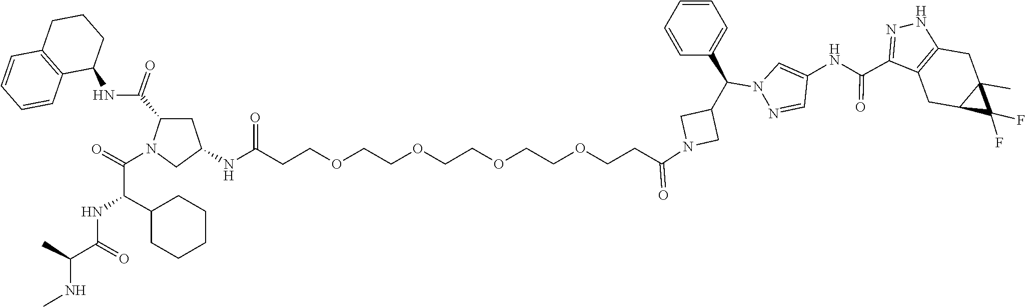 [SMILES: CN[C@@H](C)C(=O)N[C@H](C(=O)N1C[C@@H](NC(=O)CCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1] |
| 3 | 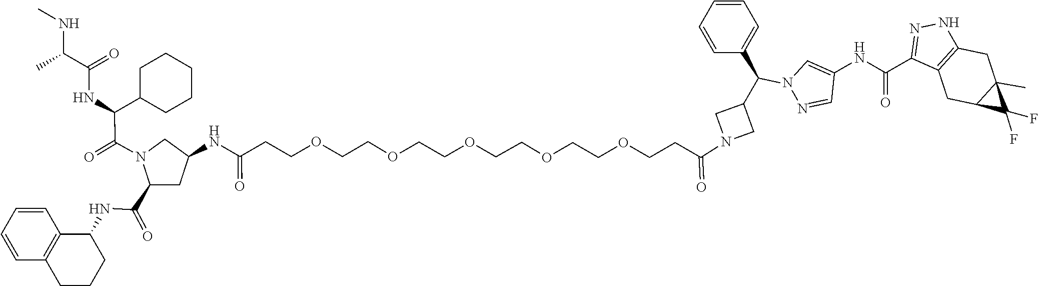 [SMILES: CN[C@@H](C)C(=O)N[C@H](C(=O)N1C[C@@H](NC(=O)CCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1] |
| 4 | 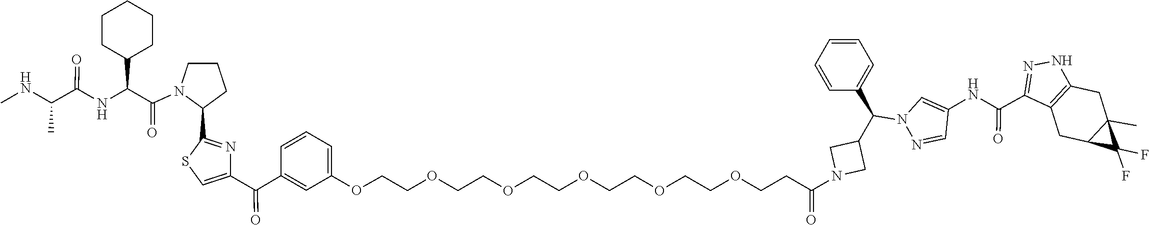 [SMILES: CN[C@@H](C)C(=O)N[C@H](C(=O)N1CCC[C@H]1c1nc(C(=O)c2cccc(OCCOCCOCCOCCOCCOCCC(=O)N3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 5 | 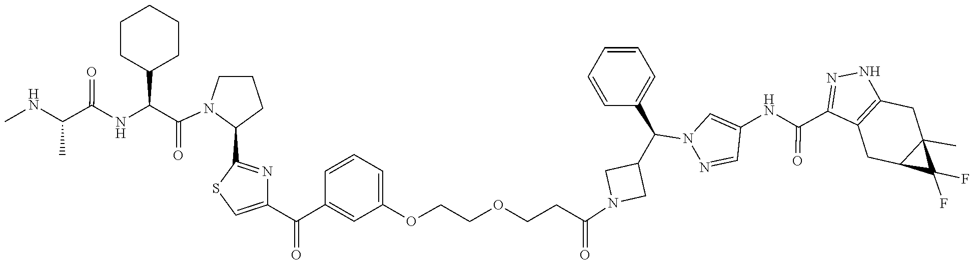 [SMILES: CN[C@@H](C)C(=O)N[C@H](C(=O)N1CCC[C@H]1c1nc(C(=O)c2cccc(OCCOCCC(=O)N3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 6 | 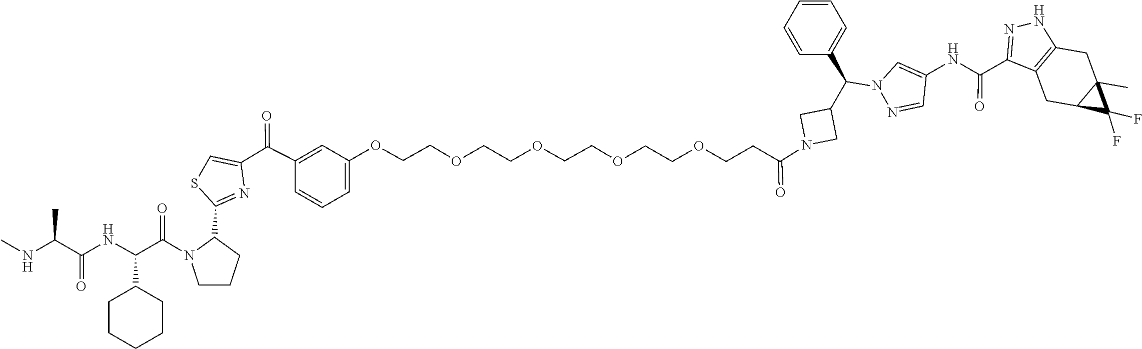 |
| 7 | 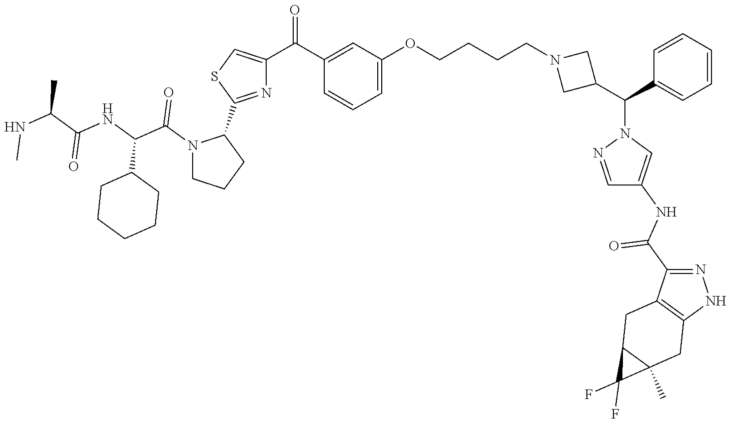 [SMILES: CN[C@@H](C)C(=O)N[C@H](C(=O)N1CCC[C@H]1c1nc(C(=O)c2cccc(OCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 8 | 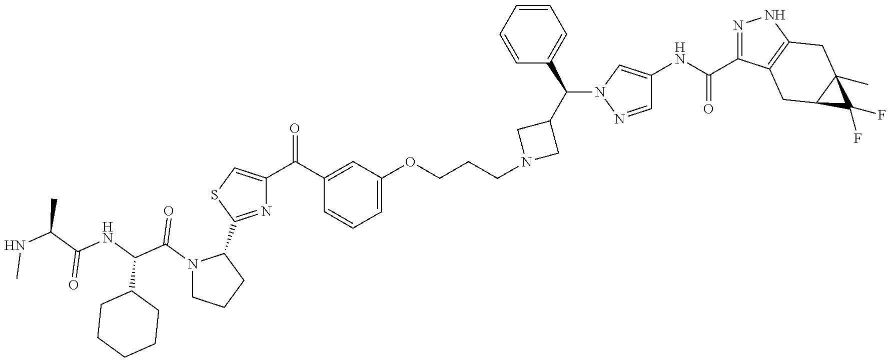 [SMILES: CN[C@@H](C)C(=O)N[C@H](C(=O)N1CCC[C@H]1c1nc(C(=O)c2cccc(OCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 9 | 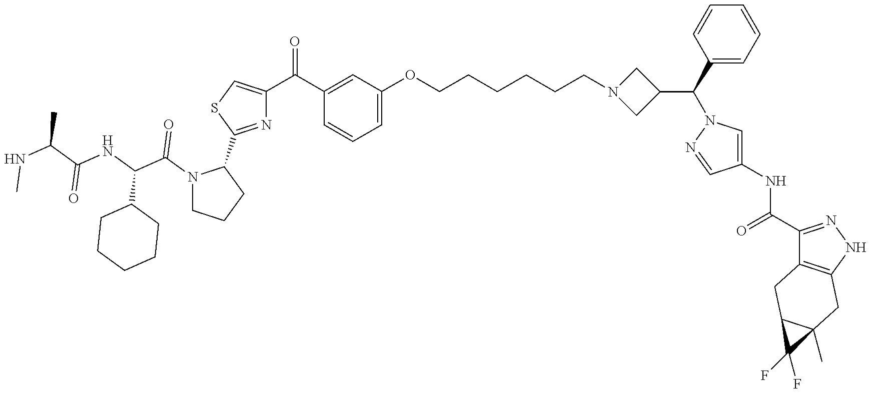 [SMILES: CN[C@@H](C)C(=O)N[C@H](C(=O)N1CCC[C@H]1c1nc(C(=O)c2cccc(OCCCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 10 | 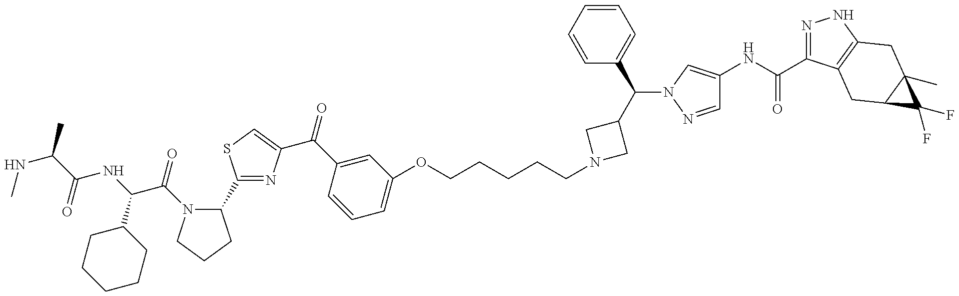 [SMILES: CN[C@@H](C)C(=O)N[C@H](C(=O)N1CCC[C@H]1c1nc(C(=O)c2cccc(OCCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 11 | 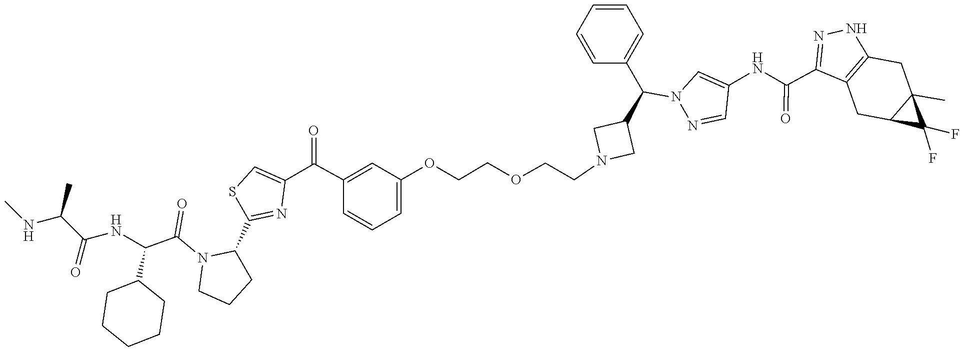 [SMILES: CN[C@@H](C)C(=O)N[C@H](C(=O)N1CCC[C@H]1c1nc(C(=O)c2cccc(OCCOCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 12 | 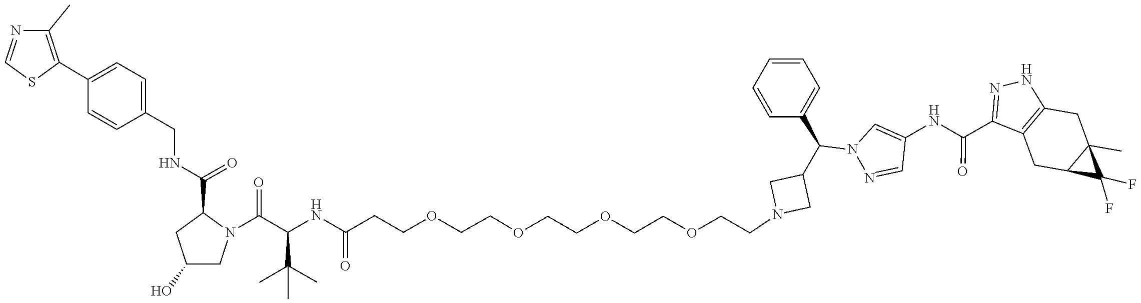 [SMILES: Cc1ncsc1-c1ccc(CNC(=O)[C@@H]2C[C@@H](O)CN2C(=O)[C@@H](NC(=O)CCOCCOCCOCCOCCN2CC([C@@H](c3ccccc3)n3cc(NC(=O)c4n[nH]c5c4C[C@@H]4C(F)(F)[C@]4(C)C5)cn3)C2)C(C)(C)C)cc1] |
| 13 | 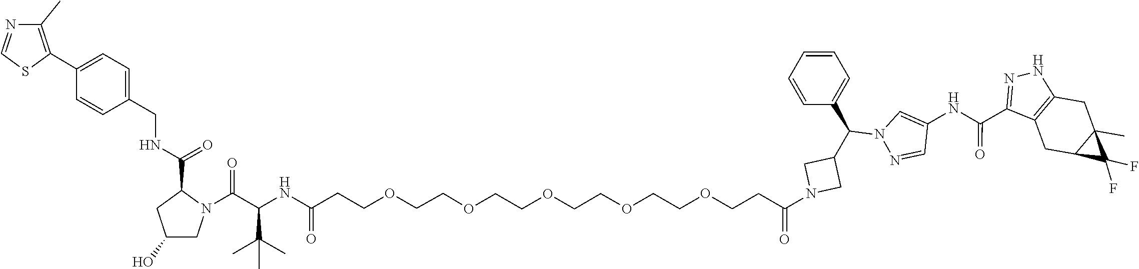 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 14 | 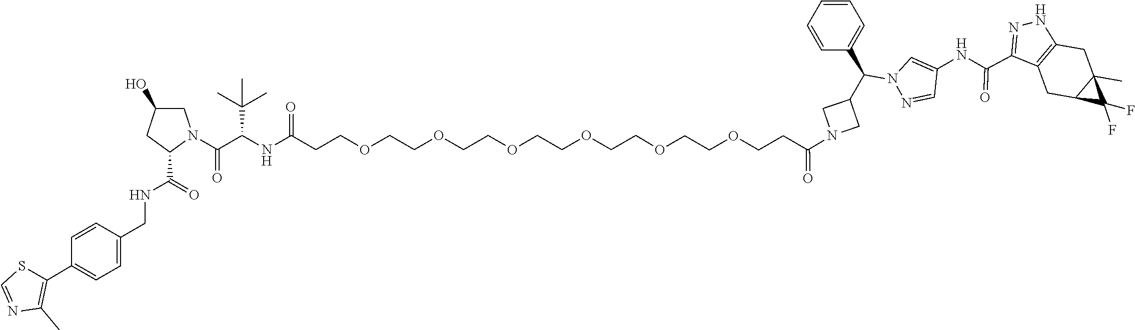 [SMILES: Cc1ncsc1-c1ccc(CNC(=O)[C@@H]2C[C@@H](O)CN2C(=O)[C@@H](NC(=O)CCOCCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 15 | 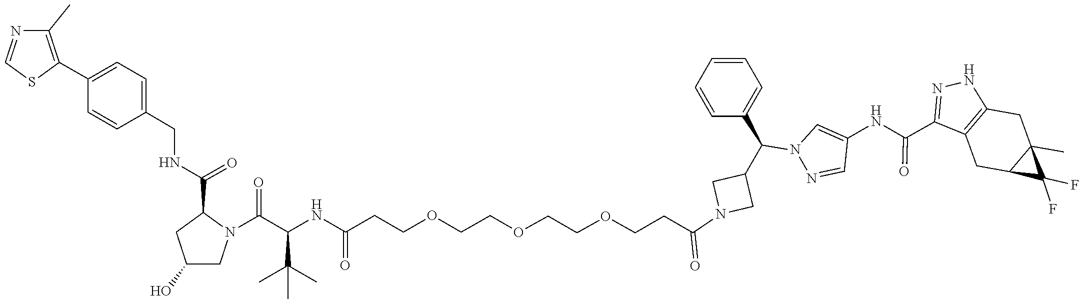 [SMILES: Cc1ncsc1-c1ccc(CNC(=O)[C@@H]2C[C@@H](O)CN2C(=O)[C@@H](NC(=O)CCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 16 | 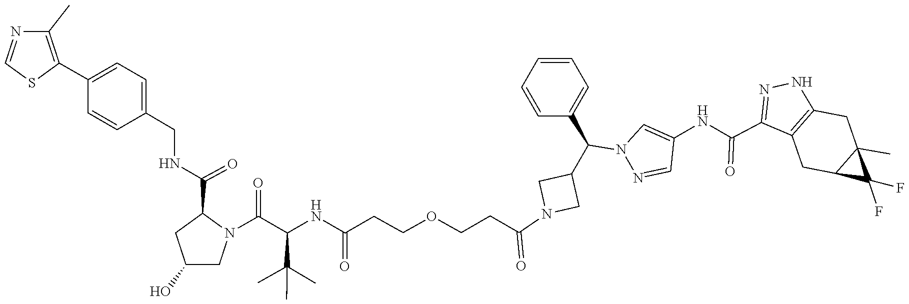 [SMILES: Cc1ncsc1-c1ccc(CNC(=O)[C@@H]2C[C@@H](O)CN2C(=O)[C@@H](NC(=O)CCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 17 | 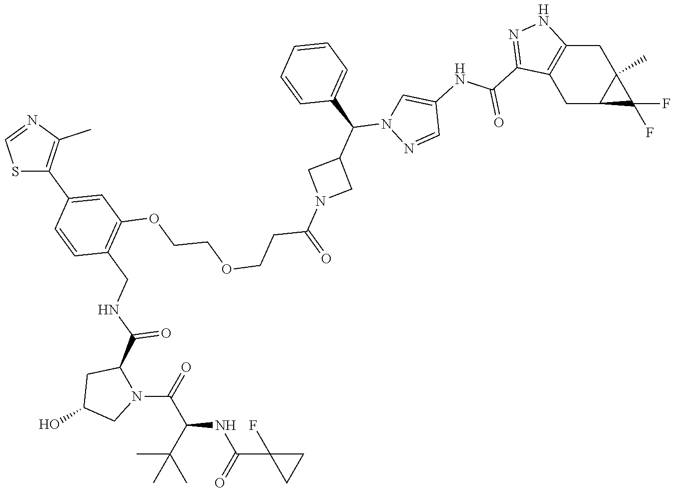 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCC(=O)N2CC([C@@H](c3ccccc3)n3cc(NC(=O)c4n[nH]c5c4C[C@@H]4C(F)(F)[C@]4(C)C5)cn3)C2)c1] |
| 18 | 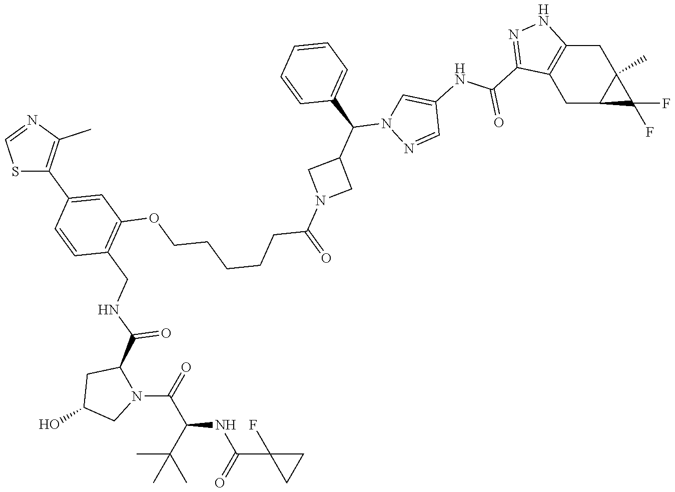 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)N2CC([C@@H](c3ccccc3)n3cc(NC(=O)c4n[nH]c5c4C[C@@H]4C(F)(F)[C@]4(C)C5)cn3)C2)c1] |
| 19 | 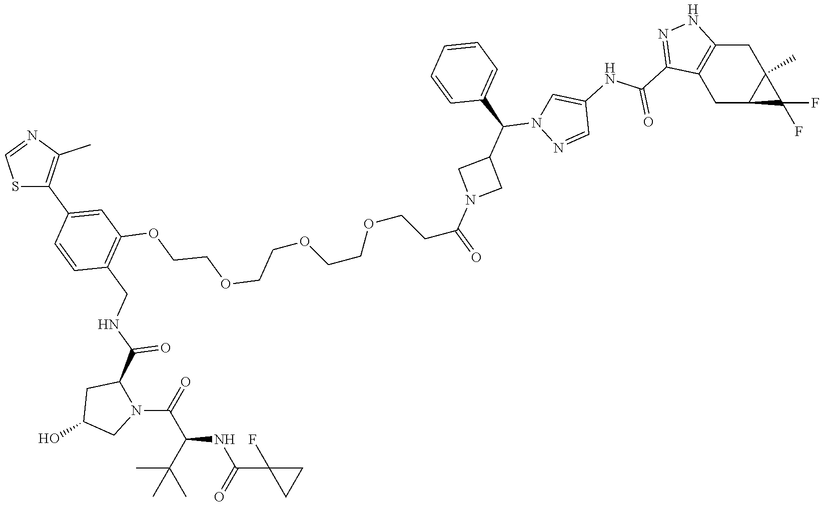 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCC(=O)N2CC([C@@H](c3ccccc3)n3cc(NC(=O)c4n[nH]c5c4C[C@@H]4C(F)(F)[C@]4(C)C5)cn3)C2)c1] |
| 20 | 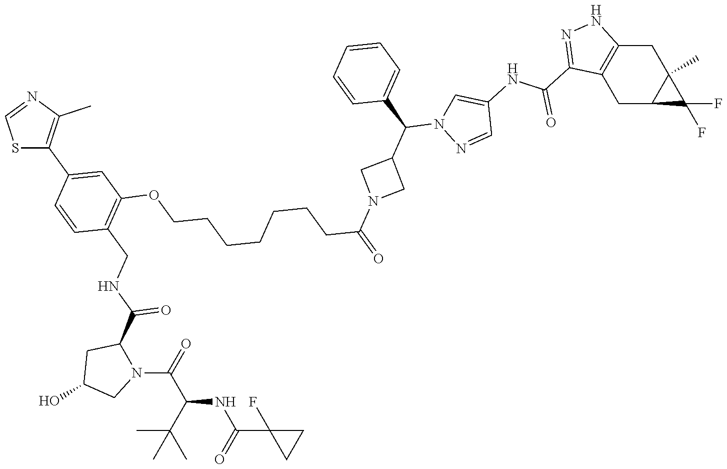 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)N2CC([C@@H](c3ccccc3)n3cc(NC(=O)c4n[nH]c5c4C[C@@H]4C(F)(F)[C@]4(C)C5)cn3)C2)c1] |
| 21 | 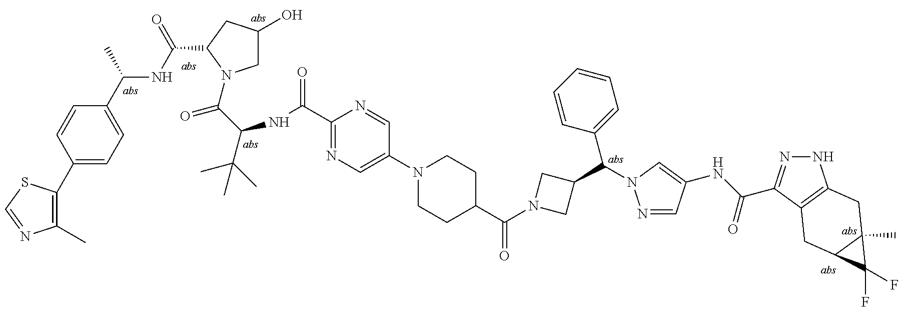 |
| 22 | 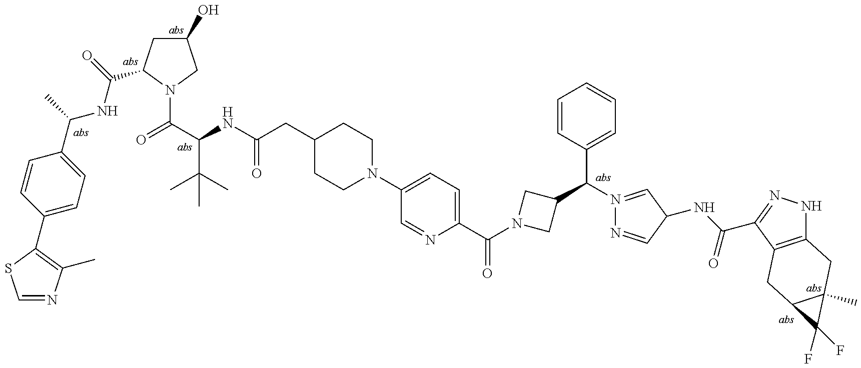 |
| 23 | 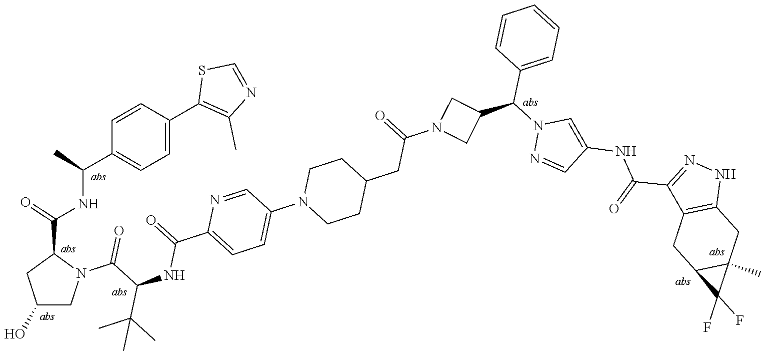 [SMILES: Cc1ncsc1-c1ccc([C@H](C)NC(=O)[C@@H]2C[C@@H](O)CN2C(=O)[C@@H](NC(=O)c2ccc(N3CCC(CC(=O)N4CC([C@@H](c5ccccc5)n5cc(NC(=O)c6n[nH]c7c6C[C@@H]6C(F)(F)[C@]6(C)C7)cn5)C4)CC3)cn2)C(C)(C)C)cc1] |
| 24 | 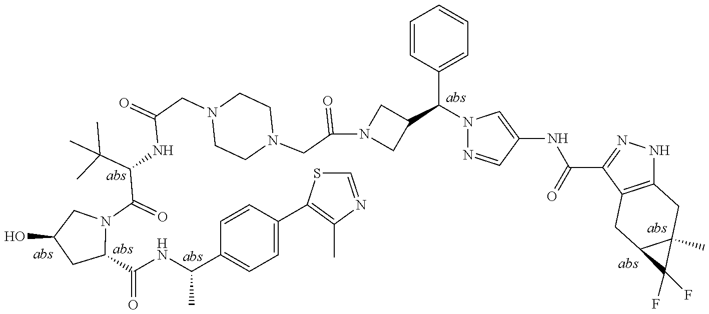 [SMILES: Cc1ncsc1-c1ccc([C@H](C)NC(=O)[C@@H]2C[C@@H](O)CN2C(=O)[C@@H](NC(=O)CN2CCN(CC(=O)N3CC([C@@H](c4ccccc4)n4cc(NC(=O)c5n[nH]c6c5C[C@@H]5C(F)(F)[C@]5(C)C6)cn4)C3)CC2)C(C)(C)C)cc1] |
| 25 | 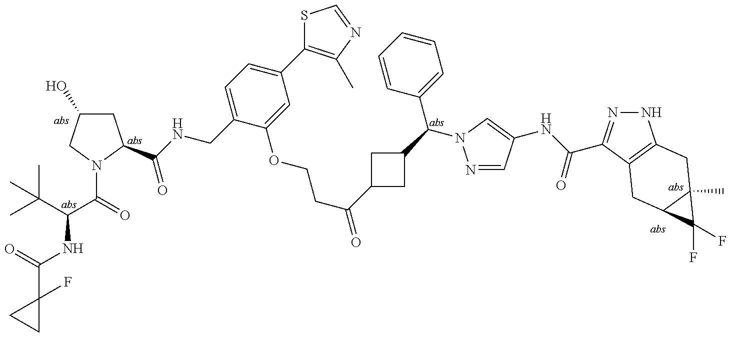 [SMILES: Cc1ncsc1-c1ccc(CNC(=O)[C@@H]2C[C@@H](O)CN2C(=O)[C@@H](NC(=O)C2(F)CC2)C(C)(C)C)c(OCCC(=O)C2CC([C@@H](c3ccccc3)n3cc(NC(=O)c4n[nH]c5c4C[C@@H]4C(F)(F)[C@]4(C)C5)cn3)C2)c1] |
| 26 | 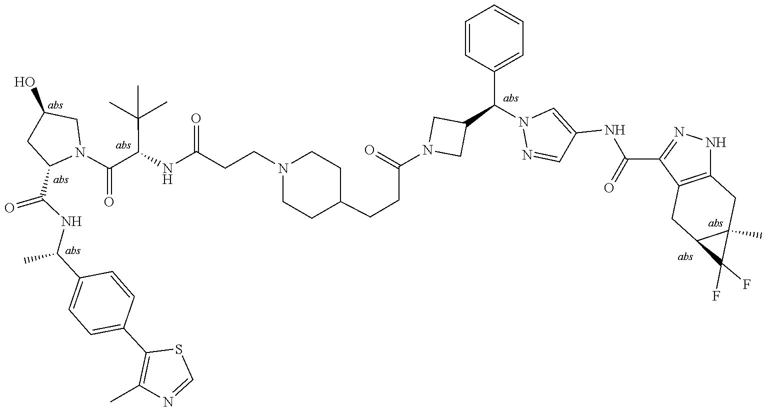 [SMILES: Cc1ncsc1-c1ccc([C@H](C)NC(=O)[C@@H]2C[C@@H](O)CN2C(=O)[C@@H](NC(=O)CCN2CCC(CCC(=O)N3CC([C@@H](c4ccccc4)n4cc(NC(=O)c5n[nH]c6c5C[C@@H]5C(F)(F)[C@]5(C)C6)cn4)C3)CC2)C(C)(C)C)cc1] |
| 27 | 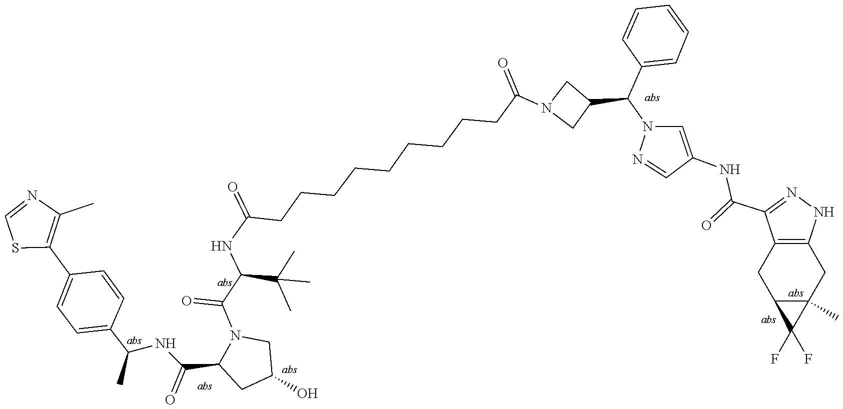 [SMILES: Cc1ncsc1-c1ccc([C@H](C)NC(=O)[C@@H]2C[C@@H](O)CN2C(=O)[C@@H](NC(=O)CCCCCCCCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 28 | 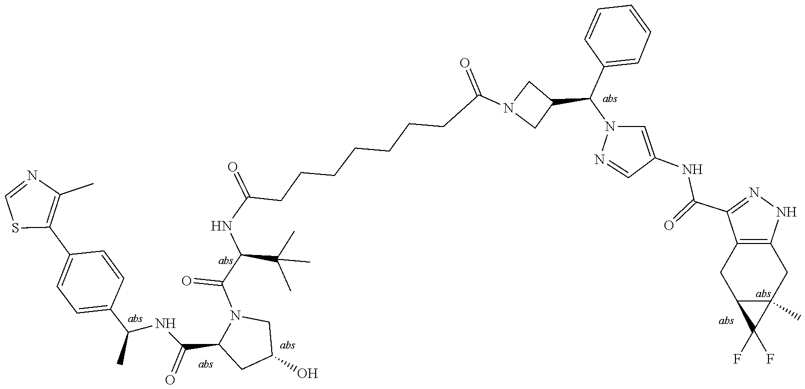 [SMILES: Cc1ncsc1-c1ccc([C@H](C)NC(=O)[C@@H]2C[C@@H](O)CN2C(=O)[C@@H](NC(=O)CCCCCCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 29 | 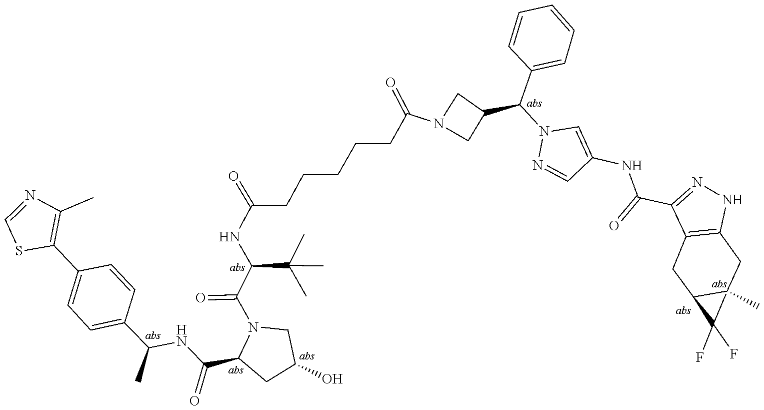 [SMILES: Cc1ncsc1-c1ccc([C@H](C)NC(=O)[C@@H]2C[C@@H](O)CN2C(=O)[C@@H](NC(=O)CCCCCC(=O)N2CC([C@@H](c3ccccc3)n3cc(NC(=O)c4n[nH]c5c4C[C@H]4[C@@H](C)C(C5)C4(F)F)cn3)C2)C(C)(C)C)cc1] |
| 30 | 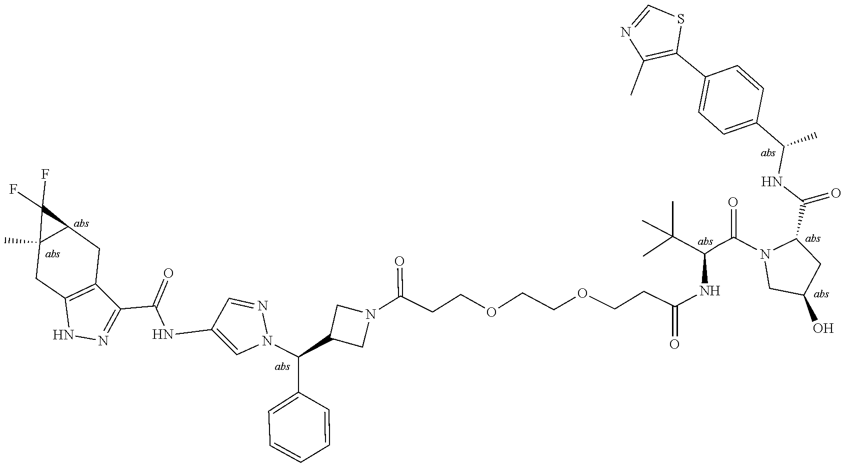 [SMILES: Cc1ncsc1-c1ccc([C@H](C)NC(=O)[C@@H]2C[C@@H](O)CN2C(=O)C[C@H](CC(C)(C)C)NC(=O)CCOCCOCCC(=O)N2CC([C@@H](c3ccccc3)n3cc(NC(=O)c4n[nH]c5c4C[C@@H]4C(F)(F)[C@]4(C)C5)cn3)C2)cc1] |
| 31 | 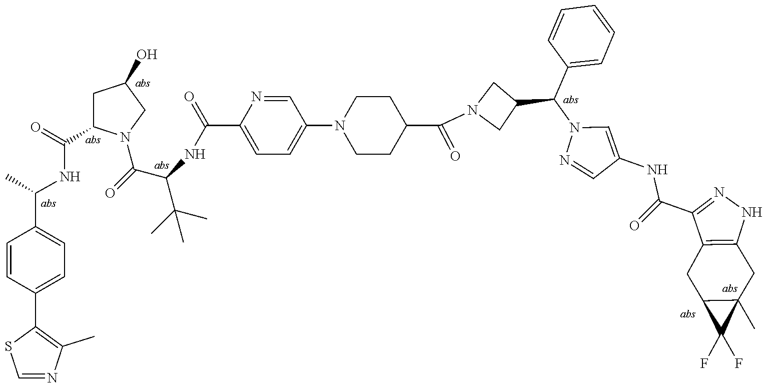 [SMILES: Cc1ncsc1-c1ccc([C@H](C)NC(=O)[C@@H]2C[C@@H](O)CN2C(=O)[C@@H](NC(=O)c2ccc(N3CCC(C(=O)N4CC([C@@H](c5ccccc5)n5cc(NC(=O)c6n[nH]c7c6C[C@@H]6C(F)(F)[C@]6(C)C7)cn5)C4)CC3)cn2)C(C)(C)C)cc1] |
| 32 | 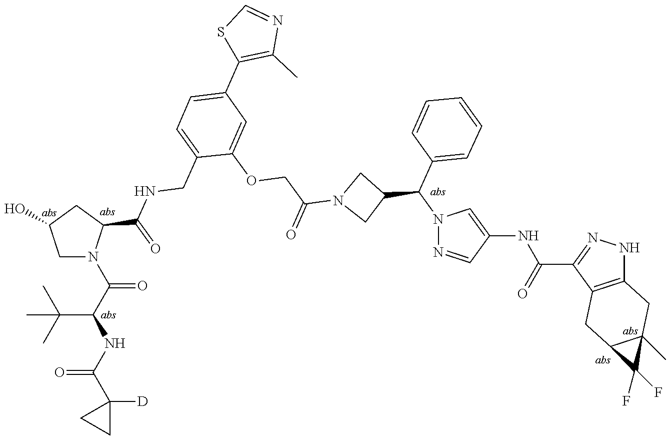 [SMILES: [2H]C1(C(=O)N[C@H](C(=O)N2C[C@H](O)C[C@H]2C(=O)NCc2ccc(-c3scnc3C)cc2OCC(=O)N2CC([C@@H](c3ccccc3)n3cc(NC(=O)c4n[nH]c5c4C[C@@H]4C(F)(F)[C@]4(C)C5)cn3)C2)C(C)(C)C)CC1] |
| 33 | 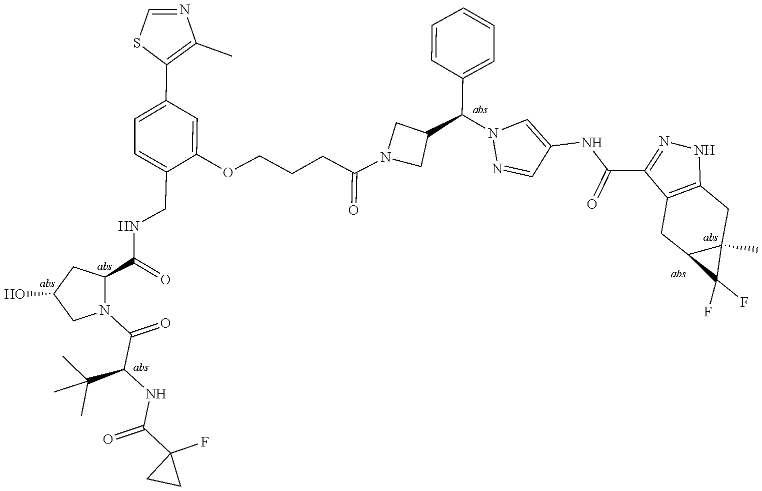 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N2CC([C@@H](c3ccccc3)n3cc(NC(=O)c4n[nH]c5c4C[C@@H]4C(F)(F)[C@]4(C)C5)cn3)C2)c1] |
| 34 | 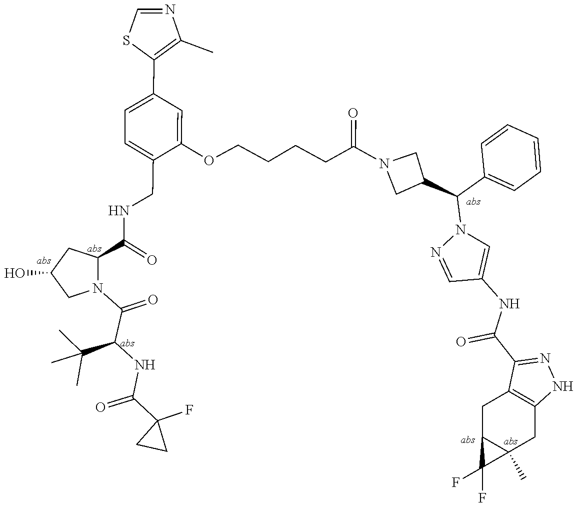 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCC(=O)N2CC([C@@H](c3ccccc3)n3cc(NC(=O)c4n[nH]c5c4C[C@@H]4C(F)(F)[C@]4(C)C5)cn3)C2)c1] |
| 35 | 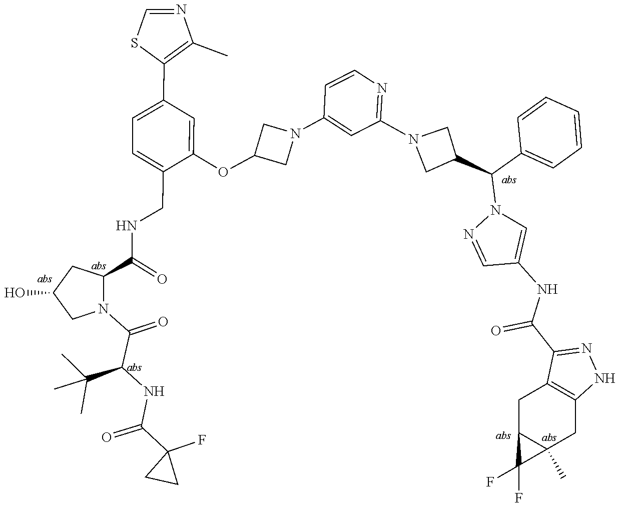 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CN(c3ccnc(N4CC([C@@H](c5ccccc5)n5cc(NC(=O)c6n[nH]c7c6C[C@@H]6C(F)(F)[C@]6(C)C7)cn5)C4)c3)C2)c1] |
| 36 | 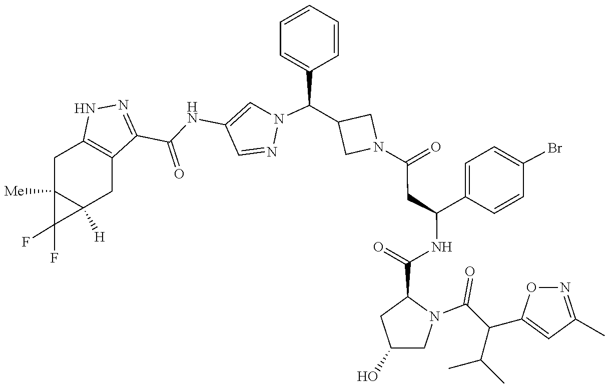 [SMILES: Cc1cc(C(C(=O)N2C[C@H](O)C[C@H]2C(=O)N[C@@H](CC(=O)N2CC([C@H](c3ccccc3)n3cc(NC(=O)c4n[nH]c5c4C[C@H]4C(F)(F)[C@@]4(C)C5)cn3)C2)c2ccc(Br)cc2)C(C)C)on1] |
| 37 | 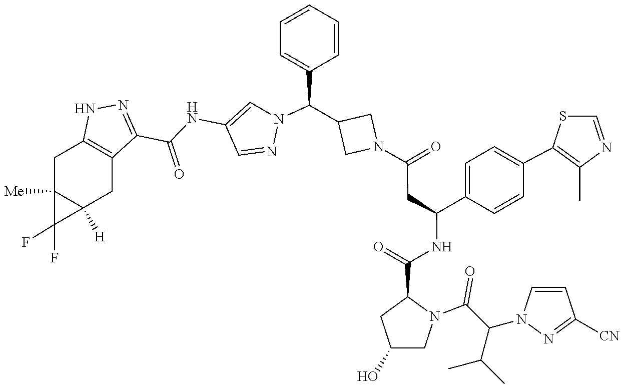 [SMILES: Cc1ncsc1-c1ccc([C@H](CC(=O)N2CC([C@H](c3ccccc3)n3cc(NC(=O)c4n[nH]c5c4C[C@H]4C(F)(F)[C@@]4(C)C5)cn3)C2)NC(=O)[C@@H]2C[C@@H](O)CN2C(=O)C(C(C)C)n2ccc(C#N)n2)cc1] |
| 38 | 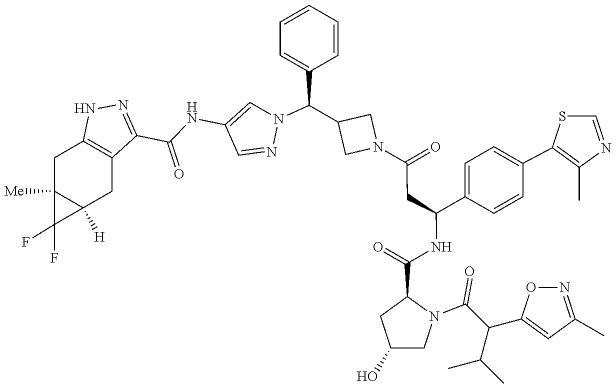 [SMILES: Cc1cc(C(C(=O)N2C[C@H](O)C[C@H]2C(=O)N[C@@H](CC(=O)N2CC([C@H](c3ccccc3)n3cc(NC(=O)c4n[nH]c5c4C[C@H]4C(F)(F)[C@@]4(C)C5)cn3)C2)c2ccc(-c3scnc3C)cc2)C(C)C)on1] |
| 39 | 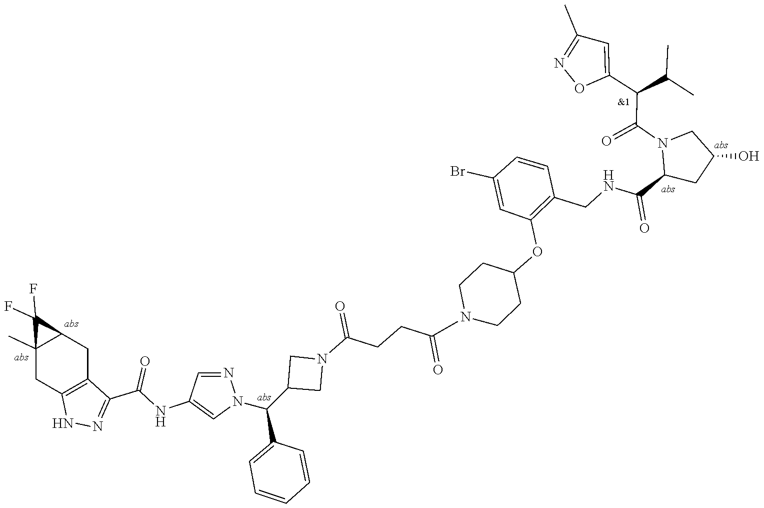 [SMILES: Cc1cc([C@H](C(=O)N2C[C@H](O)C[C@H]2C(=O)NCc2ccc(Br)cc2OC2CCN(C(=O)CCC(=O)N3CC([C@@H](c4ccccc4)n4cc(NC(=O)c5n[nH]c6c5C[C@@H]5C(F)(F)[C@]5(C)C6)cn4)C3)CC2)C(C)C)on1] |

In certain embodiments, the compound is selected from the compounds in Table 1.

### Pharmaceutical Compositions

The compounds described herein can be formulated into pharmaceutical compositions that further comprise a pharmaceutically acceptable carrier, diluent, adjuvant, or vehicle. In one embodiment, this disclosure provides a pharmaceutical composition comprising a compound described above, and a pharmaceutically acceptable carrier, diluent, adjuvant, or vehicle. In one embodiment, this disclosure provides a pharmaceutical composition comprising an effective amount of a compound of this disclosure, or a pharmaceutically acceptable salt thereof and a pharmaceutically acceptable carrier, diluent, adjuvant, or vehicle. Pharmaceutically acceptable carriers include, for example, pharmaceutical diluents, excipients, or carriers suitably selected with respect to the intended form of administration, and consistent with conventional pharmaceutical practices.

According to another embodiment, this description provides a composition comprising a compound herein, or a pharmaceutically acceptable salt thereof, and a pharmaceutically acceptable carrier, adjuvant, or vehicle. Pharmaceutical compositions of this description comprise a therapeutically effective amount of a compound of Formula (I), (Ia), (Ib), (II), (IIa), (IIb), (III), (IIIa), or (Ib), wherein a “therapeutically effective amount” is an amount that is (a) effective to measurably degrade ITK (or reduce the amount of ITK) in a biological sample or in a patient; or (b) effective in treating and/or ameliorating a disease or disorder that is mediated by ITK.

It also will be appreciated that certain compounds of this disclosure can exist in free form for treatment, or where appropriate, as a pharmaceutically acceptable derivative (e.g., a salt) thereof. According to this disclosure, a pharmaceutically acceptable derivative includes, but is not limited to, pharmaceutically acceptable prodrugs, salts, esters, salts of such esters, or any other adduct/educt or derivative that upon administration to a patient in need is capable of providing, directly or indirectly, a compound as otherwise described herein, or a metabolite, or residue thereof.

As used herein, the term “pharmaceutically acceptable salt” refers to those salts that are, within the scope of sound medical judgement, suitable for use in contact with the tissues of humans and lower animals without undue toxicity, irritation, allergic response, and the like.

Pharmaceutically acceptable salts are well known in the art. For example, S. M. Berge et al., describe pharmaceutically acceptable salts in detail in J. Pharmaceutical Sciences 1977, 66, 1-19, incorporated herein by reference. Pharmaceutically acceptable salts of the compounds of this description include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid, and perchloric acid; or with organic acids such as acetic acid, oxalic acid, maleic acid, tartaric acid, citric acid, succinic acid, or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts, and the like. Salts derived from appropriate bases include alkali metal, alkaline earth metal, ammonium (NH₄⁺) and N⁺(C₁₋₄ alkyl)₄ salts. This description also envisions the quaternization of any basic nitrogen-containing groups of the compounds disclosed herein. Water or oil-soluble or dispersable products may be obtained by such quaternization.

Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A pharmaceutically acceptable carrier may contain inert ingredients that do not unduly inhibit the biological activity of the compounds described herein. The pharmaceutically acceptable carriers should be biocompatible, for example, non-toxic, non-inflammatory, non-immunogenic, or devoid of other undesired reactions or side-effects upon administration to a subject. Standard pharmaceutical formulation techniques can be employed.

The pharmaceutically acceptable carrier, adjuvant, or vehicle, as used herein, includes any and all solvents, diluents, or other liquid vehicle, dispersion, or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions, and known techniques for the preparation thereof. Except insofar as any conventional carrier medium is incompatible with the compounds described herein, such as by producing any undesirable biological effect, or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic, or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities or renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

As used herein, the term “measurably degrade” means a measurable reduction in (a) ITK activity, between a sample comprising a compound of this description and an ITK and an equivalent sample comprising an ITK in the absence of said compound; or (b) the concentration of the ITK in a sample over time.

### Administration

The compositions of this disclosure may be administered orally, parenterally, by inhalation spray, topically, rectally, nasally, buccally, vaginally, or via an implanted reservoir. As used herein, the term “parenteral” includes subcutaneous, intravenous, intramuscular, intra-articular, intra-synovial, intrasternal, intrathecal, intraocular, intrahepatic, intralesional, and intracranial injection or infusion techniques. Compositions can be administered orally, intraperitoneally, or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans, and other emulsifying agents or bioavailability enhancers that are commonly used in the manufacture of pharmaceutically acceptable solid, liquid, or other dosage forms may also be used for the purposes of formulation.

The pharmaceutically acceptable compositions of this disclosure may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents also may be added.

Alternatively, the pharmaceutically acceptable compositions of this disclosure may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melts in the rectum or vaginal cavity and releases the active compound.

The pharmaceutically acceptable compositions of this disclosure also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax, and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

For ophthalmic use, the pharmaceutically acceptable compositions may be formulated, for example, as micronized suspensions in isotonic, pH adjusted sterile saline or other aqueous solution, or as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this disclosure also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the compositions of this disclosure are administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents also may be added.

Liquid dosage forms for oral administration include, but are not limited to, pharmaceutically acceptable emulsions, microemulsions, solutions, suspensions, syrups, and elixirs. In addition to the active compounds herein, the liquid dosage forms may contain inert diluents commonly used in the art for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound described herein is mixed with at least one inert, pharmaceutically acceptable excipient or carrier such as sodium citrate or dicalcium phosphate, and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, for example, in a certain part of the intestinal tract, optionally in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds herein also can be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms also may comprise, as is normal practice, additional substances other than inert diluents, for example, tableting lubricants and other tableting aids such a magnesium stearate and microcrystalline cellulose. In the case of capsules, tablets, and pills, the dosage forms also may comprise buffering agents. They may optionally contain opacifying agents and also can be of a composition such that they release the active ingredient(s) only, for example, in a certain part of the intestinal tract, optionally in a delayed manner. Examples of embedding compositions that can be used include polymeric substances and waxes.

The compounds of the description are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds, and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed, and like factors well known in the medical arts.

The amount of the compounds of this disclosure that may be combined with the carrier materials to produce a composition in a single dosage form will vary depending upon the host treated, the particular mode of administration, and other factors. The compositions should be formulated so that a dosage of between 0.01-100 mg/kg body weight/day of the compound or inhibitor can be administered to a patient receiving these compositions.

Depending upon the particular condition or disease to be treated or prevented, additional therapeutic agents, which are normally administered to treat or prevent that condition, also may be present in the compositions of this disclosure. As used herein, additional therapeutic agents that are normally administered to treat or prevent a particular disease, or condition, are known as “appropriate for the disease, or condition, being treated.”

The compound or composition can be administered concurrently with, prior to, or subsequent to, one or more additional therapeutically active agents. In general, each agent will be administered at a dose and/or on a time schedule determined for that agent. It will further be appreciated that the additional therapeutically active agent utilized in this combination can be administered together in a single composition or administered separately in different compositions. The particular combination to employ in a regimen will take into account compatibility of the compounds described herein with the additional therapeutically active agent and/or the desired therapeutic effect to be achieved. In general, it is expected that additional therapeutically active agents utilized in combination will be utilized at levels that do not exceed the levels at which they are utilized individually. In some embodiments, the levels utilized in combination will be lower than those utilized individually. Additional therapeutically active agents include, but are not limited to, small organic molecules such as drug compounds (e.g., compounds approved by the Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional therapeutically active agent is a cancer agent (e.g., a biotherapeutic or chemo therapeutic cancer agent). In other embodiments, the additional therapeutically active agent is an anti-inflammatory agent.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

### Methods of Use

The bifunctional compounds described herein are useful for degrading ITK in biological samples, or in patients via a ubiquitin proteolytic pathway. Thus, an embodiment of this disclosure provides a method of treating a ITK-mediated disease or disorder. As used herein, the term “ITK-mediated disease or disorder” means any disease, disorder, or other deleterious condition in which an ITK is known to play a role. In some instances, an ITK-mediated disease or disorder is a proliferative disorder or an autoimmune disorder. Examples of proliferative disorders include cancer.

In one aspect, provided herein are methods of treating or preventing cancer in a subject in need thereof. In certain embodiments, the methods comprise the step of orally administering to the subject an amount of a bifunctional compound capable of inducing proteolytic degradation of ITK. In certain embodiments, the amount is effective to treat or prevent the cancer.

In certain embodiments, the cancer is any cancer described below. In particular embodiments, the cancer comprises a solid tumor. In certain embodiments, the cancer is a B cell malignancy. In certain embodiments, the cancer is selected from the group consisting of chronic lymphocytic leukemia (CLL), small lymphocytic lymphoma (SLL), transformed CLL or Richter's transformation, small cell lymphoma, follicular lymphoma (FL), diffuse large B-cell lymphoma (DLBCL), non-Hodgkin lymphoma, mantle cell lymphoma (MCL), marginal zone lymphoma (MZL), Waldenstrom macroglobulinemia (WM), and central nervous system (CNS) lymphoma. In certain embodiments, the cancer is chronic lymphocytic leukemia. In certain embodiments, the cancer is small cell lymphoma. In certain embodiments, the cancer is follicular lymphoma. In certain embodiments, the cancer is diffuse large B-cell lymphoma. In certain embodiments, the cancer is non-Hodgkin lymphoma. In certain embodiments, the cancer is mantle cell lymphoma. In certain embodiments, the cancer is marginal zone lymphoma. In certain embodiments, the cancer is Waldenstrom macroglobulinemia. In certain embodiments, the cancer is small lymphocytic lymphoma (SLL). In certain embodiments, the cancer is CNS lymphoma. In certain embodiments, the cancer is transformed CLL or Richter's transformation. In certain embodiments, the cancer is chronic lymphocytic leukemia (CLL).

In another aspect, provided herein are methods of degrading ITK in a subject in need thereof. The methods comprise the step of orally administering to the subject an amount of a bifunctional compound described herein and capable of inducing proteolytic degradation of ITK. In certain embodiments, the amount is effective to degrade ITK in the subject. The ITK can be expressed in any cells or tissues of the subject. In certain embodiments, the ITK is expressed in splenocytes. In certain embodiments, the ITK is expressed in peripheral blood mononuclear cells.

In another aspect, provided herein are methods of preventing B cell activation in a subject in need thereof. The methods comprise the step of orally administering to the subject an amount of a bifunctional compound described herein and capable of inducing proteolytic degradation of ITK. In certain embodiments, the amount is effective to prevent B cell activation. In certain embodiments, the B cell expresses CD69. In certain embodiments, the B cell expresses CD86. In certain embodiments, the B cell expresses CD69 and CD86.

In the methods, the bifunctional compounds described herein comprise a moiety capable of specifically binding ITK and further comprise a moiety capable of recruiting a ubiquitin ligase to degrade the ITK. Particular compounds with each capability are described herein. The compounds can be administered in any form, including pharmaceutically acceptable salts and pharmaceutical compositions.

The bifunctional compound(s) described herein can be administered in any dose deemed suitable by the practitioner of skill. In certain embodiments, the dose is 0.1-1000 mg/kg. In certain embodiments, the dose is 0.1-900 mg/kg. In certain embodiments, the dose is 0.1-800 mg/kg. In certain embodiments, the dose is 0.1-700 mg/kg. In certain embodiments, the dose is 0.1-600 mg/kg. In certain embodiments, the dose is 0.1-500 mg/kg. In certain embodiments, the dose is 0.1-400 mg/kg. In certain embodiments, the dose is 0.1-300 mg/kg. In certain embodiments, the dose is 0.1-200 mg/kg. In certain embodiments, the dose is 0.1-100 mg/kg. In certain embodiments, the dose is selected from the group consisting of 100 mg/kg, 200 mg/kg, 300 mg/kg, 450 mg/kg, 600 mg/kg, 800 mg/kg, and 1000 mg/kg. In certain embodiments, the dose is about 25 mg/kg. In certain embodiments, the dose is about 50 mg/kg. In certain embodiments, the dose is about 75 mg/kg. In certain embodiments, the dose is about 100 mg/kg. In certain embodiments, the dose is about 150 mg/kg. In certain embodiments, the dose is about 200 mg/kg. In certain embodiments, the dose is about 250 mg/kg. In certain embodiments, the dose is about 300 mg/kg. In certain embodiments, the dose is about 400 mg/kg. In certain embodiments, the dose is about 450 mg/kg. In certain embodiments, the dose is about 500 mg/kg. In certain embodiments, the dose is about 600 mg/kg. In certain embodiments, the dose is about 700 mg/kg. In certain embodiments, the dose is about 750 mg/kg. In certain embodiments, the dose is about 800 mg/kg. In certain embodiments, the dose is about 900 mg/kg. In certain embodiments, the dose is about 1000 mg/kg.

The dose can be administered on a schedule deemed suitable by the person of skill in the art. In certain embodiments, the dose is administered once per day. In certain embodiments, the dose is administered twice per day. In certain embodiments, the dose is administered three times per day. In certain embodiments, the dose is administered four times per day. In certain embodiments, the dose is administered in divided doses. In certain embodiments, the dose is administered in two divided doses per day. In certain embodiments, the dose is administered in three divided doses per day. In certain embodiments, the dose is administered in four divided doses per day.

Dosing can continue for any length of time deemed suitable by the person of skill in the art. In certain embodiments, the dose is administered daily for fourteen days. In certain embodiments, the dose is administered daily for thirteen days. In certain embodiments, the dose is administered daily for twelve days. In certain embodiments, the dose is administered daily for eleven days. In certain embodiments, the dose is administered daily for ten days. In certain embodiments, the dose is administered daily for nine days. In certain embodiments, the dose is administered daily for eight days. In certain embodiments, the dose is administered daily for seven days. In certain embodiments, the dose is administered daily for six days. In certain embodiments, the dose is administered daily for five days. In certain embodiments, the dose is administered daily for four days. In certain embodiments, the dose is administered daily for three days. In certain embodiments, the dose is administered daily for two days. In certain embodiments, the dose is administered for one day.

In the dosing schedule, the doses can be administered on consecutive days or cyclicly, according to the judgment of the practitioner of skill. In certain embodiments, the doses are administered on consecutive days. In certain embodiments, the doses are administered with an interval between doses. In certain embodiments, the interval is one day. In certain embodiments, the interval is two days. In certain embodiments, the interval is three days. In certain embodiments, the interval is four days. In certain embodiments, the interval is five days. In certain embodiments, the interval is six days.

In certain embodiments, the dose is administered weekly. In certain embodiments, the dose is administered twice per week. In certain embodiments, the dose is administered three times per week.

In certain embodiments, the dose(s) are administered for a period of time with a first interval between dose(s), and then the dose(s) are re-administered for a period of time following the first interval between dose(s), wherein this dosing regimen can be repeated (i.e., cyclicly or cyclically, for example, after a second, third, etc. interval between subsequent administrations of dose(s)) according to the judgment of the practitioner of skill. For example, in one embodiment, a first dose is administered for one week, followed by a first interval of one week without the first dose administration; then, a second dose is re-administered for another week, followed by a second interval of one week without the first or second dose administration, and so on cyclically. Other perturbations for first, second, third, etc. dose(s) followed by perturbations for first, second, third, etc. interval(s), and combinations thereof, are contemplated herein as would be appreciated by the practitioner of skill and the need of the patient. For example, in one embodiment, a first dose is administered daily for one week, followed by a first interval of three weeks without the first daily dose administration; then, a second dose is re-administered biweekly for another week, followed by a second interval of four weeks without the first daily or second biweekly dose administration, and so on cyclically.

The compound can be administered by any route of administration deemed suitable by the practioner of skill. In certain embodiments, the dose is administered orally. Formulations and techniques for administration are described in detail below.

In certain embodiments, term “cancer” includes, but is not limited to, the following cancers: epidermoid Oral: buccal cavity, lip, tongue, mouth, pharynx, squamous cell carcinoma of the head and neck (HNSCC); Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung cancer (NSCLC); Gastrointestinal: gastric cancer, esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, microsatellite stable colorectal cancer (MSS CRC), rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), metastatic castrate-resistant prostate cancer (mCRPC), muscle-invasive urothelial cancer; Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma (MM), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical cancer, cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast, triple-negative breast cancer (TNBC), platinum-resistant epithelial ovarian cancer (EOC); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders (e.g., mantle cell lymphoma, Waldenstrom's macroglobulinemia, Marginal zone lymphoma, and Follicular lymphoma); Skin: malilymphgnant melanoma, basal cell carcinoma, squamous cell carcinoma, Karposi's sarcoma, keratoacanthoma, moles or dysplastic nevi, lipoma, angioma, dermatofibroma, keloids, psoriasis; Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; Adrenal glands: neuroblastoma; and metatstaic melanoma.

In certain embodiments, term “autoimmune disease” includes, but is not limited to, the following autoimmune diseases: uticaria, graft-versus-host disease (GVHID), acute graft-versus-host disease, pemphigus vulgaris, achalasia, Addison's disease, Adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, axonal and neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (Acne Inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease (chronic), Meniere's disease, microscopic polyangiitis (MPA), mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonnage-Turner syndrome, pemphigus, peripheral neuropathy, perivenous encephalomyelitis, pernicious anemia (PA), POEMS syndrome, polyarteritis nodosa, polyglandular syndromes type I, II, or III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis (giant cell arteritis), thrombocytopenic purpura (TTP), Tolosa-Hunt syndrome (THS), transverse myelitis, Type 1 diabetes, ulcerative colitis (UC), undifferentiated connective tissue disease (UCTD), uveitis, vasculitis, vitiligo, Vogt-Koyanagi-Harada Disease, and Wegener's granulomatosis (or Granulomatosis with Polyangiitis (GPA)).

In certain embodiments, term “inflammatory disease” includes, but is not limited to, the following inflammatory diseases: encephalitis, myelitis, meningitis, arachnoiditis, neuritis, dacryoadenitis, scleritis, episcleritis, keratitis, retinitis, chorioretinitis, blepharitis, conjunctivitis, uveitis, otitisexterna, otitismedia, labyrinthitis, mastoiditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascendingcholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis/osteitis/osteomyelitis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminalvesiculitis, balanitis, posthitis, balanoposthitis, chorioamnionitis, funisitis, omphalitis, insulitis, hypophysitis, thyroiditis, parathyroiditis, adrenalitis, lymphangitis, and lymphadenitis.

### Articles of Manufacture and Kits

Also provided are articles of manufacture comprising any of the compounds or pharmaceutical compositions described herein. The articles of manufacture include suitable containers or packaging materials for the compounds or pharmaceutical compositions. Examples of a suitable container include, but are not limited to, a bottle, a vial, a syringe, an intravenous bag, or a tube.

Also provided are kits comprising any of the compounds or pharmaceutical compositions described herein. The kits can contain the compounds or pharmaceutical compositions in suitable containers or packaging materials, including, but not limited to, a bottle, a vial, a syringe, an intravenous bag, or a tube. The kits can comprise the compounds or pharmaceutical compositions for administration to an individual in single-dose form or in multiple-dose form. The kits can further comprise instructions or a label for administering the compounds or pharmaceutical compositions to an individual according to any of the methods disclosed herein. The kits can further comprise equipment for administering the compounds or pharmaceutical compositions to an individual, including, but not limited to, needles, syringes, tubing, or intravenous bags. The kits can further comprise instructions for producing any of the compounds or pharmaceutiucal compositions disclosed herein.

Also provided are articles of manufacture comprising any of the compounds, vaccines, or pharmaceutical compositions described herein. The articles of manufacture include suitable containers or packaging materials for the compounds or pharmaceutical compositions. The articles of manufacture include suitable containers or packaging materials for the compounds, oncolytic viruses, or pharmaceutical compositions. Examples of a suitable container include, but are not limited to, a bottle, a vial, a syringe, an intravenous bag, or a tube.

This disclosure will be more fully understood by reference to the following Examples. They should not, however, be construed as limiting the scope of this disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional embodiments are disclosed in further detail in the following examples, which are not in any way intended to limit the scope of the claims.

### Analytical Methods and Instrumentation

Proton nuclear magnetic resonance (NMR) spectra were obtained on Bruker Ascend™ 500 MHz spectrometer. NMR spectra are reported as follows: chemical shift S (ppm), multiplicity, coupling constant J (Hz), and (relative) integration. The abbreviations s=singlet, d=doublet, t=triplet, q=quartet, m=multiplet and br=broad are used throughout. Mass spectral data were measured using the following systems: Waters Acquity i-class ultra-performance liquid chromatography (UPLC) system with Acquity Photo Diode Array Detector, Acquity Evaporative Light Scattering Detector (ELSD), and Waters ZQ Mass Spectrometer. Data was acquired using Waters MassLynx 4.1 software and purity was characterized by UV wavelength 220 nm, evaporative light scattering detection (ELSD), and electrospray positive ion (ESI) (column: Acquity UPLC BEH C18 1.7μι2.1×50 mm). Solvents used: acetonitrile/water, containing 0.1% formic acid; flow rate 0.7 mL/min. Preparatory HPLC purifications were conducted with a flow rate of 15 mL/min and detection by UV wavelength at 214 nm and 254 nm (Column: Jupiter© 10 μM Proteo 90 Å, 250×21.2 mm A, solvent: acetonitrile/water, containing a modifier such as 0.1% trifluoroacetic acid).

Abbreviations used in the examples include:

| Abbreviation | Name |
|---|---|
| CH₃CN or | acetonitrile |
| ACN | |
| aq. | aqueous |
| atm | atmospheres |
| BINAP | (1,1′-binaphthalene-2,2′-diyl)bis(diphenylphosphine) |
| Boc | t-butoxycarbonyl |
| CCl₄ | carbon tetrachloride |
| CDCl₃ | deuterated chloroform |
| CO | carbon monoxide gas |
| CO₂ | carbon dioxide |
| Cs₂CO₃ | cesium carbonate |
| CuBr | copper(I) bromide |
| Cu(OAc)₂ | copper(II) acetate |
| DCM | dichloromethane |
| DEAD | diethyl azodicarboxylate |
| DIPEA or | diisopropylethylamine |
| DIEA | |
| DMF | N,N-dimethylformamide |
| DMSO | dimethylsulfoxide |
| equiv | equivalent |
| ESI | electrospray ionization |
| Et₃N | triethylamine |
| EtOAc | ethyl acetate |
| EtOH | ethanol |
| FA | formic acid |
| FC | flash chromatography |
| h | hours |
| H₂O | water |
| HATU | 1-[bis(dimethylamino)methylene]-1H-1,2,3-triazolo[4,5-b]pyridinium 3- |
| | oxide hexafluorophosphate |
| HCl | hydrogen chloride |
| HOAc | acetic acid |
| K₂CO₃ | potassium carbonate |
| KI | potassium iodide |
| KOH | potassium hydroxide |
| KOtBu | potassium tert-butoxide |
| LiAlH₄ | lithium aluminum hydride |
| LiOH | lithium hydroxide |
| MeOH | methanol |
| min | minutes |
| N₂ | nitrogen |
| Na₂CO₃ | sodium carbonate |
| Na₂SO₄ | sodium sulfate |
| NaH | sodium hydride |
| NaHCO₃ | sodium bicarbonate |
| NaOH | sodium hydroxide |
| NBS | N-bromosuccinimide |
| NH₄Cl | ammonium chloride |
| NMR | nuclear magnetic resonance |
| Pd₂(dba)₃ | tris(dibenzylideneacetone)dipalladium(0) |
| Pd(dppf)Cl₂ | [1,1′-bis(diphenylphosphino)ferrocene]dichloropalladium(II) |
| Pd/C | palladium on carbon |
| Pd(OAc)₂ | palladium(II) acetate |
| PyBOP | benzotriazol-1-yloxytripyrrolidinophosphonium hexafluorophosphate |
| Prep-TLC | preparatory thin layer chromatography |
| RuPhos-Pd-G2 | chloro(2-dicyclohexylphosphino-2′,6′-diisopropoxy-1,1′-biphenyl)[2-(2′- |
| | amino-1,1′-biphenyl)]palladium(II) |
| sat. | saturated |
| t-BuOH | tert-butanol |
| THF | tetrahydrofuran |
| T₃P | propylphosphonic anhydride |

### General Schemes For Preparing IAP Building Blocks

### Scheme A1

IAP-targeting LHM can be generally prepared according to Scheme A1.

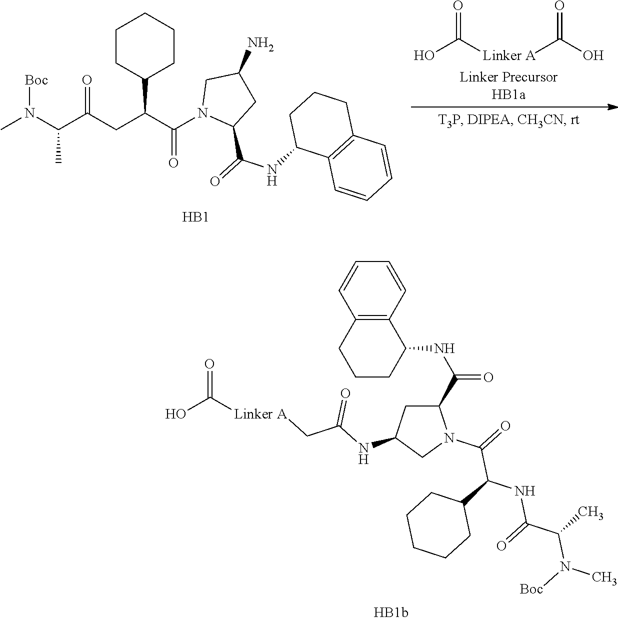

In certain embodiments, IAP-targeting LHM building blocks comprise coupling a linker precursor (HB1a) to tert-butyl (S)-1-((S)-2-((2S,4S)-4-amino-2-((R)-1,2,3,4-tetrahydronaphthalen-1-ylcarbamoyl)pyrrolidin-1-yl)-1-cyclohexyl-2-oxoethylamino)-1-oxopropan-2-yl(methyl)carbamate (HB1). Described below are detailed reaction procedures and additional examples of IAP-targeting LHM building blocks that may be prepared according to Scheme A1.

### Example 1. 3-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)propanoic acid (HB2)

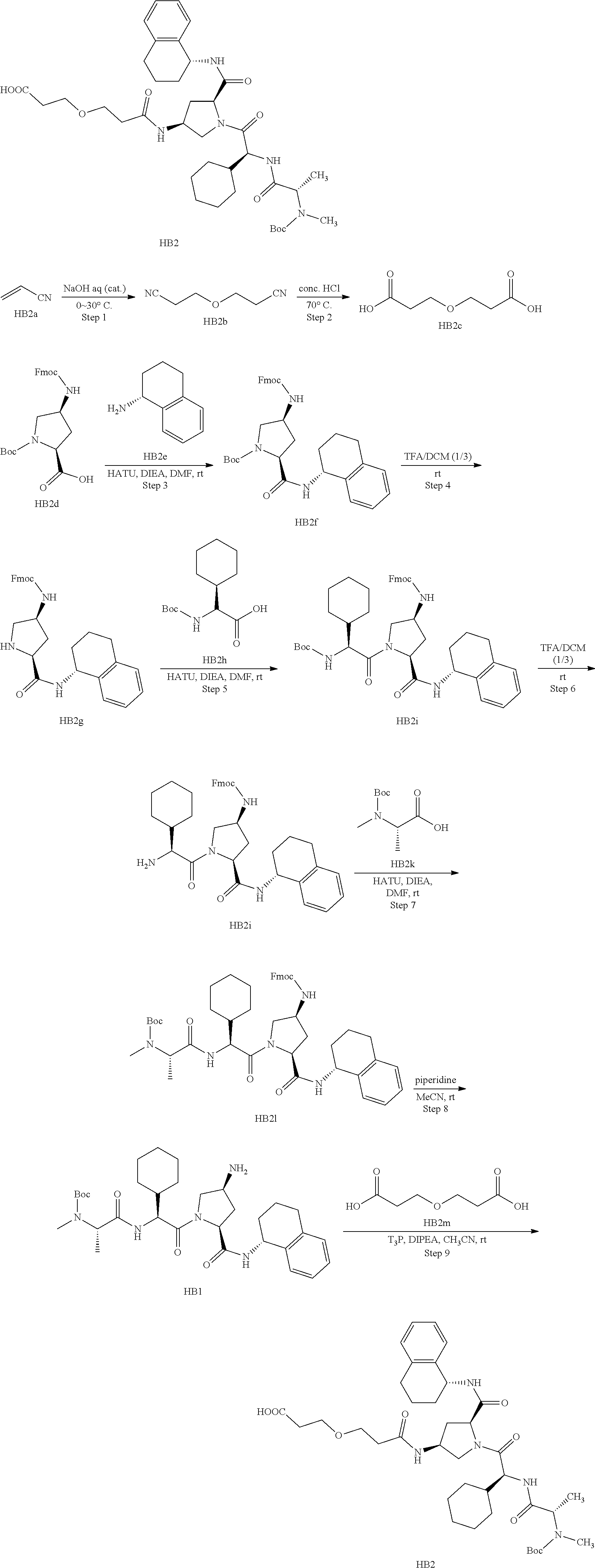

### Step 1: Synthesis of 3,3′-oxydipropanenitrile (HB2b)

To a stirred solution of aqueous NaOH (3 mL, 40% wt) was added acrylonitrile (17.5 g, 330 mmol) dropwise at 0° C. The solution was stirred at 30° C. for 16 h. When the reaction was completed, the reaction was diluted with H₂O (100 mL) and neutralized to pH 7 by HCl (2N). The aqueous solution was extracted with ethyl acetate (100 mL×3). The combined organic solution was dried over anhydrous Na₂SO₄ and concentrated to give 3,3′-oxydipropanenitrile (4.1 g, crude) as yellow oil, which was used in the next step without further purification. ¹H NMR (300 MHz, Chloroform-d) δ 3.74 (t, J=6.3 Hz, 4H), 2.65 (t, J=6.3 Hz, 4H).

### Step 2: Synthesis of 3,3′-oxydipropionic acid (HB2c)

A mixture of 3,3′-oxydipropanenitrile (4.1 g, 33 mmol) and concentrated HCl (38 mL) was stirred at 70° C. for 16 h. After cooling to room temperature, the solids were filtered out by filtration and the filtrate was concentrated under vacuum. The crude residue was purified by flash column chromatography with 30-100% ethyl acetate in petroleum ether to afford 3,3′-oxydipropionic acid (3.2 g, 12% over two steps) as yellow oil. ¹H NMR (400 MHz, DMSO-d₆) δ 12.20 (s, 2H), 3.62-3.55 (m, 4H), 2.42-2.40 (m, 4H).

### Step 3: Synthesis of (2S,4S)-tert-butyl 4-(((9H-fluoren-9-yl)methoxy)carbonylamino)-2-((R)-1,2,3,4-tetrahydronaphthalen-1-ylcarbamoyl)pyrrolidine-1-carboxylate (HB2f)

To a solution of (2S,4S)-4-(((9H-fluoren-9-yl)methoxy)carbonylamino)-1-(tert-butoxycarbonyl)pyrrolidine-2-carboxylic acid (10 g, 22.2 mmol), (R)-1,2,3,4-tetrahydronaphthalen-1-amine (3.26 g, 22.2 mmol), and DIEA (19 mL, 111 mmol) in DMF (100 mL) was added HATU (9.26 g, 24.4 mmol). The solution was stirred at room temperature for 3 h. The reaction was quenched by the addition of H₂O (200 mL) and then extracted with ethyl acetate (200 mL×3). The combined organic layer was washed with brine, dried over anhydrous Na₂SO₄, and concentrated under vacuum. The crude residue was purified by flash column chromatography with 10-50% ethyl acetate in petroleum ether to afford (2S,4S)-tert-butyl 4-(((9H-fluoren-9-yl)methoxy)carbonylamino)-2-((R)-1,2,3,4-tetrahydronaphthalen-1-ylcarbamoyl)pyrrolidine-1-carboxylate (12.0 g, 93%) as a white solid. MS (ESI) calculated for (C₃₅H₃₉N₃O₅) [M+H]⁺, 582.3; found, 582.0.

### Step 4: Synthesis of (9H-fluoren-9-yl)methyl ((3S,5S)-5-(((R)-1,2,3,4-tetrahydronaphthalen-1-yl)carbamoyl)pyrrolidin-3-yl)carbamate TFA salt (HB2g)

To a stirred solution of (2S,4S)-tert-butyl 4-(((9H-fluoren-9-yl)methoxy)carbonylamino)-2-((R)-1,2,3,4-tetrahydronaphthalen-1-ylcarbamoyl)pyrrolidine-1-carboxylate (12 g, 26.54 mmol) in DCM (120 mL) was added TFA (40 mL) at room temperature. The resulting mixture was stirred at room temperature overnight. The solvent was removed under vacuum to afford (9H-fluoren-9-yl)methyl ((3S,5S)-5-(((R)-1,2,3,4-tetrahydronaphthalen-1-yl)carbamoyl)pyrrolidin-3-yl)carbamate TFA salt (13 g, crude) as yellow oil, which was used in the next step without further purification. MS (ESI) calculated for (C₃₀H₃₁N₃O₃) [M+H]⁺, 482.2; found, 482.0.

### Step 5: Synthesis of 9H-fluoren-9-ylmethyl N-[(3S,5S)-1-[(2S)-2-[[(tert-butoxy)carbonyl]amino]-2-cyclohexylacetyl]-5-[[(1R)-1,2,3,4-tetrahydronaphthalen-1-yl]carbamoyl]pyrrolidin-3-yl]carbamate (HB2i)

To a stirred solution of (9H-fluoren-9-yl)methyl (3S,5S)-5-((R)-1,2,3,4-tetrahydronaphthalen-1-ylcarbamoyl)pyrrolidin-3-ylcarbamate TFA salt (13 g, 27.0 mmol), DIEA (23.5 mL, 135 mmol), and (S)-2-(tert-butoxycarbonylamino)-2-cyclohexylacetic acid (6.95 g, 27.0 mmol) in DMF (150 mL) was added HATU (12.33 g, 32.4 mmol). The resulting mixture was stirred at room temperature for 4 h. The reaction was quenched by the addition of H₂O (200 mL) and extracted with ethyl acetate (200 mL×3). The combined organic layer was washed with brine, dried over anhydrous Na₂SO₄, and concentrated under vacuum. The crude residue was purified by flash column chromatography with 10-40% ethyl acetate in petroleum ether to afford 9H-fluoren-9-ylmethyl N-[(3S,5S)-1-[(2S)-2-[[(tert-butoxy)carbonyl]amino]-2-cyclohexylacetyl]-5-[[(1R)-1,2,3,4-tetrahydronaphthalen-1-yl]carbamoyl]pyrrolidin-3-yl]carbamate (5.2 g, 27%) as a colorless oil. MS (ESI) calculated for (C₄₃H₅₂N₄O₆) [M+H]⁺, 721.4; found, 721.0.

### Step 6: Synthesis of (9H-fluoren-9-yl)methyl (3S,5S)-1-((S)-2-amino-2-cyclohexylacetyl)-5-((R)-1,2,3,4-tetrahydronaphthalen-1-ylcarbamoyl)pyrrolidin-3-ylcarbamate TFA salt (HB2j)

To a stirred solution of 9H-fluoren-9-ylmethyl N-[(3S,5S)-1-[(2S)-2-[[(tert-butoxy)carbonyl]amino]-2-cyclohexylacetyl]-5-[[(1R)-1,2,3,4-tetrahydronaphthalen-1-yl]carbamoyl]pyrrolidin-3-yl]carbamate (5.2 g, 7.22 mmol) in DCM (90 mL) was added TFA (30 mL). The solution was stirred at room temperature overnight. The solvents were removed under vacuum to afford (9H-fluoren-9-yl)methyl (3S,5S)-1-((S)-2-amino-2-cyclohexylacetyl)-5-((R)-1,2,3,4-tetrahydronaphthalen-1-ylcarbamoyl)pyrrolidin-3-ylcarbamate TFA salt (4.48 g, crude) as a yellow oil. MS (ESI) calculated for (C₃₈H₄₄N₄O₄) [M+H]⁺, 621.3; found, 621.0.

### Step 7: Synthesis of 9H-fluoren-9-ylmethyl N-[(3S,5S)-1-[(2S)-2-[(2S)-2-[[(tert-butoxy)carbonyl](methyl)amino]propanamido]-2-cyclohexylacetyl]-5-[[(1R)-1,2,3,4-tetrahydronaphthalen-1-yl]carbamoyl]pyrrolidin-3-yl]carbamate (HB2l)

To a stirred solution of (9H-fluoren-9-yl)methyl (3S,5S)-1-((S)-2-amino-2-cyclohexylacetyl)-5-((R)-1,2,3,4-tetrahydronaphthalen-1-ylcarbamoyl)pyrrolidin-3-ylcarbamate (4.48 g, 7.22 mmol), DIEA (4.66 g, 36.1 mmol), and (S)-2-(tert-butoxycarbonyl(methyl)amino)propanoic acid (1.46 g, 7.22 mmol) in DMF (50 mL) was added HATU (3.3 g, 8.68 mmol). The resulting mixture was stirred at room temperature for 3 h. The reaction was quenched by the addition of H₂O (100 mL) and extracted with ethyl acetate (100 mL×3). The combined organic layer was washed with brine, dried over anhydrous Na₂SO₄, and concentrated under vacuum. The crude residue was purified by flash column chromatography with 20-60% ethyl acetate in petroleum ether to afford 9H-fluoren-9-ylmethyl N-[(3S,5S)-1-[(2S)-2-[(2S)-2-[[(tert-butoxy)carbonyl](methyl)amino]propanamido]-2-cyclohexylacetyl]-5-[[(1R)-1,2,3,4-tetrahydronaphthalen-1-yl]carbamoyl]pyrrolidin-3-yl]carbamate (5.2 g, 89%) as a colorless oil. MS (ESI) calculated for (C₄₇H₅₉N₅O₇) [M+H]⁺, 806.4; found, 806.0.

### Step 8: Synthesis of tert-butyl (S)-1-((S)-2-((2S,4S)-4-amino-2-((R)-1,2,3,4-tetrahydronaphthalen-1-ylcarbamoyl)pyrrolidin-1-yl)-1-cyclohexyl-2-oxoethylamino)-1-oxopropan-2-yl(methyl)carbamate (HB1)

To a stirred solution of 9H-fluoren-9-ylmethyl N-[(3S,5S)-1-[(2S)-2-[(2S)-2-[[(tert-butoxy)carbonyl](methyl)amino]propanamido]-2-cyclohexylacetyl]-5-[[(1R)-1,2,3,4-tetrahydronaphthalen-1-yl]carbamoyl]pyrrolidin-3-yl]carbamate (5.2 g, 6.46 mmol) in acetonitrile (80 mL) was added piperidine (5.2 mL). The mixture was stirred at room temperature for one hour. The solids were filtered out by filtration and the filtrate was concentrated under vacuum. The crude residue was purified by reverse phase flash column chromatography with 5-95% acetonitrile in water to afford tert-butyl (S)-1-((S)-2-((2S,4S)-4-amino-2-((R)-1,2,3,4-tetrahydronaphthalen-1-ylcarbamoyl)pyrrolidin-1-yl)-1-cyclohexyl-2-oxoethylamino)-1-oxopropan-2-yl(methyl)carbamate (3.1656 g, 84%) as a white solid. ¹H NMR (300 MHz, DMSO-d₆) δ 8.45-8.12 (m, 1H), 7.71 (m, 1H), 7.39-6.99 (m, 4H), 4.94-4.91 (m, 1H), 4.61-4.45 (m, 1H), 4.34-4.19 (m, 2H), 3.90-3.88 (m, 1H), 3.29-3.16 (m, 1H), 2.75-2.72 (m, 5H), 2.50-2.27 (m, 1H), 2.01-1.82 (m, 4H), 1.81-1.50 (m, 9H), 1.41 (s, 9H), 1.29-0.85 (m, 9H). MS (ESI) calculated for (C₃₂H₄₉N₅O₅) [M+H]⁺, 584.4; found, 584.4.

### Step 9: Synthesis of 3-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)propanoic acid (HB2)

To a stirred solution of tert-butyl ((S)-1-(((S)-2-((2S,4S)-4-amino-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate (1.5 g, 2.57 mmol), 3,3′-oxydipropionic acid (2.78 g, 12.86 mmol), and DIEA (1.65 g, 12.86 mmol) in acetonitrile (30 mL) was added T₃P (12.3 g, 10.28 mmol, 50% in ethyl acetate) under nitrogen. The solution was stirred at 20° C. for 16 h. When the reaction was completed, the reaction was quenched by the addition of H₂O (50 mL) and the aqueous solution was extracted with ethyl acetate (50 mL×3). The combined organic layer was washed with brine, dried over anhydrous Na₂SO₄, and concentrated under vacuum. The crude residue was purified by reverse phase flash column chromatography with 5-50% acetonitrile in water to afford 3-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)propanoic acid (1.0929 g, 58%) as a white solid. ¹H NMR (400 MHz, Methanol-d₄) δ 7.48-7.36 (m, 1H), 7.23-7.03 (m, 3H), 5.07-5.06 (m, 1H), 4.63-4.30 (m, 4H), 4.21-4.18 (m, 1H), 3.72-3.67 (m, 4H), 3.55-3.51 (m, 1H), 2.91 (s, 3H), 2.91-2.73 (m, 2H), 2.67-2.41 (m, 5H), 2.04-1.61 (m, 11H), 1.49 (s, 9H), 1.38-1.00 (m, 8H). MS (ESI) calculated for (C₃₈H₅₇N₅O₉) [M+H]⁺, 728.4; found, 728.7.

### Example 2. Synthesis of 16-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-16-oxo-4,7,10,13-tetraoxahexadecanoic acid (HB3)

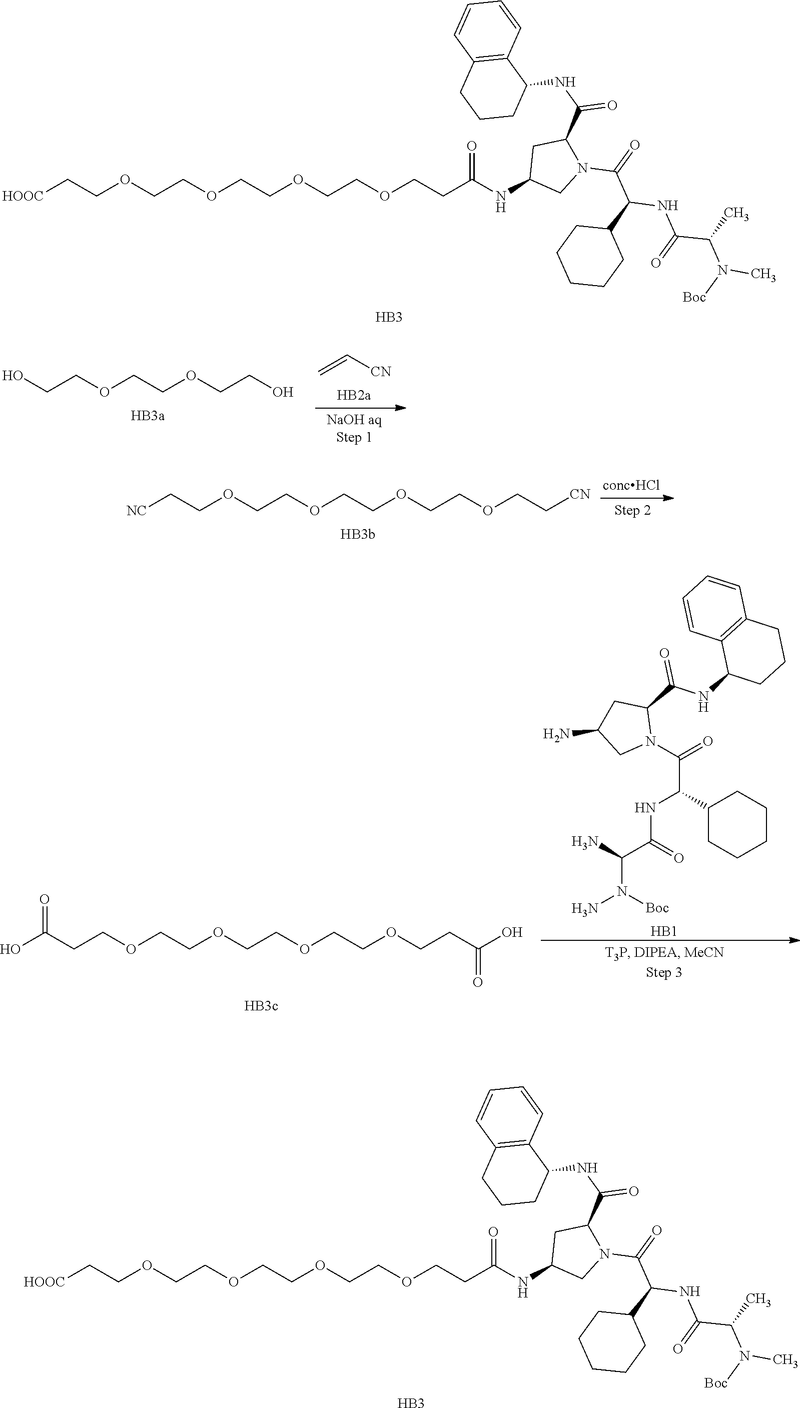

### Step 1: Synthesis of 4,7,10,13-tetraoxahexadecanedinitrile (HB3b)

To a stirred solution of 2,2′-(ethane-1,2-diylbis(oxy))bis(ethan-1-ol) (15 g, 99.90 mmol) and NaOH aqueous (1.2 mL, 40% wt) was added acrylonitrile (12.2 g, 230 mmol) dropwise at 0° C. The solution was stirred at 30° C. for 16 h. When the reaction was complete, the reaction was quenched by the addition of H₂O (100 mL), neutralized to pH 7 by HCl (1 N). The aqueous solution was extracted with ethyl acetate (100 mL×3). The combined organic solution was dried over Na₂SO₄, filtered, and concentrated to afford 4,7,10,13-tetraoxahexadecanedinitrile (15 g, 59%) as a yellow oil. ¹H NMR (300 MHz, Chloroform-d) δ 3.72 (t, J=6.3 Hz, 4H), 3.69-3.62 (m, 12H), 2.62 (t, J=6.3 Hz, 4H).

### Step 2: Synthesis of 4,7,10,13-tetraoxahexadecanedioic acid (HB3c)

Concentrated HCl (68 mL) was added to 4,7,10,13-tetraoxahexadecanedinitrile (15 g, 58.60 mmol). The solution was stirred at 70° C. overnight. When the reaction was completed, the mixture was filtered. The filtrate was concentrated to afford a residue which was purified by flash column chromatography with 30-100% ethyl acetate in petroleum ether to afford 4,7,10,13-tetraoxahexadecanedioic acid (10.0 g,) as a yellow oil. ¹H NMR (400 MHz, DMSO-d₆) δ 12.18 (s, 2H), 3.60 (t, J=6.4 Hz, 4H), 3.51-3.48 (m, 12H), 2.44 (t, J=6.4 Hz, 4H).

### Step 3: Synthesis of 16-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-16-oxo-4,7,10,13-tetraoxahexadecanoic acid (HB3)

To a stirred solution of tert-butyl ((S)-1-(((S)-2-((2S,4S)-4-amino-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate (1.5 g, 2.57 mmol), 4,7,10,13-tetraoxahexadecanedioic acid (5.0 g, 12.90 mmol), and DIPEA (1.7 g, 12.86 mmol) in acetonitrile (30 mL) under nitrogen was added T₃P (6.5 g, 10.28 mmol). The solution was stirred at 20° C. for 16 h. When the reaction was completed, the reaction was quenched by the addition of H₂O (50 mL). The aqueous solution was extracted with ethyl acetate (30 mL×3). The combined organic solution was dried over Na₂SO₄ and filtered to give a residue which was purified by reverse phase flash chromatography (FC) with 5-50% acetonitrile in H₂O to afford 16-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-16-oxo-4,7,10,13-tetraoxahexadecanoic acid (511.2 mg, 23%) as a white solid. ¹H NMR (400 MHz, Methanol-d₄) δ 7.44-7.38 (m, 1H), 7.21-7.12 (m, 2H), 7.12-7.06 (m, 1H), 5.06 (t, J=6.0 Hz, 1H), 4.67-4.38 (m, 4H), 4.22-4.18 (m, 1H), 3.74-3.70 (m, 4H), 3.68-3.59 (m, 11H), 3.55-3.50 (m, 1H), 2.91-2.73 (m, 5H), 2.60-2.41 (m, 5H), 1.93-1.83 (m, 6H), 1.82-1.66 (m, 5H), 1.49 (s, 9H), 1.39-0.98 (m, 9H). MS (ESI) calculated for (C₄₄H₆₉N₅O₁₂) [M+1]⁺, 860.7; found, 860.7.

IAP-targeting LHM can be generally prepared according to Scheme B1

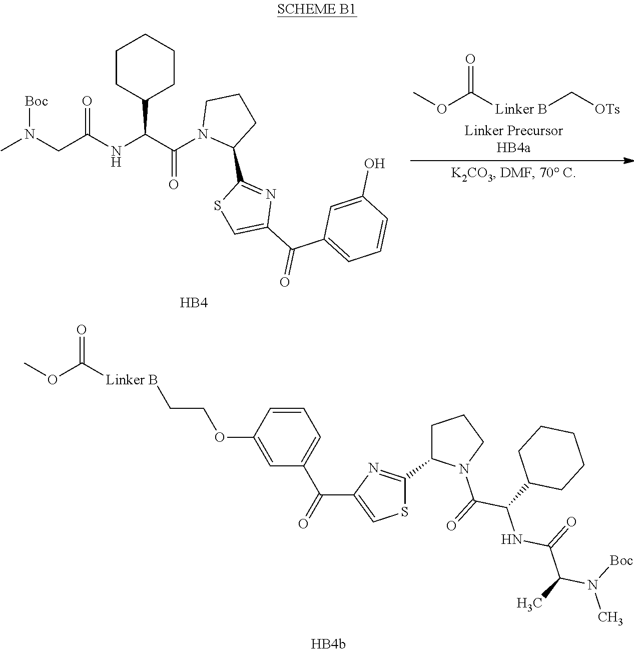

In certain embodiments, IAP-targeting LHM building blocks comprise coupling a linker precursor (HB4a) to tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (HB4). Described below are detailed reaction procedures and additional examples of IAP-targeting LHM building blocks that may be prepared according to Scheme B1.

### Example 3. tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (HB4)

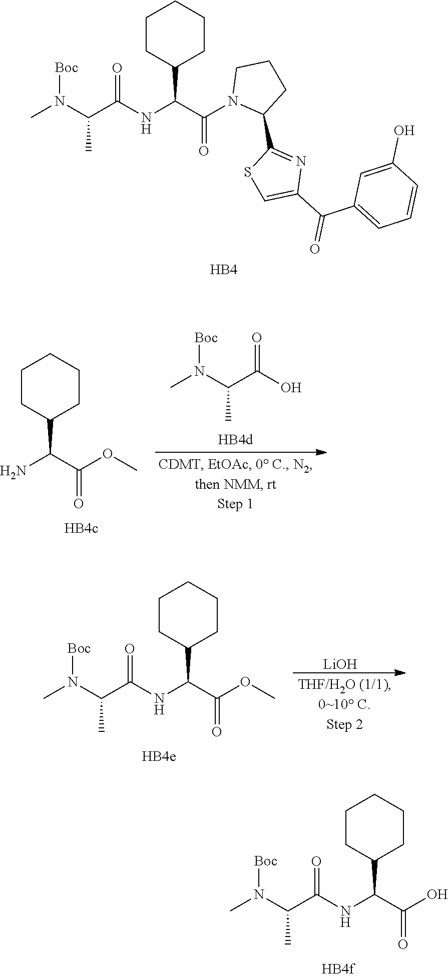

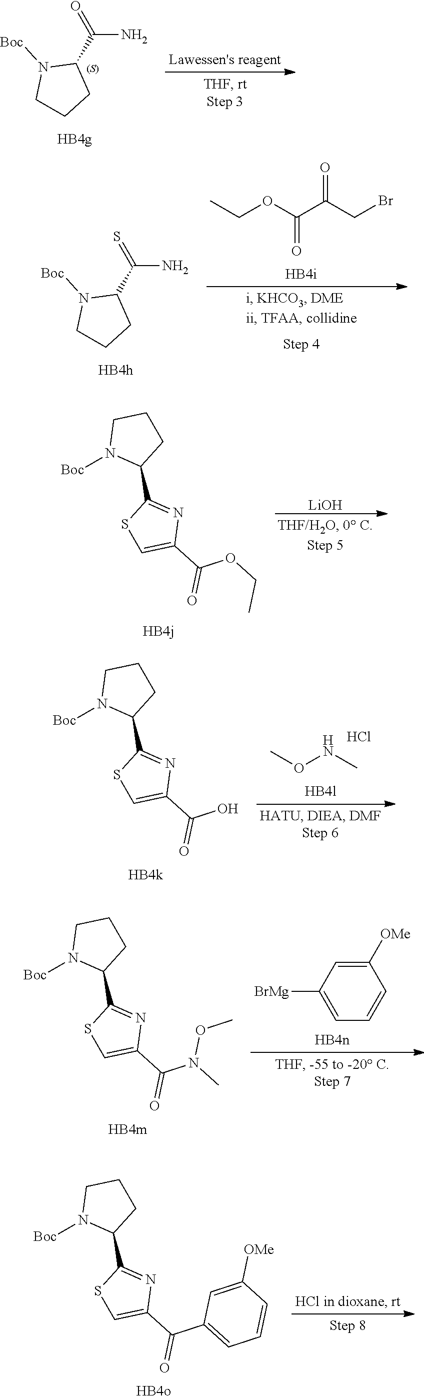

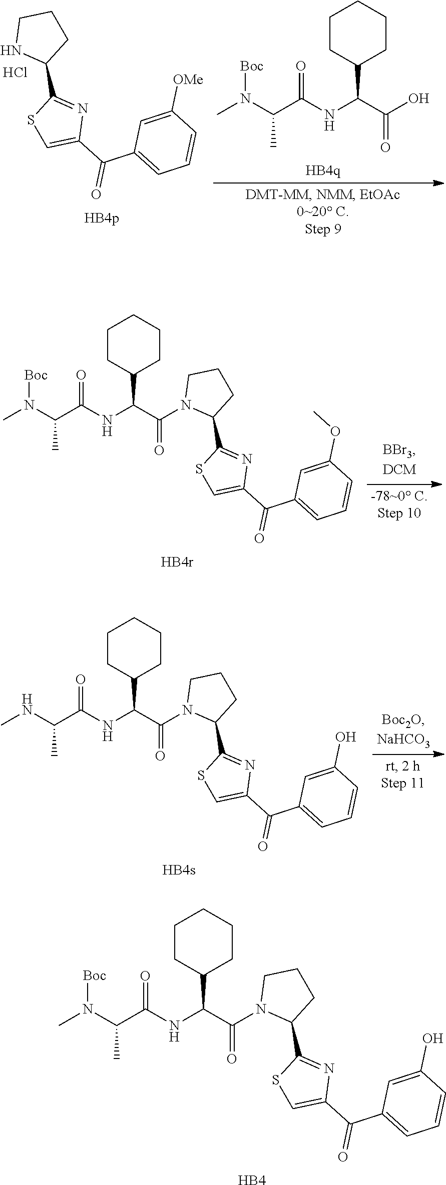

### Step 1: Synthesis of methyl (S)-2-((S)-2-((tert-butoxycarbonyl)(methyl)amino)propanamido)-2-cyclohexylacetate (HB4e)

A solution of (S)-methyl-2-amino cyclohexyl acetate hydrochloride (70.0 g, 0.34 mol) and (S)-2-(tert-butoxycarbonyl(methyl)amino)propanoic acid (69.0 g, 0.34 mol) in ethyl acetate (300 mL) was treated with 2-chloro-4,6-dimethoxy-1,3,5-triazine (CDMT) (64.7 g, 0.37 mol) under nitrogen. The reaction mixture was cooled to 0° C. and treated with N-methylmorpholine (85.8 g, 0.85 mol). The reaction mixture was warmed to room temperature and stirred for 4 h. The solid precipitate was filtered out and rinsed with ethyl acetate. The filtrate was washed with saturated NaHCO₃ aqueous solution and then 10% citric acid and then brine, dried over anhydrous sodium sulfate, and concentrated under vacuum to afford methyl (S)-2-((S)-2-((tert-butoxycarbonyl)(methyl)amino)propanamido)-2-cyclohexylacetate (85.0 g, 71%) as an off-white solid. MS (ESI) calculated for (C₁₈H₃₂N₂O₅) [M+H]⁺, 357.2; found, 357.0.

### Step 2: Synthesis of (S)-2-((S)-2-((tert-butoxycarbonyl)(methyl)amino)propanamido)-2-cyclohexylacetic acid (HB4f)

To a solution of methyl (S)-2-((S)-2-((tert-butoxycarbonyl)(methyl)amino)propanamido)-2-cyclohexylacetate (85.0 g, 0.24 mol) in THE (1.2 L) was added a solution of LiOH H₂O (25.2 g, 0.60 mol) in water (1.2 L) while maintaining the temperature of the mixture at 0-10° C. under nitrogen. The resulting mixture was stirred at 0-10° C. for 3 h. The organic solvent was removed under vacuum and the pH value of aqueous phase was adjusted to ˜3 via citric acid. The mixture was extracted with ethyl acetate twice. The combined organic layer was washed with brine, dried over anhydrous sodium sulfate, and concentrated under vacuum to afford (S)-2-((S)-2-((tert-butoxycarbonyl)(methyl)amino)propanamido)-2-cyclohexylacetic acid (100 g, crude) as a colorless oil, which was used in the next step without further purification. MS (ESI) calculated for (C₁₇H₃₀N₂O₅) [M−H]⁺, 341.2; found, 341.0.

### Step 3: Synthesis of tert-butyl (S)-2-carbamothioylpyrrolidine-1-carboxylate (HB4h)

To a solution of tert-butyl (2S)-2-carbamoylpyrrolidine-1-carboxylate (100 g, 466.72 mmol) in tetrahydrofuran (1.2 L) was added Lawesson's reagent (113 g, 279.70 mmol). The resulting mixture was stirred at room temperature for 16 h. The mixture was then diluted with saturated NaHCO₃ aqueous solution and extracted with ethyl acetate. The combined organic layer was washed with brine, dried over anhydrous sodium sulfate, and concentrated under vacuum to afford tert-butyl (S)-2-carbamothioylpyrrolidine-1-carboxylate (110 g, crude) as a white solid, which was used in the next step without further purification. MS (ESI) calculated for (C₁₀H₁₈N₂O₂S) [M+H]⁺, 231.1; found, 231.0.

### Step 4: Synthesis of ethyl (S)-2-(1-(tert-butoxycarbonyl)pyrrolidin-2-yl)thiazole-4-carboxylate (HB4j)

To a mixture of tert-butyl (S)-2-carbamothioylpyrrolidine-1-carboxylate (100.0 g, 0.44 mol) and potassium bicarbonate (348.0 g, 3.48 mol) in dimethoxyethane (1.5 L) was added ethyl 3-bromo-2-oxopropanoate (253.1 g, 1.30 mol) dropwise at room temperature. The resulting mixture was stirred at room temperature for one hour and then cooled to 0° C. Then trifluoroacetic acid (365.4 g, 1.74 mol) and collidine (298.2 g, 2.78 mol) were added dropwise to the above solution at 0° C. The resulting mixture was stirred at room temperature for 8 h. The reaction was quenched by the addition of water and the aqueous phase was extracted with dichloromethane. The combined organic layer was washed with HCl (0.5 N) and brine, dried over anhydrous sodium sulfate, and concentrated under vacuum. The crude residue was purified by flash column chromatography with 10-30% ethyl acetate in petroleum ether to afford ethyl (S)-2-(1-(tert-butoxycarbonyl)pyrrolidin-2-yl)thiazole-4-carboxylate (51.5 g, 34% over two steps) as a brown solid. MS (ESI) calculated for (C₁₅H₂₂N₂O₄S) [M+H]⁺, 327.1; found, 327.0.

### Step 5: Synthesis of (S)-2-(1-(tert-butoxycarbonyl)pyrrolidin-2-yl)thiazole-4-carboxylic acid (HB4k)

To a mixture of ethyl (S)-2-(1-(tert-butoxycarbonyl)pyrrolidin-2-yl)thiazole-4-carboxylate (51.5 g, 0.16 mol) in THE (300 mL) and water (200 mL) was added a solution of lithium hydroxide hydrate (26.5 g, 0.63 mol) in water (100 mL) dropwise at 0° C. The resulting mixture was stirred at 0° C. for 5 h. The organic layer was removed under vacuum. The residue was diluted with water (200 mL) and the pH value was adjusted to three via HCl (6N). The solution was extracted with ethyl acetate. The combined organic layer was washed with brine, dried over anhydrous sodium sulfate, and concentrated under vacuum to afford (S)-2-(1-(tert-butoxycarbonyl)pyrrolidin-2-yl)thiazole-4-carboxylic acid (45.0 g, 95%) as a light brown solid. MS (ESI) calculated for (C₁₃H₁₈N₂O₄S) [M−H]⁻, 297.1; found, 297.0.

### Step 6: Synthesis of tert-butyl (S)-2-(4-(methoxy(methyl)carbamoyl)thiazol-2-yl)pyrrolidine-1-carboxylate (HB4m)

A mixture of (S)-2-(1-(tert-butoxycarbonyl)pyrrolidin-2-yl)thiazole-4-carboxylic acid (90.0 g, 0.30 mol), methoxy(methyl)amine hydrogen chloride (43.6 g, 0.45 mol), HATU (114.0 g, 0.30 mol), and DIEA (96.7 g, 0.75 mol) in DMF (500 mL) was stirred at room temperature for 16 h. The mixture was diluted with water and the aqueous phase was extracted with ethyl acetate. The combined organic layer was washed with brine, dried over anhydrous sodium sulfate, and concentrated under vacuum. The crude residue was purified by flash column chromatography with 40-80% ethyl acetate in petroleum ether to afford tert-butyl (S)-2-(4-(methoxy(methyl)carbamoyl)thiazol-2-yl)pyrrolidine-1-carboxylate (60.0 g, 59%) as a light yellow oil. MS (ESI) calculated for (C₁₅H₂₃N₃O₄S) [M+H]⁺, 342.1; found, 342.0.

### Step 7: Synthesis of tert-butyl (S)-2-(4-(3-methoxybenzoyl)thiazol-2-yl)pyrrolidine-1-carboxylate (HB4o)

To a solution of tert-butyl (S)-2-(4-(methoxy(methyl)carbamoyl)thiazol-2-yl)pyrrolidine-1-carboxylate (30.0 g, 88.0 mmol) in anhydrous THE (300 mL) was added (3-methoxyphenyl)magnesium bromide (1 M in THF, 530 mL, 0.53 mol) dropwise at −55° C. under nitrogen. The resulting mixture was stirred for 4 h below −20° C. The reaction was then quenched by the addition of saturated NH₄Cl aqueous solution at 0° C. cautiously. The mixture was extracted with ethyl acetate. The combined organic layer was washed with brine, dried over anhydrous sodium sulfate, and concentrated under vacuum. The crude residue was purified by flash column chromatography with 10-50% ethyl acetate in petroleum ether to afford tert-butyl (S)-2-(4-(3-methoxybenzoyl)thiazol-2-yl)pyrrolidine-1-carboxylate (24 g, 70%) as a light yellow oil. MS (ESI) calculated for (C₂₀H₂₄N₂O₄S) [M+H]⁺, 389.1; found, 389.0.

### Step 8: Synthesis of (S)-(3-methoxyphenyl)(2-(pyrrolidin-2-yl)thiazol-4-yl)methanone HCl salt (HB4p)

A mixture of tert-butyl (S)-2-(4-(3-methoxybenzoyl)thiazol-2-yl)pyrrolidine-1-carboxylate (24 g, 61.8 mmol) in HCl (4 M in dioxane, 200 mL) was stirred at room temperature for 2 h. The solvent was removed under vacuum to afford (S)-(3-methoxyphenyl)(2-(pyrrolidin-2-yl)thiazol-4-yl)methanone HCl salt (26 g, crude) as a yellow oil, which was used in the next step without further purification. MS (ESI) calculated for (C₂₀H₁₆N₂O₂S) [M+H]⁺, 289.1; found, 289.0.

### Step 9: Synthesis of tert-butyl ((S)-1-(((S)-1-cyclohexyl-2-((S)-2-(4-(3-methoxybenzoyl)thiazol-2-yl)pyrrolidin-1-yl)-2-oxoethyl)amino)-1-oxopropan-2-yl)(methyl)carbamate

To a solution of 4-[(3-methoxyphenyl)carbonyl]-2-[(2S)-pyrrolidin-2-yl]-1,3-thiazole (25 g, 86.70 mmol) and (2S)-2-[(2S)-2-[[(tert-butoxy)carbonyl](methyl)amino]propanamido]-2-cyclohexylacetic acid (29.7 g, 86.73 mmol) in ethyl acetate (400 mL) was added 4-(4,6-dmethoxy-1,3,5-triazine-2-yl)-4-methyl morpholinium chloride (DMT-MM) (26.35 g, 95.47 mmol) and 4-methylmorpholine (21.9 g, 216.83 mmol) at 0° C. The resulting mixture was stirred at room temperature for 3 h. The reaction was then quenched by the addition of water and the aqueous phase was extracted with ethyl acetate. The combined organic layer was washed with brine, dried over anhydrous sodium sulfate, and concentrated under vacuum. The crude residue was purified by flash column chromatography with 0-30% ethyl acetate in petroleum ether to afford tert-butyl ((S)-1-(((S)-1-cyclohexyl-2-((S)-2-(4-(3-methoxybenzoyl)thiazol-2-yl)pyrrolidin-1-yl)-2-oxoethyl)amino)-1-oxopropan-2-yl)(methyl)carbamate (24 g, 46%) as a light yellow oil. MS (ESI) calculated for (C₃₂H₄₄N₄O₆S) [M+H]⁺, 613.3; found, 613.0.

### Step 10: Synthesis of (S)—N—((S)-1-cyclohexyl-2-((S)-2-(4-(3-hydroxybenzoyl)thiazol-2-yl)pyrrolidin-1-yl)-2-oxoethyl)-2-(methylamino)propanamide (HB4r)

To a solution of tert-butyl ((S)-1-(((S)-1-cyclohexyl-2-((S)-2-(4-(3-methoxybenzoyl)thiazol-2-yl)pyrrolidin-1-yl)-2-oxoethyl)amino)-1-oxopropan-2-yl)(methyl)carbamate (9.0 g, 14.69 mmol) in dichloromethane (120 mL) was added BBr₃ (10.9 g, 44.1 mmol) dropwise at −78° C. The resulting mixture was stirred below 0° C. for 4 h under nitrogen. The reaction was then quenched by the addition of water cautiously and the aqueous phase was extracted with dichloromethane. The combined organic layer was washed with brine, dried over anhydrous sodium sulfate, and concentrated under vacuum to afford (S)—N—((S)-1-cyclohexyl-2-((S)-2-(4-(3-hydroxybenzoyl)thiazol-2-yl)pyrrolidin-1-yl)-2-oxoethyl)-2-(methylamino)propanamide (9 g, crude) as a light brown oil, which was used in the next step without further purification. MS (ESI) calculated for (C₂₆H₃₄N₄O₄S) [M+H]⁺, 499.2; found, 499.0.

### Step 11: Synthesis of tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (HB4)

To a solution of (S)—N—((S)-1-cyclohexyl-2-((S)-2-(4-(3-hydroxybenzoyl)thiazol-2-yl)pyrrolidin-1-yl)-2-oxoethyl)-2-(methylamino)propanamide (10 g, 20.05 mmol) and sodium bicarbonate (3.6 g, 43.21 mmol) in dioxane (120 mL) was added a solution of Boc₂O (5.6 g, 25.48 mmol) in dioxane (30 mL) dropwise at 0° C. The mixture was stirred at room temperature for 2 h. The reaction was diluted with water and the aqueous phase was extracted with ethyl acetate. The combined organic layer was washed with brine, dried over anhydrous sodium sulfate and concentrated under vacuum. The crude residue was purified by flash column chromatography with 10-50% ethyl acetate in petroleum ether to afford tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (5.2 g, 59% over two steps) as a light yellow oil. MS (ESI) calculated for (C₃₁H₄₂N₄O₆S) [M+H]⁺, 599.3; found, 599.3. ¹H NMR (300 MHz, Chloroform-d) δ 8.60 (br s, 1H), 8.09 (d, J=2.0 Hz, 1H), 7.78-7.54 (m, 2H), 7.38-7.34 (m, 1H), 7.11-7.08 (m, 1H), 6.79 (br s, 1H), 5.68-5.47 (m, 1H), 4.85-4.64 (m, 2H), 4.00-3.59 (m, 2H), 2.80 (s, 3H), 2.58-2.09 (m, 4H), 1.87-1.58 (m, 6H), 1.50 (s, 9H), 1.36 (d, J=7.1 Hz, 3H), 1.18-0.81 (m, 5H).

### Example 4. 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoic acid (HB5)

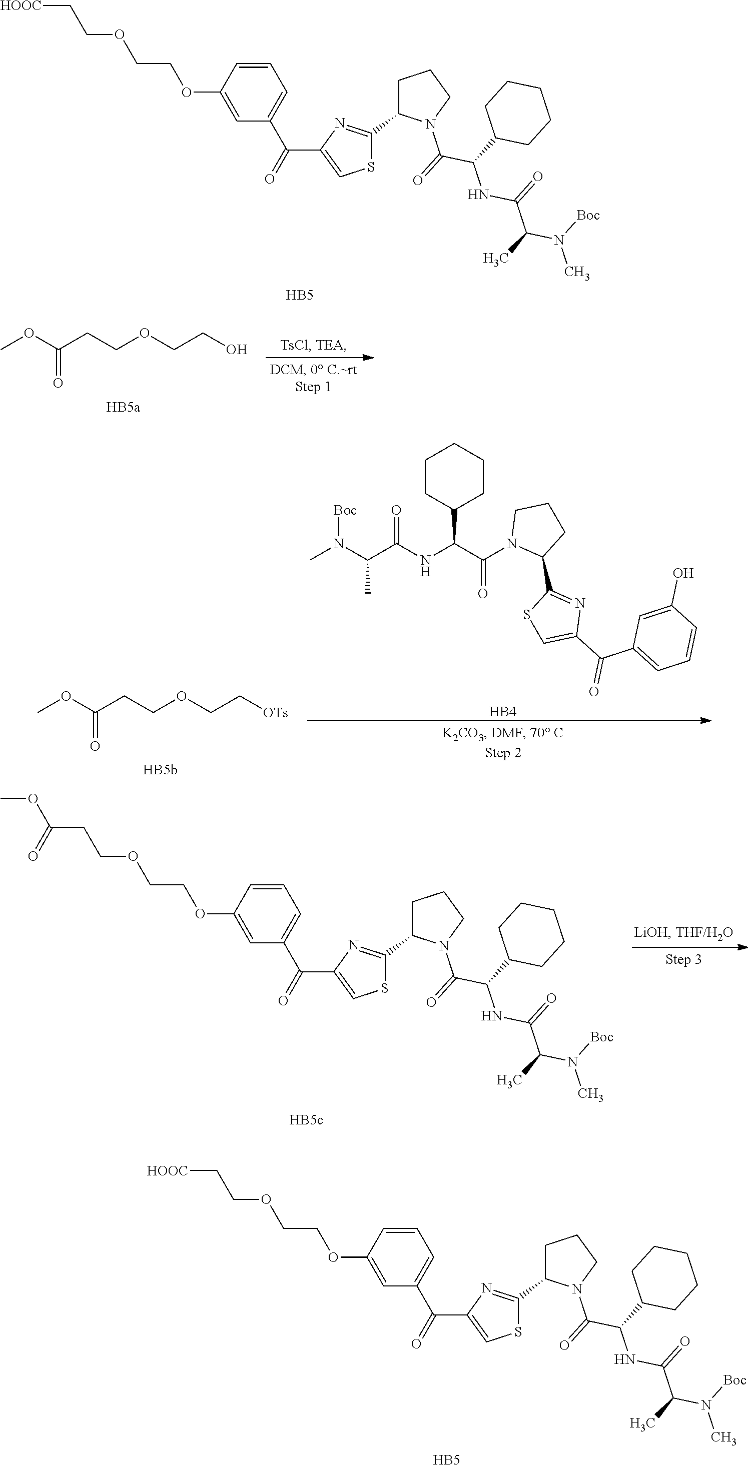

### Step 1: Synthesis of methyl 3-[2-[(4-methylbenzenesulfonyl)oxy]ethoxy]propanoate (HB5b)

To a solution of methyl 3-(2-hydroxyethoxy)propanoate (1.00 g, 6.7 mmol) in dichloromethane (15 mL) was added triethylamine (1.72 g, 13.2 mmol) and p-TsCl (1.54 g, 8.1 mmol) at room temperature. The mixture was stirred at room temperature for 16 h. The reaction mixture was quenched by the addition of water and the aqueous phase was extracted with dichloromethane. The combined organic layer was washed with brine, dried over anhydrous sodium sulfate, filtered, and concentrated under vacuum. The residue was purified by flash column chromatography with 0-50% ethyl acetate in petroleum ether to afford methyl 3-[2-[(4-methylbenzenesulfonyl)oxy]ethoxy]propanoate (1.05 g, 51%) as a yellow oil. MS (ESI) calculated for (C₁₃H₁₈O₆S) [M+H]⁺, 303.1; found, 303.0.

### Step 2: Synthesis of methyl 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoate (HB5c)

To a solution of methyl 3-[2-[(4-methylbenzenesulfonyl)oxy]ethoxy]propanoate (1.05 g, 3.5 mmol) in N,N-dimethylformamide (10 mL) was added tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (1.32 g, 2.2 mmol) and potassium carbonate (607 mg, 4.4 mmol). The mixture was stirred at 70° C. for 16 h. After cooling to room temperature, the reaction mixture was diluted with water and the aqueous phase was extracted with ethyl acetate. The combined organic layer was washed with brine, dried over anhydrous sodium sulfate, filtered, and concentrated under vacuum. The residue was purified by flash column chromatography with 0-50% ethyl acetate in petroleum ether to afford methyl 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoate (1.0 g, 62%) as a light yellow oil. MS (ESI) calculated for (C₃₆H₅₀N₄O₉S) [M+H]⁺, 715.3; found, 715.0.

### Step 3: Synthesis of 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoic acid (HB5)

To a solution of 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoic acid (1.0 g, 1.37 mmol) in tetrahydrofuran (5 mL) and H₂O (5 mL) was added lithium hydroxide hydrate (115 mg, 2.75 mmol). The mixture was stirred at room temperature for 5 h. The reaction mixture was diluted with water and adjusted to pH -3 via HCl (2 N). The mixture was extracted with ethyl acetate. The combined organic layer was washed with brine, dried over anhydrous sodium sulfate, and concentrated under vacuum. The crude residue was purified by reverse phase flash column chromatography with 5-55% acetonitrile in water to afford 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoic acid (733.9 mg, 75%) as a white solid. ¹H NMR (300 MHz, Methanol-d₄) δ 8.33 (s, 1H), 7.77-7.65 (m, 2H), 7.52-7.39 (m, 1H), 7.26-7.24 (m, 1H), 5.70-5.46 (m, 1H), 4.71-4.42 (m, 2H), 4.28-4.16 (m, 2H), 4.05-3.72 (m, 6H), 2.80 (s, 3H), 2.49 (t, J=7.2 Hz, 2H), 2.44-2.03 (m, 4H), 1.89-1.55 (m, 6H), 1.49 (s, 9H), 1.37-1.35 (m, 3H), 1.30-0.95 (m, 5H). MS (ESI) calculated for (C₃₆H₅₀N₄O₉S) [M+H]⁺, 715.3; found, 715.5.

### Example 5. 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oic acid (HB6)

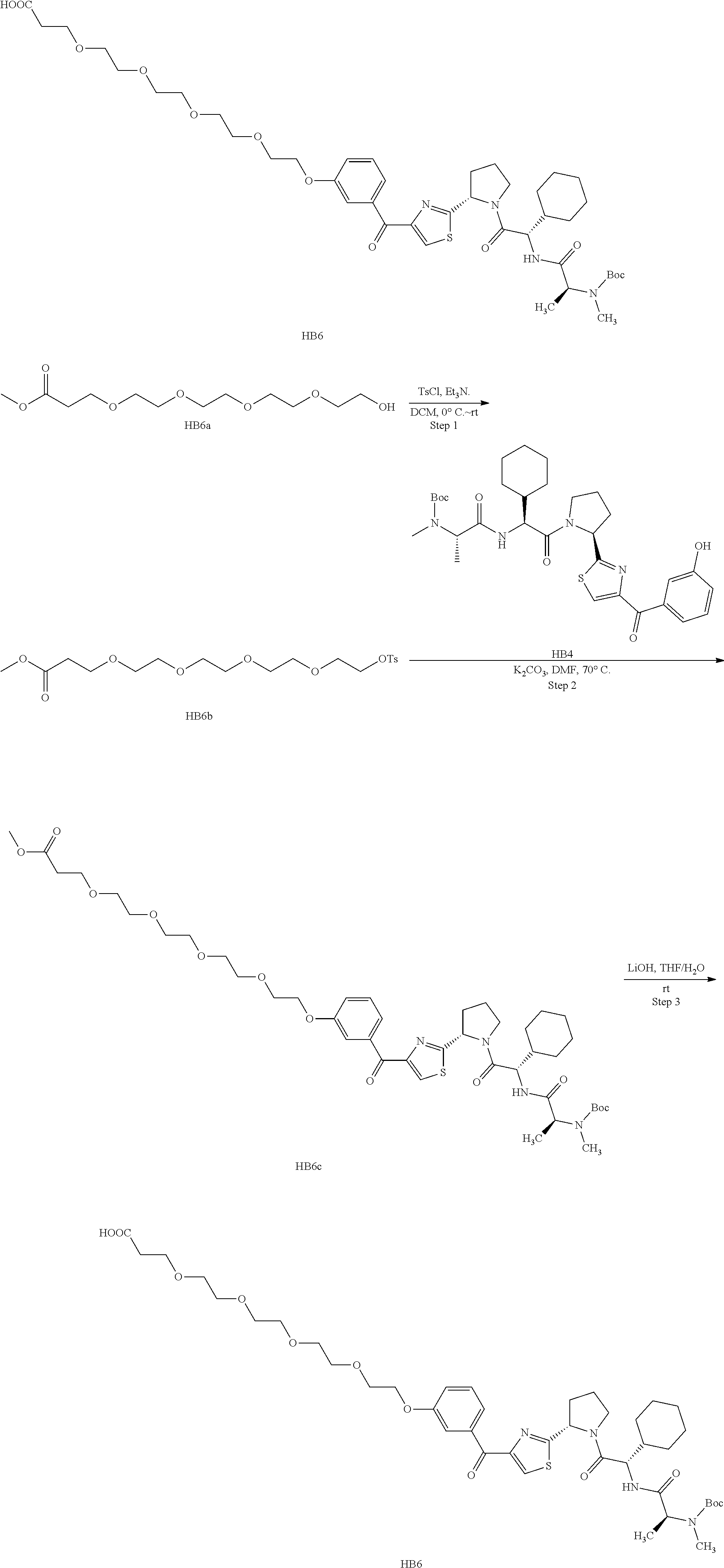

### Step 1: Synthesis of methyl 1-[(4-methylbenzenesulfonyl)oxy]-3,6,9,12-tetraoxapentadecan-15-oate (HB6b)

To a solution of methyl 1-hydroxy-3,6,9,12-tetraoxapentadecan-15-oate (970 mg, 3.46 mmol) in dichloromethane (20 mL) was added triethylamine (700 mg, 6.93 mmol) and p-TsCl (990 mg, 5.19 mmol). The mixture was stirred at room temperature for 16 h. The reaction mixture was diluted with water and the aqueous phase was extracted with ethyl acetate. The combined organic layer was washed with brine, dried over anhydrous sodium sulfate, filtered, and concentrated under vacuum. The residue was purified by flash column chromatography with 0-50% ethyl acetate in petroleum ether to afford methyl 1-[(4-methylbenzenesulfonyl)oxy]-3,6,9,12-tetraoxapentadecan-15-oate (1.28 g, 85%) as a light yellow oil. MS (ESI) calculated for (C₁₉H₃₀O₉S) [M+H]⁺, 435.2; found, 435.0.

### Step 2: Synthesis of methyl 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oate (HB6c)

To a solution of methyl 1-[(4-methylbenzenesulfonyl)oxy]-3,6,9,12-tetraoxapentadecan-15-oate (1.28 g, 2.95 mmol) in N,N-dimethylformamide (10 mL) was added tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (1.17 g, 1.96 mmol) and potassium carbonate (370 mg, 2.68 mmol). The mixture was stirred at 50° C. for 16 h. The reaction mixture was then diluted with water and the aqueous phase was extracted with ethyl acetate. The combined organic layer was washed with brine, dried over anhydrous sodium sulfate, filtered, and concentrated under vacuum. The residue was purified by flash column chromatography with 0-50% ethyl acetate in petroleum ether to afford methyl 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oate (1.2 g, 60%) as a light yellow oil. MS (ESI) calculated for (C₄₃H₆₄N₄O₁₂S) [M+H]⁺, 861.4; found, 861.0.

### Step 3: Synthesis of 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oic acid (HB6)

To a solution of 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oate (1.2 g, 1.39 mmol) in tetrahydrofuran (10 mL) and H₂O (10 mL) was added lithium hydroxide hydrate (140 mg, 3.33 mmol). The mixture was stirred at room temperature for 16 h. The reaction mixture was diluted with water and the pH was adjusted to ˜3 via HCl (2 N). The aqueous phase was extracted with ethyl acetate. The combined organic layer was washed with brine, dried over anhydrous sodium sulfate, filtered, and concentrated under vacuum. The residue was purified by flash column chromatography with 10-80% acetonitrile in water to afford 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oic acid (805.2 mg, 68%) as a white solid. ¹H NMR (300 MHz, Methanol-d₄) δ 8.35 (s, 1H), 7.80-7.69 (m, 2H), 7.50-7.42 (m, 1H), 7.26-7.23 (m, 1H), 5.50-5.45 (m, 1H), 4.56-4.53 (m, 2H), 4.32-4.16 (m, 2H), 4.05-3.85 (m, 4H), 3.77-3.66 (m, 6H), 3.66-3.56 (m, 8H), 2.80 (s, 3H), 2.45 (t, J=7.2 Hz, 2H), 2.41-2.08 (m, 3H), 1.88-1.55 (m, 6H), 1.49 (s, 9H), 1.36 (d, J=7.2 Hz, 3H), 1.08-1.04 (m, 6H). MS (ESI) calculated for (C₄₂H₆₂N₄O₁₂S) [M+H]⁺, 847.4; found, 847.8.

### General Schemes For Preparing VHL Building Blocks

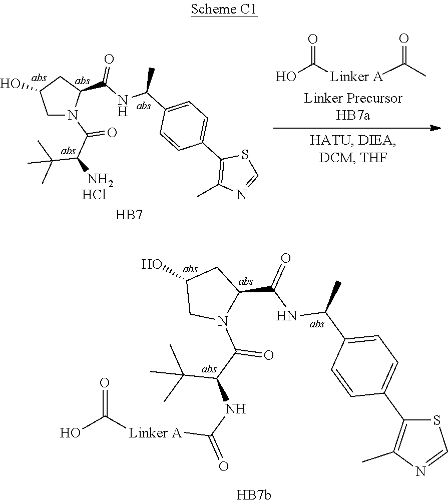

In certain embodiments, VHL-targeting LHM building blocks comprise coupling a linker precursor (HB7a) to (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide hydrochloride (HB7). Described below are detailed reaction procedures and additional examples of VHL-targeting LHM building blocks that may be prepared according to Scheme C1.

### General Procedure 1: Amide Formation

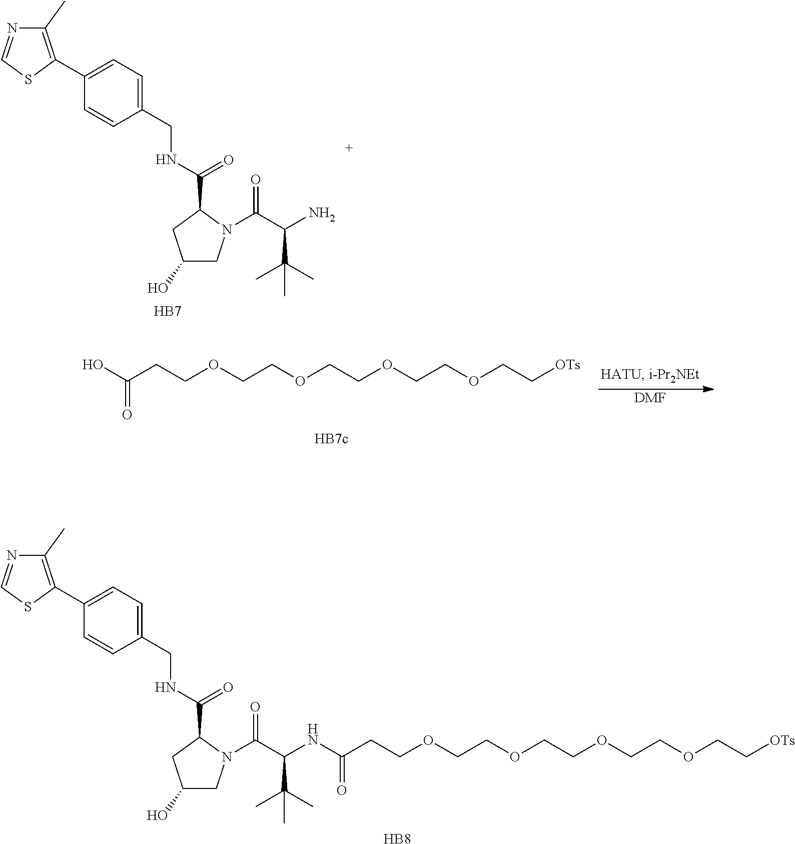

### Scheme 1: Synthesis of Compound HB8 via Amide Formation

To a solution of acid (250 mg, 487.55 μmol, 1 equiv) and amine (227.70 mg, 487.55 μmol, 1 equiv, HCl) in DMF (5 mL) was added HATU (203.92 mg, 536.30 μmol, 1.1 eq) at 25° C., and then DIPEA (252.05 mg, 1.95 mmol, 339.69 μL, 4 equiv) was added. The mixture was stirred at 25° C. for 2 hr. The mixture was poured into water (20 mL). The aqueous phase was extracted with EtOAc (20 mL, 3×). The combined organic phase was washed with brine (20 mL), dried over anhydrous Na₂SO₄, filtered, and concentrated under vacuum. The crude product (500 mg) was taken into the next step without purification.

An exemplary amide coupling is provided in the scheme immediately above where (2S,4R)-1-[(2S)-2-amino-3,3-dimethyl-butanoyl]-4-hydroxy-N-[[4-(4-methylthiazol-5-yl)phenyl]methyl]pyrrolidine-2carboxamide (HB7) (227.70 mg, 487.55 μmol, 1 equiv, HCl) and 3-[2-[2-[2-[2-(p-tolylsulfonyloxy)ethoxy]ethoxy]ethoxy]ethoxy]propanoic acid (HB7c) (250 mg, 487.55 μmol, 1 equiv) were treated as described above to provide 2-[2-[2-[2-[3-[[(1S)-1-[(2S,4R)-4-hydroxy-2-[[4-(4-methylthiazol-5-yl)phenyl]methylcarbamoyl]pyrrolidine-1carbonyl]-2,2-dimethyl-propyl]amino]-3-oxo-propoxy]ethoxy]ethoxy]ethoxy]ethyl 4-methylbenzenesulfonate (Compound HB8). LCMS: calculated for C₄₀H₅₆N₄O₁₁S₂ requires 832, found: m/z=833 [M+H]⁺.

General Procedure 2: Amide formation

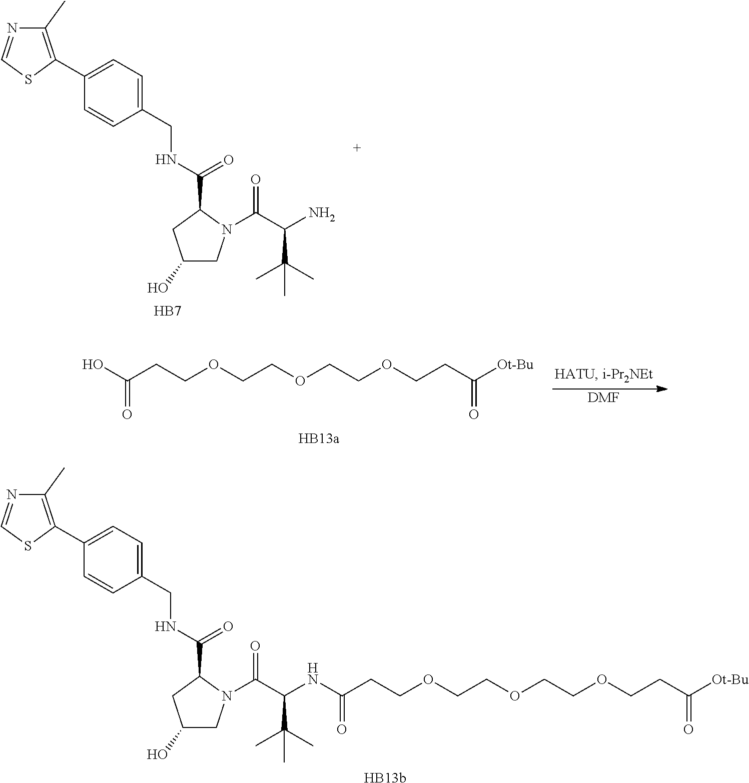

### Scheme 2: Synthesis of Compound HB13b Via Amide Formation

To a mixture of acid (649 mg, 2.12 mmol, 1.5 equiv), amine (659.59 mg, 1.41 mmol, 1 equiv, HCl), and HATU (644.41 mg, 1.69 mmol, 1.2 equiv) in DMF (10 mL) was added DIPEA (730.11 mg, 5.65 mmol, 983.98 μL, 4 equiv) in one portion under N₂ at 25° C. The mixture was stirred at 25° C. for 3 hr, and then poured into water (30 mL). The aqueous phase was extracted with ethyl acetate (20 mL, 3×). The combined organic phase was washed with brine (10 mL, 3×), dried with anhydrous Na₂SO₄, filtered, and concentrated under vacuum to afford the crude amide product (668 mg) as a yellow oil.

An exemplary amide coupling is provided in the scheme immediately above where (2S,4R)1-[(2S)-2-amino-3,3-dimethyl-butanoyl]-4-hydroxy-N-[[4-(4-methylthiazol-5-yl)phenyl]methyl]pyrrolidine-2-carboxamide (HB7) (659.59 mg, 1.41 mmol, 1 equiv, HCl) and 3-[2-[2-(3-tert-butoxy-3-oxo-propoxy)ethoxy]ethoxy]propanoic acid (HB13a) (649 mg, 2.12 mmol, 1.5 equiv) were treated as described above to provide tert-butyl 3-[2-[2-[3-[[(1S)-1-[(2S,4R)-4-hydroxy-2-[[4-(4-methylthiazol-5-yl)phenyl]methylcarbamoyl]pyrrolidine1-carbonyl]-2,2-dimethyl-propyl]amino]-3-oxo-propoxy]ethoxy]ethoxy]propanoate (Compound HB13b).

Other amide containing compounds of this disclosure synthesized using General Procedure 2 are Compounds HB30a, HB31a, and HB32a.

General Procedure 3: t-Bu Deprotection

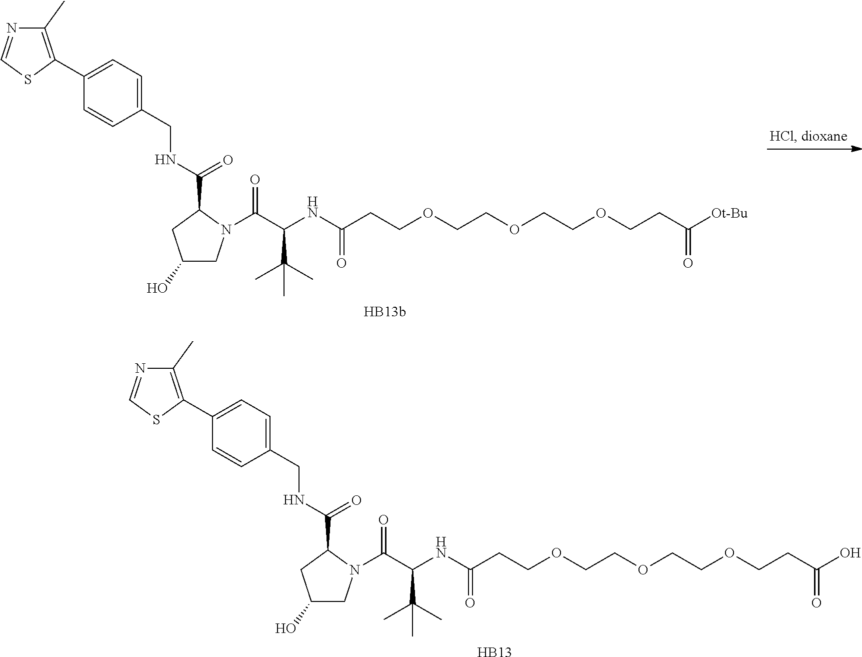

Scheme 3: Synthesis of Compound HB13 Via t-Bu Deprotection

A mixture of t-Bu ester (107 mg, 148.84 μmol, 1 equiv) in HCl:dioxane (4 M, 3 mL, 80.62 equiv) was stirred at 25° C. for 2 hr. The mixture was then concentrated under vacuum.

The residue was purified by prep-HPLC (column: Phenomenex Synergi C18 150*25*10 μm; mobile phase: [water (0.225% FA) ACN]; B %: 21%-55%, 22 min) to afford the carboxylic acid product (30 mg, 45.04 μmol, 30.26%) as a white gum.

An exemplary t-Bu deprotection is provided in the scheme immediately above where tert-butyl 3-[2-[2-[3-[[(1S)-1-[(2S,4R)-4-hydroxy-2-[[4-(4-methylthiazol-5yl)phenyl]methylcarbamoyl]pyrrolidine-1-carbonyl]-2,2-dimethyl-propyl]amino]-3-oxo-propoxy]ethoxy]ethoxy]propanoate (HB13b) (107 mg, 148.84 μmol, 1 equiv) was treated as described above to provide 3-[2-[2-[3-[[(1S)-1-[(2S,4R)-4-hydroxy-2-[[4-(4-methylthiazol-5-yl)phenyl]methylcarbamoyl]pyrrolidine-1carbonyl]-2,2-dimethyl-propyl]amino]-3-oxo-propoxy]ethoxy]ethoxy]propanoic acid (Compound HB13) (30 mg, 45.04 μmol, 30.26%). LCMS: Calculated C₃₂H₄₆N₄O₉S requires 662, found: m/z=685 [M+Na]⁺.

Other carboxylic acid containing compounds of this disclosure synthesized using General Procedure 3 are Compounds HB30, HB31, and HB32.

### Example 6. (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide;hydrochloride (HB7)

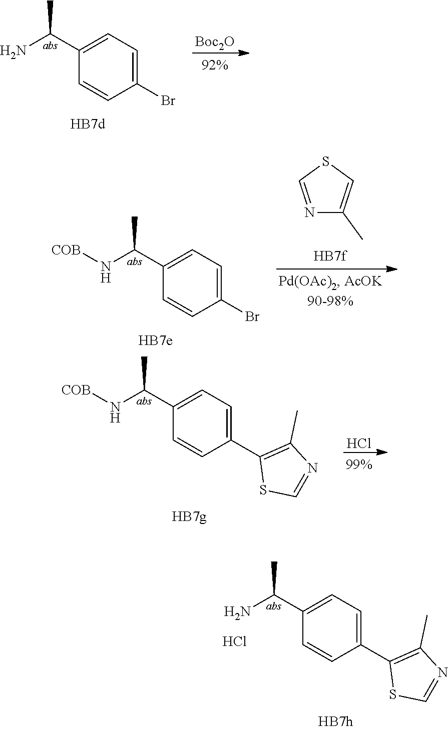

### Synthesis of tert-butyl N-[(1S)-1-(4-bromophenyl)ethyl]carbamate (HB7e)

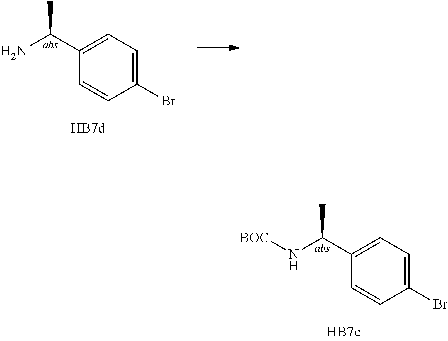

To a solution of (1S)-1-(4-bromophenyl)ethanamine (25.0 g, 125 mmol) and BOC anhydride (32.7 g, 150 mmol) in DCM (250 mL) at 0° C. was added TEA (34.8 mL, 250 mmol). The reaction mixture was stirred at 0° C. for 15 min and then 18 h at rt. The mixture was diluted with water (250 mL) and the layers were separated. The aqueous layer was extracted with EtOAc (2×100 mL). The combined organic layers were washed with brine (100 mL), dried with (Na₂SO₄), filtered, and concentrated under reduced pressure. The resulting solid was triturated with hexanes (400 mL), filtered, and washed with hexanes (500 mL) to afford the title compound HB7e as a solid (34.5 g, 92%). MS (ESI) [M-t-Bu]⁺ 244.0, 246.0.

### Synthesis of tert-butyl N-[(1S)-1-[4-(4-methylthiazol-5-yl)phenyl]ethyl]carbamate (HB7g)

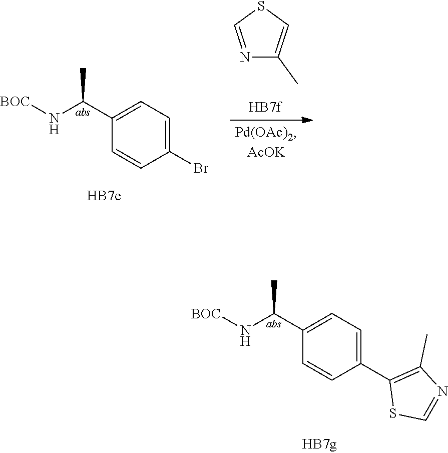

A mixture of tert-butyl N-[(1S)-1-(4-bromophenyl)ethyl]carbamate (HB7e) (15.0 g, 50.0 mmol), potassium acetate (9.81 g, 100 mmol), and Pd(OAc)₂ (112 mg, 0.50 mmol) in DMA (100 mL) at rt was added 4-methylthiazole (9.10 mL, 100 mmol). The mixture was purged with nitrogen and then put under vacuum (3× cycle) and then stirred at 120° C. for 2 h. The mixture was cooled to rt and diluted with water (250 mL). The resulting solid was filtered and washed with water (500 mL). The solid was dried in a vacuum oven at 65° C. for 18 h to afford the title compound HB7g (15.6 g, 98%). MS (ESI) [M+H]⁺ 319.2.

### Synthesis of (1S)-1-[4-(4-methylthiazol-5-yl)phenyl]ethanamine;hydrochloride (HB7h)

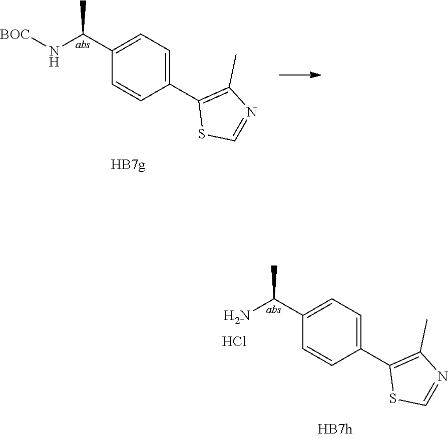 [SMILES: Cc1ncsc1-c1ccc([C@H](C)NC(=O)OC(C)(C)C)cc1 ; Cl ; Cc1ncsc1-c1ccc([C@H](C)N)cc1]

To a solution of tert-butyl N-[(1S)-1-[4-(4-methylthiazol-5-yl)phenyl]ethyl]carbamate (HB7g) (17.4 g, 54.6 mmol) in DCM (200 mL) at 0° C. was added HCl (4 M in dioxane, 200 mL, 800 mmol) and the mixture was warmed to rt and stirred for 3 h. The mixture was diluted with ether (50 mL) and the resulting solid was filtered. The solid was washed with ether (500 mL) and dried to afford the title compound HB7h as a solid (15.0 g, quant.). ¹H NMR (400 MHz, DMSO-d₆) δ 9.11 (s, 1H), 8.69 (br s, 3H), 7.67-7.62 (m, 2H), 7.58-7.53 (m, 2H), 4.44 (dt, J=11.9, 5.9 Hz, 1H), 2.47 (s, 3H), 1.55 (d, J=6.8 Hz, 3H). MS (ESI) [M+H]⁺202.2.

### Example 7. Methyl (2S,4R)-1-[(2S)-2-(tert-butoxycarbonylamino)-3,3-dimethyl-butanoyl]-4-hydroxy-pyrrolidine-2-carboxylate (HB7)

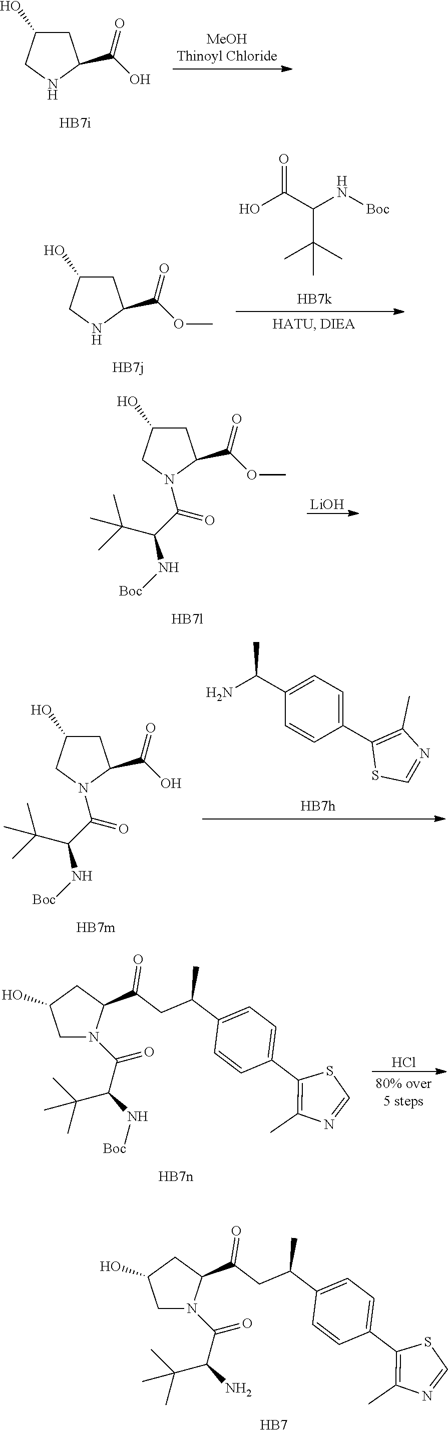

### Synthesis of methyl (2S,4R)-1-[(2S)-2-(tert-butoxycarbonylamino)-3,3-dimethyl-butanoyl]-4-hydroxy-pyrrolidine-2-carboxylate (HB7l)

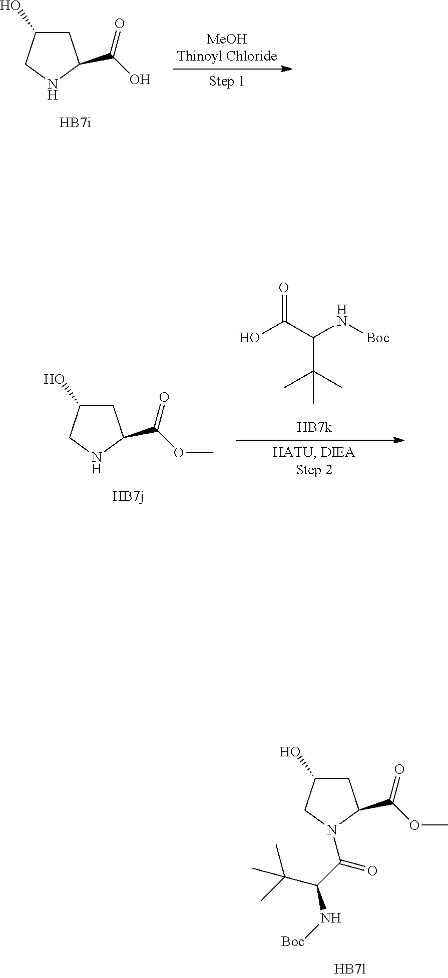

Step 1: To a solution of (2S,4R)-4-hydroxypyrrolidine-2-carboxylic acid (10.0 g, 76.3 mmol) in MeOH (300 mL) at 0° C. was added SOCl₂ (10.0 mL, 137 mmol) under nitrogen. The mixture was warmed to rt and then stirred for 18 h. The volatiles were evaporated under reduced pressure to afford the title compound HB7j, which was used in the next step without further purification.
Step 2: To the above HB7j in DCM (250 mL) at rt, were sequentially added (2R)-2-[(tert-butoxycarbonylamino)methyl]-3,3-dimethyl-butanoic acid (18.7 g, 80.9 mmol) and HATU (43.5 g, 114 mmol). The mixture was cooled to 0° C. and then DIEA (65 mL, 380 mmol) was slowly added over 15 min. The reaction mixture was warmed to rt and then stirred for 20 h. The mixture was diluted with 5% citric acid (400 mL) and DCM (200 mL) and the layers were separated. The aqueous layer was extracted with DCM (300 mL). The combined organic layers were washed with 1 M NaOH (2×200 mL), brine (200 mL), and then dried (Na₂SO₄), filtered, and concentrated under reduce pressure to afford the title compound HB71, which was used in the next step without further purification. MS (ESI) [M−BOC]⁺ 259.3.

### Synthesis of (2S,4R)-1-[(2S)-2-(tert-butoxycarbonylamino)-3,3-dimethyl-butanoyl]-4-hydroxy-pyrrolidine-2-carboxylic acid (HB7m)

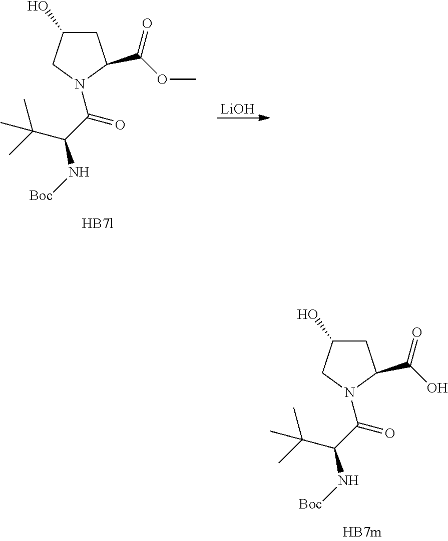

To a solution of methyl (2S,4R)-1-[(2S)-2-(tert-butoxycarbonylamino)-3,3-dimethyl-butanoyl]-4-hydroxy-pyrrolidine-2-carboxylate (HB71) (27.4 g, 76.4 mmol) in MeOH (372 mL) and THE (372 mL) at rt was added lithium hydroxide monohydrate (7.40 g, 176 mmol) and the mixture was stirred at rt for 48 h. The volatiles were evaporated under reduced pressure.

The residue was diluted with 1 M NaOH (300 mL) and washed with ether (250 mL). The aqueous layer was acidified to pH 4 and extracted with EtOAc (2×300 mL). The pH was then adjusted to 1 and the mixture was extracted with EtOAc (3×300 mL). The combined organic layers were washed with brine (300 mL) and then dried (Na₂SO₄), filtered, and reduced under reduced pressure to afford the title compound HB7m as a foam (31 g), which was used in the next step without further purification. MS (ESI) [M−t−Bu]⁺ 289.1.

### Synthesis of tert-butyl N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamate (HB7n)

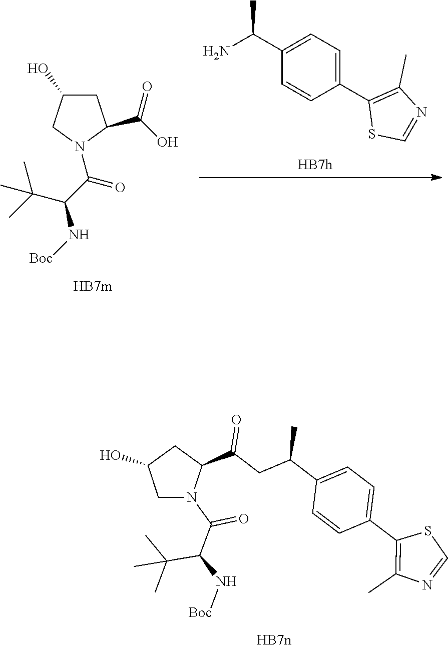 [SMILES: CC(C)(C)OC(=O)N[C@H](C(=O)N1C[C@H](O)C[C@H]1C(=O)O)C(C)(C)C ; Cc1ncsc1-c1ccc([C@H](C)N)cc1 ; Cc1ncsc1-c1ccc([C@H](C)CC(=O)[C@@H]2C[C@@H](O)CN2C(=O)[C@@H](NC(=O)OC(C)(C)C)C(C)(C)C)cc1]

To a mixture of (2S,4R)-1-[(2S)-2-(tert-butoxycarbonylamino)-3,3-dimethyl-butanoyl]-4-hydroxy-pyrrolidine-2-carboxylic acid (HB7m) (27.3 g, 79.2 mmol), (1S)-1-[4-(4-methylthiazol-5-yl)phenyl]ethanamine hydrochloride (HB7h) (20.2 g, 79.2 mmol), and HATU (45.2 g, 119 mmol) in DCM (775 mL) at 0° C. was slowly added DIEA (68.0 mL, 396 mmol), and the mixture was stirred for 20 h. The mixture was then diluted with 5% citric acid (500 mL) and the layers were separated. The organic layer was washed with 1 M NaOH (2×300 mL) and brine (300 mL) and then dried (Na₂SO₄), filtered, and concentrated under reduced pressure. The resulting solid was dissolved into a minimal amount of MeOH and then water was added until precipitation was observed. The resulting solids were filtered, washed with ether (400 mL), and then dried in a vacuum oven at 60° C. to afford the title compound HB7n as a solid (34 g, 79%). MS (ESI) [M+H]⁺ 545.3.

### Synthesis of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide;hydrochloride (HB7)

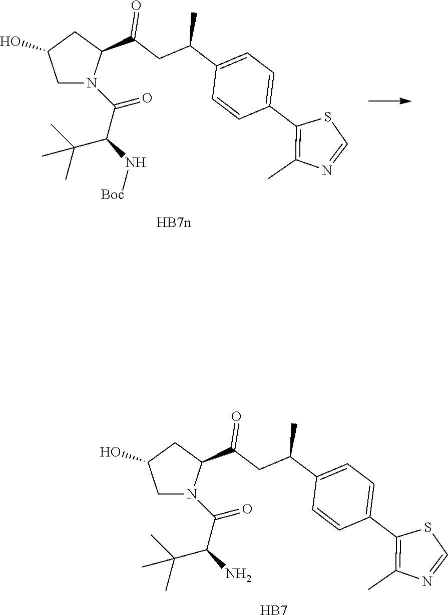

To a solution of tert-butyl N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamate (HB7h) (34.0 g, 62.0 mmol) in DCM (200 mL) at 0° C. was added an HCl solution (4 M in dioxane, 200 mL, 800 mmol) and the mixture was warmed to rt and then stirred for 15 min. The mixture was then diluted with MeOH (150 mL) and the mixture was further stirred for 30 min. The volatiles were evaporated under reduced pressure and coevaporated with PhMe (2×100 mL) to afford the title compound HB7 as a solid (30.6 g, 92%, contained 9% PhMe by weight). ¹H NMR (500 MHz, DMSO-d₆) δ 9.04 (s, 1H), 8.59 (d, J=7.8 Hz, 1H), 8.09 (d, J=4.3 Hz, 3H), 7.47-7.43 (m, 2H), 7.42-7.37 (m, 2H), 4.93 (p, J=7.0 Hz, 1H), 4.55 (t, J=8.4 Hz, 1H), 4.33 (br s, 1H), 3.91 (q, J=5.7 Hz, 1H), 3.73 (d, J=10.6 Hz, 1H), 3.50 (dd, J=10.9, 3.9 Hz, 1H), 2.70 (s, 1H), 2.47 (s, 3H), 2.12 (dd, J=12.9, 7.7 Hz, 1H), 1.81-1.72 (m, 1H), 1.39 (d, J=7.0 Hz, 3H), 1.03 (s, 9H). MS (ESI) [M+H]⁺ 445.2.

### Example 8. 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid (HB9)

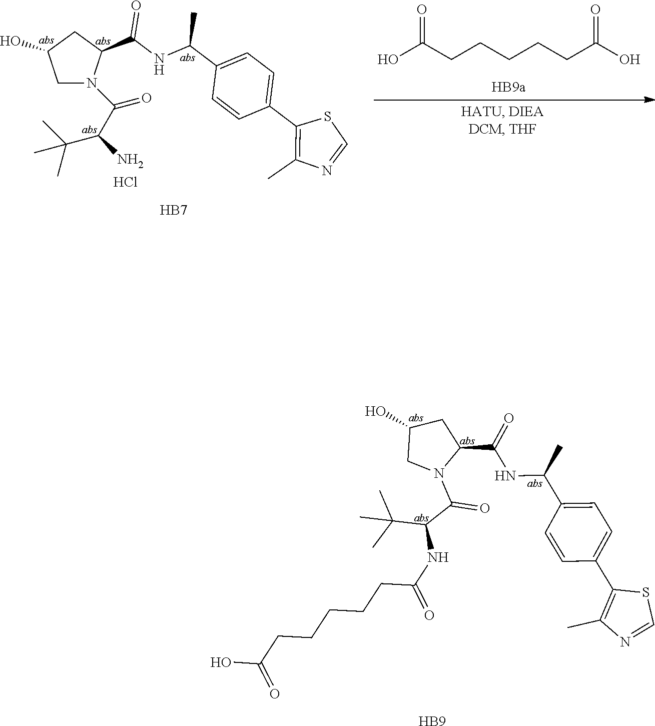

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide;hydrochloride (HB7) (1.75 g, 3.64 mmol), heptanedioic acid (874 mg, 5.46 mmol), and HATU (1.94 g, 5.09 mmol) in DCM (70.0 mL) at 0° C. was added DIEA (3.11 mL, 18.2 mmol) and the reaction mixture was stirred for 2 h. The mixture was diluted with 1 M NaOH (50 mL) and stirred for one hour. The layers were separated and the organic layer was extracted with 1 M NaOH (2×30 mL). The combined aqueous layers were acidified to pH 5-6 and extracted with EtOAc (5×50 mL). The combined organic layers were dried (Na₂SO₄), filtered, and concentrated under reduced pressure. The material was further purified by reverse phase chromatography on a C18 column using a 10-60% gradient of MeCN and water (contains 0.1% ammonium formate:formic acid) to afford 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid as a solid (0.924 g, 43%). ¹H NMR (500 MHz, DMSO-d₆) δ 8.99 (s, 1H), 8.37 (d, J=7.8 Hz, 1H), 7.79 (d, J=9.3 Hz, 1H), 7.46-7.41 (m, 2H), 7.40-7.36 (m, 2H), 4.92 (p, J=7.0 Hz, 1H), 4.52 (d, J=9.4 Hz, 1H), 4.43 (t, J=8.1 Hz, 1H), 4.30-4.26 (m, 1H), 3.65-3.57 (m, 2H), 3.46-3.33 (m, 1H), 2.46 (s, 3H), 2.28-2.20 (m, 1H), 2.18 (t, J=7.4 Hz, 2H), 2.15-2.06 (m, 1H), 2.04-1.97 (m, 1H), 1.80 (ddd, J=12.9, 8.5, 4.7 Hz, 1H), 1.54-1.42 (m, 4H), 1.38 (d, J=7.0 Hz, 3H), 1.28-1.20 (m, 2H), 0.94 (s, 9H). MS (ESI) [M+H]⁺587.3.

### Example 9. Synthesis of 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic acid (HB10)

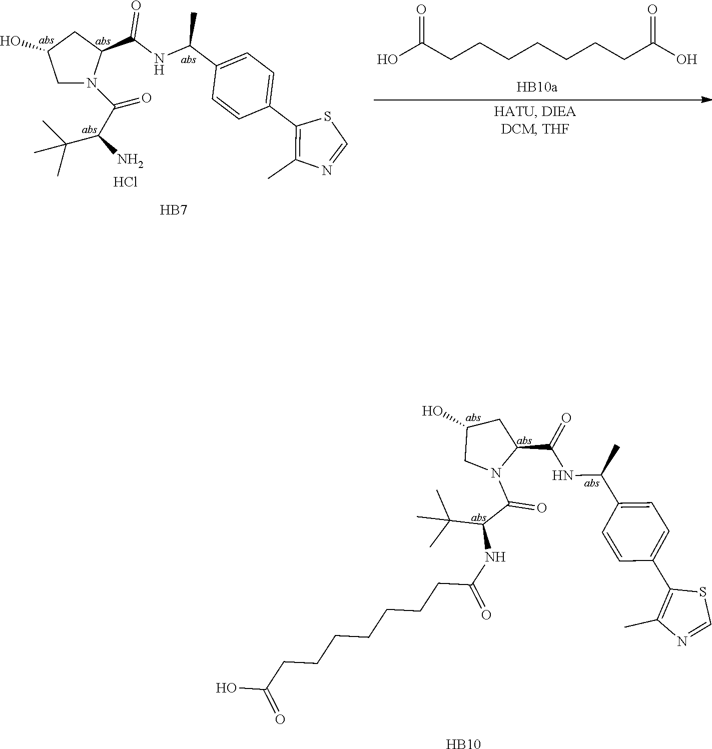

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide hydrochloride HB7 (2.0 g, 4.2 mmol), nonanedioic acid (1.2 g, 6.2 mmol), and HATU (2.1 g, 5.4 mmol) in DCM (20 mL) and THE (20 mL) at 0° C. was added DIEA (3.56 mL, 20.8 mmol) and the reaction mixture was stirred for 2 h. The mixture was diluted with 1 M NaOH (50 mL) and stirred for one hour. The mixture was acidified to pH 5 and the aqueous layer was extracted with EtOAc (5×50 mL). The combined organic layers were dried (Na₂SO₄), filtered, and concentrated under reduced pressure. The material was purified by reverse phase chromatography on a C18 column using a 10-40% gradient of MeCN and water (contained 0.1% ammonium formate:formic acid) to afford 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic acid as a solid (1.00 g, 39%). ¹H NMR (500 MHz, DMSO-d₆) δ 8.99 (s, 1H), 8.37 (d, J=7.8 Hz, 1H), 7.78 (d, J=9.3 Hz, 1H), 7.47-7.42 (m, 2H), 7.40-7.36 (m, 2H), 5.10 (br s, 1H), 4.97-4.88 (m, 1H), 4.52 (d, J=9.3 Hz, 1H), 4.43 (t, J=8.0 Hz, 1H), 4.33-4.24 (m, 1H), 3.66-3.54 (m, 2H), 2.46 (s, 3H), 2.28-2.22 (m, 1H), 2.19 (t, J=7.4 Hz, 2H), 2.14-2.07 (m, 1H), 2.04-1.98 (m, 1H), 1.83-1.76 (m, 1H), 1.54-1.41 (m, 4H), 1.38 (d, J=7.0 Hz, 3H), 1.31-1.19 (m, 6H), 0.94 (s, 9H). MS (ESI) [M+H]⁺615.7.

### Example 10. Synthesis of 11-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-11-oxoundecanoic acid (HB1l)

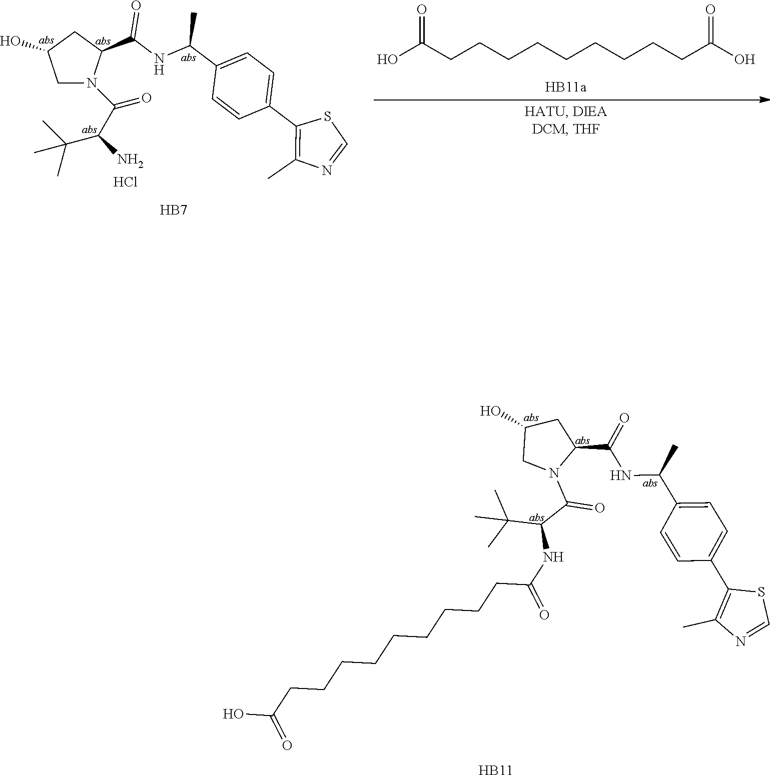

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide hydrochloride HB7 (2.0 g, 4.2 mmol), undecanedioic acid (1.4 g, 6.2 mmol), and HATU (2.4 g, 6.2 mmol) in DCM (20 mL) and THF (20 mL) at 0° C. was added DIEA (3.56 mL, 20.8 mmol) and the reaction mixture was stirred for 2 h. The mixture was then diluted with 1 M NaOH (50 mL) and stirred for one hour. The mixture was acidified to pH 5 and the aqueous layer was extracted with EtOAc (5×50 mL). The combined organic layers were dried (Na₂SO₄), filtered, and concentrated under reduced pressure. The material was purified by reverse phase chromatography on a C18 column using a 10-40% gradient of MeCN and water (contained 0.1% ammonium formate:formic acid) to afford 11-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-11-oxoundecanoic acid as a solid (832 mg, 31%). ¹H NMR (500 MHz, DMSO-d₆) δ 8.99 (s, 1H), 8.37 (d, J=7.8 Hz, 1H), 7.77 (d, J=9.4 Hz, 1H), 7.46-7.42 (m, 2H), 7.41-7.36 (m, 2H), 5.09 (br s, 1H), 4.95-4.88 (m, 1H), 4.52 (d, J=9.4 Hz, 1H), 4.43 (t, J=8.0 Hz, 1H), 4.31-4.25 (m, 1H), 3.67-3.54 (m, 2H), 2.46 (s, 3H), 2.30-2.21 (m, 1H), 2.19 (t, J=7.4 Hz, 2H), 2.14-2.06 (m, 1H), 2.04-1.98 (m, 1H), 1.80 (ddd, J=12.9, 8.4, 4.6 Hz, 1H), 1.54-1.42 (m, 4H), 1.38 (d, J=7.0 Hz, 3H), 1.30-1.18 (m, 10H), 0.94 (s, 9H). MS (ESI) [M+H]⁺643.4.

### Example 11. tert-butyl 5-[4-(2-methoxy-2-oxoethyl)piperidin-1-yl]pyridine-2-carboxylate (HB12c)

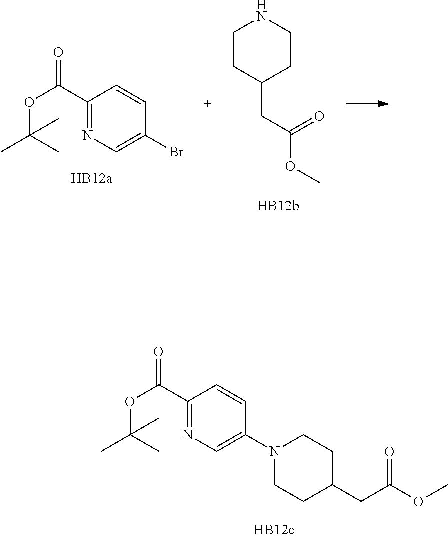

A mixture of methyl 2-(piperidin-4-yl)acetate (451.94 mg, 2.87 mmol), tert-butyl 5-bromopyridine-2-carboxylate (742.00 mg, 2.87 mmol), [2′-(methylamino)-[1,1′-biphenyl]-2-yl]palladiumylium dicyclohexyl({2′,6′-diisopropoxy-[1,1′-biphenyl]-2-yl})phosphane mesylate (122.23 mg, 0.14 mmol), and cesium carbonate (1873.26 mg, 5.75 mmol) was degassed and backfilled with N₂ five times. Dioxane (10 mL) was added and the mixture was allowed to stir at 100° C. for 3 h. The mixture was filtered through silica gel washed with EtOAc, concentrated, and purified by MPLC (10-100% EtOAc:hexanes) to afford tert-butyl 5-[4-(2-methoxy-2-oxoethyl)piperidin-1-yl]pyridine-2-carboxylate (0.7110 g, 74.0%). LCMS: C₁₈H₂₆N₂O₄ requires: 334, found: m/z=335 [M+H]⁺.

### Example 12. 5-[4-(2-methoxy-2-oxoethyl)piperidin-1-yl]pyridine-2-carboxylic acid (HB12d)

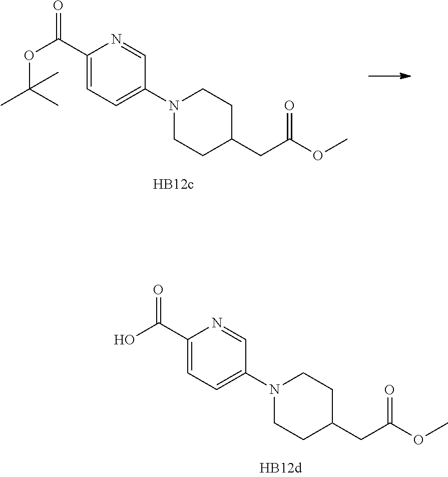 [SMILES: COC(=O)CC1CCN(c2ccc(C(=O)OC(C)(C)C)nc2)CC1 ; COC(=O)CC1CCN(c2ccc(C(=O)O)nc2)CC1]

A mixture of tert-butyl 5-[4-(2-methoxy-2-oxoethyl)piperidin-1-yl]pyridine-2-carboxylate (352.00 mg, 1.05 mmol), in CH₂Cl₂ (10 mL) and TFA (2 mL) was allowed to stir at rt for 6 h. The volatiles were removed under vacuum to afford 5-[4-(2-methoxy-2-oxoethyl)piperidin-1-yl]pyridine-2-carboxylic acid (0.2900 g, 99.0%). LCMS: C₁₄H₁₈N₂O₄ requires: 278, found: m/z=279 [M+H]⁺.

### Example 13. {1-[6-(tert-butoxycarbonyl)pyridin-3-yl]piperidin-4-yl}acetic acid (HB12e)

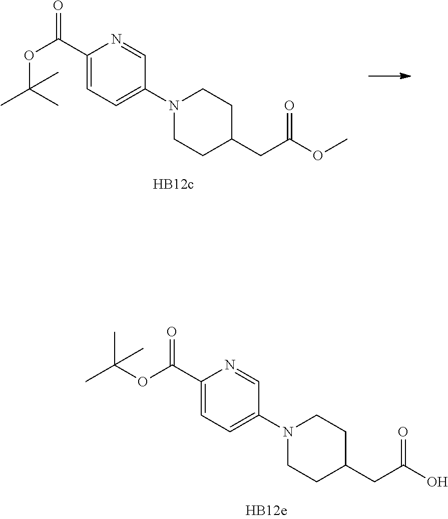

To a mixture of tert-butyl 5-[4-(2-methoxy-2-oxoethyl)piperidin-1-yl]pyridine-2-carboxylate (359.00 mg, 1.07 mmol), in THF, H₂O, and EtOH was added lithium hydroxide hydrate (135.13 mg, 3.22 mmol) and EtOH and the mixture was allowed to stir at rt for 2 h. The mixture was concentrated and purified by reverse phase MPLC (10-100% MeCN in H₂O) to afford {1-[6-(tert-butoxycarbonyl)pyridin-3-yl]piperidin-4-yl}acetic acid (0.3300 g, 95.9%). LCMS: C₁₇H₂₄N₂O₄ requires: 320, found: m/z=321 [M+H]⁺.

### Example 14. tert-butyl 5-(4-(ethoxycarbonyl)piperidin-1-yl)pyrimidine-2-carboxylate (HB12h)

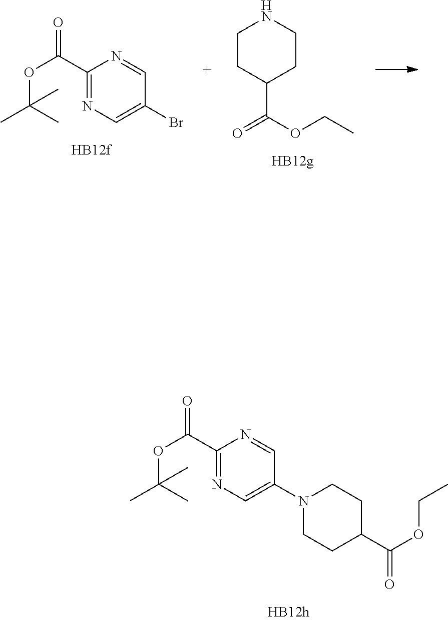 [SMILES: CC(C)(C)OC(=O)c1ncc(Br)cn1 ; CCOC(=O)C1CCNCC1 ; CCOC(=O)C1CCN(c2cnc(C(=O)OC(C)(C)C)nc2)CC1]

HB12h was prepared using the same procedure for tert-butyl 5-[4-(2-methoxy-2-oxoethyl)piperidin-1-yl]pyridine-2-carboxylate (HB12c) except with tert-butyl 5-bromopyrimidine-2-carboxylate and ethyl piperidine-4-carboxylate to provide tert-butyl 5-(4-(ethoxycarbonyl)piperidin-1-yl)pyrimidine-2-carboxylate (0.708 g, 81%). LCMS: C₁₇H₂₅N₃O₄ requires: 335, found: m/z=336 [M+H]⁺.

### Example 15. 5-(4-(ethoxycarbonyl)piperidin-1-yl)pyrimidine-2-carboxylic acid (HB12i)

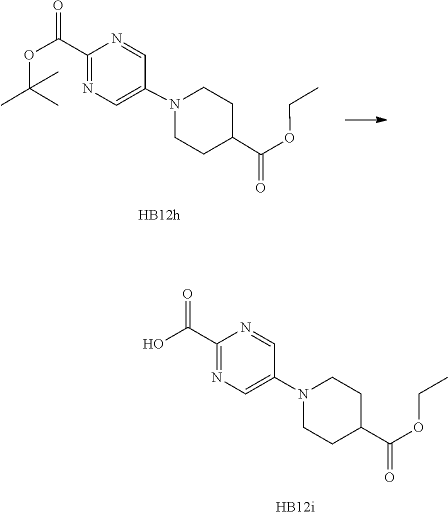 [SMILES: CCOC(=O)C1CCN(c2cnc(C(=O)OC(C)(C)C)nc2)CC1 ; CCOC(=O)C1CCN(c2cnc(C(=O)O)nc2)CC1]

HB12i was prepared using the same procedure for 5-[4-(2-methoxy-2-oxoethyl)piperidin-1-yl]pyridine-2-carboxylic acid (HB12d) except with tert-butyl 5-(4-(ethoxycarbonyl)piperidin-1-yl)pyrimidine-2-carboxylate to provide 5-(4-(ethoxycarbonyl)piperidin-1-yl)pyrimidine-2-carboxylic acid. LCMS: C₁₃H₁₇N₃O₄ requires: 279, found: m/z=280 [M+H]⁺.

### Example 16. methyl 2-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidin-4-yl)acetate (HB12j)

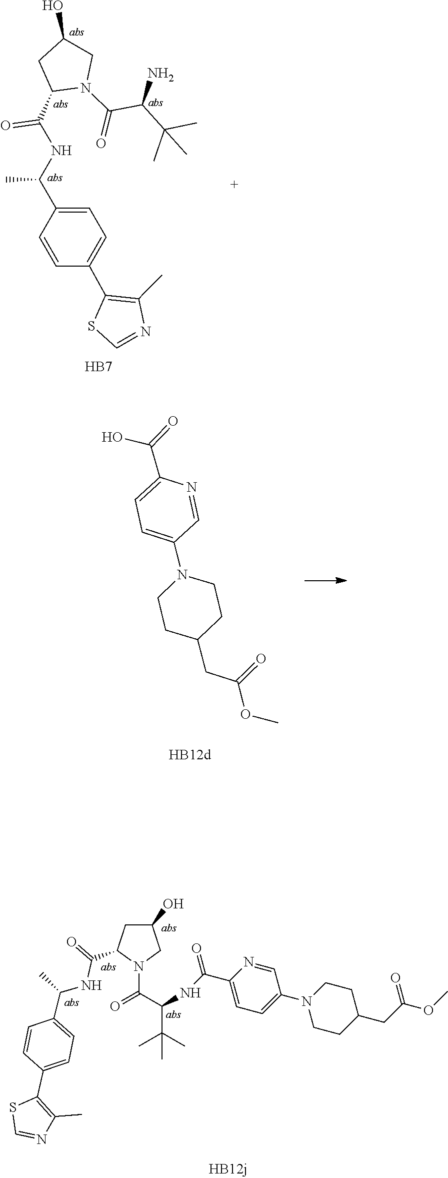 [SMILES: Cc1ncsc1-c1ccc([C@H](C)NC(=O)[C@@H]2C[C@@H](O)CN2C(=O)[C@@H](N)C(C)(C)C)cc1 ; "HB7" ; COC(=O)CC1CCN(c2ccc(C(=O)O)nc2)CC1 ; COC(=O)CC1CCN(c2ccc(C(=O)N[C@H](C(=O)N3C[C@H](O)C[C@H]3C(=O)N[C@@H](C)c3ccc(-c4scnc4C)cc3)C(C)(C)C)nc2)CC1]

A mixture of PyBOP hexafluoro-lambda5-phosphanuide (646.71 mg, 1.24 mmol), 5-[4-(2-methoxy-2-oxoethyl)piperidin-1-yl]pyridine-2-carboxylic acid (292.65 mg, 1.05 mmol), (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide (425.00 mg, 0.96 mmol), N,N-diisopropylethylamine (0.69 mL, 0.49 g, 3.82 mmol), and DMF (5 mL) was allowed to stir at rt for one hour. EtOAc and H₂O were then added. The organic layer was separated and dried with MgSO₄, filtered, and concentrated to provide methyl 2-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidin-4-yl)acetate which was used in the next step without further purification. LCMS: C₃₇H₄₈N₆O₆S requires: 704, found: m/z=705 [M+H]⁺.

### Example 17. tert-butyl 5-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)picolinate (HB12k)

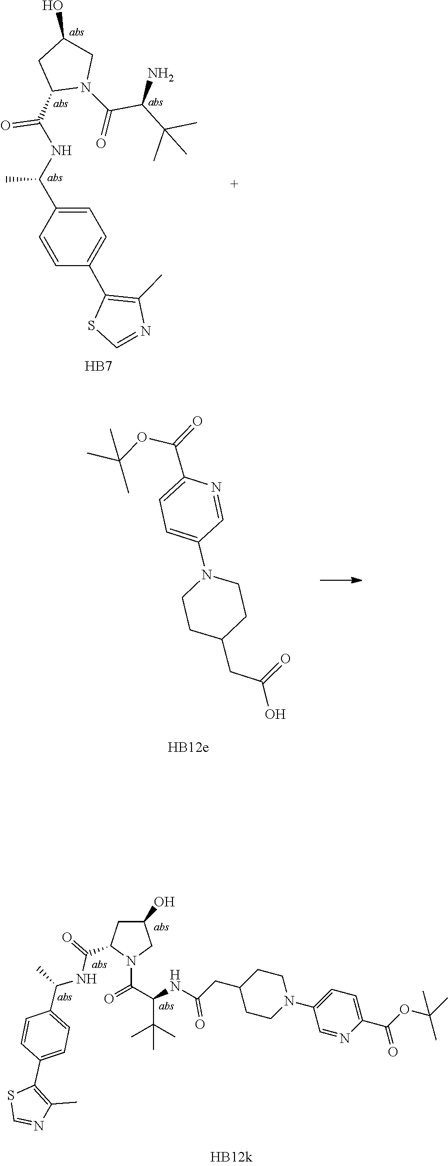 [SMILES: Cc1ncsc1-c1ccc([C@H](C)NC(=O)[C@@H]2C[C@@H](O)CN2C(=O)[C@@H](N)C(C)(C)C)cc1 ; "HB7" ; CC(C)(C)OC(=O)c1ccc(N2CCC(CC(=O)O)CC2)cn1 ; Cc1ncsc1-c1ccc([C@H](C)NC(=O)[C@@H]2C[C@@H](O)CN2C(=O)[C@@H](NC(=O)CC2CCN(c3ccc(C(=O)OC(C)(C)C)nc3)CC2)C(C)(C)C)cc1]

HB12k was prepared according to the same procedure for methyl 2-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidin-4-yl)acetate (HB12j) except with 2-(1-(6-(tert-butoxycarbonyl)pyridin-3-yl)piperidin-4-yl)acetic acid to provide tert-butyl 5-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)picolinate. LCMS: C₄₀H₅₄N₆O₆S requires: 704, found: m/z=705 [M+H]⁺.

### Example 18. ethyl 1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyrimidin-5-yl)piperidine-4-carboxylate (HB12l)

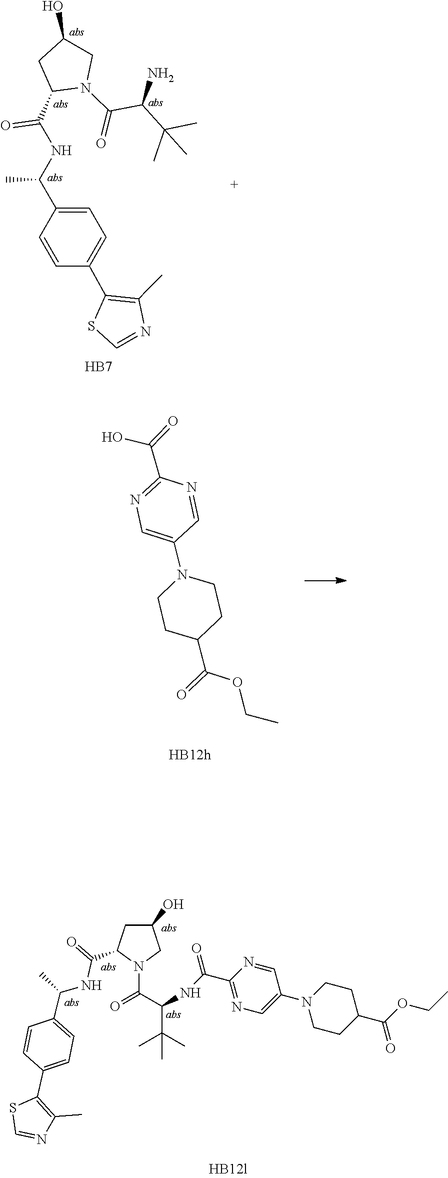 [SMILES: Cc1ncsc1-c1ccc([C@H](C)NC(=O)[C@@H]2C[C@@H](O)CN2C(=O)[C@@H](N)C(C)(C)C)cc1 ; "HB7" ; CCOC(=O)C1CCN(c2cnc(C(=O)O)nc2)CC1 ; CCOC(=O)C1CCN(c2cnc(C(=O)N[C@H](C(=O)N3C[C@H](O)C[C@H]3C(=O)N[C@@H](C)c3ccc(-c4scnc4C)cc3)C(C)(C)C)nc2)CC1]

HB12l was prepared according to the same procedure for methyl 2-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidin-4-yl)acetate (HB12j) except with 5-(4-(ethoxycarbonyl)piperidin-1-yl)pyrimidine-2-carboxylic acid to provide ethyl 1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyrimidin-5-yl)piperidine-4-carboxylate. LCMS: C₃₆H₄₇N₇O₆S requires: 705, found: m/z=706 [M+H]⁺.

### Example 19. 2-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidin-4-yl)acetic acid (HB12)

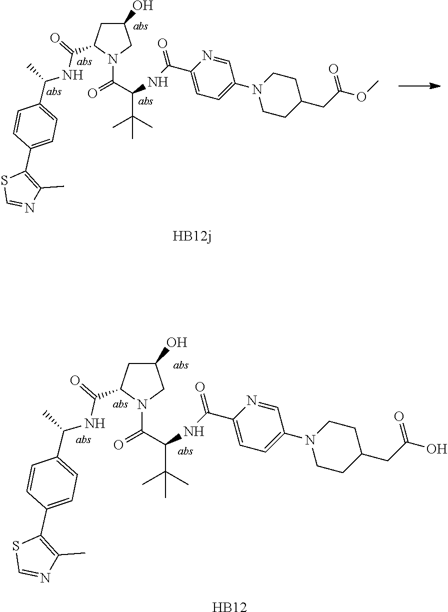 [SMILES: COC(=O)CC1CCN(c2ccc(C(=O)N[C@H](C(=O)N3C[C@H](O)C[C@H]3C(=O)N[C@@H](C)c3ccc(-c4scnc4C)cc3)C(C)(C)C)nc2)CC1 ; Cc1ncsc1-c1ccc([C@H](C)NC(=O)[C@@H]2C[C@@H](O)CN2C(=O)[C@@H](NC(=O)c2ccc(N3CCC(CC(=O)O)CC3)cn2)C(C)(C)C)cc1 ; "HB12"]

HB12 was prepared according to the same procedure for {1-[6-(tert-butoxycarbonyl)pyridin-3-yl]piperidin-4-yl}acetic acid (HB12e) except with methyl 2-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidin-4-yl)acetate to provide 2-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidin-4-yl)acetic acid (0.247 g, 38%). LCMS: C₃₆H₄₆N₆O₆S requires: 690, found: m/z=691 [M+H]⁺.

### Example 20. 5-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)picolinic acid (HB14)

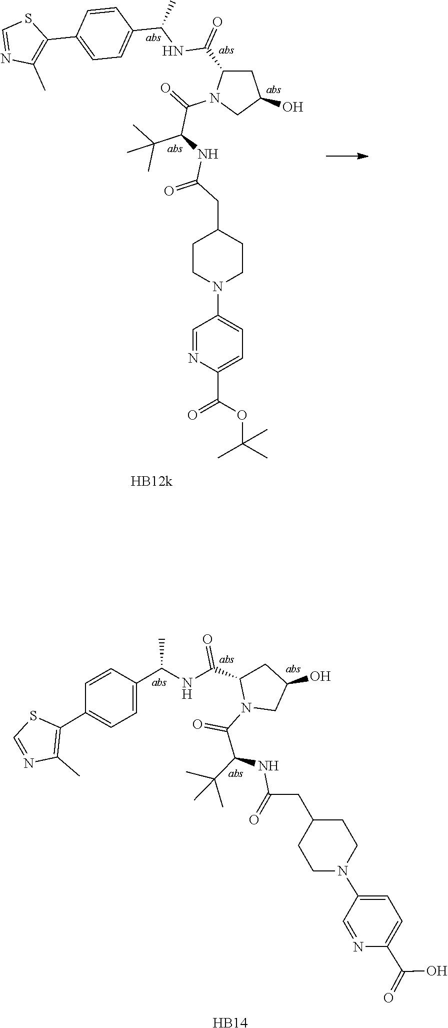 [SMILES: Cc1ncsc1-c1ccc([C@H](C)NC(=O)[C@@H]2C[C@@H](O)CN2C(=O)[C@@H](NC(=O)CC2CCN(c3ccc(C(=O)OC(C)(C)C)nc3)CC2)C(C)(C)C)cc1 ; Cc1ncsc1-c1ccc([C@H](C)NC(=O)[C@@H]2C[C@@H](O)CN2C(=O)[C@@H](NC(=O)CC2CCN(c3ccc(C(=O)O)nc3)CC2)C(C)(C)C)cc1 ; "HB14"]

HB14 was prepared using the same procedure for 5-[4-(2-methoxy-2-oxoethyl)piperidin-1-yl]pyridine-2-carboxylic acid (HB12d) except with tert-butyl 5-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)picolinate to provide 5-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)picolinic acid (0.130 g, 67%). LCMS: C₃₆H₄₆N₆O₆S requires: 690, found: m/z=691 [M+H]⁺.

### Example 21. 1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyrimidin-5-yl)piperidine-4-carboxylic acid (HB15)

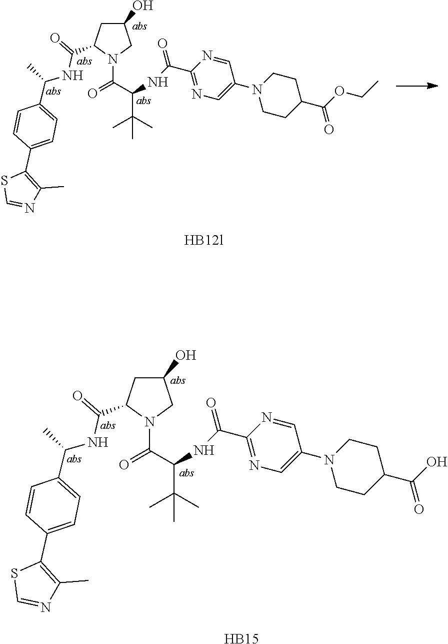 [SMILES: CCOC(=O)C1CCN(c2cnc(C(=O)N[C@H](C(=O)N3C[C@H](O)C[C@H]3C(=O)N[C@@H](C)c3ccc(-c4scnc4C)cc3)C(C)(C)C)nc2)CC1 ; Cc1ncsc1-c1ccc([C@H](C)NC(=O)[C@@H]2C[C@@H](O)CN2C(=O)[C@@H](NC(=O)c2ncc(N3CCC(C(=O)O)CC3)cn2)C(C)(C)C)cc1 ; "HB15"]

HB15 was prepared according to the same procedure for {1-[6-(tert-butoxycarbonyl)pyridin-3-yl]piperidin-4-yl}acetic acid (HB12e) except with ethyl 1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyrimidin-5-yl)piperidine-4-carboxylate to provide 1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyrimidin-5-yl)piperidine-4-carboxylic acid (0.817 g, 99%). LCMS: C₃₄H₄₃N₇O₆S requires: 677, found: m/z=678 [M+H]⁺.

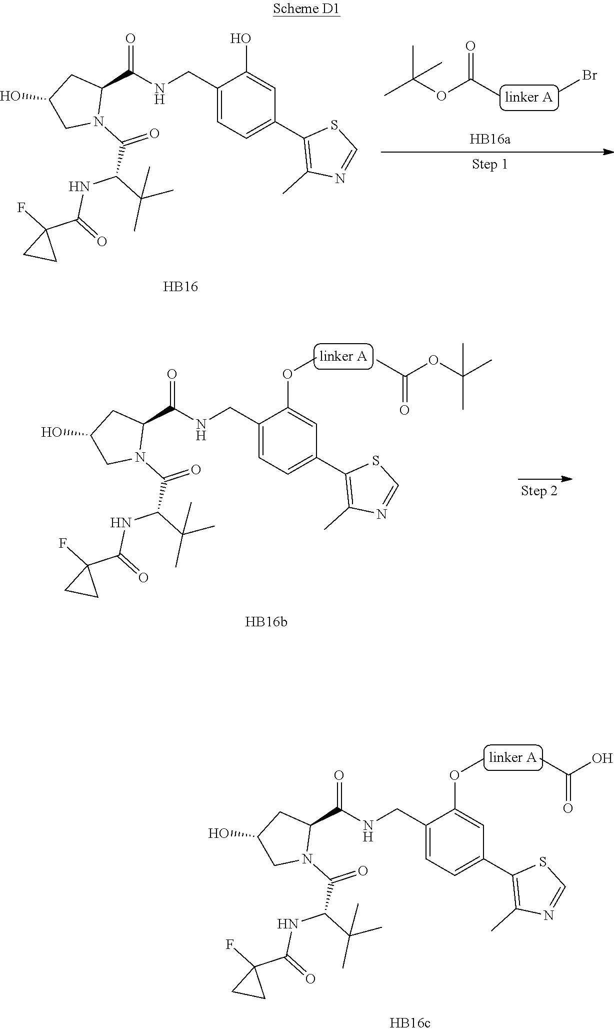

Scheme D1 begins with coupling a linker precursor to a VHL-targeting LHM, namely, (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (HB16). The VHL-targeting LHM was prepared according to the following steps.

### Example 22. (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (HB16)

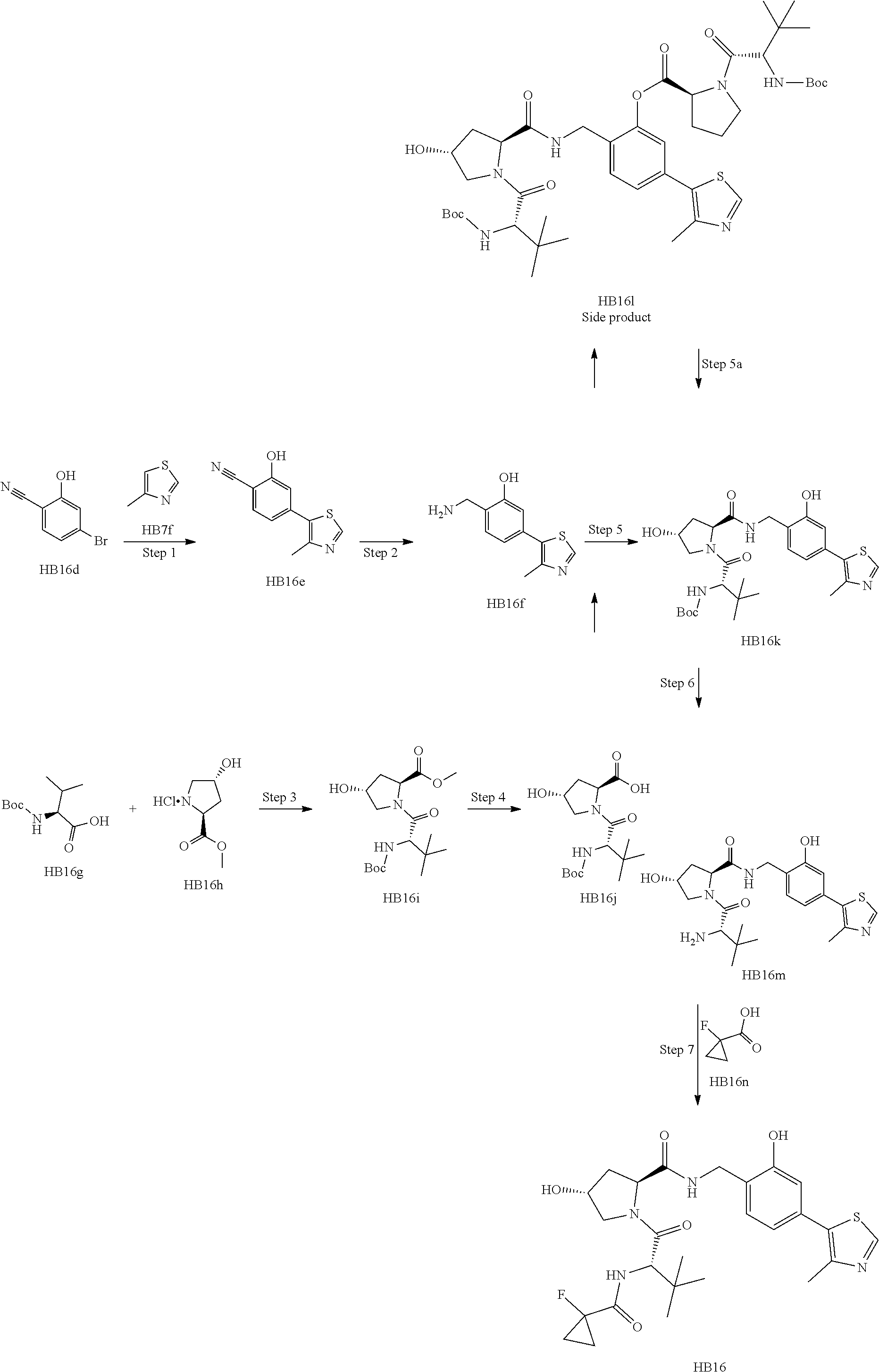

### Step 1: Synthesis of 2-hydroxy-4-(4-methyl-1,3-thiazol-5-yl)benzonitrile (HB16e)

A solution of 4-bromo-2-hydroxybenzonitrile (25 g, 126.25 mmol), 4-methylthiazole (25.035 g, 252.5 mmol, 2.0 equiv), and anhydrous KOAc (24.78 g, 252.5 mmol) in DMF (210.42 mL, 0.6 M) was barbotated with argon in an ultra-sonic bath for 10 min. Then, Pd(OAc)₂ (0.567 g, 2.52 mmol) was added. The resulting mixture was stirred at 110° C. for 5 h under argon. Pd(OAc)₂ (0.283 g, 1.26 mmol) was then added after one hour, two hours, and three hours (i.e., total amount of Pd(OAc)₂ (1.417 g, 6.31 mmol)). The reaction mixture was cooled down to rt, filtered through Celite, diluted with water, and extracted with ethyl acetate. The combined organic layers were washed with brine, dried over anhydrous sodium sulfate, and concentrated under reduced pressure. The residue was purified by silica gel column chromatography (DCM:MeOH) to provide 2-hydroxy-4-(4-methyl-1,3-thiazol-5-yl)benzonitrile (17.64 g, 64.6%) as a yellow solid. ¹H NMR (300 MHz, DMSO-d₆) δ 11.36 (s, 1H), 9.08 (s, 1H), 7.71 (d, J=8.1 Hz, 1H), 7.14 (d, J=1.6 Hz, 1H), 7.08 (dd, J=8.0, 1.7 Hz, 1H), 2.50 (s, 3H). LCMS: C₁₁H₈N₂OS requires: 216.3, found: m/z=217.49 [M+H]⁺.

### Step 2: Synthesis of 2-(aminomethyl)-5-(4-methyl-1,3-thiazol-5-yl)phenol (HB16f)

To a solution of LAH (1 M in THF, 203.9 mL, 203.92 mmol) was added a solution of 2-hydroxy-4-(4-methyl-1,3-thiazol-5-yl)benzonitrile (17.64 g, 81.57 mmol) in THE (203.92 mL, 0.4 M) slowly under argon at −10° C. After complete addition, the reaction mixture was allowed to slowly warm to room temperature over five hours. The reaction was quenched by the addition of Na₂SO₄·10 H₂O and then was concentrated under reduced pressure. The residue was purified by silica gel column chromatography (DCM:MeOH) to provide 2-(aminomethyl)-5-(4-methyl-1,3-thiazol-5-yl)phenol (9.18 g, 52%) as an amber oil. ¹H NMR (300 MHz, DMSO-d₆) δ 8.96 (s, 1H), 7.23-7.15 (m, 1H), 6.87-6.81 (m, 2H), 3.88 (s, 2H), 2.45 (s, 3H). LCMS: C₁₁H₁₂N₂OS requires: 220.3, found: m/z=221.5 [M+H]⁺.

### Step 3: Synthesis of methyl (2S,4R)-1-[(2S)-2-{[(tert-butoxy)carbonyl]amino}-3,3-dimethylbutanoyl]-4-hydroxypyrrolidine-2-carboxylate (HB16i)

To a solution of methyl (2S)-2-{[(tert-butoxy)carbonyl]amino}-3,3-dimethylbutanoic acid (41.0 g, 0.177 mol) and DIPEA (46.3 mL, 0.266 mol) in anhydrous THE (1770 mL, 0.1 M) was added HATU (70.8 g, 0.186 mol) as a solid in portions at 10° C. to form an activated ester within 30 min. In a separate reactor, a solution of (2S,4R)-4-hydroxypyrrolidine-2-carboxylate hydrochloride (48.0 g, 1.266 mol) and DIPEA (46.3 mL, 0.266 mol, 1.5 equiv) was prepared and cooled down to −45° C. under an inert atmosphere. The solution of activated ester was added dropwise at −45 to −40° C. over 0.5 h and the reaction was left to slowly warm up to room temperature overnight. Water (˜500 mL) was added in a single portion to quench the reaction and the volatiles were removed under vacuum. The resulting oily residue was extracted with EtOAc (3×400 mL), washed with sat. aqueous NaHCO₃ (250 mL), 10% aqueous KHSO₄ (250 mL), and brine (300 mL), dried over MgSO₄, filtered, and evaporated to give a crude which was purified by flash chromatography. Concentration of corresponding fractions gave methyl (2S,4R)-1-[(2S)-2-{[(tert-butoxy)carbonyl]amino}-3,3-dimethylbutanoyl]-4-hydroxypyrrolidine-2-carboxylate as a pale yellow oil (64 g, 99%). ¹H NMR (300 MHz, DMSO-d₆) δ 6.54 (d, J=9.3 Hz, 1H), 5.23 (d, J=3.8 Hz, 1H), 4.42-4.29 (m, 2H), 4.16 (d, J=9.4 Hz, 1H), 3.71-3.61 (m, 2H), 2.11 (dd, J=12.2, 9.2 Hz, 1H), 1.95-1.85 (m, 1H), 1.38 (s, 10H), 0.94 (s, 9H). LCMS: C₁₇H₃₀N₂O₆ requires: 358.44, found: m/z=359.3 [M+H]⁺.

### Step 4: Synthesis of (2S,4R)-1-[(2S)-2-{[(tert-butoxy)carbonyl]amino}-3,3-dimethylbutanoyl]-4-hydroxypyrrolidine-2-carboxylic acid (HB16j)

To a solution of methyl (2S,4R)-1-[(2S)-2-{[(tert-butoxy)carbonyl]amino}-3,3-dimethylbutanoyl]-4-hydroxypyrrolidine-2-carboxylate (63.54 g, 0.177 mol) in THE (220 mL, 0.8 M) was added LiOH H₂O (14.88 g, 0.355 mol) as an aqueous solution (86 mL, 0.2 M) in one portion at room temperature. The reaction was left to stir at room temperature for 3 h and monitored by TLC/UPLC. Once the reaction was completed, 10% aqueous KHSO₄ was added until pH -3. The THF was concentrated by rotovap and the resulting residue was extracted with EtOAc (3×400 mL). The combined organic fractions were washed with 10% aqueous KHSO₄ (200 mL), brine (300 mL), and dried over MgSO₄, filtered, and evaporated to dryness. A viscous pale yellow oily residue was sonicated with anhydrous THE (300 mL) to give an off-white precipitate, which was filtered and dried under vacuum at 50° C. yielding (2S,4R)-1-[(2S)-2-{[(tert-butoxy)carbonyl]amino}-3,3-dimethylbutanoyl]-4-hydroxypyrrolidine-2-carboxylic acid (69.6g, including THE (˜15% by weight)). ¹H NMR (300 MHz, DMSO-d₆) δ 12.43 (s, 1H), 6.49 (d, J=9.4 Hz, 1H), 5.18 (d, J=3.7 Hz, 1H), 4.33 (br s, 1H), 4.26 (t, J=8.4 Hz, 1H), 4.16 (d, J=9.4 Hz, 1H), 3.69-3.52 (m, 2H), 2.18-2.02 (m, 1H), 1.89 (ddd, J=13.2, 9.1, 4.6 Hz, 1H), 1.38 (s, 9H), 0.94 (s, 9H). LCMS: C₁₆H₂₈N₂O₆ requires: 344.4, found: m/z=345.2 [M+H]⁺.

### Step 5: Synthesis of tert-butyl N-[(2S)-1-[(2S,4R)-4-hydroxy-2-({[2-hydroxy-4-(4-methyl-1,3-thiazol-5-yl)phenyl]methyl}carbamoyl)pyrrolidin-1-yl]-3,3-dimethyl-1-oxobutan-2-yl]carbamate (HB16k)

To a solution of (2S,4R)-1-[(2S)-2-{[(tert-butoxy)carbonyl]amino}-3,3-dimethylbutanoyl]-4-hydroxypyrrolidine-2-carboxylic acid (14.352 g, 41.67 mmol) in DMF (138.9 mL, 0.3 M) cooled in an ice-water bath under argon was added DIPEA (10.89 mL, 62.51 mmol) and HATU (16.644 g, 43.76 mmol). The resulting mixture was allowed to warm to room temperature over 0.5 h and was then slowly added dropwise to a solution of 2-(aminomethyl)-5-(4-methyl-1,3-thiazol-5-yl)phenol (9.180 g, 41.67 mmol) and DIPEA (7.26 mL, 42.67 mmol) in DMF (83.34 mL, 0.5 M) at −40° C. under argon. After the addition was complete, the reaction mixture remained in the cooling bath and was allowed to slowly warm to room temperature over five hours. The reaction was then quenched by the addition of water (5 mL) and concentrated under reduced pressure. The residue was purified by silica gel flash chromatography (DCM:MeOH) to provide (2S,4R)-1-[(2S)-2-{[(tert-butoxy)carbonyl]amino}-3,3-dimethylbutanoyl]-4-hydroxypyrrolidine-2-carboxylic acid (13.36 g, 58.64%) as a yellowish solid. LCMS: C₂₇H₃₈N₄O₆S requires: 546.7, found: m/z=547.9 [M+H]⁺.

2-({[(2S,4R)-1-[(2S)-2-{[(tert-butoxy)carbonyl]amino}-3,3-dimethylbutanoyl]-4-hydroxypyrrolidin-2-yl]formamido}methyl)-5-(4-methyl-1,3-thiazol-5-yl)phenyl (2S)-1-(2-{[(tert-butoxy)carbonyl]amino}-3,3-dimethylbutanoyl)pyrrolidine-2-carboxylate (the double-acylated side product) was further isolated after purification by flash chromatography. ¹H NMR (300 MHz, Chloroform-d) δ 9.28 (br s, 1H), 8.70 (s, 1H), 8.11 (t, J=6.6 Hz, 1H), 7.13 (d, J=7.8 Hz, 1H), 6.98 (d, J=1.8 Hz, 1H), 6.88 (dd, J=7.7, 1.8 Hz, 1H), 5.19 (d, J=8.9 Hz, 1H), 4.77 (t, J=7.9 Hz, 1H), 4.51 (dd, J=15.0, 6.9 Hz, 2H), 4.12 (td, J=20.4, 8.4 Hz, 3H), 3.57 (dd, J=11.4, 3.6 Hz, 1H), 2.85 (br s, 2H), 2.53 (m, 4H), 2.11 (dd, J=13.5, 8.1 Hz, 1H), 1.56-1.43 (m, 2H), 1.41 (s, 9H), 0.84 (s, 9H). One acyl group can be cleaved according to Step 5a.

### Step 5a: Synthesis of tert-butyl N-[(2S)-1-[(2S,4R)-4-hydroxy-2-({[2-hydroxy-4-(4-methyl-1,3-thiazol-5-yl)phenyl]methyl}carbamoyl)pyrrolidin-1-yl]-3,3-dimethyl-1-oxobutan-2-yl]carbamate (HB16k)

To a solution of 2-({[(2S,4R)-1-[(2S)-2-{[(tert-butoxy)carbonyl]amino}-3,3-dimethylbutanoyl]-4-hydroxypyrrolidin-2-yl]formamido}methyl)-5-(4-methyl-1,3-thiazol-5-yl)phenyl (2S)-1-(2-{[(tert-butoxy)carbonyl]amino}-3,3-dimethylbutanoyl)pyrrolidine-2-carboxylate (3 g, 3.5 mmol) in MeOH (70 mL, 0.05 M) was added K₂CO₃ (0.484 g, 3.5 mmol). The reaction mixture was left to stir at rt for 12 h. The reaction mixture was then concentrated, and the residue was diluted with water, neutralized with KHSO₄, and extracted with DCM (3×). The combined organic layer was dried with Na₂SO₄ and concentrated under reduced pressure. The obtained residue was purified by silica gel flash chromatography (5% DCM:MeOH) to provide tert-butyl N-[(2S)-1-[(2S,4R)-4-hydroxy-2-({[2-hydroxy-4-(4-methyl-1,3-thiazol-5-yl)phenyl]methyl}carbamoyl)pyrrolidin-1-yl]-3,3-dimethyl-1-oxobutan-2-yl]carbamate (2.14 g, 99%) as a yellowish solid. ¹H NMR (300 MHz, Chloroform-d) δ 9.29 (s, 1H), 8.80 (s, 1H), 8.19 (s, 1H), 7.14 (d, J=7.8 Hz, 1H), 6.98 (d, J=1.8 Hz, 1H), 6.87 (dd, J=7.7, 1.8 Hz, 1H), 5.14 (d, J=8.9 Hz, 1H), 4.81 (t, J=7.9 Hz, 1H), 4.56 (q, J=7.8 Hz, 2H), 4.12 (td, J=13.6, 12.6, 4.7 Hz, 3H), 3.56 (dd, J=11.4, 3.5 Hz, 1H), 2.56 (s, 4H), 2.19-2.05 (m, 1H), 0.83 (s, 10H). LCMS: C₂₇H₃₈N₄O₆S requires: 546.7, found: m/z=547.2 [M+H]⁺.

### Step 6: Synthesis of (2S,4R)-1-[(2S)-2-amino-3,3-dimethylbutanoyl]-4-hydroxy-N-{[2-hydroxy-4-(4-methyl-1,3-thiazol-5-yl)phenyl]methyl}pyrrolidine-2-carboxamide (HB16m)

To a solution of tert-butyl N-[(2S)-1-[(2S,4R)-4-hydroxy-2-({[2-hydroxy-4-(4-methyl-1,3-thiazol-5-yl)phenyl]methyl}carbamoyl)pyrrolidin-1-yl]-3,3-dimethyl-1-oxobutan-2-yl]carbamate (5.27 g, 9.64 mmol) in DCM (48.2 mL, 0.2 M) cooled in an ice-water bath was added HCl (2 M in Et₂O, 38.56 mL, 77.12 mmol). The reaction mixture was then stirred at room temperature for two hours. The solid was triturated on an ultra-sonic bath, filtered, washed with DCM, and dried under vacuum to provide (2S,4R)-1-[(2S)-2-amino-3,3-dimethylbutanoyl]-4-hydroxy-N-{[2-hydroxy-4-(4-methyl-1,3-thiazol-5-yl)phenyl]methyl}pyrrolidine-2-carboxamide (5.05 g, 99%) as a white solid. ¹H NMR (300 MHz, D₂O) δ 9.50 (d, J=1.0 Hz, 1H), 7.30 (d, J=7.8 Hz, 1H), 7.04-6.89 (m, 2H), 4.58 (dd, J=9.9, 7.6 Hz, 1H), 4.52 (s, 1H), 4.44-4.23 (m, 2H), 4.08 (s, 1H), 3.80 (d, J=11.9 Hz, 1H), 3.68 (dd, J=11.9, 3.4 Hz, 1H), 3.46 (q, J=7.1 Hz, 1H), 2.45 (s, 3H), 2.28 (dd, J=13.9, 7.7 Hz, 1H), 2.01 (ddd, J=14.0, 9.9, 4.2 Hz, 1H), 1.08 (t, J=7.1 Hz, 2H), 0.98 (s, 9H). LCMS: C₂₂H₃₀N₄O₄S requires: 446.6, found: m/z=447.7 [M+H]⁺.

### Step 7: Synthesis of (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (HB16)

To a solution of 1-fluorocyclopropane-1-carboxylic acid (1.337 g, 12.85 mmol) in DMF (128 mL, 0.1 M) cooled in an ice-water bath was added HATU (5.129 g, 13.49 mmol) and DIPEA (3.36 mL, 19.27 mmol). The resulting mixture was allowed to warm to room temperature over 0.5 h and was then added dropwise to a solution of (2S,4R)-1-[(2S)-2-amino-3,3-dimethylbutanoyl]-4-hydroxy-N-{[2-hydroxy-4-(4-methyl-1,3-thiazol-5-yl)phenyl]methyl}pyrrolidine-2-carboxamide (6.674 g, 12.85 mmol) and DIPEA (7.83 mL, 44.97 mmol) in DMF (42 mL, 0.3 M) at −40° C. After addition, the reaction mixture remained in the cooling bath and slowly warmed to room temperature over sixteen hours. The reaction was then diluted with water and extracted with ethyl acetate. The combined organic layers were washed with brine, dried over anhydrous sodium sulfate, and concentrated under reduced pressure. The residue was purified by silica gel column chromatography (DCM:MeOH) to provide (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (5.05 g, 74%) as a yellow solid. ¹H NMR (300 MHz, Chloroform-d) δ 9.29 (s, 1H), 8.70 (s, 1H), 8.09 (dd, J=7.5, 5.5 Hz, 1H), 7.13 (d, J=7.8 Hz, 1H), 7.01 (dd, J=8.5, 3.7 Hz, 1H), 6.98 (d, J=1.8 Hz, 1H), 6.88 (dd, J=7.7, 1.8 Hz, 1H), 4.73 (t, J=7.9 Hz, 1H), 4.53 (br s, 1H), 4.51-4.40 (m, 2H), 4.18 (dd, J=14.6, 5.4 Hz, 1H), 3.99 (d, J=11.3 Hz, 1H), 3.63 (dd, J=11.2, 3.7 Hz, 1H), 2.53 (s, 3H), 2.47 (ddd, J=12.9, 7.9, 4.6 Hz, 1H), 2.15-2.01 (m, 1H), 1.36-1.22 (m, 4H), 0.91 (s, 9H). LCMS: C₂₆H₃₃N₄O₅SF requires: 532.6, found: m/z=533.8 [M+H]⁺.

Described below are additional examples of VHL-targeting LHM building blocks that may be prepared according to Scheme D1.

### Example 23. 6-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)hexanoic acid (HB17)

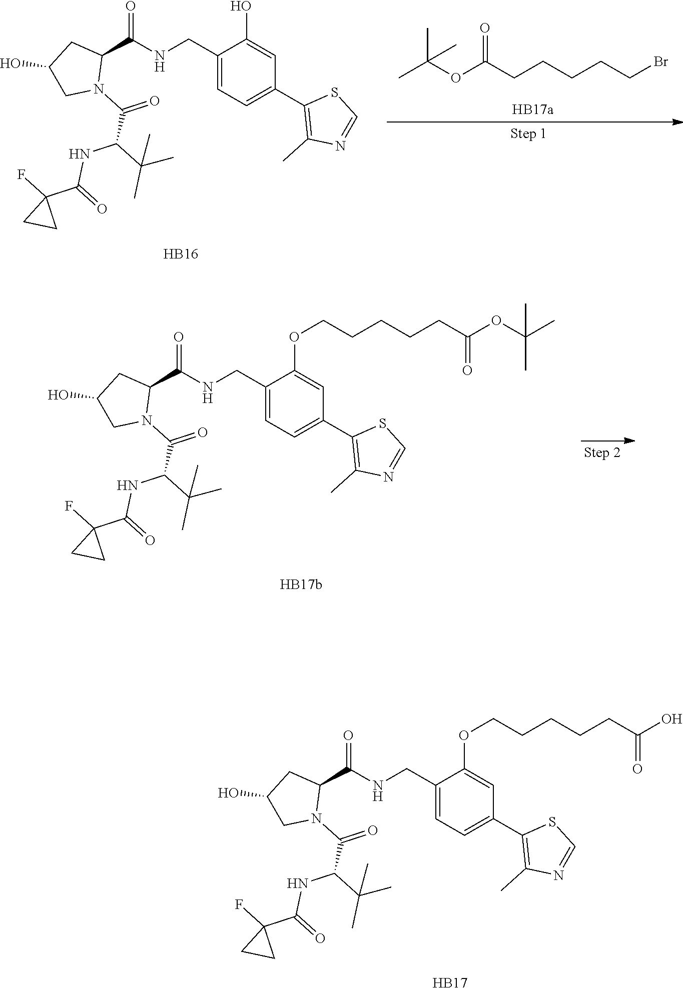

### Step 1: Synthesis of tert-butyl 6-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]hexanoate (HB17b)

To a solution of (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (1.29 g, 2.42 mmol, 1.0 equiv) in anhydrous DMF (16 mL, 0.15 M) was added Cs₂CO₃ (1.184 g, 3.63 mmol, 1.5 equiv) and tert-butyl 6-bromohexanoate (0.85 g, 3.4 mmol, 1.4 equiv). The reaction mixture was purged with argon, sealed, and stirred at 25° C. for sixteen hours. The solids were filtered, washed with EtOAc (5 mL), and discarded. The filtrate was diluted with water (60 mL) and extracted with ethyl acetate (3×20 mL). The combined organic layers were washed with brine (20 mL), dried over Na₂SO₄, filtered, and concentrated under reduced pressure. The crude was purified by flash chromatography (hexane:ethyl acetate) to give the desired product as a white solid (1.38 g, 81.1%). ESI(+) [M+H]⁺=703.8.

### Step 2: Synthesis of 6-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]hexanoic acid (HB17)

To a solution of tert-butyl 6-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl) phenoxy]hexanoate (1.38 g, 1.96 mmol, 1.0 equiv) in anhydrous DCM (147.3 mL, 0.4 M) was added HCl (2 M in diethyl ether, 30 mL). The reaction mixture was then stirred overnight at room temperature. Solvent was evaporated under reduced pressure to give a residue, which was dissolved in THF (10 mL) and triturated with aqueous ammonia (3 M, 5 mL) for 10 min and then concentrated again. The crude was purified by reverse phase flash chromatography to give 6-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)hexanoic acid (614 mg, 48%) as an off-white amorphous solid. LCMS (254 nm), Rₜ=2.59 min, 95.62% purity. ¹H NMR (300 MHz, Methanol-d₄) δ 8.86 (s, 1H), 7.50 (dd, J=19.7, 9.1 Hz, 2H), 7.07-6.92 (m, 2H), 4.80-4.66 (m, 1H), 4.63 (t, J=8.3 Hz, 1H), 4.50 (d, J=3.2 Hz, 1H), 4.42 (d, J=9.6 Hz, 1H), 4.07 (t, J=6.2 Hz, 2H), 3.91-3.62 (m, 2H), 2.48 (s, 3H), 2.34 (t, J=7.2 Hz, 2H), 2.27-2.02 (m, 2H), 1.87 (p, J=6.6 Hz, 2H), 1.65 (dp, J=33.1, 8.5, 7.8 Hz, 4H), 1.47-1.18 (m, 5H), 1.03 (s, 10H). ESI(+)=647.13 [M+H]⁺.

### Example 24. HVB5: 8-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)octanoic acid (HB18)

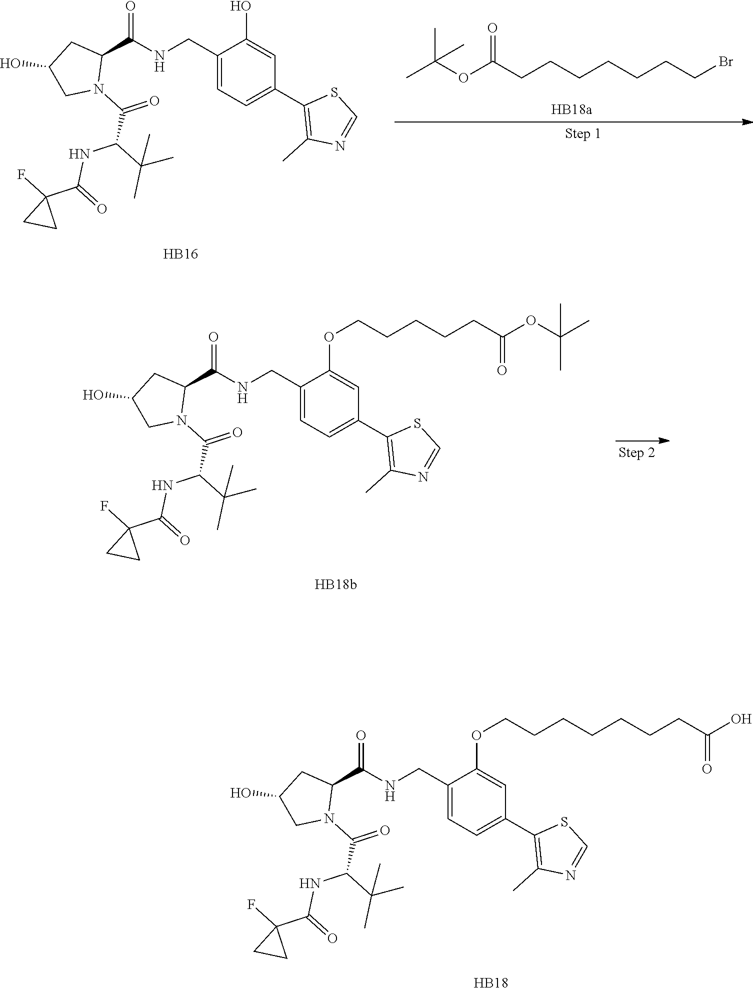

HB18 was prepared according to the same method as HB17, except that HB17a was replaced with HB18a. LCMS: C₃₄H₄₇N₄O₇S requires: 674, found: m/z=675 [M+H]⁺.

### Example 25. 3-{2-[2-({[(2S,4R)-1-[(2S)-2-[(1-fluorocyclopropyl)formamido]-3-methylbutanoyl]-4-hydroxypyrrolidin-2-yl]formamido}methyl)-5-(4-methyl-1,3-thiazol-5-yl)phenoxy]ethoxy}propanoic acid (HB19)

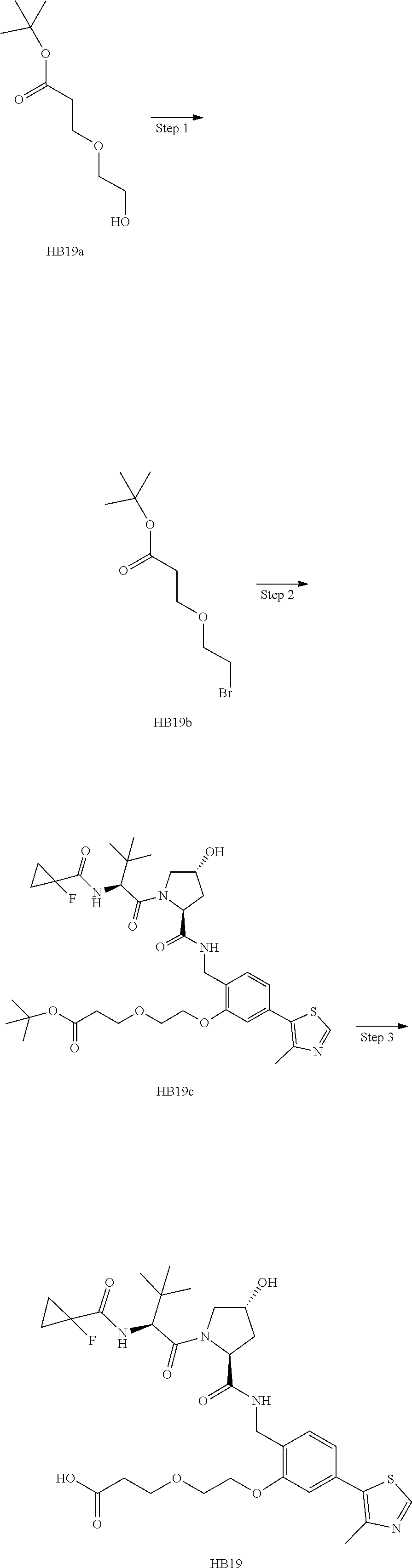

### Step 1: Synthesis of tert-butyl 3-(2-bromoethoxy)propanoate (HB19b)

A solution of tert-butyl 3-(2-hydroxyethoxy)propanoate (3.0 g, 15.7 mmol, 1 equiv) and carbon tetrabromide (3.9 g, 11.87 mmol, 1.5 equiv) in dichloromethane (15 mL, 1 M) was prepared in a 50 mL flask and cooled to 0° C. Triphenylphosphine (3.1 g, 11.87 mmol, 1.5 equiv) was added via powder funnel in portions over 30 min with vigorous stirring. Upon addition of the phosphine, the colorless solution turned a pale brown color and was stirred for an additional 2 h at room temperature. The mixture was concentrated and quickly added to stirring hexane (50 mL).

The white precipitate was filtered, the remaining solution was concentrated, and the obtained residue was purified by flash column chromatography (eluted DCM:MeOH 9:1) to give 4.1 g of HB19b as a white solid (62.8%).

### Step 2: Synthesis of tert-butyl 3-{2-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]ethoxy}propanoate (HB19c)

To a solution of (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (1.5 g, 2.82 mmol, 1.0 equiv) in DMF (18.77 mL, 0.15 M) was added Cs₂CO₃ (1.376 g, 4.22 mmol, 1.5 equiv) and tert-butyl 3-(2-bromoethoxy)propanoate (2.18 g, 3.94 mmol, 1.4 equiv). The resulting mixture was stirred at room temperature overnight. The reaction mixture was diluted with water and extracted with EtOAc (3 times). The organic layers were combined and dried using Na₂SO₄, concentrated, and the residue was purified by flash column chromatography (eluted with DCM:MeOH 9:1) to give the desired product as a pale yellow oil (1.8 g, quantitative yield). UPLC (12 min, 254 nm): Rₜ=6.25 min, 100% purity, ESI [M+H]⁺=705.55.

### Step 3: Synthesis of 3-{2-[2-({[(2S,4R)-1-[(2S)-2-[(1-fluorocyclopropyl)formamido]-3-methylbutanoyl]-4-hydroxypyrrolidin-2-yl]formamido}methyl)-5-(4-methyl-1,3-thiazol-5-yl)phenoxy]ethoxy}propanoic acid (HB19)

To a solution of tert-butyl 3-{2-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]ethoxy}propanoate (1.8 g, 2.64 mmol, 1 equiv) in DCM (17.6 mL, 0.15 M) at 0° C. was added dropwise TFA (13.2 mL). The reaction mixture was left to stir at room temperature for one hour. The reaction mixture was concentrated, the residue was diluted with aqueous NH₄OH (50 mL or until pH=11), left stirring in an ultrasonic bath for 0.5 h, and then for one hour. The resulting slurry was concentrated and purified by reverse phase chromatography twice: first, eluted with ACN:H₂O to give 0.3 g of the desired product; and second, eluted with ACN:H₂O (0.1% formic acid) to give 1 g of the desired product. After neutralization with saturated ammonium hydroxide, the product was isolated as the ammonium salt, which was released with formic acid during the second purification. The desired products were combined (1.3 g, 76%). ¹H NMR (300 MHz, Chloroform-d) δ 8.70 (s, 1H), 7.37 (d, J=7.8 Hz, 2H), 7.09-7.03 (m, 1H), 6.99 (dd, J=7.7, 1.6 Hz, 1H), 6.91 (d, J=1.6 Hz, 1H), 4.76 (t, J=8.1 Hz, 1H), 4.64-4.51 (m, 3H), 4.41 (dd, J=14.3, 5.2 Hz, 1H), 4.20 (t, J=4.2 Hz, 2H), 4.03 (d, J=11.3 Hz, 1H), 3.89 (td, J=8.6, 7.8, 4.4 Hz, 4H), 3.77 (dd, J=11.3, 3.7 Hz, 1H), 2.66 (ddd, J=19.7, 14.9, 5.1 Hz, 2H), 2.54 (s, 3H), 2.33-2.14 (m, 2H), 1.41-1.23 (m, 4H), 1.03 (s, 9H). LCMS (254 nm): Rₜ=2.29 min, 99% purity, ESI(+) [M+H]⁺=649.10.

### Example 26. 3-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)propanoic acid (HB20)

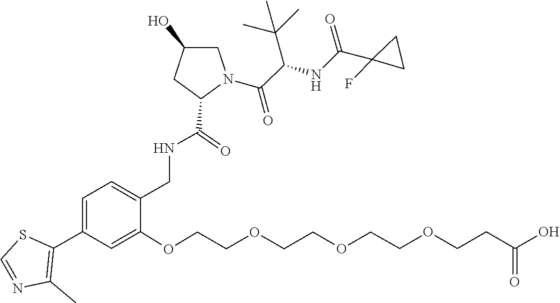 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCC(=O)O)c1]

HB20 was prepared in an analogous manner as HB19 except substituting tert-butyl 3-(2-hydroxyethoxy)propanoate with tert-butyl 3-{2-[2-(2-bromoethoxy)ethoxy]ethoxy}propanoate in Step 1 to obtain the title compound as a white solid. ¹H NMR (300 MHz, DMSO-d₆) δ 8.98 (s, 1H), 8.51 (t, J=6.0 Hz, 1H), 7.41 (d, J=7.8 Hz, 1H), 7.31 (dd, J=9.2, 2.9 Hz, 1H), 7.04 (d, J=1.7 Hz, 1H), 6.97 (dd, J=7.7, 1.6 Hz, 1H), 5.19 (s, 1H), 4.60 (d, J=9.1 Hz, 1H), 4.51 (t, J=8.2 Hz, 1H), 4.35 (s, 1H), 4.28 (d, J=6.1 Hz, 1H), 4.25-4.14 (m, 3H), 3.79 (dd, J=5.8, 3.4 Hz, 2H), 3.66-3.46 (m, 12H), 2.46 (s, 3H), 2.42 (t, J=6.3 Hz, 2H), 2.10 (dd, J=13.0, 8.0 Hz, 1H), 1.92 (ddd, J=13.1, 9.0, 4.4 Hz, 1H), 1.49-1.28 (m, 2H), 1.21 (tq, J=8.4, 4.6, 3.8 Hz, 2H), 0.96 (s, 9H). LCMS (254 nm): Rₜ=2.27 min, 96.35% purity, ESI [M+H]⁺=736.88.

### Example 27. 2-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]acetic acid (HB21)

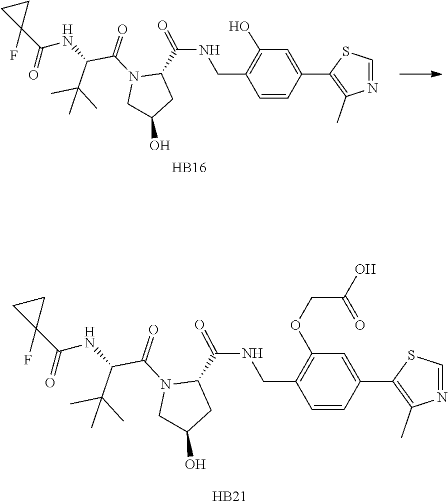

Prepared analogously to HB18, but with tert-butyl bromoacetate in place of tert-butyl 6-bromooctanoate. LCMS: C₂₈H₃₅FN₄O₇S requires: 590.22, found: m/z=591.3 [M+H]⁺.

### Example 28. tert-butyl 4-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]butanoate (HB22)

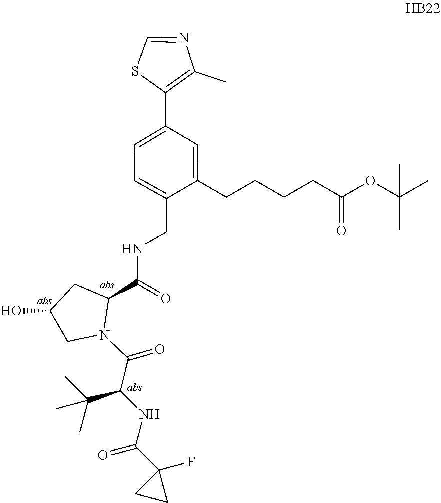 [SMILES: Cc1ncsc1-c1ccc(CNC(=O)[C@@H]2C[C@@H](O)CN2C(=O)[C@@H](NC(=O)C2(F)CC2)C(C)(C)C)c(CCCCC(=O)OC(C)(C)C)c1]

(2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (519.00 mg, 0.97 mmol) and cesium carbonate (0.63 g, 1.94 mmol) were dissolved in dimethylformamide (4.00 mL) in a 20 mL scintillation vial. Tert-butyl 4-bromobutanoate (190.00 μL, 242.06 mg, 1.08 mmol) was added and the reaction turned cloudy yellow. The reaction was allowed to stir at rt for 2 h. The reaction was diluted with ethyl acetate and washed with 0.5 M HCl. The organic layer was dried over MgSO₄, filtered, and concentrated to a yellow oil and carried forward without purification. LCMS: C₃₄H₄₇FN₄O₇S requires 674.3, found m/z=675.5 [M+H]⁺.

### Example 29. 4-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]butanoic acid (HB23)

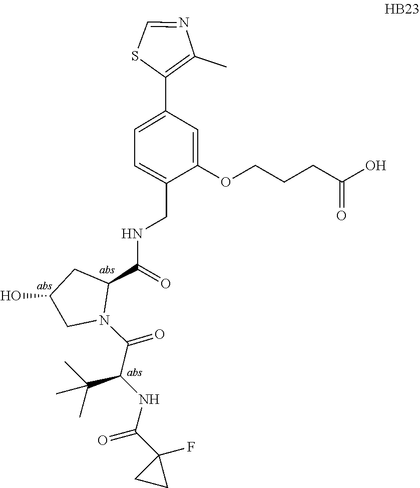 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)O)c1]

In a 4 dram vial, a solution of tert-butyl 4-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]butanoate (HB22) (654.59 mg, 0.97 mmol) in trifluoroacetic acid (1.50 mL, 2.23 g, 19.59 mmol) and methylene chloride (4.00 mL, 5.32 g, 62.64 mmol) was allowed to stir at rt for 4 h. By LCMS, conversion to product was observed, but a TFA ester of the secondary alcohol also formed. Volatiles were removed in vacuo and the resulting yellow-orange oil was stirred with aqueous ammonia (20%, 2.5 mL) for one hour. A yellow-orange oil separated out. The aqueous phase was extracted with ethyl acetate. The combined organic phases were concentrated. The resulting yellow oil was purified by reverse phase-HPLC (5%-100% MeCN in H₂O), concentrated, and lyophilized to provide 4-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]butanoic acid (0.1117 g, 18.6%) as a fluffy white powder. ¹H NMR (500 MHz, Methanol-d₄) δ 8.87 (s, 1H), 7.50 (dd, J=26.2, 8.2 Hz, 2H), 7.01 (d, J=6.1 Hz, 2H), 4.75 (d, J=7.9 Hz, 1H), 4.64 (t, J=8.5 Hz, 1H), 4.53-4.37 (m, 3H), 4.13 (t, J=6.0 Hz, 2H), 3.94-3.77 (m, 2H), 2.55 (t, J=7.1 Hz, 2H), 2.49 (s, 2H), 2.24 (dd, J=13.3, 7.9 Hz, 1H), 2.14 (dt, J=15.7, 7.7 Hz, 3H), 1.44-1.23 (m, 4H), 1.04 (s, 9H). LCMS: C₃₀H₃₉FN₄O₇S requires 618.3, found m/z=619.5 [M+H]⁺.

### Example 30. tert-butyl 5-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]pentanoate (HB24)

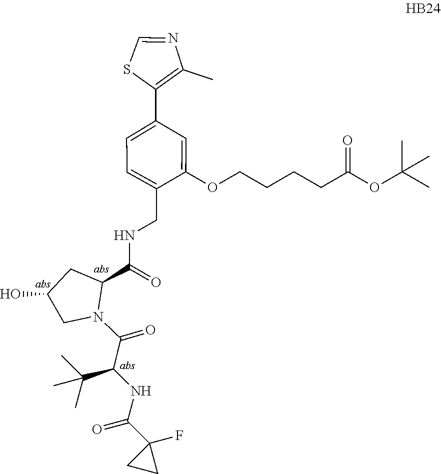 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCC(=O)OC(C)(C)C)c1]

(2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (535.30 mg, 1.01 mmol) and cesium carbonate (0.65 g, 1.99 mmol) were dissolved in dimethylformamide (3.00 mL) in a 20 mL scintillation vial. Tert-butyl 5-bromopentanoate (253.70 mg, 1.07 mmol) was dissolved in dry dimethylformamide (1.00 mL) and added to the reaction, which turned cloudy yellow. The reaction was allowed to stir at rt for 4 h. The reaction was diluted with ethyl acetate and washed with 0.5 M HCl. The organic layer was dried over Na₂SO₄, filtered, and concentrated to a yellow oil and carried forward without purification. LCMS: C₃₅H₄₉FN₄O₇S requires 688.3, found m/z=689.6 [M+H]⁺.

### Example 31. 5-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]pentanoic acid (HB25)

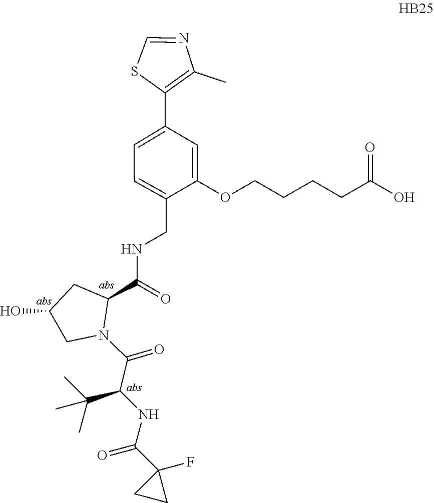 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCC(=O)O)c1]

In a 4 dram vial, a solution of tert-butyl 5-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]pentanoate (695.75 mg, 1.01 mmol) in trifluoroacetic acid (0.50 mL, 0.75 g, 6.53 mmol) and methylene chloride (2.00 mL, 2.66 g, 31.32 mmol) was allowed to stir at rt for 96 h. Additional trifluoroacetic acid (1.5 mL, 19.6 mmol) was then added. After four more hours, the starting material was consumed. Volatiles were removed in vacuo and the reaction was purified by reverse phase-HPLC (5%-100% MeCN in H₂O), concentrated, and lyophilized overnight to provide 5-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]pentanoic acid (0.1291 g, 18.4%) as a fluffy white powder. ¹H NMR (500 MHz, Methanol-d₄) δ 8.86 (s, 1H), 7.53 (dd, J=9.5, 3.4 Hz, 1H), 7.47 (d, J=7.7 Hz, 1H), 6.99 (d, J=7.3 Hz, 2H), 4.75 (d, J=8.9 Hz, 1H), 4.65 (t, J=8.3 Hz, 1H), 4.54-4.36 (m, 3H), 4.08 (t, J=5.9 Hz, 2H), 3.89-3.75 (m, 2H), 2.48 (s, 3H), 2.40 (t, J=7.0 Hz, 2H), 2.24 (ddt, J=13.2, 7.6, 1.9 Hz, 1H), 2.12 (ddd, J=13.3, 9.0, 4.4 Hz, 1H), 2.03 (s, 2H), 1.87 (tdd, J=15.1, 7.5, 4.3 Hz, 3H), 1.44-1.21 (m, 2H), 1.04 (s, 9H). LCMS: C₃₁H₄₁FN₄O₇S requires 632.3, found m/z=633.5 [M+H]⁺.

### Example 32. tert-butyl 3-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]azetidine-1-carboxylate (HB26)

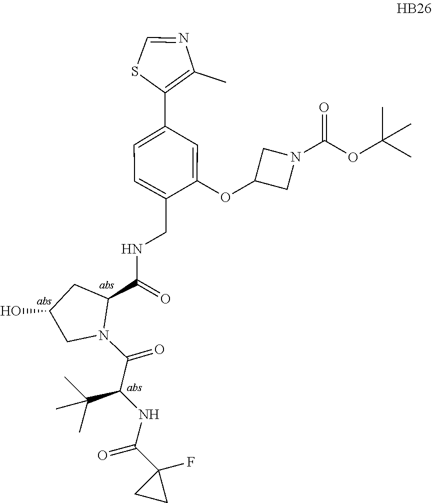 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CN(C(=O)OC(C)(C)C)C2)c1]

(2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (512.00 mg, 0.96 mmol) and cesium carbonate (0.63 g, 1.92 mmol) were dissolved in dimethylformamide (4.00 mL, 3.76 g, 51.44 mmol) in a 20 mL scintillation vial. Tert-butyl 3-bromoazetidine-1-carboxylate (272.36 mg, 1.15 mmol) was dissolved in dimethylformamide (1 mL) and added dropwise. The reaction was stirred at rt for 20 h and then heated at 80° C. for 20 h. The reaction was diluted with ethyl acetate and washed with brine. The organic layer was dried over Na₂SO₄, filtered, and concentrated to an orange oil and carried forward without purification. LCMS: C₃₄H₄₆FN₅O₇S requires 687.3, found m/z=688.6 [M+H]⁺.

### Example 33. (2S,4R)—N-{[2-(azetidin-3-yloxy)-4-(4-methyl-1,3-thiazol-5-yl)phenyl]methyl}-1-[(2S)-2-[(1-fluorocyclopropyl)formamido]-3,3-dimethylbutanoyl]-4-hydroxypyrrolidine-2-carboxamide (HB27)

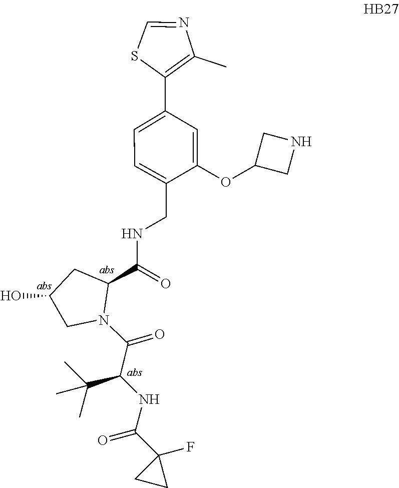 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CNC2)c1]

tert-butyl 3-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]azetidine-1-carboxylate (660.32 mg, 0.96 mmol) was dissolved in methylene chloride (2.11 mL, 2.80 g, 33.02 mmol) in a 20 mL scintillation vial. Hydrogen chloride (2.11 mL, 0.31 g, 8.44 mmol) was then added dropwise. The reaction was allowed to stir at rt for 3 h. The reaction was concentrated to a yellow foam and carried forward without purification. LCMS: C₂₉H₃₈FN₅O₅S requires 587.3, found m/z=588.5 [M+H]⁺.

### Example 34. (2S,4R)-1-[(2S)-2-[(1-fluorocyclopropyl)formamido]-3,3-dimethylbutanoyl]-N-[(2-{[1-(2-fluoropyridin-4-yl)azetidin-3-yl]oxy}-4-(4-methyl-1,3-thiazol-5-yl)phenyl)methyl]-4-hydroxypyrrolidine-2-carboxamide (HB28)

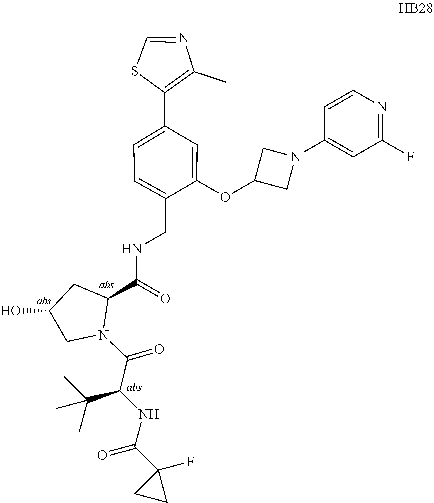 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CN(c3ccnc(F)c3)C2)c1]

(2S,4R)—N-{[2-(azetidin-3-yloxy)-4-(4-methyl-1,3-thiazol-5-yl)phenyl]methyl}-1-[(2S)-2-[(1-fluorocyclopropyl)formamido]-3,3-dimethylbutanoyl]-4-hydroxypyrrolidine-2-carboxamide (282.10 mg, 0.48 mmol) and 2,4-difluoropyridine (43.50 μL, 55.24 mg, 0.48 mmol) were dissolved in dimethylformamide (2.40 mL, 2.26 g, 30.86 mmol) at 0° C. and N,N-diisopropylethylamine (0.17 mL, 0.12 g, 0.96 mmol) was added dropwise while stirring. The reaction was warmed to rt and stirred for 20 h. The reaction was diluted with water and extracted with ethyl acetate. The combined organic extracts were washed with brine, dried over Na₂SO₄, filtered, and concentrated. The crude was purified by flash chromatography on a 12 g column, eluted by gradient elution with 0 to 10% MeOH:CH₂Cl₂ to provide (2S,4R)-1-[(2S)-2-[(1-fluorocyclopropyl)formamido]-3,3-dimethylbutanoyl]-N-[(2-{[1-(2-fluoropyridin-4-yl)azetidin-3-yl]oxy}-4-(4-methyl-1,3-thiazol-5-yl)phenyl)methyl]-4-hydroxypyrrolidine-2-carboxamide (0.1130 g, 34.5%) as a white foam. ¹H NMR (500 MHz, Chloroform-d) δ 8.67 (s, 1H), 7.78 (d, J=5.8 Hz, 1H), 7.48 (t, J=6.2 Hz, 1H), 7.40 (d, J=7.8 Hz, 1H), 7.06 (dd, J=9.1, 3.6 Hz, 1H), 7.03-6.96 (m, 1H), 6.59-6.50 (m, 1H), 6.14 (d, J=5.7 Hz, 1H), 5.78 (d, J=1.9 Hz, 1H), 5.14 (t, J=3.9 Hz, 1H), 4.65 (t, J=7.7 Hz, 1H), 4.57 (d, J=9.0 Hz, 1H), 4.46 (q, J=6.6, 5.5 Hz, 3H), 4.37 (dd, J=8.9, 6.3 Hz, 2H), 3.86 (d, J=11.1 Hz, 1H), 3.63 (dd, J=11.1, 4.1 Hz, 1H), 2.36 (ddd, J=12.8, 7.7, 4.8 Hz, 1H), 2.08-2.03 (m, 1H), 1.33-1.24 (m, 1H), 0.92 (s, 10H). ¹⁹F NMR (471 MHz, Chloroform-d) δ −69.66, −197.29. LCMS: C₂₉H₃₈FN₅O₅S requires 682.3, found m/z=683.5 [M+H]⁺.

### Example 35. 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (HB29)

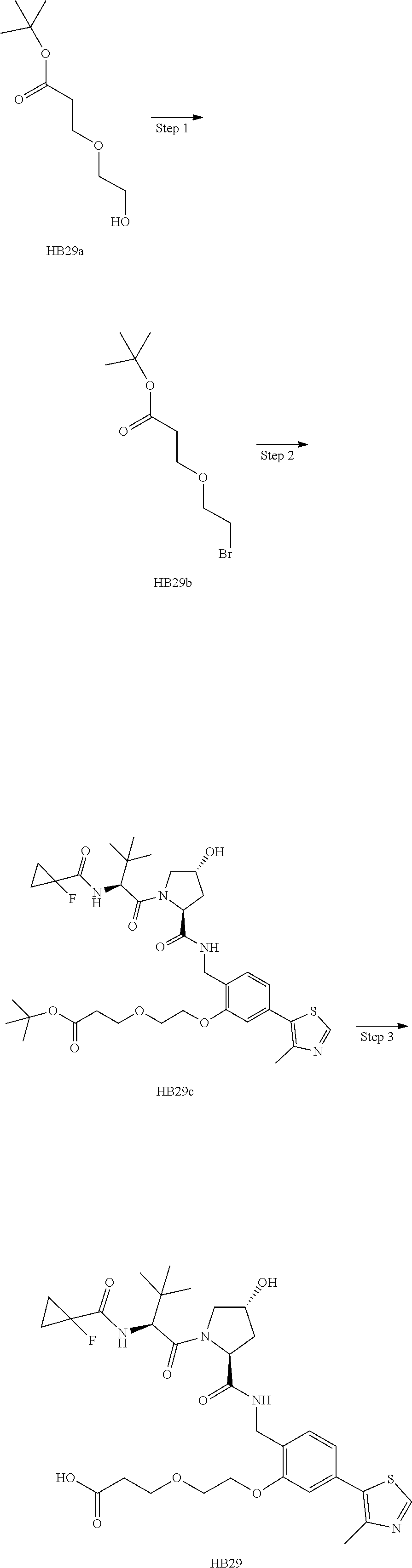

### Step 1: Synthesis of tert-butyl 3-(2-bromoethoxy)propanoate (HB29b)

A solution of tert-butyl 3-(2-hydroxyethoxy)propanoate (3.0 g, 15.7 mmol, 1 equiv) and carbon tetrabromide (3.9 g, 11.87 mmol, 1.5 equiv) in dichloromethane (15 mL, 1 M) was prepared in a 50 mL flask and cooled to 0° C. Triphenylphosphine (3.1 g, 11.87 mmol, 1.5 equiv) was added via powder funnel in portions over 30 min with vigorous stirring. Upon addition of the phosphine, the colorless solution turned a pale brown color and was stirred for an additional 2 h at room temperature. The mixture was concentrated and quickly added to stirring hexane (50 mL). The white precipitate was filtered, the remaining solution was concentrated, and the obtained residue was purified by flash column chromatography (eluted DCM:MeOH 9:1) to give the desired product as a white solid (4.1 g, 62.8%).

### Step 2: Synthesis of tert-butyl 3-{2-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]ethoxy}propanoate (HB29c)

To a solution of (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (1.5 g, 2.82 mmol, 1.0 equiv) in DMF (18.77 mL, 0.15 M) was added Cs₂CO₃ (1.376 g, 4.22 mmol, 1.5 equiv) and tert-butyl 3-(2-bromoethoxy)propanoate (2.18 g, 3.94 mmol, 1.4 equiv). The resulting mixture was stirred at room temperature overnight. The reaction mixture was diluted with water and extracted with EtOAc (3 times). The organic layers were combined and dried under Na₂SO₄, concentrated, and the residue was purified by flash column chromatography eluted with DCM:MeOH 9:1 to give the desired product as a pale yellow oil (1.8 g, quantitative yield). UPLC (12 min, 254 nm): Rₜ=6.25 min, 100% purity, ESI [M+H]⁺=705.55.

### Step 3: Synthesis of 3-{2-[2-({[(2S,4R)-1-[(2S)-2-[(1-fluorocyclopropyl)formamido]-3-methylbutanoyl]-4-hydroxypyrrolidin-2-yl]formamido}methyl)-5-(4-methyl-1,3-thiazol-5-yl)phenoxy]ethoxy}propanoic acid (HB29)

To a solution of tert-butyl 3-{2-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]ethoxy}propanoate (1.8 g, 2.64 mmol, 1 equiv) in DCM (17.6 mL, 0.15 M) at 0° C. was added dropwise TFA (13.2 mL). The reaction mixture was left to stir at room temperature for one hour. The reaction mixture was concentrated, the residue was diluted with aqueous NH₄OH (50 mL or until pH=11), left stirring in an ultrasonic bath for 0.5 h, and then for one hour. The resulting slurry was concentrated and purified by reverse phase chromatography twice: first, eluted with ACN:H₂O to give 0.3 g of the desired product; and second, eluted with ACN:H₂O (0.1% formic acid) to give 1 g of the desired product.

After neutralization with saturated ammonium hydroxide, the product was isolated as the ammonium salt, which was released with formic acid during the second purification. The desired products were combined (1.3 g, 76%). LCMS (254 nm): Rₜ=2.29 min, 99% purity, ESI(+) [M+H]⁺=649.10. ¹H NMR (300 MHz, Chloroform-d) δ 8.70 (s, 1H), 7.37 (d, J=7.8 Hz, 2H), 7.09-7.03 (m, 1H), 6.99 (dd, J=7.7, 1.6 Hz, 1H), 6.91 (d, J=1.6 Hz, 1H), 4.76 (t, J=8.1 Hz, 1H), 4.64-4.51 (m, 3H), 4.41 (dd, J=14.3, 5.2 Hz, 1H), 4.20 (t, J=4.2 Hz, 2H), 4.03 (d, J=11.3 Hz, 1H), 3.89 (td, J=8.6, 7.8, 4.4 Hz, 4H), 3.77 (dd, J=11.3, 3.7 Hz, 1H), 2.66 (ddd, J=19.7, 14.9, 5.1 Hz, 2H), 2.54 (s, 3H), 2.33-2.14 (m, 2H), 1.41-1.23 (m, 4H), 1.03 (s, 9H).

### Example 36. (S)-24-((2S,4R)-4-hydroxy-2-((4-(4-methylthiazol-5-yl)benzyl)carbamoyl)pyrrolidine-1-carbonyl)-25,25-dimethyl-22-oxo-4,7,10,13,16,19-hexaoxa-23-azahexacosanoic acid (HB30)

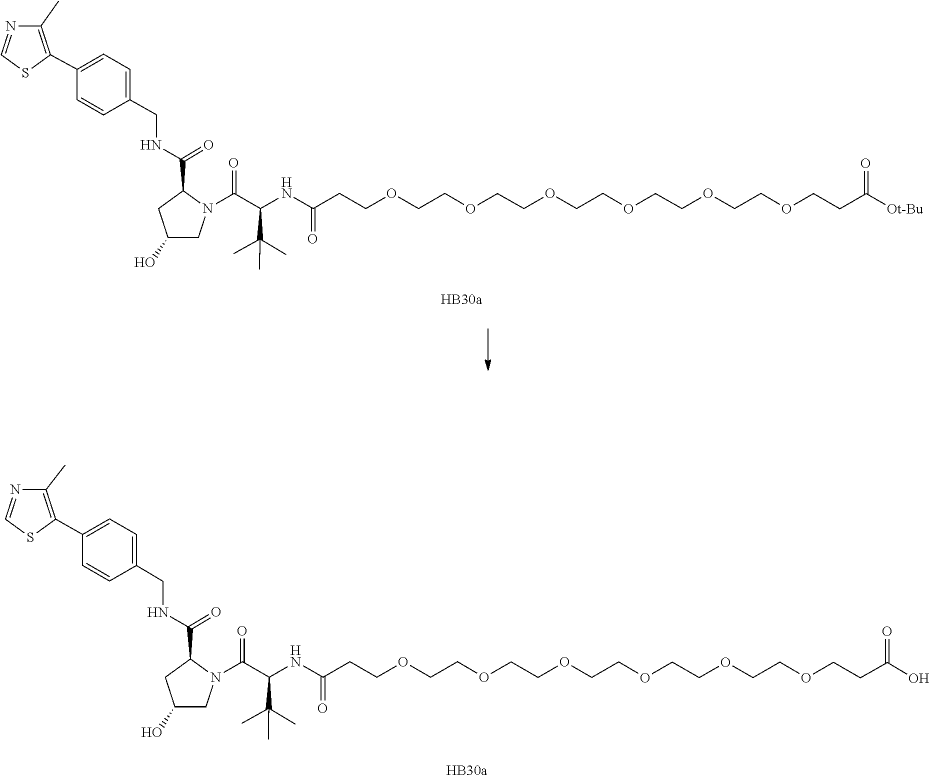 [SMILES: Cc1ncsc1-c1ccc(CNC(=O)[C@@H]2C[C@@H](O)CN2C(=O)[C@@H](NC(=O)CCOCCOCCOCCOCCOCCOCCC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCOCCC(=O)O)C(C)(C)C)cc1]

Using General Procedure 2, (2S,4R)1-[(2S)-2-amino-3,3-dimethyl-butanoyl]-4-hydroxy-N-[[4-(4-methylthiazol-5-yl)phenyl]methyl]pyrrolidine-2-carboxamide (HB7) was treated with 2,2-dimethyl-4-oxo-3,7,10,13,16,19,22-heptaoxapentacosan-25-oic acid to afford tert-butyl (S)-24-((2S,4R)-4-hydroxy-2-((4-(4-methylthiazol-5-yl)benzyl)carbamoyl)pyrrolidine-1-carbonyl)-25,25-dimethyl-22-oxo-4,7,10,13,16,19-hexaoxa-23-azahexacosanoate (HB30a). Deprotection of the N-Boc protecting group in tert-butyl (S)-24-((2S,4R)-4-hydroxy-2-((4-(4-methylthiazol-5-yl)benzyl)carbamoyl)pyrrolidine-1-carbonyl)-25,25-dimethyl-22-oxo-4,7,10,13,16,19-hexaoxa-23-azahexacosanoate (HB30a) using General Procedure 3 affords (S)-24-((2S,4R)-4-hydroxy-2-((4-(4-methylthiazol-5-yl)benzyl)carbamoyl)pyrrolidine-1-carbonyl)-25,25-dimethyl-22-oxo-4,7,10,13,16,19-hexaoxa-23-azahexacosanoic acid (HB30).

### Example 37. (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (HB31)

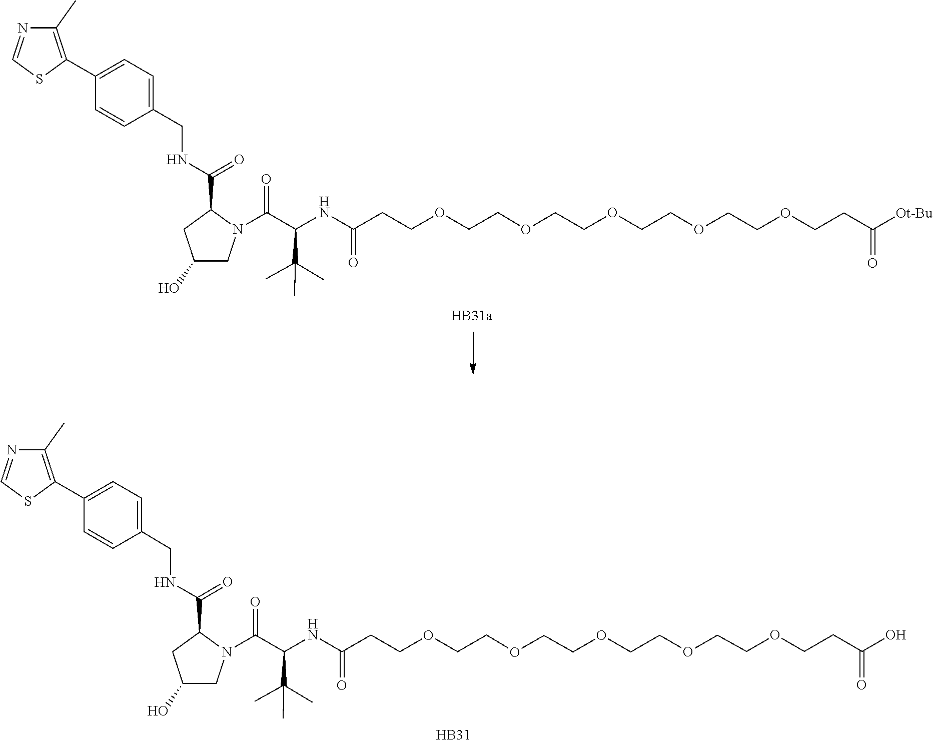

Using General Procedure 2, (2S,4R)1-[(2S)-2-amino-3,3-dimethyl-butanoyl]-4-hydroxy-N-[[4-(4-methylthiazol-5-yl)phenyl]methyl]pyrrolidine-2-carboxamide (HB7) was treated with 2,2-dimethyl-4-oxo-3,7,10,13,16,19-hexaoxadocosan-22-oic acid to afford tert-butyl (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoate (HB31a). Deprotection of the N-Boc protecting group in tert-butyl (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoate (HB31a) using General Procedure 3 affords (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (HB31).

### Example 38. 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (HB32)

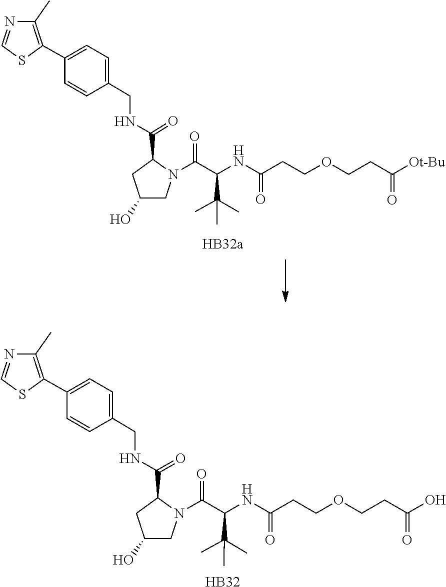 [SMILES: Cc1ncsc1-c1ccc(CNC(=O)[C@@H]2C[C@@H](O)CN2C(=O)[C@@H](NC(=O)CCOCCC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCOCCC(=O)O)C(C)(C)C)cc1 ; "HB32"]

Using General Procedure 2, (2S,4R)1-[(2S)-2-amino-3,3-dimethyl-butanoyl]-4-hydroxy-N-[[4-(4-methylthiazol-5-yl)phenyl]methyl]pyrrolidine-2-carboxamide (HB7) was treated with 3-(3-(tert-butoxy)-3-oxopropoxy)propanoic acid to afford tert-butyl 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoate (HB32a). Deprotection of the N-Boc protecting group in tert-butyl 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoate (HB32a) using General Procedure 3 affords 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (HB32).

IAP-targeting LHM can be generally prepared according to Scheme B1-II

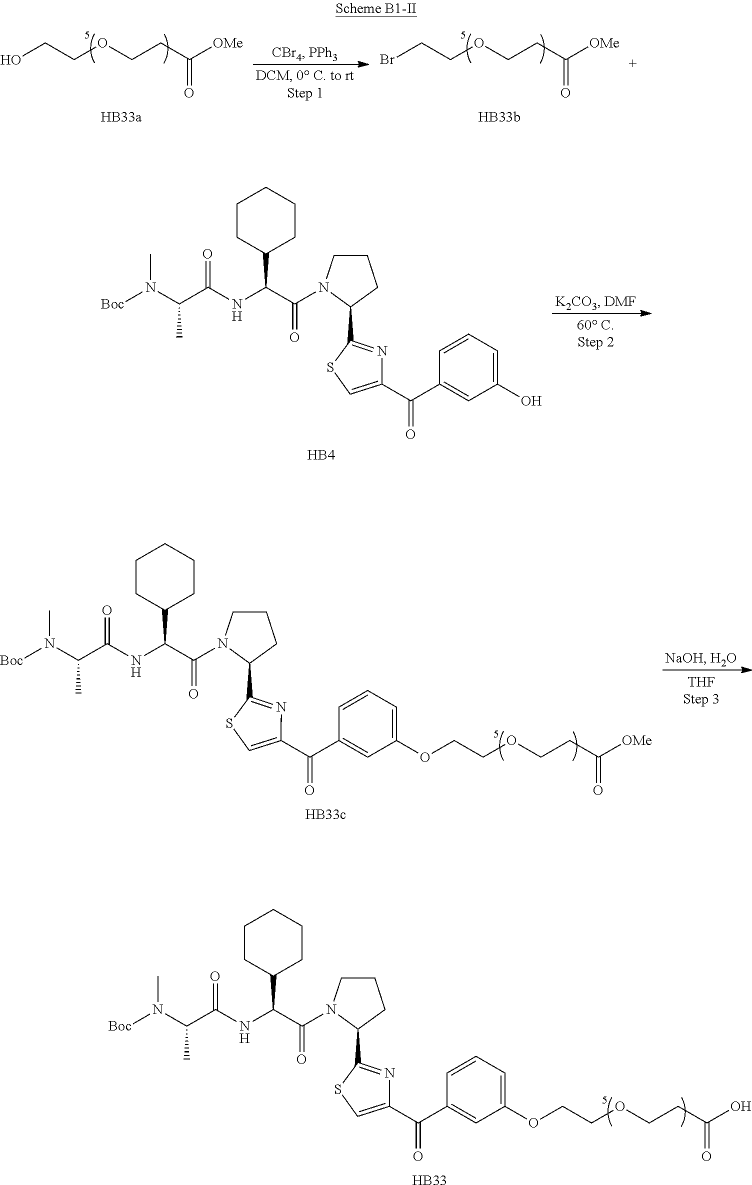

Step 1: Into a 50 mL 3-necked round-bottom flask purged and maintained with an inert atmosphere of nitrogen was placed alcohol HB33a (1.0 equiv), PPh₃ (2.0 equiv), and CH₂Cl₂ (10V). The resulting solution was stirred for 15 min at 0° C. To this was added CBr₄ (2.0 equiv). The resulting solution was stirred for an additional 4 h at 25° C. To the reaction was then added petroleum ether, and the solids were filtered out. The reaction was then quenched by the addition of water. The resulting solution was extracted with EtOAc. The organic phase was washed with brine. The organic phase was dried over anhydrous sodium sulfate and concentrated to afford bromo ester HB33b. That was used in the next step directly without further purification.
Step 2: Into a 50 mL 3-necked round-bottom flask purged and maintained with an inert atmosphere of nitrogen was placed tert-butyl N-[(1S)-1-[[(1S)-1-cyclohexyl-2-[(2S)-2-[4-(3-hydroxybenzoyl)-1,3-thiazol-2-yl]pyrrolidin-1-yl]-2-oxoethyl]carbamoyl]ethyl]-N-methylcarbamate (HB4) (1.0 equiv), bromo ester HB33b (2.0 equiv), and K₂CO₃ (5.0 equiv) in DMF (10V). The resulting solution was stirred for 12 h at 60° C. The reaction was then quenched by the addition of H₂O. The resulting mixture was extracted with ethyl acetate and the organic phase was washed with brine. The organic phase was dried over anhydrous sodium sulfate and concentrated. The residue was purified by reverse phase column chromatography with the following conditions: Mobile Phase A: Water (0.1% NH₄HCO₃), Mobile Phase B: ACN; Flow rate: 70 mL/min; Gradient: 8% B to 80% B in 30 min; 254/220 nm.
Step 3: Into a 50 mL 3-necked round-bottom flask purged and maintained with an inert atmosphere of nitrogen, was placed ester HB33c (1.00 equiv) in THE (10V) and 6 N aq. NaOH (1V) and H₂O (8V) was added at room temperature. The resulting solution was stirred at room temperature for 2 h. After the addition of 4 N aq. AcOH (5V) and H₂O (50V), the resulting solution was extracted with ethyl acetate and the organic phase was washed with brine. The mixture was dried over anhydrous sodium sulfate, filtered, and concentrated.

### Example 39. 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12,15-pentaoxaoctadecan-18-oic acid (HB33)

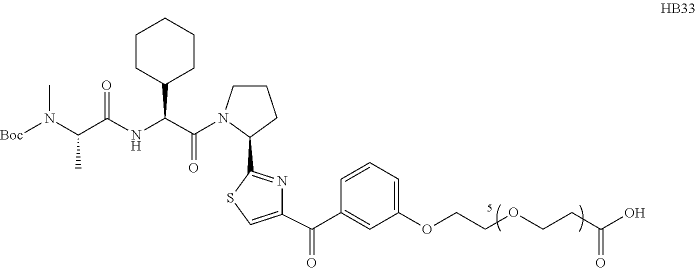

According to Scheme B1-II, 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12,15-pentaoxaoctadecan-18-oic acid (HB33) was obtained as a brown oil (45%) from methyl 1-hydroxy-3,6,9,12,15-pentaoxaoctadecan-18-oate (HB33a). LC-MS: (ES, m/z): 891 [M+H]⁺. ¹H-NMR (400 MHz, DMSO-d₆): δ 8.49 (s, 1H), 7.82 (s, 1H), 7.71-7.62 (m, 2H), 7.47 (t, J=8.0 Hz, 1H), 7.26 (ddd, J=8.3, 2.7, 1.0 Hz, 1H), 5.39 (dd, J=7.9, 3.0 Hz, 1H), 4.44 (t, J=7.8 Hz, 1H), 4.21-4.14 (m, 2H), 3.83-3.73 (m, 4H), 3.67 (s, 1H), 3.64-3.52 (m, 6H), 3.56-3.44 (m, 13H), 2.76 (s, 3H), 2.40 (t, J=6.4 Hz, 2H), 2.31-2.17 (m, 1H), 2.04 (t, J=6.8 Hz, 2H), 1.62 (s, 6H), 1.56 (s, 2H), 1.39 (s, 11H), 1.23 (s, 3H), 1.07 (s, 7H), 0.95 (s, 1H).

### Example 40. tert-butyl N-[(1S)-1-{[(1S)-1-cyclohexyl-2-[(2S)-2-(4-{3-[2-(2-hydroxyethoxy)ethoxy]benzoyl}-1,3-thiazol-2-yl)pyrrolidin-1-yl]-2-oxoethyl]carbamoyl}ethyl]-N-methylcarbamate (HB34)

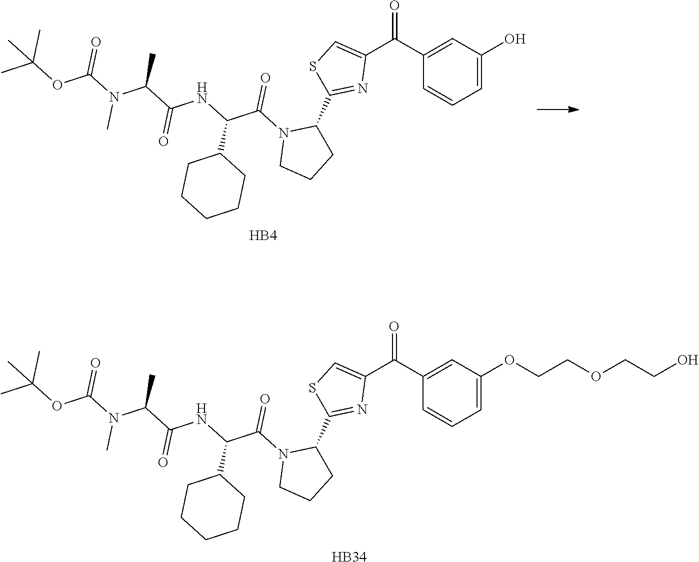

Into a 250 mL 3-necked round-bottom flask purged and maintained with an inert atmosphere of nitrogen was placed tert-butyl N-[(1S)-1-[[(1S)-1-cyclohexyl-2-[(2S)-2-[4-(3-hydroxybenzoyl)-1,3-thiazol-2-yl]pyrrolidin-1-yl]-2-oxoethyl]carbamoyl]ethyl]-N-methylcarbamate (HB4) (1.0 equiv), 2-(2-bromoethoxy)ethan-1-ol (2.0 equiv), and Cs₂CO₃ (3.0 equiv) in DMF (10V). The resulting solution was stirred for 12 h at 100° C. The resulting mixture was concentrated under vacuum and quenched by the addition of water. The resulting solution was extracted with ethyl acetate and the organic layers were combined, dried over anhydrous sodium sulfate, and concentrated under vacuum. This resulted in tert-butyl N-[(1S)-1-{[(1S)-1-cyclohexyl-2-[(2S)-2-(4-{3-[2-(2-hydroxyethoxy)ethoxy]benzoyl}-1,3-thiazol-2-yl)pyrrolidin-1-yl]-2-oxoethyl]carbamoyl}ethyl]-N-methylcarbamate (HB34) as a yellow oil and was used directly in the next step without further purification.

### Example 41. [4—({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperazin-1-yl]acetic acid (HB35)

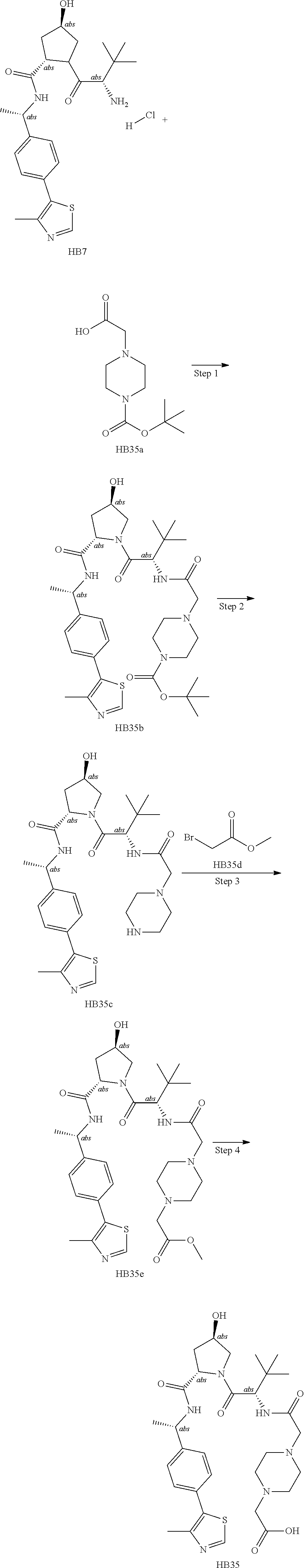

### Step 1: Synthesis of tert-butyl 4-({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperazine-1-carboxylate (HB35b)

A mixture of (1R,4S)-2-[(2S)-2-amino-3,3-dimethylbutanoyl]-4-hydroxy-N-[(1S)-1-[4-(4-methyl-1,3-thiazol-5-yl)phenyl]ethyl]cyclopentane-1-carboxamide hydrochloride (HB7) (1769 mg, 3.68 mmol), [4-(tert-butoxycarbonyl)piperazin-1-yl]acetic acid (HB35a) (900 mg, 3.68 mmol), HATU (1751 mg, 4.61 mmol), DIPEA (2.57 mL, 14.7 mmol), and DMF (44 mL) was allowed to stir at rt overnight. CH₂Cl₂ and sat. aq. NaHCO₃ were added. The organic layer was separated and dried with MgSO₄, filtered, concentrated, and purified by MPLC (20% MeOH in CH₂Cl₂) to afford tert-butyl 4-({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperazine-1-carboxylate (HB35b) (2.00 g, 80%). LCMS: C₃₄H₅₀N₆O₆S requires: 670, found: m/z=671 [M+H]⁺.

### Step 2: Synthesis of (2S,4R)-1-((S)-3,3-dimethyl-2-(2-(piperazin-1-yl)acetamido)butanoyl)-4-hydroxy-N—((S)-1-(4-(4-methylthiazol-5-yl)phenyl)ethyl)pyrrolidine-2-carboxamide (HB35c)

A mixture of tert-butyl 4-({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperazine-1-carboxylate (HB35b) (2.10 g, 3.13 mmol), 4 M HCl in dioxane (7 mL, 28 mmol) and CH₂Cl₂ (13 mL) was allowed to stir at rt overnight. The volatiles were removed, and the crude (2S,4R)-1-((S)-3,3-dimethyl-2-(2-(piperazin-1-yl)acetamido)butanoyl)-4-hydroxy-N—((S)-1-(4-(4-methylthiazol-5-yl)phenyl)ethyl)pyrrolidine-2-carboxamide (HB35c) was taken into the next step without further purification. LCMS: C₂₉H₄₂N₆O₄S requires: 570, found: m/z=571 [M+H]⁺.

### Step 3: Synthesis of methyl 2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)acetate (HB35e)

A mixture of (2S,4R)-1-[(2S)-3,3-dimethyl-2-[2-(piperazin-1-yl)acetamido]butanoyl]-4-hydroxy-N-[(1S)-1-[4-(4-methyl-1,3-thiazol-5-yl)phenyl]ethyl]pyrrolidine-2-carboxamide dihydrochloride (HB35c) (1344 mg, 1.57 mmol), methyl 2-bromoacetate (HB35d) (264 mg, 1.72 mmol), potassium carbonate (433 mg, 3.13 mmol), and DMF (10 mL) was allowed to stir at rt overnight. Water and CH₂Cl₂ were then added. The organic layer was separated and dried with MgSO₄, filtered, concentrated, and purified by MPLC (1% MeOH in CH₂Cl₂) to afford methyl 2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)acetate (HB35e) (589 mg, 59%). LCMS: C₃₂H₄₆N₆O₆S requires: 642, found: m/z=643 [M+H]⁺.

### Step 4: Synthesis of [4-({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperazin-1-yl]acetic acid (HB35)

A mixture of methyl 2-[4-({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperazin-1-yl]acetate (HB35e) (589 mg, 0.92 mmol), LiOH monohydrate (42.3 mg, 1.01 mmol), THE (3 mL), and water (1.5 mL) was allowed to stir at rt overnight. The mixture was then concentrated to afford the lithium salt of [4-({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperazin-1-yl]acetic acid (HB35) (566 mg, 98%). LCMS: C₃₁H₄₄N₆O₆S requires: 628, found: m/z=629 [M+H]⁺.

### Example 42. 3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoic acid (HB36)

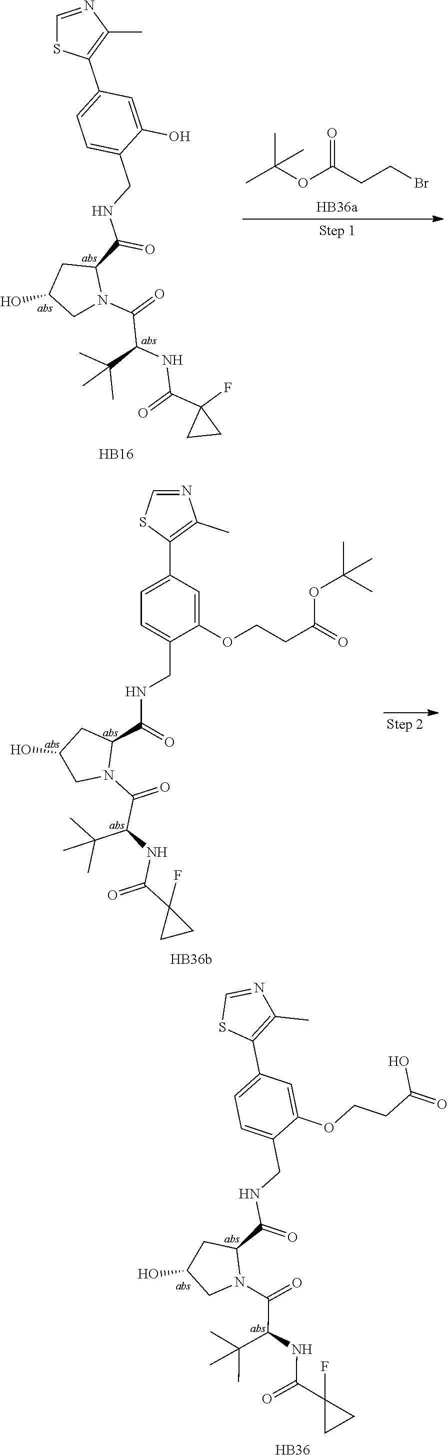

### Step 1: Synthesis of tert-butyl 3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoate (HB36b)

A mixture of (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (HB16) (274 mg, 0.51 mmol), tert-butyl 3-bromopropanoate (HB36a) (161 mg, 0.77 mmol), potassium carbonate (142 mg, 1.03 mmol), and DMF (5 mL) was allowed to stir at 80° C. for 15 h. EtOAc and H₂O were then added. The organic layer was dried with MgSO₄, filtered, concentrated, and tert-butyl 3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoate (HB36b) was carried into the next step without purification. LCMS: C₃₃H₄₅FN₄O₇S requires: 660, found: m/z=661 [M+H]⁺.

### Step 2: Synthesis of 3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoic acid (HB36)

A mixture of tert-butyl 3-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]propanoate (HB36b) (340 mg, 0.51 mmol), CH₂Cl₂ (5 mL), and TFA (1 mL) was allowed to stir at rt for 2 h. The volatiles were removed and 3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoic acid (HB36) was carried into the next step without purification. LCMS: C₂₉H₃₇FN₄O₇S requires: 604, found: m/z=605 [M+H]⁺.

### Example 43. 1-(6-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}pyridin-3-yl)piperidine-4-carboxylic acid (HB37)

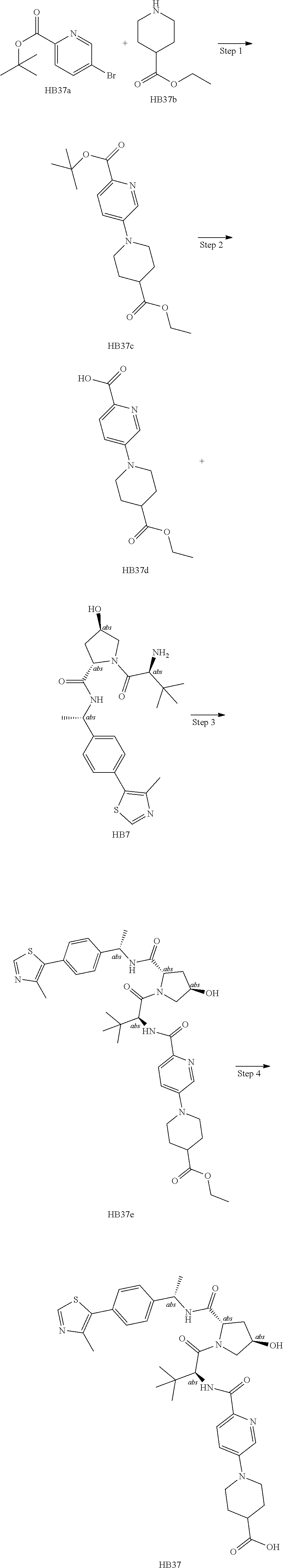

### Step 1: Synthesis of tert-butyl 5-[4-(ethoxycarbonyl)piperidin-1-yl]pyridine-2-carboxylate (HB37c)

A mixture of tert-butyl 5-bromopyridine-2-carboxylate (HB37a) (1.01 g, 3.92 mmol), ethyl piperidine-4-carboxylate (HB37b) (800.5 mg, 5.09 mmol), [2′-(methylamino)-[1,1′-biphenyl]-2-yl]palladiumylium dicyclohexyl({2′,6′-diisopropoxy-[1,1′-biphenyl]-2-yl})phosphane mesylate (166.5 mg, 0.20 mmol), and cesium carbonate (2552 mg, 7.83 mmol) was degassed and backfilled with N₂ five times. Dioxane (20 mL) was added and the mixture was allowed to stir at 100° C. for 3 h. The mixture was then filtered through celite, washed with EtOAc, concentrated, and purified by MPLC (10-100% EtOAc in hexanes) to afford tert-butyl 5-[4-(ethoxycarbonyl)piperidin-1-yl]pyridine-2-carboxylate (HB37c) (1.3050 g, 99.6%). LCMS: C₁₈H₂₆N₂O₄ requires: 334, found: m/z=335 [M+H]⁺.

### Step 2: Synthesis of 5-[4-(ethoxycarbonyl)piperidin-1-yl]pyridine-2-carboxylic acid (HB37d)

A mixture of tert-butyl 5-[4-(ethoxycarbonyl)piperidin-1-yl]pyridine-2-carboxylate (HB37c) (682 mg, 2.4 mmol), CH₂Cl₂ (20 mL), and TFA (4 mL) was allowed to stir at rt for 6 h. The volatiles were removed and the mixture was dried to afford 5-[4-(ethoxycarbonyl)piperidin-1-yl]pyridine-2-carboxylic acid (HB37d) (0.5500 g, 96.9%). LCMS: C₁₄H₁₈N₂O₄ requires: 278, found: m/z=279 [M+H]⁺.

### Step 3: Synthesis of ethyl 1-(6-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}pyridin-3-yl)piperidine-4-carboxylate (HB37e)

A mixture of 5-[4-(ethoxycarbonyl)piperidin-1-yl]pyridine-2-carboxylic acid (39.25 mg, 0.14 mmol), (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide (HB37d) (57.00 mg, 0.13 mmol), [(dimethylamino)({[1,2,3]triazolo[4,5-b]pyridin-3-yloxy})methylidene]dimethylazanium hexafluoro-lambda5-phosphanuide (73.12 mg, 0.19 mmol), N,N-diisopropylethylamine (0.09 mL, 0.51 mmol), and DMF (1 mL) was allowed to stir at rt for one hour. EtOAc and H₂O were then added. The organic layer was dried with MgSO₄, filtered, concentrated, and carried into the next step without purification. LCMS: C₃₇H₄₈N₆O₆S requires: 704, found: m/z=705 [M+H]⁺.

### Step 4: Synthesis of 1-(6-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}pyridin-3-yl)piperidine-4-carboxylic acid (HB37)

A mixture of ethyl 1-(6-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}pyridin-3-yl)piperidine-4-carboxylate (HB37e) (90.00 mg, 0.13 mmol), THE (0.5 mL), H₂O (0.5 mL), EtOH (0.5 mL), and lithiumhydroxide monohydrate (16.07 mg, 0.38 mmol) was allowed to stir at rt for 2 h. The volatiles were then removed. EtOAc and 0.5 M HCl were added. The organic layer was dried with MgSO₄, filtered, concentrated, and carried into the next step without purification. LCMS: C₃₅H₄₄N₆O₆S requires: 676, found: m/z=677 [M+H]⁺.

### Example 44. 3-[1-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)piperidin-4-yl]propanoic acid (HB38)

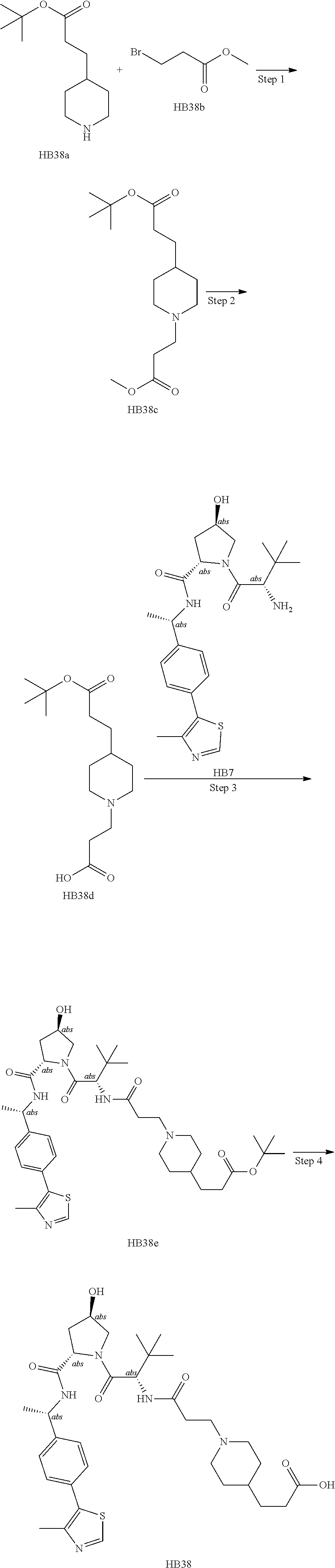

### Step 1: Synthesis of methyl 3-{4-[3-(tert-butoxy)-3-oxopropyl]piperidin-1-yl}propanoate (HB38c)

A mixture of tert-butyl 3-(piperidin-4-yl)propanoate (HB38a) (500 mg, 2.34 mmol), methyl 3-bromopropanoate (HB38b) (431 mg, 2.58 mmol), K₂CO₃ (648 mg, 4.69 mmol), and DMF (15 mL) was allowed to stir at rt overnight. CH₂Cl₂ and water were then added. The organic layer was dried with MgSO₄, filtered, and concentrated to afford methyl 3-{4-[3-(tert-butoxy)-3-oxopropyl]piperidin-1-yl}propanoate (HB38c) (700 mg, 99%). LCMS: C₁₆H₂₉NO₄ requires: 299, found: m/z=300 [M+H]⁺.

### Step 2: Synthesis of 3-{4-[3-(tert-butoxy)-3-oxopropyl]piperidin-1-yl}propanoic acid (HB38d)

A mixture of methyl 3-{4-[3-(tert-butoxy)-3-oxopropyl]piperidin-1-yl}propanoate (HB38c) (700 mg, 2.34 mmol), LiOH (113 mg, 2.69 mmol), THE (9 mL), and water (4.5 mL) was allowed to stir at rt overnight. The mixture was then concentrated to afford 3-{4-[3-(tert-butoxy)-3-oxopropyl]piperidin-1-yl}propanoic acid (HB38d) (650 mg, 97%). LCMS: C₁₅H₂₇NO₄ requires: 285, found: m/z=286 [M+H]⁺.

### Step 3: Synthesis of 3-[1-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)piperidin-4-yl]propanoic acid (HB38e)

A mixture of (1R,4S)-2-[(2S)-2-amino-3,3-dimethylbutanoyl]-4-hydroxy-N-[(1S)-1-[4-(4-methyl-1,3-thiazol-5-yl)phenyl]ethyl]cyclopentane-1-carboxamide hydrochloride (HB7) (600 mg, 1.25 mmol), 3-{4-[3-(tert-butoxy)-3-oxopropyl]piperidin-1-yl}propanoic acid (HB38d) (357 mg, 1.25 mmol), HATU (594 mg, 1.56 mmol), DIPEA (0.65 mL, 3.75 mmol), and DMF (15 mL) was allowed to stir at rt for 16 h. EtOAc and water were then added. The organic layer was dried with MgSO₄, filtered, concentrated, and purified by MPLC (1-20% MeOH in CH₂Cl₂) to afford tert-butyl 3-[1-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)piperidin-4-yl]propanoate (HB38e) (600 mg, 67%). LCMS: C₃₈H₅₇N₅O₆S requires: 711, found: m/z=712 [M+H]⁺.

### Step 4: Synthesis of 3-[1-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)piperidin-4-yl]propanoic acid (HB38)

A mixture of tert-butyl 3-[1-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)piperidin-4-yl]propanoate (HB38e) (600 mg, 0.84 mmol), HCl (4 M in dioxane, 3.2 mL, 12.6 mmol), and CH₂Cl₂ (5 mL) was allowed to stir at rt overnight. The volatiles were removed and the mixture was purified by reverse phase MPLC (acetonitrile and H₂O) to afford 3-[1-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)piperidin-4-yl]propanoic acid (HB38) (400 mg, 72%). LCMS: C₃₄H₄₉N₅O₆S requires: 655, found: m/z=656 [M+H]⁺.

### Example 45. 3-[2-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethoxy)ethoxy]propanoic acid (HB39)

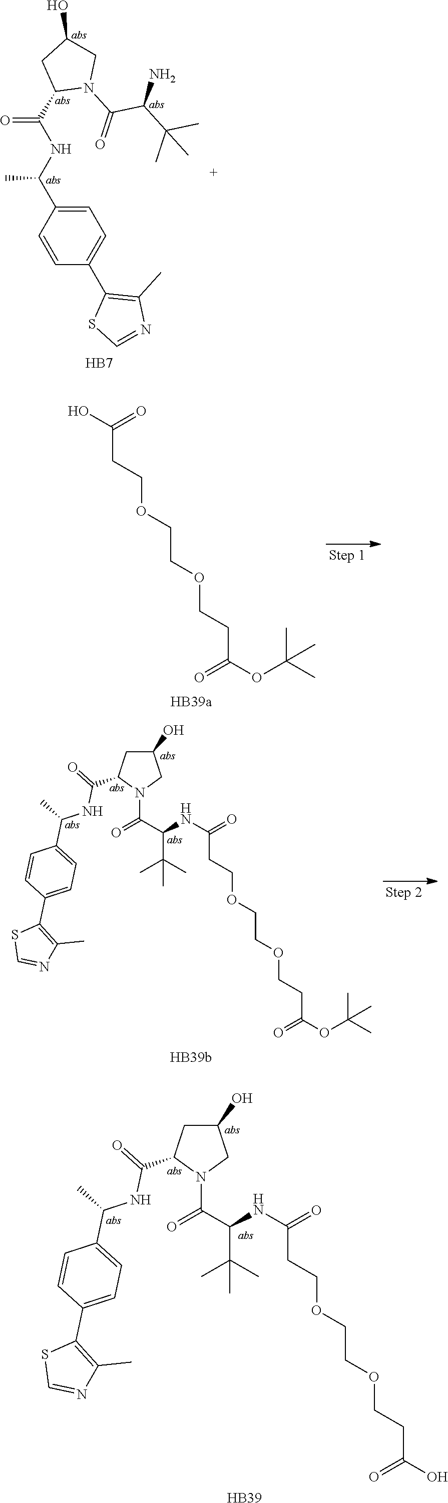

### Step 1: Synthesis of tert-butyl 3-[2-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethoxy)ethoxy]propanoate (HB39b)

A mixture of 3-{2-[3-(tert-butoxy)-3-oxopropoxy]ethoxy}propanoic acid (HB39a) (26 mg, 0.10 mmol), (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide (HB7) (40 mg, 0.09 mmol), HATU (51.3 mg, 0.13 mmol), N,N-diisopropylethylamine (0.06 mL, 0.36 mmol), and DMF (1 mL) was allowed to stir at rt for one hour. EtOAc and H₂O were then added. The organic layer was dried with MgSO₄, filtered, concentrated, and carried into the next step without purification. LCMS: C₃₅H₅₂N₄O₈S requires: 688, found: m/z=689 [M+H]⁺.

### Step 2: Synthesis of 3-[2-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethoxy)ethoxy]propanoic acid (HB39)

A mixture of tert-butyl 3-[2-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethoxy)ethoxy]propanoate (HB39b) (62 mg, 0.09 mmol), CH₂Cl₂ (1 mL), and TFA (0.2 mL) was allowed to stir at rt for 2 h. The volatiles were removed and the material was carried into the next step without purification. LCMS: C₃₁H₄₄N₄O₈S requires: 632, found: m/z=633 [M+H]⁺.

### Example 46. tert-butyl (S)-3-((4-amino-1H-pyrazol-1-yl)(phenyl)methyl)azetidine-1-carboxylate (BBX₁)

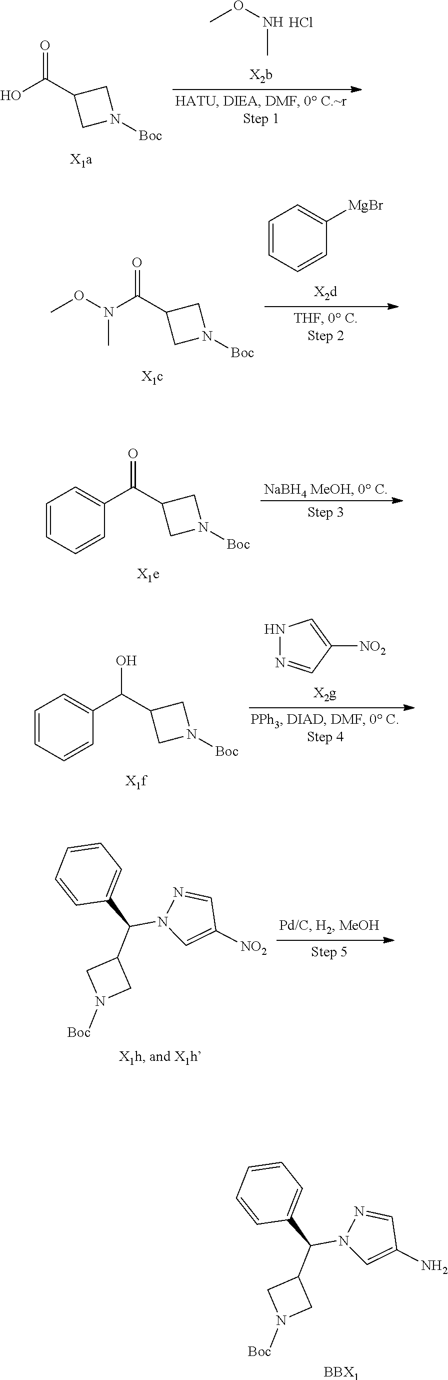

### Step 1: Synthesis of tert-butyl 3-[methoxy(methyl)carbamoyl]azetidine-1-carboxylate (X₁c)

To a mixture of 1-[(tert-butoxy)carbonyl]azetidine-3-carboxylic acid (50 g, 248.48 mmol) and DIEA (299.7 g, 2.32 mol) in N,N-dimethylformamide (500 mL) was added methoxy(methyl)amine hydrochloride (48.0 g, 494.41 mmol) and HATU (115.3 g, 303.24 mmol). The resulting mixture was stirred at room temperature for 16 h. The reaction mixture was quenched with water and extracted with ethyl acetate. The combined organic layers were washed with water, brine, dried over anhydrous sodium sulfate, and concentrated under vacuum to afford tert-butyl 3-[methoxy(methyl)carbamoyl]azetidine-1-carboxylate (200 g, crude) as a light yellow oil, which was used in the next step without further purification. LCMS: C₁₁H₂₀N₂O₄ requires: 244, found: m/z=245 [M+H]⁺.

### Step 2: Synthesis of tert-butyl 3-benzoylazetidine-1-carboxylate (X₁e)

To a solution of tert-butyl 3-[methoxy(methyl)carbamoyl]azetidine-1-carboxylate (200 g, 818 mmol,) in tetrahydrofuran (2 L) was added phenylmagnesium bromide (614 mL, 2 M in THF, 1.23 mol) dropwise at 0° C. under nitrogen. The resulting solution was stirred at room temperature for one hour, and then quenched with saturated NH₄Cl solution at 0-5° C. The solids were filtered out and the aqueous solution was extracted with ethyl acetate. The organic layer was washed with brine, dried over anhydrous sodium sulfate, and concentrated under vacuum. The crude residue was purified by flash column chromatography with 0-10% ethyl acetate in petroleum ether to afford tert-butyl 3-benzoylazetidine-1-carboxylate (59 g, 48% over two steps) as a white solid. LCMS: C₁₅H₁₉NO₃ requires: 261, found: m/z=262 [M+H]⁺.

### Step 3: Synthesis of tert-butyl 3-benzoylazetidine-1-carboxylate (X₁f)

To a solution of tert-butyl 3-benzoylazetidine-1-carboxylate (54.8 g, 209.70 mmol) in methanol (540 mL) was added NaBH₄ (16.0 g, 419.4 mmol) in portions at 0-5° C. The resulting mixture was stirred at 0-5° C. for 2 h. The reaction mixture was quenched by the addition of water maintaining the temperature at 0-5° C., and then extracted with ethyl acetate. The organic was washed with brine, dried over anhydrous sodium sulfate, and concentrated under vacuum to afford tert-butyl 3-[hydroxy(phenyl)methyl]azetidine-1-carboxylate (53 g, 96%) as yellow oil. ¹H NMR (300 MHz, DMSO-d₆) δ 7.45-7.09 (m, 5H), 5.55 (d, J=4.8 Hz, 1H), 4.64 (dd, J=7.2, 4.5 Hz, 1H), 3.80-2.65 (m, 4H), 2.76-2.74 (m, 1H), 1.38 (s, 9H). LCMS: C₁₅H₂₁NO₃ requires: 263, found: m/z=264 [M+H]⁺.

### Step 4: Synthesis of tert-butyl (S)-3-((4-nitro-1H-pyrazol-1-yl)(phenyl)methyl)azetidine-1-carboxylate and tert-butyl (R)-3-((4-nitro-1H-pyrazol-1-yl)(phenyl)methyl)azetidine-1-carboxylate (X₁h, and X₁h′)

To a mixture of tert-butyl 3-[hydroxy(phenyl)methyl]azetidine-1-carboxylate (50 g, 205 mmol), 4-nitro-1H-pyrazole (30 g, 260 mmol), and PPh₃ (80.5 g, 307 mmol) in THE (500 mL) was added DIAD (62 g, 307 mmol) dropwise at 0° C. under nitrogen. The resulting mixture was stirred at room temperature for 16 h under nitrogen. The reaction mixture was quenched by water and extracted with ethyl acetate. The combined organic layers were washed with water and brine, dried over anhydrous sodium sulfate, and concentrated under vacuum. The crude residue was purified by flash column chromatography with 0-10% ethyl acetate in petroleum ether to afford tert-butyl 3-[(4-nitro-1H-pyrazol-1-yl)(phenyl)methyl]azetidine-1-carboxylate (53 g, 72%) as a yellow oil. The racemic product (10 g) was separated by prep-chiral-SFC under the following conditions: [Column (R,R)WHELK-01; Column temperature 35° C.; Co-Solvent IPA (0.1% DIEA) 50.56%; Co-Solvent flow rate 90 mL/min; Total flow 178 mL/min; Back pressure 1500 psi; Detector, UV 220 nm] to afford tert-butyl (S)-3-((4-nitro-1H-pyrazol-1-yl)(phenyl)methyl)azetidine-1-carboxylate (4.5 g) as a yellow syrup with the shorter retention time on chiral-SFC and tert-butyl (R)-3-((4-nitro-1H-pyrazol-1-yl)(phenyl)methyl)azetidine-1-carboxylate (4.3 g) as a yellow syrup with the longer retention time on chiral-SFC. ¹H NMR (300 MHz, Chloroform-d) δ 8.11-7.99 (m, 2H), 7.48-7.37 (m, 3H), 7.34-7.30 (m, 2H), 5.41 (d, J=10.5 Hz, 1H), 4.17-4.06 (m, 1H), 3.95-3.94 (m, 1H), 3.80 (d, J=4.8 Hz, 1H), 3.67-3.47 (m, 2H), 1.42 (s, 9H). LCMS: C₁₈H₂₂N₄O₄ requires: 358, found: m/z=359 [M+H]⁺.

### Step 5: Synthesis of (S)-tert-butyl 3-((4-amino-1H-pyrazol-1-yl)(phenyl)methyl)azetidine-1-carboxylate (BBX₁)

To a solution of 3-[(S)-(4-nitro-1H-pyrazol-1-yl)(phenyl)methyl]azetidine-1-carboxylate (4.5 g, 13.67 mmol) in methanol (50 mL) was added palladium on carbon (dry, 0.5 g) under nitrogen. The resulting mixture was stirred at room temperature for 2 h under H₂ (2 atm). The solids were then filtered out. The filtrate was concentrated under vacuum to afford tert-butyl 3-[(S)-(4-amino-1H-pyrazol-1-yl)(phenyl)methyl]azetidine-1-carboxylate (4.0 g, crude) as a red syrup. LCMS: C₁₈H₂₄N₄O₂ requires: 328, found: m/z=329 [M+H]⁺.

### Example 47. (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂)

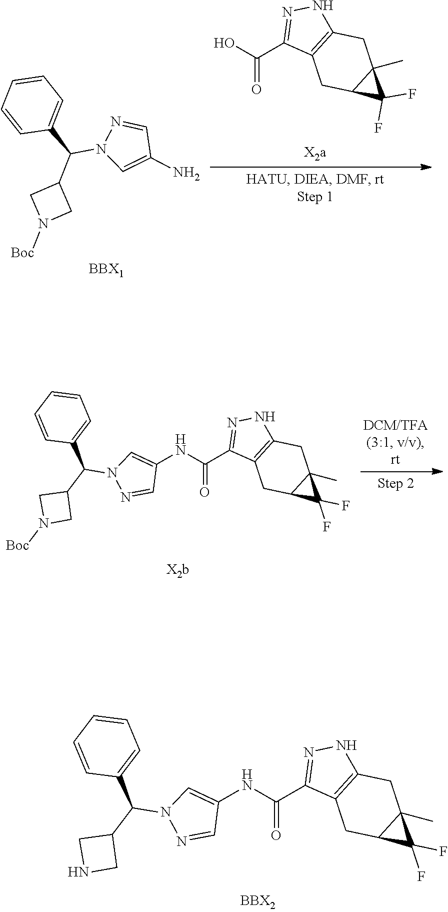

### Step 1: Synthesis of tert-butyl 3-((S)-(4-((4aS,5aR)-5,5-difluoro-5a-methyl-1,4,4a,5,5a,6-hexahydrocyclopropafindazole-3-carboxamido)-1H-pyrazol-1-yl)(phenyl)methyl)azetidine-1-carboxylate (X₂b)

To a stirred solution of (4aS,5aR)-5,5-difluoro-5a-methyl-1,4,4a,5,5a,6-hexahydrocyclopropafindazole-3-carboxylic acid (210 mg, 0.92 mmol), tert-butyl (S)-3-((4-amino-1H-pyrazol-1-yl)(phenyl)methyl)azetidine-1-carboxylate (302 mg, 0.92 mmol), and HATU (524.4 mg, 1.38 mmol) in N,N-dimethylformamide (2 mL) was added DIEA (237.4 mg, 1.84 mmol). The resulting solution was stirred overnight at room temperature. The reaction was then quenched by the addition water and extracted with ethyl acetate. The combined organic layers were dried over anhydrous sodium sulfate and concentrated under vacuum. The residue was purified by flash column chromatography with 0-50% ethyl acetate in petroleum ether to afford tert-butyl 3-((S)-(4-((4aS,5aR)-5,5-difluoro-5a-methyl-1,4,4a,5,5a,6-hexahydrocyclopropafindazole-3-carboxamido)-1H-pyrazol-1-yl)(phenyl)methyl)azetidine-1-carboxylate (340 mg, 68%) as a white solid. ¹H NMR (300 MHz, DMSO-d₆) δ 12.98 (s, 1H), 10.20 (s, 1H), 8.08 (s, 1H), 7.97 (s, 1H), 7.43-7.31 (m, 5H), 5.69 (d, J=10.4 Hz, 1H), 3.88-3.83 (m, 2H), 3.61-3.53 (m, 3H), 3.05 (s, 3H), 2.92-2.79 (m, 1H), 1.81-1.76 (m, 1H), 1.33 (s, 12H). LCMS: C₂₈H₃₂F₂N₆O₃ requires: 538, found: m/z=539 [M+H]⁺.

### Step 2: Synthesis of (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂)

A solution of tert-butyl 3-((S)-(4-((4aS,5aR)-5,5-difluoro-5a-methyl-1,4,4a,5,5a,6-hexahydrocyclopropafindazole-3-carboxamido)-1H-pyrazol-1-yl)(phenyl)methyl)azetidine-1-carboxylate (340 mg, 0.63 mmol) in dichloromethane (3 mL) and trifluoroacetic acid (1 mL) was stirred at room temperature for 2 h. The resulting mixture was then concentrated under vacuum. The crude product was purified by reverse phase flash chromatography with 10-40% MeCN in water (0.5% HCl). After concentration, the product was neutralized with 10% aq. Na₂CO₃ and extracted with propan-2-ol:CHCl₃ (1:5). The organic layer was dried over anhydrous sodium sulfate and concentrated under vacuum to afford (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (116.1 mg, 43%) as a white solid. ¹H NMR (300 MHz, DMSO-d₆) δ 12.99 (s, 1H), 10.16 (s, 1H), 8.06 (s, 1H), 7.61 (s, 1H), 7.34-7.24 (m, 5H), 5.62 (d, J=11.1 Hz, 1H), 3.69-3.58 (m, 1H), 3.47-3.22 (m, 5H), 3.05 (s, 3H), 2.84-2.77 (m, 1H), 1.81-1.73 (m, 1H), 1.34 (s, 3H). ¹⁹F NMR (282 MHz, DMSO-d₆) δ −133.52, −134.06, −145.45, −145.99. LCMS: C₂₃H₂₄F₂N₆O requires: 438, found: m/z=439 [M+H]⁺.

The following General Procedure Schemes 4-8 illustrate the bond formations by which the IAP and VHL harnesses may be coupled with the ITK hooks to afford the named IAP-based and VHL-based ITK compounds with corresponding chemical structures.

### General Procedure 4: Amide Formation and Subsequent Boc Deprotection

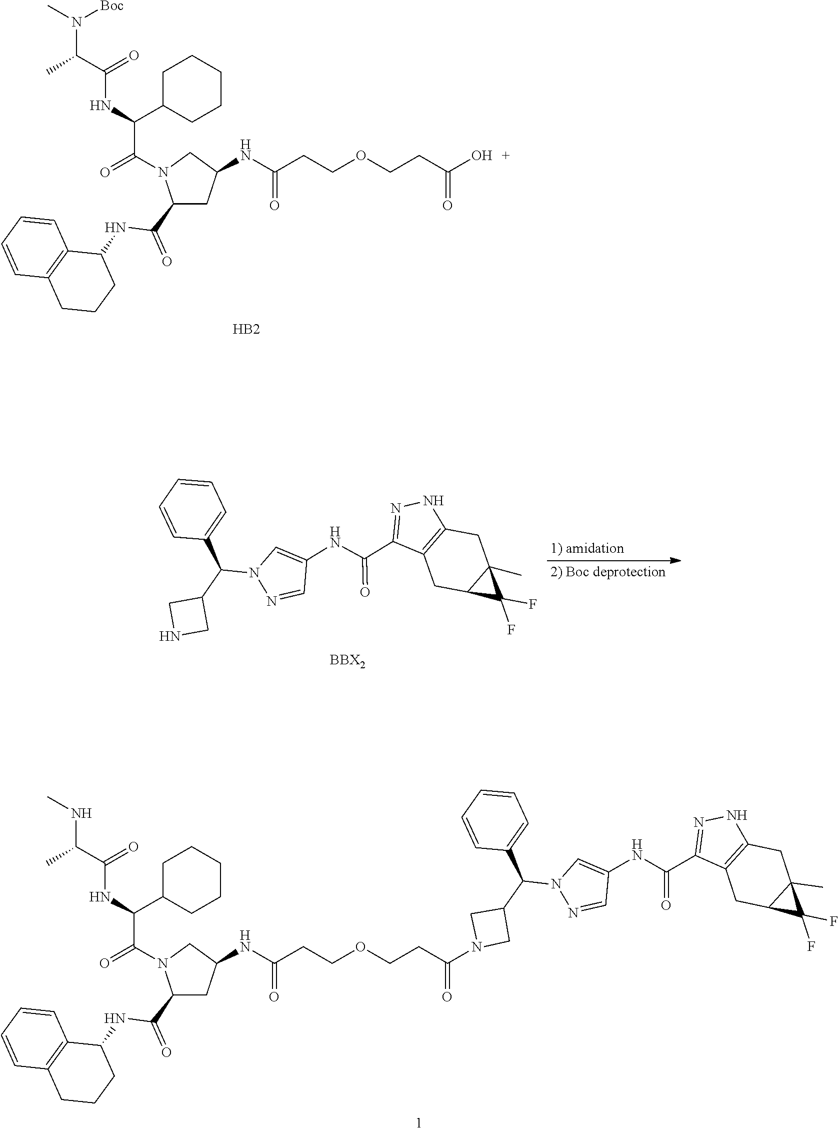

### Scheme 4: Synthesis of Compound 1 via Amide Formation and Subsequent Boc Deprotection

A mixture of amine (15 mg, 0.03 mmol), acid (37 mg, 0.05 mmol), HATU (20 mg, 0.05 mmol), and i-Pr₂NEt (18 μL, 0.1 mmol) in DMF (200 μL) was allowed to stir at room temperature for one hour. The reaction mixture was purified by HPLC (H₂O:MeCN with 0.1% TFA) to afford the amide product. HATU or BOP was typically used as a coupling reagent, but any suitable coupling agent can be employed.

A mixture of the resulting t-Bu carbamate and HCl (4 M in dioxane, 200 μL) was stirred at room temperature for 0.5 h. The reaction mixture was then diluted with 10% MeOH in DCM and then basified with 28-30% aqueous NH₄OH. The organic layer was washed with H₂O and saturated aqueous NaCl, dried over Na₂SO₄, filtered, concentrated under reduced pressure, and purified by HPLC (H₂O:MeCN with 0.1% TFA) to afford the amine product (7 mg, 0.006 mmol, 17%).

An exemplary amide coupling is provided in the scheme immediately above where (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa]indazole-3-carboxamide (BBX₂) (15 mg, 0.034 mmol) and 3-(3-(((1R,4R)-3-((S)-2-((S)-2-((tert-butoxycarbonyl)(methyl)amino)propanamido)-2-cyclohexylacetyl)-4-(((R)-1,2,3,4-tetrahydronaphthalen-1-yl)carbamoyl)cyclopentyl)amino)-3-oxopropoxy)propanoic acid (HB2) (37 mg, 0.051 mmol) were treated as described above to provide (4aS,5aR)—N-(1-((1S)-(1-(3-(3-(((1R,4R)-3-((S)-2-cyclohexyl-2-((S)-2-(methylamino)propanamido)acetyl)-4-(((R)-1,2,3,4-tetrahydronaphthalen-1-yl)carbamoyl)cyclopentyl)amino)-3-oxopropoxy)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (Compound 1).

Other amide containing compounds of this disclosure synthesized using General Procedure 4 are Compounds 2 and 3.

### General Procedure 5: Amide Formation and Subsequent Boc Deprotection

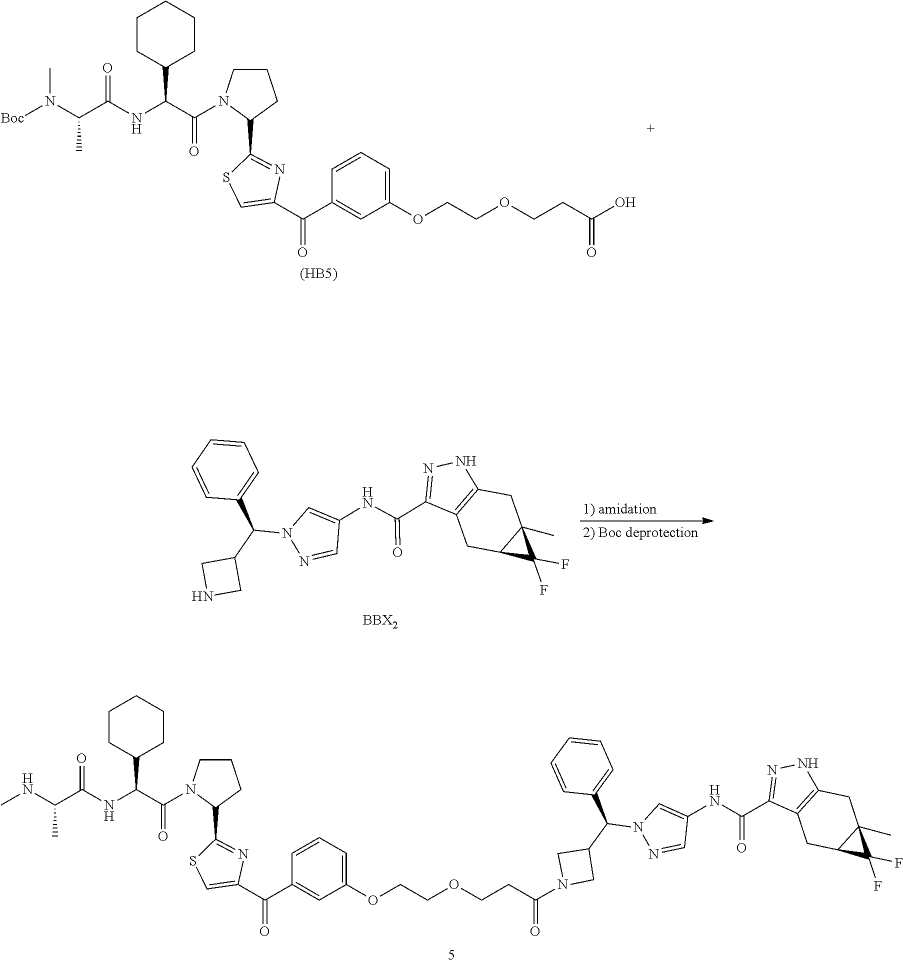

### Scheme 5: Synthesis of Compound 5 via Amide Formation and Subsequent Boc Deprotection

A mixture of amine (15 mg, 0.034 mmol), acid (49 mg, 0.068 mmol), HATU (26 mg, 0.068 mmol), and i-Pr₂NEt (24 μL, 0.14 mmol) in DMF (200 μL) was allowed to stir at room temperature for one hour. The reaction mixture was purified by HPLC (H₂O:MeCN with 0.1% TFA) to afford the amide product. HATU or BOP was typically used as a coupling reagent, but any suitable coupling agent can be employed. TFA (100 μL) was added to a solution of the resulting t-Bu carbamate in DCM (100 μL). After the reaction was stirred at room temperature for 15 min, the reaction was quenched with the addition of 28-30% aqueous NH₄OH. The aqueous layer was extracted with 10% MeOH in DCM. The combined organic layer was dried over Na₂SO₄, filtered, concentrated under reduced pressure, and purified by HPLC (H₂O:MeCN with 0.1% TFA) to afford the amine product (17 mg, 0.016 mmol, 46%).

An exemplary amide coupling is provided in the scheme immediately above where (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) (15 mg, 0.034 mmol) and 3-(2-(3-(2-((R)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoic acid (HB5) (49 mg, 0.068 mmol) were treated as described above to provide (4aS,5aR)—N-(1-((S)-(1-(3-(2-(3-(2-((R)-1-((S)-2-cyclohexyl-2-((S)-2-(methylamino)propanamido)acetyl)pyrrolidin-2-yl)thiazole-4-carbonyl)phenoxy)ethoxy)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (Compound 5).

Other amide containing compounds of this disclosure synthesized using General Procedure 5 are Compounds 4 and 6.

### General Procedure 6: Amide Formation

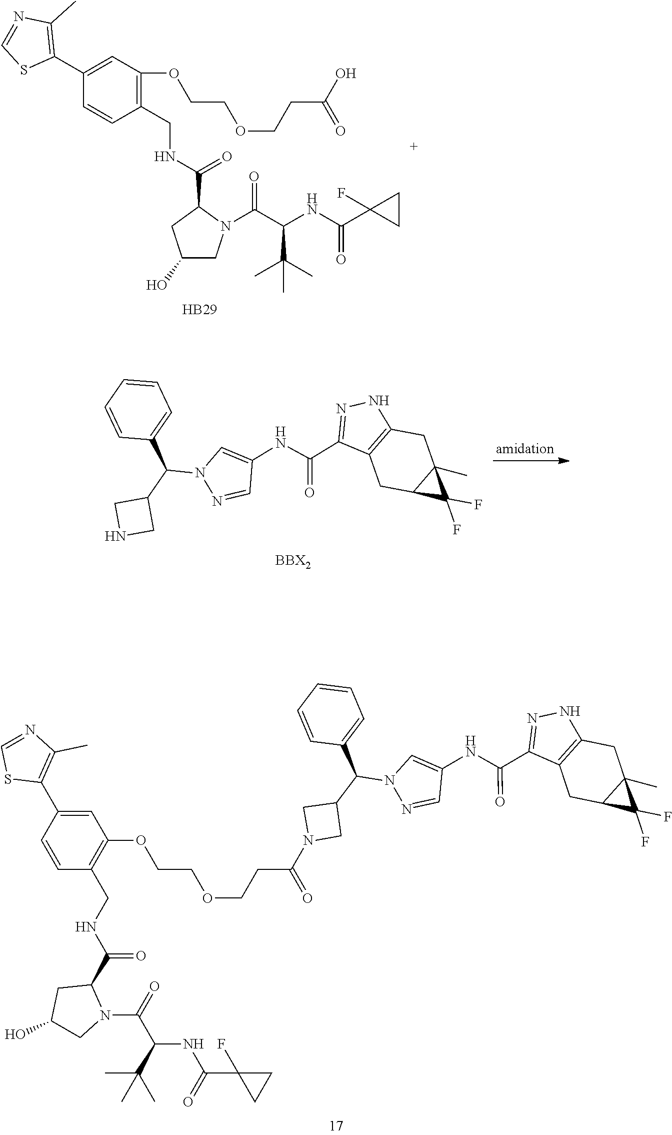 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCC(=O)O)c1 ; "HB29" ; C[C@@]12Cc3[nH]nc(C(=O)Nc4cnn([C@H](c5ccccc5)C5CNC5)c4)c3C[C@@H]1C2(F)F ; Cc1ncsc1-c1ccc(CNC(=O)[C@@H]2C[C@@H](O)CN2C(=O)[C@@H](NC(=O)C2(F)CC2)C(C)(C)C)c(OCCOCCC(=O)N2CC([C@@H](c3ccccc3)n3cc(NC(=O)c4n[nH]c5c4C[C@@H]4C(F)(F)[C@]4(C)C5)cn3)C2)c1]

### Scheme 6: Synthesis of Compound 17 Via Amide Formation

A mixture of amine (20 mg, 0.05 mmol), acid (30 mg, 0.05 mmol), BOP (26 mg, 0.06 mmol), and i-Pr₂NEt (40 μL, 0.23 mmol) in DMF (250 μL) was allowed to stir at room temperature for 16 h. The reaction mixture was purified by HPLC (H₂O:MeCN with 0.1% TFA) to afford the amide product (30 mg, 0.02 mmol, 52%). An exemplary amide coupling is provided in the scheme immediately above where (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) (20 mg, 0.05 mmol) and 3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)propanoic acid (HB29) (26 mg, 0.06 mmol) were treated as described above to provide (4aS,5aR)-5,5-difluoro-N-(1-((S)-(1-(3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (Compound 17). HATU or BOP was typically used as a coupling reagent, but any suitable coupling agent can be employed.

Other amide containing compounds of this disclosure synthesized using General Procedure 6 are Compounds 18, 19, and 20.

### General Procedure 7: Amide Formation

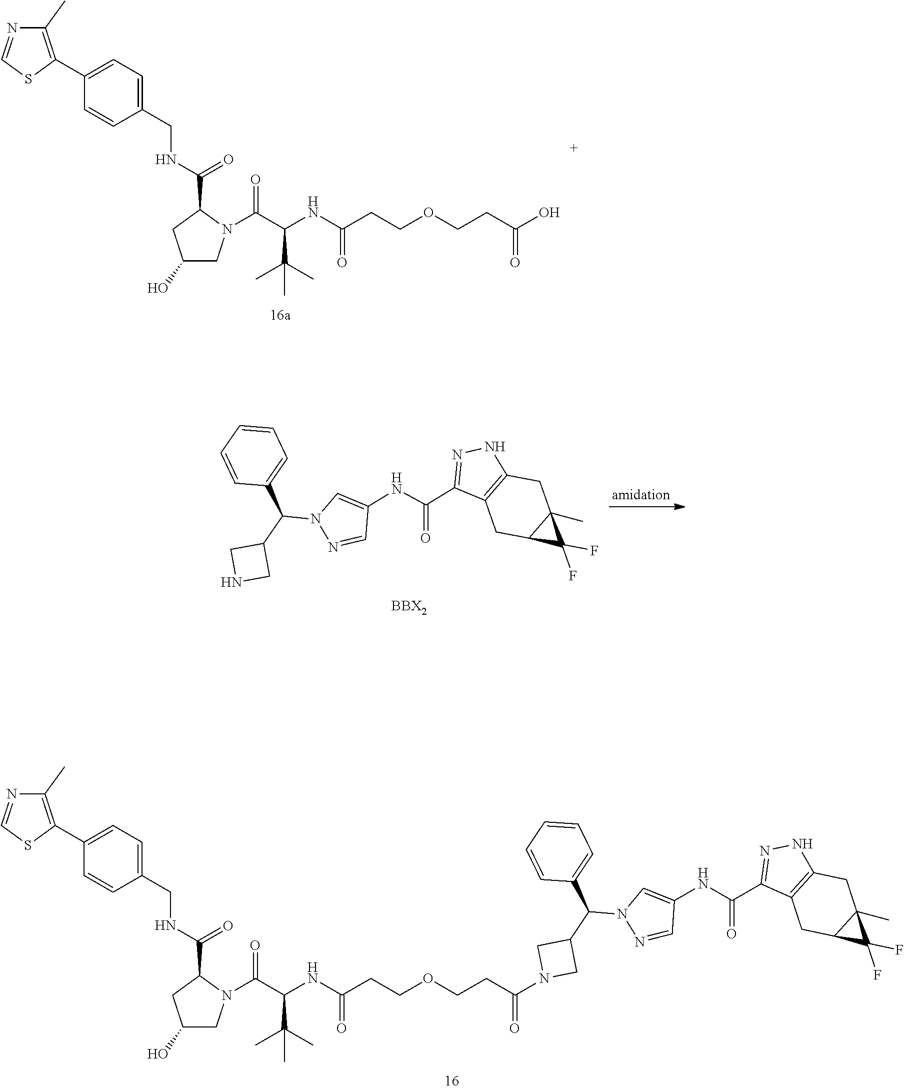 [SMILES: Cc1ncsc1-c1ccc(CNC(=O)[C@@H]2C[C@@H](O)CN2C(=O)[C@@H](NC(=O)CCOCCC(=O)O)C(C)(C)C)cc1 ; C[C@@]12Cc3[nH]nc(C(=O)Nc4cnn([C@H](c5ccccc5)C5CNC5)c4)c3C[C@@H]1C2(F)F ; Cc1ncsc1-c1ccc(CNC(=O)[C@@H]2C[C@@H](O)CN2C(=O)[C@@H](NC(=O)CCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

### Scheme 7: Synthesis of Compound 16 Via Amide Formation

A mixture of amine (20 mg, 0.05 mmol), acid (26 mg, 0.05 mmol), HATU (17 mg, 0.05 mmol), and i-Pr₂NEt (24 μL, 0.14 mmol) in DMF (200 μL) was allowed to stir at room temperature for one hour. The reaction mixture was purified by HPLC (H₂O:MeCN with 0.1% TFA) to afford the amide product (15 mg, 0.02 mmol, 34%). An exemplary amide coupling is provided in the scheme immediately above where (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) (20 mg, 0.05 mmol) and 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (16a) (26 mg, 0.05 mmol) were treated as described above to provide (4aS,5aR)-5,5-difluoro-N-(1-((S)-(1-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoyl)azetidin-3-yl)(phenyl)methyl)-1H-pyrazol-4-yl)-5a-methyl-1,4,4a,5,5a,6-hexahydrocyclopropafindazole-3-carboxamide (Compound 16). HATU or BOP was typically used as a coupling reagent, but any suitable coupling agent can be employed.

Other amide containing compounds of this disclosure synthesized using General Procedure 7 are Compounds 13, 14, and 15.

### General Procedure 8: Displacement

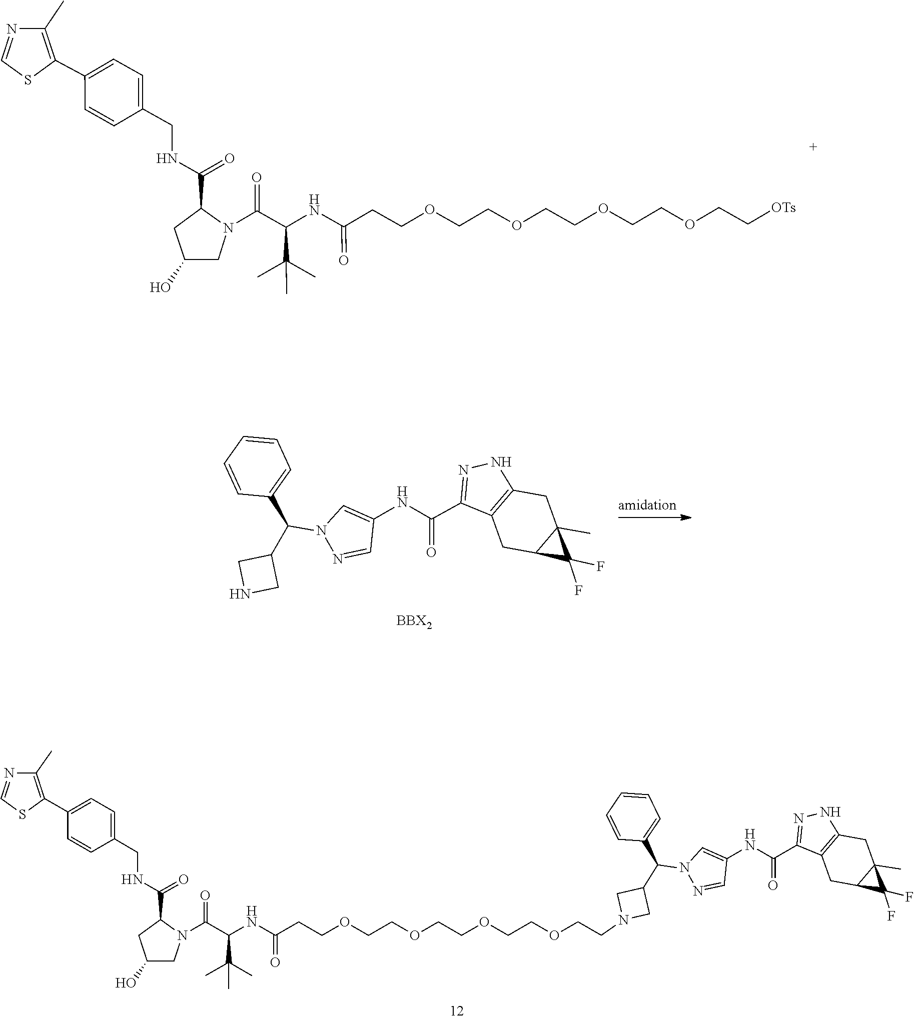 [SMILES: Cc1ccc(S(=O)(=O)OCCOCCOCCOCCOCCC(=O)N[C@H](C(=O)N2C[C@H](O)C[C@H]2C(=O)NCc2ccc(-c3scnc3C)cc2)C(C)(C)C)cc1 ; C[C@@]12Cc3[nH]nc(C(=O)Nc4cnn([C@H](c5ccccc5)C5CNC5)c4)c3C[C@@H]1C2(F)F ; Cc1ncsc1-c1ccc(CNC(=O)[C@@H]2C[C@@H](O)CN2C(=O)[C@@H](NC(=O)CCOCCOCCOCCOCCN2CC([C@@H](c3ccccc3)n3cc(NC(=O)c4n[nH]c5c4C[C@@H]4C(F)(F)[C@]4(C)C5)cn3)C2)C(C)(C)C)cc1]

### Scheme 8: Synthesis of Compound 12 Via Displacement

A mixture of amine (20 mg, 0.046 mmol), tosylate (38 mg, 0.046 mmol), KI (7.7 mg, 0.046 mmol), and i-Pr₂NEt (24 μL, 0.14 mmol) in DMF (200 μL) was stirred at 70° C. for 2 d. HPLC (20-70% MeCN:H₂O) afforded the amine product (3.9 mg, 0.0035 mmol, 8%). An exemplary amide coupling is provided in the scheme immediately above where (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) (20 mg, 0.046 mmol) and (S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl 4-methylbenzenesulfonate (HB8) (38 mg, 0.046 mmol) were treated as described above to provide (4aS,5aR)-5,5-difluoro-N-(1-((S)-(1-((S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl)azetidin-3-yl)(phenyl)methyl)-1H-pyrazol-4-yl)-5a-methyl-1,4,4a,5,5a,6-hexahydrocyclopropa[f]indazole-3-carboxamide (Compound 12).

### Example 48. (4aS,5aR)—N-(1-((S)-(1-(3-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (1)

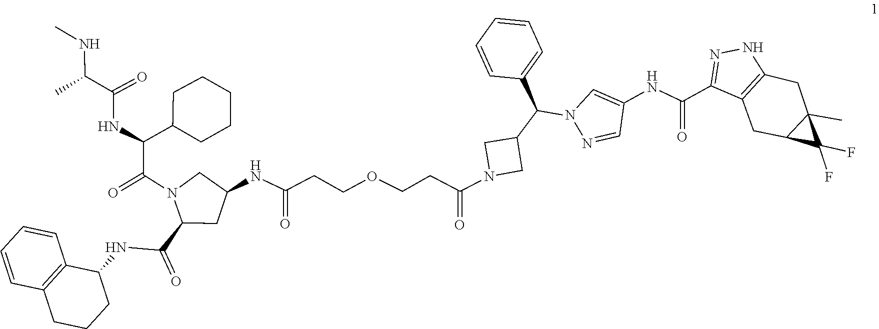 [SMILES: CN[C@@H](C)C(=O)N[C@H](C(=O)N1C[C@@H](NC(=O)CCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1]

Using General Procedure 4, 3-(3-(((1R,4R)-3-((S)-2-((S)-2-((tert-butoxycarbonyl)(methyl)amino)propanamido)-2-cyclohexylacetyl)-4-(((R)-1,2,3,4-tetrahydronaphthalen-1-yl)carbamoyl)cyclopentyl)amino)-3-oxopropoxy)propanoic acid (HB2) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) to afford (4aS,5aR)—N-(1-((S)-(1-(3-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (1). ¹H NMR (500 MHz, (CD₃)₂SO) δ 12.95 (s, 1H), 10.16 (s, 1H), 8.37 (dd, J=8.7, 5.6 Hz, 1H), 8.16 (dt, J=7.7, 4.6 Hz, 1H), 8.06 (s, 1H), 7.90 (dd, J=8.8, 2.5 Hz, 1H), 7.65 (d, J=2.3 Hz, 1H), 7.41-7.27 (m, 5H), 7.17-7.04 (m, 3H), 5.66 (dd, J=11.0, 4.2 Hz, 1H), 4.92 (s, 1H), 4.38 (t, J=8.2 Hz, 1H), 4.31-4.04 (m, 4H), 3.89-3.76 (m, 2H), 3.74-3.62 (m, 1H), 3.62-3.44 (m, 5H), 3.10-2.98 (m, 3H), 2.95 (q, J=6.8 Hz, 1H), 2.80 (d, J=17.2 Hz, 1H), 2.71 (d, J=6.4 Hz, 2H), 2.63 (t, J=1.9 Hz, 1H), 2.32-2.19 (m, 4H), 2.16 (s, 3H), 1.89-1.52 (m, 14H), 1.34 (s, 3H), 1.23 (s, 2H), 1.20-1.04 (m, 4H), 1.04-0.88 (m, 1H). LCMS: C₅₆H₇₁F₂11O₇ requires: 1048, found: m/z=1049 [M+H]⁺.

### Example 49. (4aS,5aR)—N-(1-((S)-(1-(16-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-16-oxo-4,7,10,13-tetraoxahexadecanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (2)

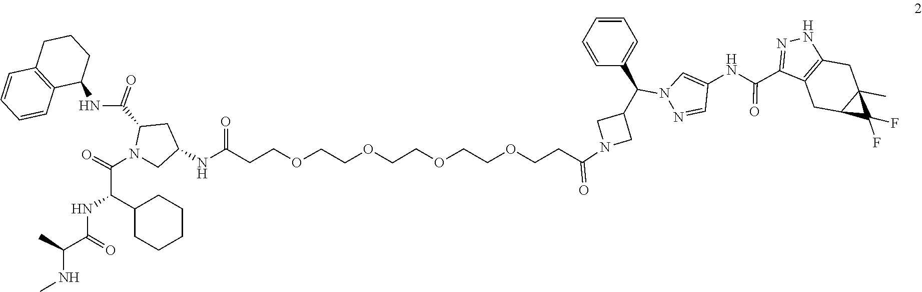 [SMILES: CN[C@@H](C)C(=O)N[C@H](C(=O)N1C[C@@H](NC(=O)CCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1]

Using General Procedure 4, 16-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-16-oxo-4,7,10,13-tetraoxahexadecanoic acid (HB3) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)—N-(1-((S)-(1-(16-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-16-oxo-4,7,10,13-tetraoxahexadecanoyl)azetidin-3-yl)(phenyl)methyl)-1H-pyrazol-4-yl)-5,5-difluoro-5a-methyl-1,4,4a,5,5a,6-hexahydrocyclopropafindazole-3-carboxamide (2). ¹H NMR (500 MHz, (CD₃)₂SO) δ 12.95 (s, 1H), 10.16 (s, 1H), 8.38 (d, J=8.7 Hz, 1H), 8.18 (d, J=7.4 Hz, 1H), 8.06 (s, 1H), 7.65 (d, J=1.4 Hz, 1H), 7.37 (tt, J=7.8, 6.1 Hz, 4H), 7.33-7.28 (m, 2H), 7.17-7.05 (m, 3H), 5.64 (dd, J=11.0, 6.2 Hz, 1H), 4.93 (q, J=8.1 Hz, 1H), 4.38 (t, J=8.2 Hz, 1H), 4.32-4.16 (m, 2H), 4.11 (q, J=6.1 Hz, 2H), 3.89-3.82 (m, 1H), 3.82-3.75 (m, 1H), 3.58 (ddd, J=14.3, 6.9, 4.2 Hz, 4H), 3.48 (dt, J=12.0, 3.1 Hz, 13H), 3.31-3.25 (m, 1H), 3.06-2.98 (m, 3H), 2.80 (d, J=17.2 Hz, 1H), 2.41-2.33 (m, 1H), 2.32-2.20 (m, 7H), 1.90-1.80 (m, 2H), 1.80-1.54 (m, 5H), 1.34 (s, 3H), 1.24 (s, 1H), 1.20-1.06 (m, 4H), 1.05-0.90 (m, 1H). LCMS: C₆₂H₈₃F₂N₁₁O₁₀ requires: 1180, found: m/z=1181 [M+H]⁺.

### Example 50. (4aS,5aR)—N-(1-((S)-(1-(19-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-19-oxo-4,7,10,13,16-pentaoxanonadecanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (3)

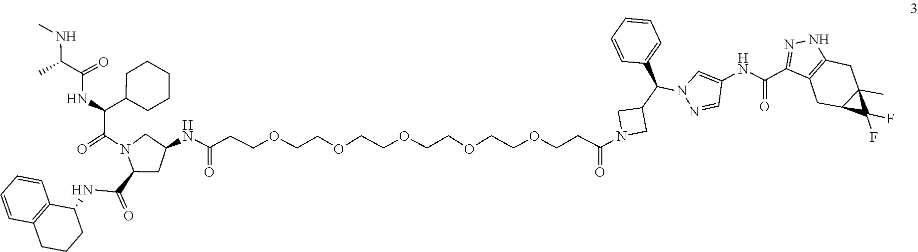 [SMILES: CN[C@@H](C)C(=O)N[C@H](C(=O)N1C[C@@H](NC(=O)CCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1]

Using General Procedure 4, 19-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-19-oxo-4,7,10,13,16-pentaoxanonadecanoic acid (HBxx) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)—N-(1-((S)-(1-(19-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-19-oxo-4,7,10,13,16-pentaoxanonadecanoyl)azetidin-3-yl)(phenyl)methyl)-1H-pyrazol-4-yl)-5,5-difluoro-5a-methyl-1,4,4a,5,5a,6-hexahydrocyclopropafindazole-3-carboxamide (3). ¹H NMR (500 MHz, (CD₃)₂SO) δ 12.95 (s, 1H), 10.16 (s, 1H), 8.38 (d, J=8.7 Hz, 1H), 8.18 (d, J=7.4 Hz, 1H), 8.06 (s, 1H), 7.91 (d, J=8.7 Hz, 1H), 7.65 (s, 1H), 7.42-7.29 (m, 5H), 7.17-7.05 (m, 3H), 5.64 (dd, J=11.0, 6.1 Hz, 1H), 4.93 (d, J=5.1 Hz, 1H), 4.38 (t, J=8.1 Hz, 1H), 4.32-4.16 (m, 2H), 4.10 (q, J=8.6 Hz, 1H), 3.85 (t, J=8.8 Hz, 1H), 3.79 (t, J=8.0 Hz, 1H), 3.63-3.53 (m, 4H), 3.48 (dt, J=7.5, 2.3 Hz, 16H), 3.31-3.25 (m, 1H), 3.04 (s, 2H), 3.02-2.91 (m, 2H), 2.80 (d, J=17.1 Hz, 1H), 2.76-2.67 (m, 2H), 2.42-2.33 (m, 1H), 2.33-2.20 (m, 3H), 2.16 (s, 2H), 1.85 (dd, J=10.3, 5.0 Hz, 3H), 1.80-1.50 (m, 1H), 1.34 (s, 3H), 1.23 (s, 1H), 1.15 (dd, J=13.1, 8.9 Hz, 1H), 1.08 (d, J=6.9 Hz, 3H), 1.05-0.90 (m, 1H). LCMS: C₆₄H₈₇F₂N₁₁O₁₁ requires: 1224, found: m/z=1225 [M+H]⁺.

### Example 51. (4aS,5aR)—N-(1-((S)-(1-(1-(3-(2-((R)-1-((S)-2-cyclohexyl-2-((S)-2-(methylamino)propanamido)acetyl)pyrrolidin-2-yl)thiazole-4-carbonyl)phenoxy)-3,6,9,12,15-pentaoxaoctadecan-18-oyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (4)

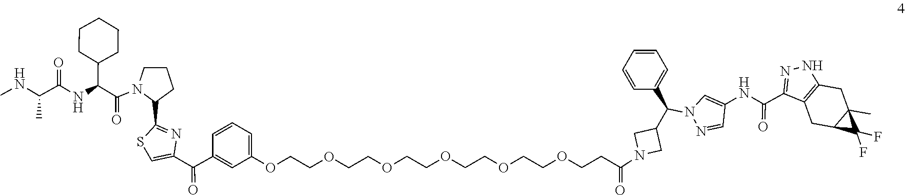

Using General Procedure 5, 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12,15-pentaoxaoctadecan-18-oic acid (HB33) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)—N-(1-((S)-(1-(1-(3-(2-((R)-1-((S)-2-cyclohexyl-2-((S)-2-(methylamino)propanamido)acetyl)pyrrolidin-2-yl)thiazole-4-carbonyl)phenoxy)-3,6,9,12,15-pentaoxaoctadecan-18-oyl)azetidin-3-yl)(phenyl)methyl)-1H-pyrazol-4-yl)-5,5-difluoro-5a-methyl-1,4,4a,5,5a,6-hexahydrocyclopropafindazole-3-carboxamide (4). ¹H NMR (500 MHz, (CD₃)₂SO) δ 12.95 (s, 1H), 10.16 (s, 1H), 8.48 (s, 1H), 8.06 (s, 1H), 7.91 (d, J=8.8 Hz, 1H), 7.70-7.61 (m, 3H), 7.48-7.43 (m, 1H), 7.40-7.27 (m, 5H), 7.25 (dt, J=8.2, 2.2 Hz, 1H), 5.63 (dt, J=13.4, 6.8 Hz, 1H), 5.39 (dd, J=8.1, 3.0 Hz, 1H), 4.49 (dd, J=8.9, 6.7 Hz, 1H), 4.21-4.08 (m, 3H), 3.85 (t, J=7.8 Hz, 1H), 3.82-3.73 (m, 5H), 3.65 (d, J=9.0 Hz, 1H), 3.61-3.41 (m, 25H), 3.10-2.93 (m, 4H), 2.80 (d, J=17.1 Hz, 1H), 2.30-2.21 (m, 2H), 2.18 (s, 3H), 2.10-1.97 (m, 2H), 1.80-1.65 (m, 1H), 1.62 (d, J=11.0 Hz, 3H), 1.54 (d, J=10.5 Hz, 2H), 1.34 (s, 3H), 1.23 (s, 1H), 1.10 (d, J=6.8 Hz, 3H), 1.07-0.80 (m, 6H). LCMS: C₆₂H₈₀F₂N₁₀O₁₁S requires: 1211, found: m/z=1212 [M+H]⁺.

### Example 52. (4aS,5aR)—N-(1-((S)-(1-(3-(2-(3-(2-((S)-1-((S)-2-cyclohexyl-2-((S)-2-(methylamino)propanamido)acetyl)pyrrolidin-2-yl)thiazole-4-carbonyl)phenoxy)ethoxy)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (5)

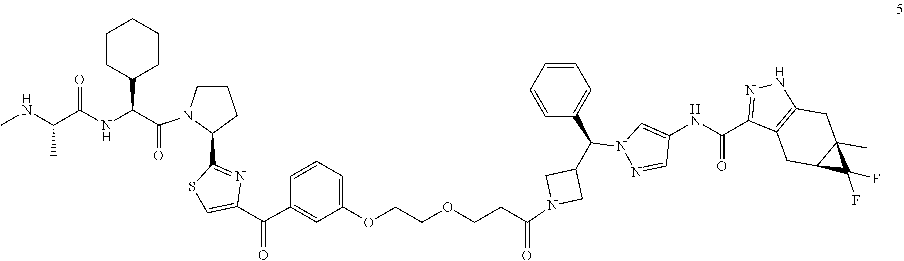 [SMILES: CN[C@@H](C)C(=O)N[C@H](C(=O)N1CCC[C@H]1c1nc(C(=O)c2cccc(OCCOCCC(=O)N3CC([C@@H](c4ccccc4)n4cc(NC(=O)c5n[nH]c6c5C[C@@H]5C(F)(F)[C@]5(C)C6)cn4)C3)c2)cs1)C1CCCCC1]

Using General Procedure 5, 3-(2-(3-(2-((S)-1-((S)-2-((S)—N-(tert-butoxycarbonyl)-2-(methylamino)propanamido)-2-cyclohexylacetyl)pyrrolidin-2-yl)thiazole-4-carbonyl)phenoxy)ethoxy)propanoic acid (HB5) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)—N-(1-((S)-(1-(3-(2-(3-(2-((S)-1-((S)-2-cyclohexyl-2-((S)-2-(methylamino)propanamido)acetyl)pyrrolidin-2-yl)thiazole-4-carbonyl)phenoxy)ethoxy)propanoyl)azetidin-3-yl)(phenyl)methyl)-1H-pyrazol-4-yl)-5,5-difluoro-5a-methyl-1,4,4a,5,5a,6-hexahydrocyclopropafindazole-3-carboxamide (5). ¹H NMR (500 MHz, (CD₃)₂SO) δ 12.95 (s, 1H), 10.15 (s, 1H), 8.48 (d, J=3.4 Hz, 1H), 8.05 (d, J=1.7 Hz, 1H), 7.90 (d, J=8.9 Hz, 1H), 7.65 (d, J=13.7 Hz, 3H), 7.46 (td, J=8.0, 3.5 Hz, 1H), 7.38-7.27 (m, 5H), 7.24 (d, J=8.2 Hz, 1H), 5.62 (dd, J=11.1, 2.1 Hz, 1H), 5.38 (dd, J=8.0, 3.0 Hz, 1H), 4.49 (t, J=7.8 Hz, 1H), 4.22-4.07 (m, 3H), 3.88-3.81 (m, 1H), 3.79 (t, J=6.7 Hz, 3H), 3.73 (s, 3H), 3.66 (t, J=6.4 Hz, 4H), 3.59-3.46 (m, 1H), 3.04 (s, 3H), 3.02-2.92 (m, 2H), 2.80 (d, J=17.2 Hz, 1H), 2.35-2.20 (m, 2H), 2.19-2.15 (m, 3H), 2.09-1.96 (m, 3H), 1.80-1.48 (m, 8H), 1.34 (s, 3H), 1.23 (s, 1H), 1.17-0.82 (m, 7H). LCMS: C₅₄H₆₄F₂N₁₀O₇S requires: 1035, found: m/z=1036 [M+H]⁺.

### Example 53. (4aS,5aR)—N-(1-((S)-(1-(1-(3-(2-((S)-1-((S)-2-cyclohexyl-2-((S)-2-(methylamino)propanamido)acetyl)pyrrolidin-2-yl)thiazole-4-carbonyl)phenoxy)-3,6,9,12-tetraoxapentadecan-15-oyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (6)

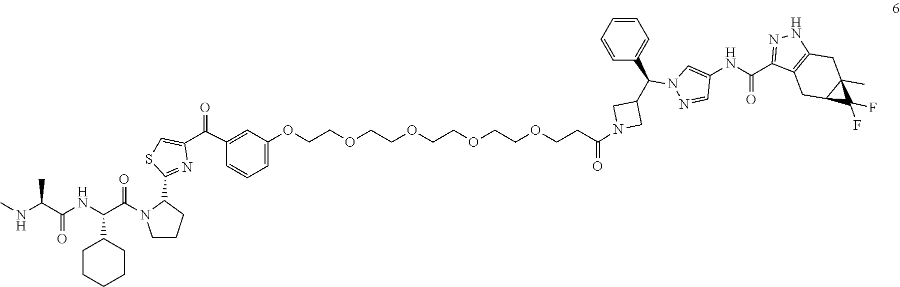

Using General Procedure 5, 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oic acid (HB6) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)—N-(1-((S)-(1-(1-(3-(2-((S)-1-((S)-2-cyclohexyl-2-((S)-2-(methylamino)propanamido)acetyl)pyrrolidin-2-yl)thiazole-4-carbonyl)phenoxy)-3,6,9,12-tetraoxapentadecan-15-oyl)azetidin-3-yl)(phenyl)methyl)-1H-pyrazol-4-yl)-5,5-difluoro-5a-methyl-1,4,4a,5,5a,6-hexahydrocyclopropafindazole-3-carboxamide (6). ¹H NMR (500 MHz, (CD₃)₂SO) δ 12.95 (s, 1H), 10.16 (s, 1H), 8.48 (d, J=1.0 Hz, 1H), 8.06 (s, 1H), 7.69-7.61 (m, 3H), 7.45 (td, J=8.0, 1.6 Hz, 1H), 7.40-7.27 (m, 5H), 7.27-7.22 (m, 1H), 5.63 (dt, J=12.5, 6.3 Hz, 1H), 5.39 (dd, J=8.0, 3.1 Hz, 1H), 4.49 (dd, J=8.7, 6.8 Hz, 1H), 4.21-4.07 (m, 3H), 3.88-3.73 (m, 5H), 3.66 (d, J=4.9 Hz, 1H), 3.62-3.42 (m, 11H), 3.10-2.97 (m, 3H), 2.80 (d, J=17.0 Hz, 1H), 2.63 (p, J=1.8 Hz, 1H), 2.36 (p, J=1.8 Hz, 2H), 2.31-2.13 (m, 6H), 2.10-1.97 (m, 2H), 1.78-1.67 (m, 1H), 1.62 (s, 3H), 1.55 (d, J=10.7 Hz, 1H), 1.34 (s, 3H), 1.24 (s, 5H), 1.16 (d, J=6.9 Hz, 3H), 1.12-0.90 (m, 2H). LCMS: C₆₀H₇₆F₂N₁₀O₁₀S requires: 1167, found: m/z=1168 [M+H]⁺.

### Example 54. (4aS,5aR)—N-(1-((S)-(1-(4-(3-(2-((S)-1-((S)-2-cyclohexyl-2-((S)-2-(methylamino)propanamido)acetyl)pyrrolidin-2-yl)thiazole-4-carbonyl)phenoxy)butyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (7)

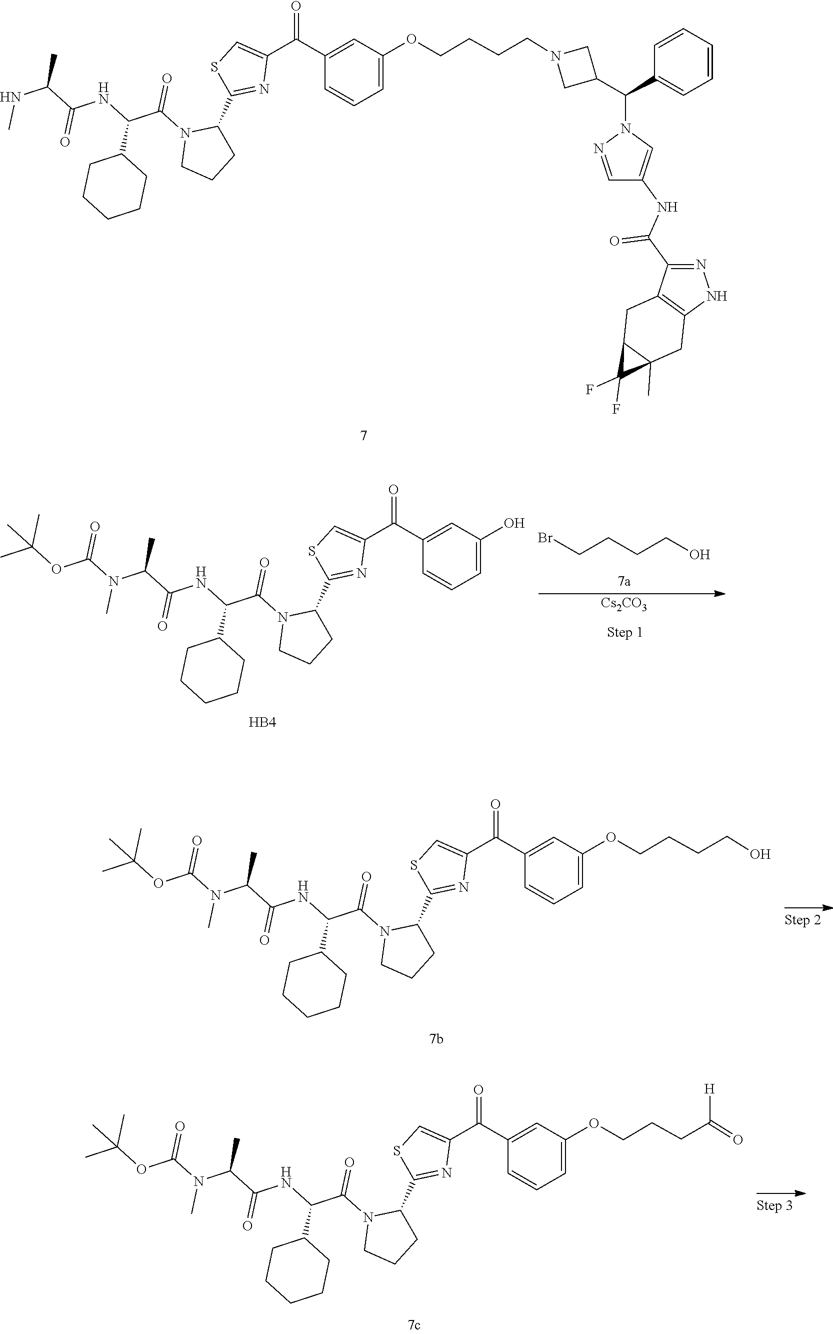

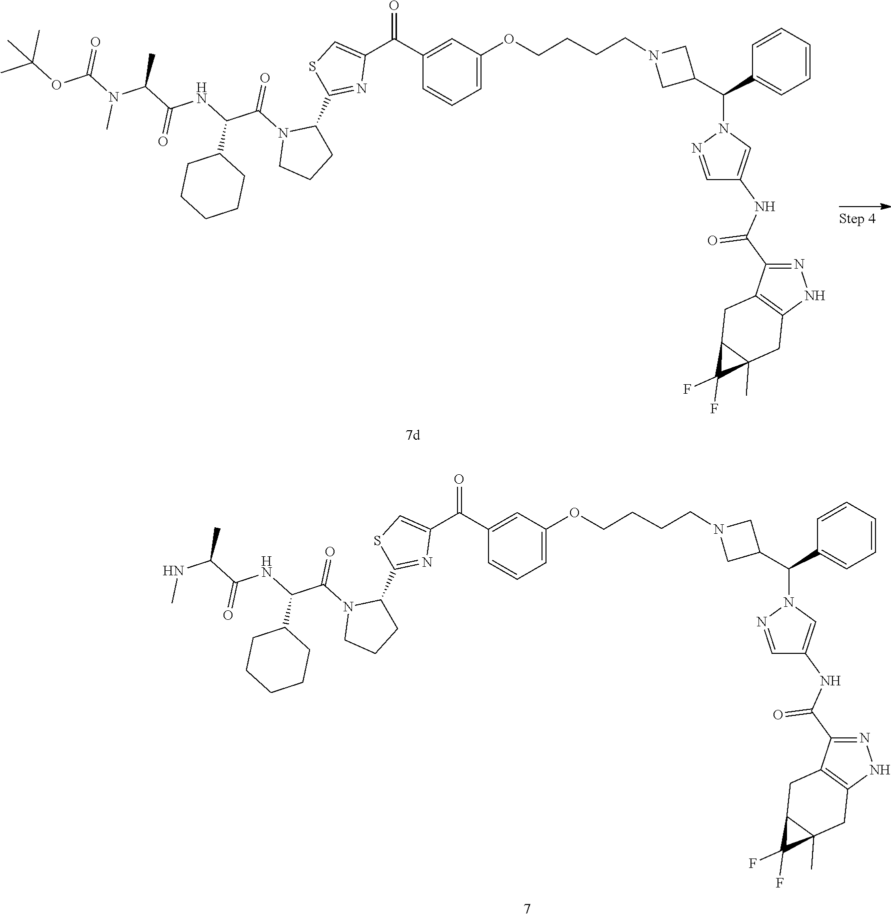 [SMILES: C[C@@H](C(=O)N[C@H](C(=O)N1CCC[C@H]1c1nc(C(=O)c2cccc(OCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1)N(C)C(=O)OC(C)(C)C ; CN[C@@H](C)C(=O)N[C@H](C(=O)N1CCC[C@H]1c1nc(C(=O)c2cccc(OCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1]

### Step 1: Synthesis of tert-butyl N-[(1S)-1-{[(1S)-1-cyclohexyl-2-[(2S)-2-{4-[3-(4-hydroxybutoxy)benzoyl]-1,3-thiazol-2-yl}pyrrolidin-1-yl]-2-oxoethyl]carbamoyl}ethyl]-N-methylcarbamate (7b)

To a solution of tert-butyl N-[(1S)-1-{[(1S)-1-cyclohexyl-2-[(2S)-2-[4-(3-hydroxybenzoyl)-1,3-thiazol-2-yl]pyrrolidin-1-yl]-2-oxoethyl]carbamoyl}ethyl]-N-methylcarbamate (70.00 mg, 0.12 mmol) and 4-bromobutan-1-ol (17.89 mg, 0.12 mmol) in DMF was added cesium carbonate (114.27 mg, 0.35 mmol) and the reaction was stirred at room temperature for three hours. The reaction mixture was then diluted with EtOAc and washed with water twice, dried, and concentrated. ISCO silica gel column purification eluting with MeOH:DCM (0-10%) provided tert-butyl N-[(1S)-1-{[(1S)-1-cyclohexyl-2-[(2S)-2-{4-[3-(4-hydroxybutoxy)benzoyl]-1,3-thiazol-2-yl}pyrrolidin-1-yl]-2-oxoethyl]carbamoyl}ethyl]-N-methylcarbamate (56 mg, 71%). LCMS: C₃₅H₅₀N₄O₇S requires: 671, found: m/z=694 [M+Na]⁺.

### Step 2: Synthesis of tert-butyl N-[(1S)-1-{[(1S)-1-cyclohexyl-2-oxo-2-[(2S)-2-{4-[3-(4-oxobutoxy)benzoyl]-1,3-thiazol-2-yl}pyrrolidin-1-yl]ethyl]carbamoyl}ethyl]-N-methylcarbamate (7c)

To a solution of tert-butyl N-[(1S)-1-{[(1S)-1-cyclohexyl-2-[(2S)-2-{4-[3-(4-hydroxybutoxy)benzoyl]-1,3-thiazol-2-yl}pyrrolidin-1-yl]-2-oxoethyl]carbamoyl}ethyl]-N-methylcarbamate (30.00 mg, 0.04 mmol) in DCM was added Dess-Martin reagent 1,1-bis(acetyloxy)-3-oxo-3H-1lambda5,2-benziodaoxol-1-yl acetate (18.97 mg, 0.04 mmol) and the reaction was stirred for one hour. LCMS showed no starting material left after one hour. The reaction mixture was then quenched with Na₂S₂O₃ (0.5 N solution, 0.5 mL) and NaHCO₃ (0.5 N aqueous solution, 0.5 mL) and stirred for fifteen minutes. The reaction mixture was extracted with DCM, dried over Na₂SO₄, filtered, and concentrated. The crude product was used in the next step without purification. LCMS: C₃₅H₄₈N₄O₇S requires: 669, found: m/z=670 [M+H]⁺.

### Step 3: Synthesis of tert-butyl N-[(1S)-1-{[(1S)-2-[(2S)-2-{4-[3-(4-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}butoxy)benzoyl]-1,3-thiazol-2-yl}pyrrolidin-1-yl]-1-cyclohexyl-2-oxoethyl]carbamoyl}ethyl]-N-methylcarbamate (7d)

To a solution of tert-butyl N-[(1S)-1-{[(1S)-1-cyclohexyl-2-oxo-2-[(2S)-2-{4-[3-(4-oxobutoxy)benzoyl]-1,3-thiazol-2-yl}pyrrolidin-1-yl]ethyl]carbamoyl}ethyl]-N-methylcarbamate (30.00 mg, 0.04 mmol) and (4aS,5aR)—N-{1-[(S)-(azetidin-3-yl)(phenyl)methyl]-1H-pyrazol-4-yl}-5,5-difluoro-5a-methyl-1H,4H,4aH,5H,5aH,6H-cyclopropa]indazole-3-carboxamide (19.67 mg, 0.04 mmol) in DCM (1 mL) was added acetic acid (4.04 mg, 0.07 mmol) and sodium bis(acetyloxy)boranuidyl acetate ([(AcO)₃BH]⁻Na⁺, 14.26 mg, 0.07 mmol) and the reaction was stirred for one hour. Prep-HPLC purification provided tert-butyl N-[(1S)-1-{[(1S)-2-[(2S)-2-{4-[3-(4-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}butoxy)benzoyl]-1,3-thiazol-2-yl}pyrrolidin-1-yl]-1-cyclohexyl-2-oxoethyl]carbamoyl}ethyl]-N-methylcarbamate (10 mg, 20.4%). LCMS: C₅₈H₇₂F₂N₁₀O₇S requires: 1091, found: m/z=1092 [M+H]⁺.

### Step 4: Synthesis of (4aS,5aR)—N-{1-[(S)-{1-[4-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)butyl]azetidin-3-yl}(phenyl)methyl]pyrazol-4-yl}-5,5-difluoro-5a-methyl-1H,4H,4aH,6H-cyclopropa]indazole-3-carboxamide (7)

tert-butyl N-[(1S)-1-{[(1S)-2-[(2S)-2-{4-[3-(4-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}butoxy)benzoyl]-1,3-thiazol-2-yl}pyrrolidin-1-yl]-1-cyclohexyl-2-oxoethyl]carbamoyl}ethyl]-N-methylcarbamate (10 mg, 0.01 mmol) was dissolved in DCM (1 mL) and trifluoroacetic acid (1 mL). After twenty minutes, the trifluoroacetic acid and DCM were evaporated. The crude product was purified by HPLC eluting with acetonitrile and water to afford (4aS,5aR)—N-{1-[(S)-{1-[4-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)butyl]azetidin-3-yl}(phenyl)methyl]pyrazol-4-yl}-5,5-difluoro-5a-methyl-1H,4H,4aH,6H-cyclopropa]indazole-3-carboxamide as a white solid (5 mg, 55%). ¹HNMR (500 MHz, Methanol-d₄) δ 8.30 (s, 1H), 7.76-7.72 (m, 1H), 7.69 (s, 1H), 7.61 (dd, J=2.7, 1.5 Hz, 1H), 7.46-7.31 (m, 6H), 7.23-7.18 (m, 1H), 5.66 (d, J=8.5 Hz, 1H), 5.46 (dd, J=7.6, 3.6 Hz, 1H), 4.54 (dd, J=14.6, 7.5 Hz, 1H), 4.40 (s, 1H), 4.15 (d, J=55.2 Hz, 5H), 3.96 (d, J=8.3 Hz, 2H), 3.92-3.83 (m, 2H), 3.18-3.00 (m, 5H), 2.78 (d, J=17.3 Hz, 1H), 2.66 (s, 3H), 2.27 (d, J=45.5 Hz, 3H), 1.87 (s, 2H), 1.69 (dd, J=66.8, 13.7 Hz, 11H), 1.48 (d, J=7.0 Hz, 3H), 1.38 (s, 4H), 1.30-0.99 (m, 7H). LCMS: C₅₃H₆₄F₂N₁₀O₅S requires: 991, found: m/z=992 [M+H]⁺.

### Example 55. (4aS,5aR)—N-(1-((S)-(1-(3-(3-(2-((S)-1-((S)-2-cyclohexyl-2-((S)-2-(methylamino)propanamido)acetyl)pyrrolidin-2-yl)thiazole-4-carbonyl)phenoxy)propyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (8)

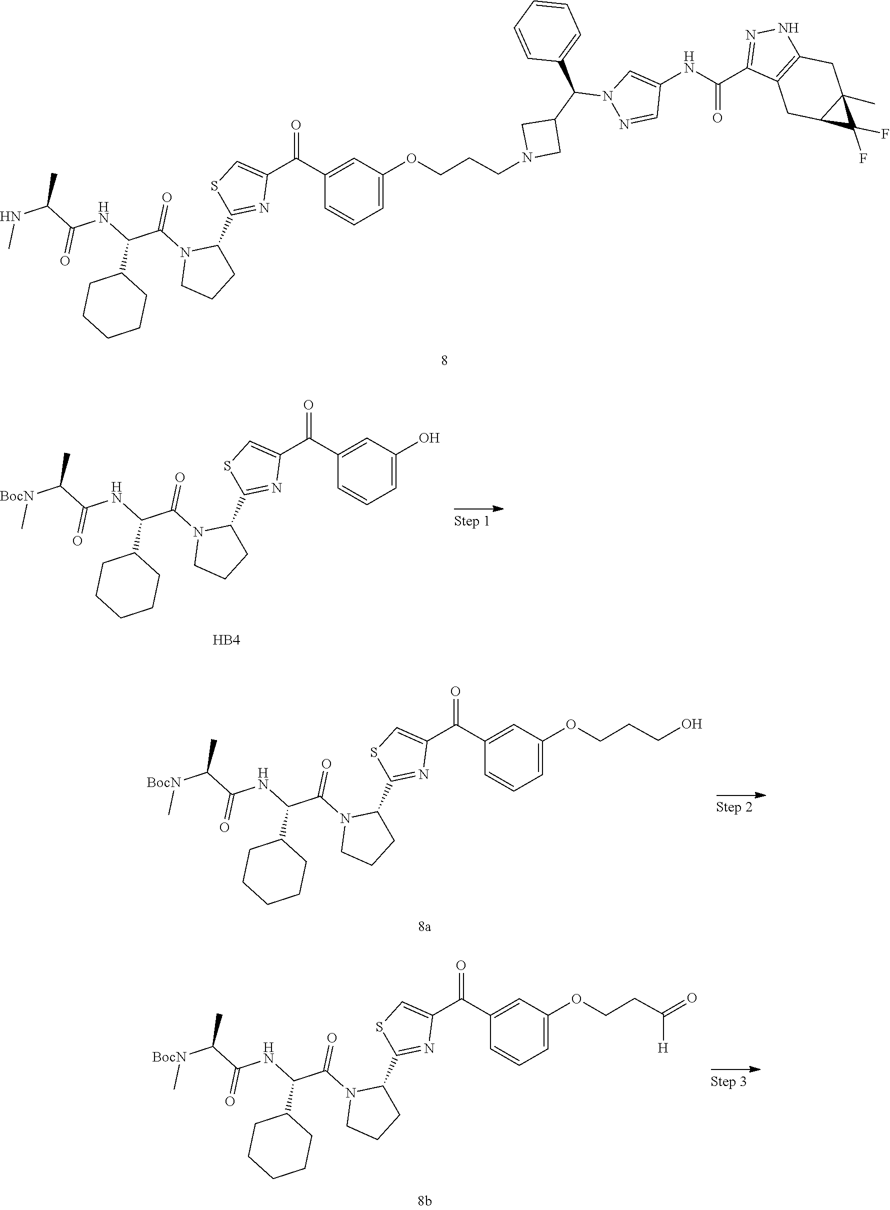

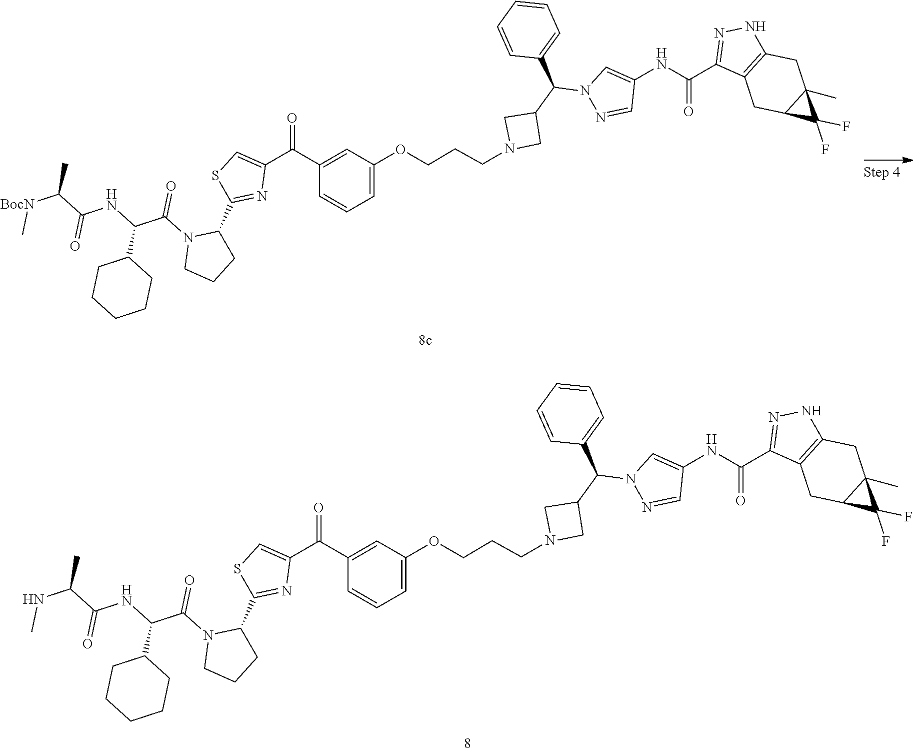 [SMILES: C[C@@H](C(=O)N[C@H](C(=O)N1CCC[C@H]1c1nc(C(=O)c2cccc(OCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1)N(C)C(=O)OC(C)(C)C ; CN[C@@H](C)C(=O)N[C@H](C(=O)N1CCC[C@H]1c1nc(C(=O)c2cccc(OCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1]

### Step 1: Synthesis of tert-butyl N-[(1S)-1-{[(1S)-1-cyclohexyl-2-[(2S)-2-{4-[3-(4-hydroxypropoxy)benzoyl]-1,3-thiazol-2-yl}pyrrolidin-1-yl]-2-oxoethyl]carbamoyl}ethyl]-N-methylcarbamate (8a)

To a solution of tert-butyl N-[(1S)-1-{[(1S)-1-cyclohexyl-2-[(2S)-2-[4-(3-hydroxybenzoyl)-1,3-thiazol-2-yl]pyrrolidin-1-yl]-2-oxoethyl]carbamoyl}ethyl]-N-methylcarbamate (HB4) (70.00 mg, 0.12 mmol) and 4-bromopropan-1-ol (16.3 mg, 0.12 mmol) in DMF was added cesium carbonate (114.27 mg, 0.35 mmol) and the reaction was stirred at room temperature for three hours. The reaction mixture was diluted with EtOAc and washed with water twice, dried over sodium sulfate, and concentrated. ISCO silica gel column purification eluting with MeOH:DCM (0-10%) provided tert-butyl N-[(1S)-1-{[(1S)-1-cyclohexyl-2-[(2S)-2-{4-[3-(4-hydroxypropoxy)benzoyl]-1,3-thiazol-2-yl}pyrrolidin-1-yl]-2-oxoethyl]carbamoyl}ethyl]-N-methylcarbamate (8a) (56 mg, 73%). LCMS: C₃₄H₄₈N₄O₇S requires: 657, found: m/z=680 [M+Na]⁺.

### Step 2: Synthesis of tert-butyl N-[(1S)-1-{[(1S)-1-cyclohexyl-2-oxo-2-[(2S)-2-{4-[3-(4-oxopropoxy)benzoyl]-1,3-thiazol-2-yl}pyrrolidin-1-yl]ethyl]carbamoyl}ethyl]-N-methylcarbamate (8b)

To a solution of tert-butyl N-[(1S)-1-{[(1S)-1-cyclohexyl-2-[(2S)-2-{4-[3-(4-hy4roxypropoxy)benzoyl]-1,3-thiazol-2-yl}pyrrolidin-1-yl]-2-oxoethyl]carbamoyl}ethyl]-N-methylcarbamate (8a) (40.00 mg, 0.06 mmol) in DCM was added Dess-Martin reagent 1,1-bis(acetyloxy)-3-oxo-3H-1lambda5,2-benziodaoxol-1-yl acetate (25.8 mg, 0.06 mmol) and the reaction was stirred for one hour. LCMS showed no starting material left after one hour. The reaction mixture was quenched with Na₂S₂O₃ (0.5 N solution, 0.5 mL) and NaHCO₃ aqueous solution (0.5 N, 0.5 mL) and stirred for fifteen minutes. The reaction mixture was extracted with DCM, dried over Na₂SO₄, filtered, and concentrated. The crude product was used in the next step without purification. LCMS: C₃₄H₄₆N₄O₇S requires: 655, found: m/z=656 [M+H]⁺.

### Step 3: Synthesis of tert-butyl N-[(1S)-1-{[(1S)-2-[(2S)-2-{4-[3-(4-{3-[(S)-{4-[(4aS,5aR)-5,5-difluoro-5a-methyl-1H,4H,4aH,6H-cyclopropa[f]indazole-3-amido]pyrazol-1-yl}(phenyl)methyl]azetidin-1-yl}propoxy)benzoyl]-1,3-thiazol-2-yl}pyrrolidin-1-yl]-1-cyclohexyl-2-oxoethyl]carbamoyl}ethyl]-N-methylcarbamate (8c)

To a solution of tert-butyl N-[(1S)-1-{[(1S)-1-cyclohexyl-2-oxo-2-[(2S)-2-{4-[3-(4-oxopropoxy)benzoyl]-1,3-thiazol-2-yl}pyrrolidin-1-yl]ethyl]carbamoyl}ethyl]-N-methylcarbamate (8b) (30.00 mg, 0.04 mmol) and (4aS,5aR)—N-{1-[(S)-(azetidin-3-yl)(phenyl)methyl]-1H-pyrazol-4-yl}-5,5-difluoro-5a-methyl-1H,4H,4aH,5H,5aH,6H-cyclopropafindazole-3-carboxamide (BBX₂) (30.13 mg, 0.07 mmol) in DCM (1 mL) was added acetic acid (4.13 mg, 0.07 mmol) and sodium triacetoxyborohydride (14.56 mg, 0.07 mmol) and the reaction was stirred for one hour. Prep-HPLC purification provided tert-butyl N-[(1S)-1-{[(1S)-2-[(2S)-2-{4-[3-(4-{3-[(S)-{4-[(4aS,5aR)-5,5-difluoro-5a-methyl-1H,4H,4aH,6H-cyclopropafindazole-3-amido]pyrazol-1-yl}(phenyl)methyl]azetidin-1-yl}propoxy)benzoyl]-1,3-thiazol-2-yl}pyrrolidin-1-yl]-1-cyclohexyl-2-oxoethyl]carbamoyl}ethyl]-N-methylcarbamate (8c) (12 mg, 24.3%). LCMS: C₅₇H₇₀F₂N₁₀O₇S requires: 1077, found: m/z=1078 [M+H]⁺.

### Step 4: Synthesis of (4aS,5aR)—N-{1-[(S)-{1-[4-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)propyl]azetidin-3-yl}(phenyl)methyl]pyrazol-4-yl}-5,5-difluoro-5a-methyl-1H,4H,4aH,6H-cyclopropafindazole-3-carboxamide (8)

tert-butyl N-[(1S)-1-{[(1S)-2-[(2S)-2-{4-[3-(4-{3-[(S)-{4-[(4aS,5aR)-5,5-difluoro-5a-methyl-1H,4H,4aH,6H-cyclopropafindazole-3-amido]pyrazol-1-yl}(phenyl)methyl]azetidin-1-yl}propoxy)benzoyl]-1,3-thiazol-2-yl}pyrrolidin-1-yl]-1-cyclohexyl-2-oxoethyl]carbamoyl}ethyl]-N-methylcarbamate (8c) (12 mg, 0.01 mmol) was dissolved in DCM (1 mL) and trifluoroacetic acid (1 mL). After twenty minutes, trifluoroacetic acid and DCM were evaporated. The crude product was purified by HPLC eluting with acetonitrile and water to afford (4aS,5aR)—N-{1-[(S)-{1-[4-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)propyl]azetidin-3-yl}(phenyl)methyl]pyrazol-4-yl}-5,5-difluoro-5a-methyl-1H,4H,4aH,6H-cyclopropafindazole-3-carboxamide (8) as a white solid (2 mg, 13%). LCMS: C₅₂H₆₂F₂N₁₀O₅S requires: 977, found: m/z=978 [M+H]⁺.

### Example 56. (4aS,5aR)—N-(1-((S)-(1-(6-(3-(2-((S)-1-((S)-2-cyclohexyl-2-((S)-2-(methylamino)propanamido)acetyl)pyrrolidin-2-yl)thiazole-4-carbonyl)phenoxy)hexyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (9)

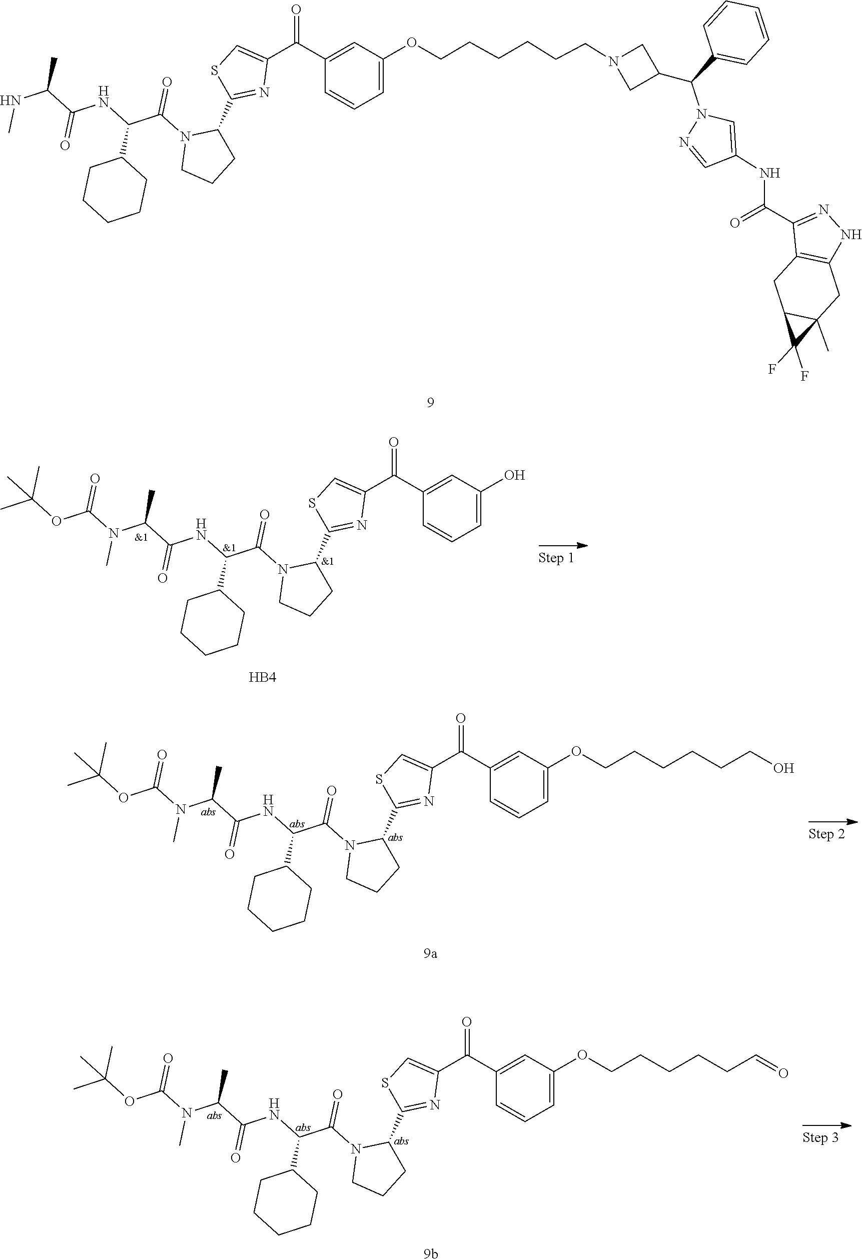

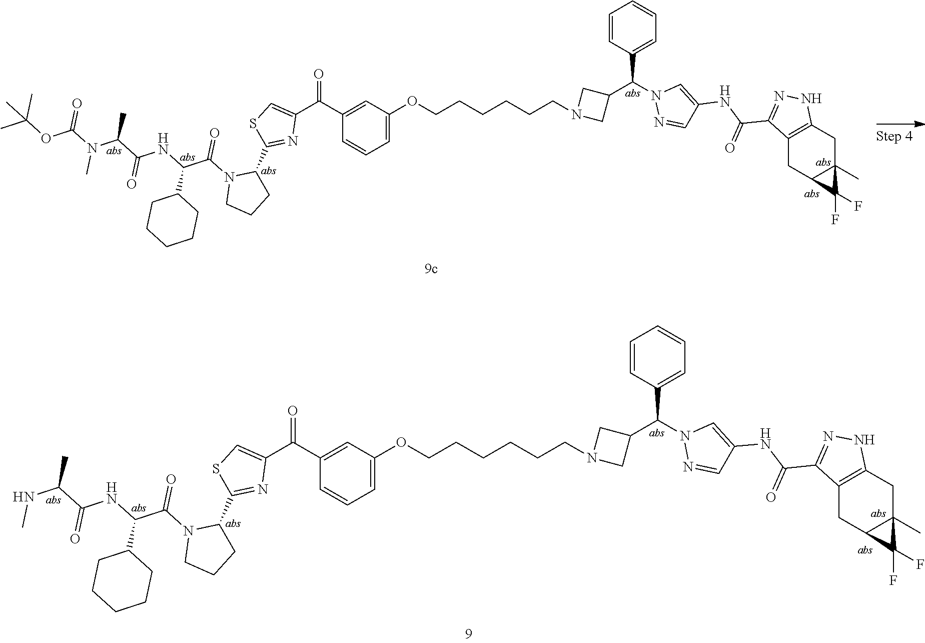 [SMILES: C[C@@H](C(=O)N[C@H](C(=O)N1CCC[C@H]1c1nc(C(=O)c2cccc(OCCCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1)N(C)C(=O)OC(C)(C)C ; CN[C@@H](C)C(=O)N[C@H](C(=O)N1CCC[C@H]1c1nc(C(=O)c2cccc(OCCCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1]

### Step 1: Synthesis of tert-butyl N-[(1S)-1-{[(1S)-1-cyclohexyl-2-[(2S)-2-(4-{3-[(5-hydroxyhexyl)oxy]benzoyl}-1,3-thiazol-2-yl)pyrrolidin-1-yl]-2-oxoethyl]carbamoyl}ethyl]-N-methylcarbamate (9a)

To a solution of tert-butyl N-[(1S)-1-{[(1S)-1-cyclohexyl-2-[(2S)-2-[4-(3-hydroxybenzoyl)-1,3-thiazol-2-yl]pyrrolidin-1-yl]-2-oxoethyl]carbamoyl}ethyl]-N-methylcarbamate (HB4) (70.00 mg, 0.12 mmol) and 4-bromopentan-1-ol (21.17 mg, 0.12 mmol) in DMF was added cesium carbonate (114.27 mg, 0.35 mmol) and the reaction was stirred at room temperature for three hours. The reaction mixture was then diluted with EtOAc and washed with water twice, dried, and concentrated. ISCO silica gel column purification eluting with MeOH:DCM (0-10%) provided tert-butyl N-[(1S)-1-{[(1S)-1-cyclohexyl-2-[(2S)-2-(4-{3-[(5-hydroxyhexyl)oxy]benzoyl}-1,3-thiazol-2-yl)pyrrolidin-1-yl]-2-oxoethyl]carbamoyl}ethyl]-N-methylcarbamate (9a) (57 mg, 69%). LCMS: C₃₇H₅₄N₄O₇S requires: 699, found: m/z=722 [M+Na]⁺.

### Step 2: Synthesis of tert-butyl N-[(1S)-1-{[(1S)-1-cyclohexyl-2-oxo-2-[(2S)-2-(4-{3-[(5-oxohexyl)oxy]benzoyl}-1,3-thiazol-2-yl)pyrrolidin-1-yl]ethyl]carbamoyl}ethyl]-N-methylcarbamate (9b)

To a solution of tert-butyl N-[(1S)-1-{[(1S)-1-cyclohexyl-2-[(2S)-2-(4-{3-[(5-hydroxyhexyl)oxy]benzoyl}-1,3-thiazol-2-yl)pyrrolidin-1-yl]-2-oxoethyl]carbamoyl}ethyl]-N-methylcarbamate (9a) (30.00 mg, 0.04 mmol) in DCM was added Dess-Martin reagent 1,1-bis(acetyloxy)-3-oxo-3H-1lambda5,2-benziodaoxol-1-yl acetate (18.21 mg, 0.04 mmol) and the reaction was stirred for one hour. LCMS showed no starting material left after one hour. The reaction mixture was quenched with Na₂S₂O₃ (0.5 N solution, 0.5 mL) and NaHCO₃ aqueous solution (0.5 N, 0.5 mL) and stirred for fifteen minutes. The reaction mixture was then extracted with DCM, dried over Na₂SO₄, filtered, and concentrated. The crude product was used in the next step without purification. LCMS: C₃₇H₅₂N₄O₇S requires: 697, found: m/z=698 [M+H]⁺.

### Step 3: Synthesis of tert-butyl N-[(1S)-1-{[(1S)-2-[(2S)-2-(4-{3-[(5-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}hexyl)oxy]benzoyl}-1,3-thiazol-2-yl)pyrrolidin-1-yl]-1-cyclohexyl-2-oxoethyl]carbamoyl}ethyl]-N-methylcarbamate (9c)

To a solution of tert-butyl N-[(1S)-1-{[(1S)-1-cyclohexyl-2-oxo-2-[(2S)-2-(4-{3-[(5-oxohexyl)oxy]benzoyl}-1,3-thiazol-2-yl)pyrrolidin-1-yl]ethyl]carbamoyl}ethyl]-N-methylcarbamate (9b) (30.00 mg, 0.04 mmol) and (4aS,5aR)—N-{1-[(S)-(azetidin-3-yl)(phenyl)methyl]-1H-pyrazol-4-yl}-5,5-difluoro-5a-methyl-1H,4H,4aH,5H,5aH,6H-cyclopropa[indazole-3-carboxamide (BBX₂) (18.88 mg, 0.04 mmol) in DCM (1 mL) was added acetic acid (3.88 mg, 0.06 mmol) and sodium triacetoxyborohydride (13.69 mg, 0.06 mmol) and the reaction was stirred for one hour. Prep-HPLC purification provided tert-butyl N-[(1S)-1-{[(1S)-2-[(2S)-2-(4-{3-[(5-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}hexyl)oxy]benzoyl}-1,3-thiazol-2-yl)pyrrolidin-1-yl]-1-cyclohexyl-2-oxoethyl]carbamoyl}ethyl]-N-methylcarbamate (9c) (15 mg, 31%) LCMS: C₅₉H₇₄F₂N₁₀O₇S requires: 1119, found: m/z=1142 [M+Na]⁺.

### Step 4: Synthesis of (4aS,5aR)—N-{1-[(S)-{1-[5-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)hexyl]azetidin-3-yl}(phenyl)methyl]pyrazol-4-yl}-5,5-difluoro-5a-methyl-1H,4H,4aH,6H-cyclopropa]indazole-3-carboxamide (9)

tert-butyl N-[(1S)-1-{[(1S)-2-[(2S)-2-(4-{3-[(5-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}hexyl)oxy]benzoyl}-1,3-thiazol-2-yl)pyrrolidin-1-yl]-1-cyclohexyl-2-oxoethyl]carbamoyl}ethyl]-N-methylcarbamate (9c) (15 mg, 0.01 mmol) was dissolved in DCM (1 mL) and trifluoroacetic acid (1 mL). After twenty minutes, trifluoroacetic acid and DCM were evaporated. The crude product was purified by HPLC eluting with acetonitrile and water to afford (4aS,5aR)—N-{1-[(S)-{1-[5-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)hexyl]azetidin-3-yl}(phenyl)methyl]pyrazol-4-yl}-5,5-difluoro-5a-methyl-1H,4H,4aH,6H-cyclopropa]indazole-3-carboxamide (9) as a white solid (13 mg, 95%). ¹H NMR (500 MHz, Methanol-d₄) δ 8.31 (d, J=16.8 Hz, 1H), 7.92 (s, 1H), 7.69 (s, 2H), 7.61 (t, J=2.1 Hz, 1H), 7.46-7.28 (m, 5H), 7.18 (ddd, J=8.3, 2.7, 1.0 Hz, 1H), 5.67 (t, J=11.1 Hz, 1H), 5.46 (dd, J=7.9, 3.4 Hz, 1H), 4.60-4.48 (m, 1H), 4.37 (d, J=10.0 Hz, 1H), 4.24-3.81 (m, 8H), 3.21-3.01 (m, 4H), 2.83-2.72 (m, 1H), 2.66 (s, 2H), 2.40-2.18 (m, 2H), 2.18-2.05 (m, 1H), 1.89-1.51 (m, 12H), 1.51-1.32 (m, 7H), 1.29-0.97 (m, 5H). LCMS: C₅₅H₆₈F₂N₁₀O₅S requires: 1019, found: m/z=1020 [M+H]⁺.

### Example 57. (4aS,5aR)—N-(1-((S)-(1-(5-(3-(2-((S)-1-((S)-2-cyclohexyl-2-((S)-2-(methylamino)propanamido)acetyl)pyrrolidin-2-yl)thiazole-4-carbonyl)phenoxy)pentyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (10)

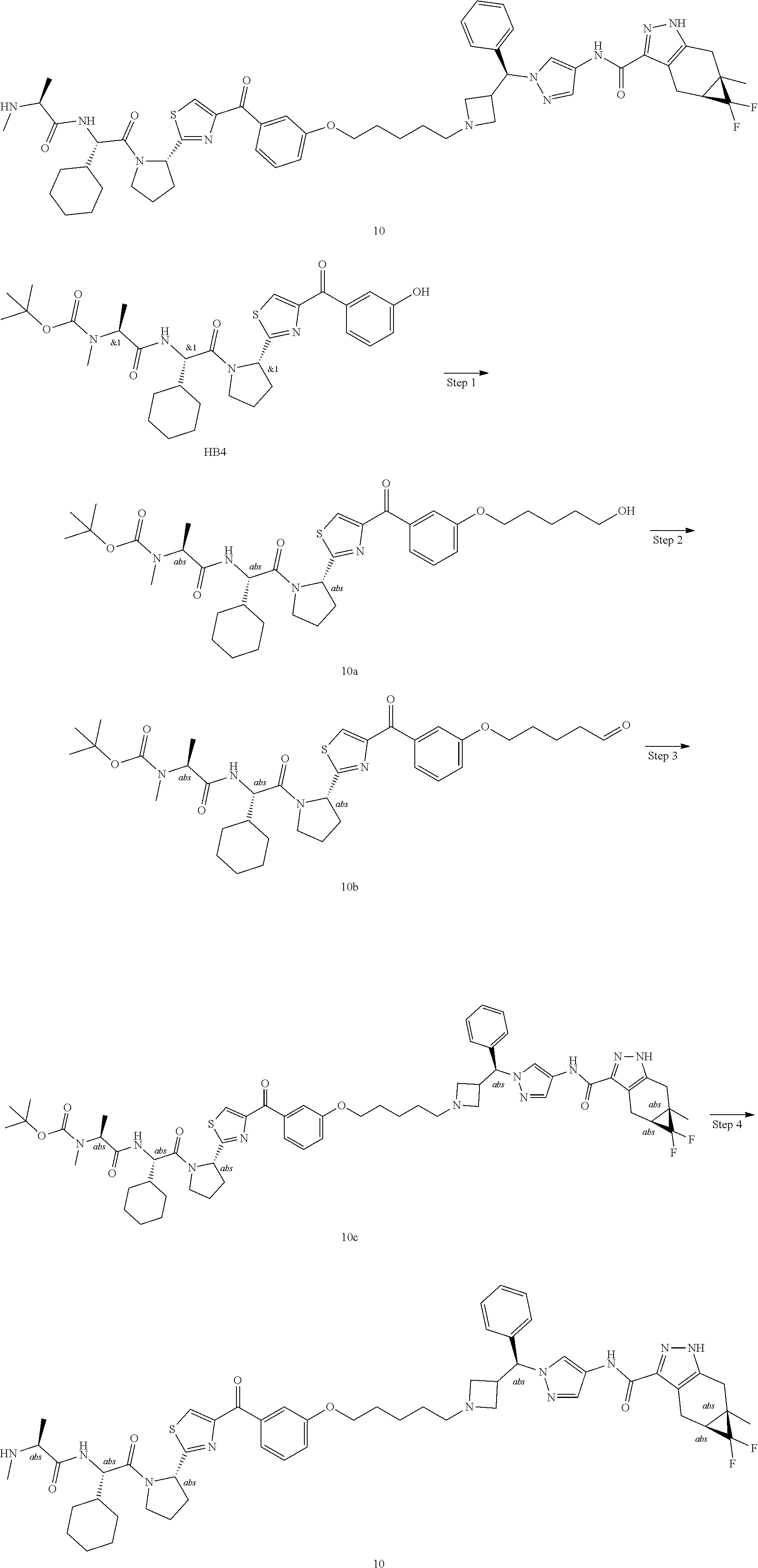

### Step 1: Synthesis of tert-butyl N-[(1S)-1-{[(1S)-1-cyclohexyl-2-[(2S)-2-(4-{3-[(5-hydroxypentyl)oxy]benzoyl}-1,3-thiazol-2-yl)pyrrolidin-1-yl]-2-oxoethyl]carbamoyl}ethyl]-N-methylcarbamate (10a)

To a solution of tert-butyl N-[(1S)-1-{[(1S)-1-cyclohexyl-2-[(2S)-2-[4-(3-hydroxybenzoyl)-1,3-thiazol-2-yl]pyrrolidin-1-yl]-2-oxoethyl]carbamoyl}ethyl]-N-methylcarbamate (70.00 mg, 0.12 mmol) and 4-bromopentan-1-ol (HB4) (19.53 mg, 0.12 mmol) in DMF (1 mL) was added cesium carbonate (114.27 mg, 0.35 mmol) and the reaction was stirred at room temperature for three hours. The reaction mixture was then diluted with EtOAc and washed with water twice, dried, and concentrated. ISCO silica gel column purification eluting with MeOH:DCM (0-10%) provided tert-butyl N-[(1S)-1-{[(1S)-1-cyclohexyl-2-[(2S)-2-(4-{3-[(5-hydroxypentyl)oxy]benzoyl}-1,3-thiazol-2-yl)pyrrolidin-1-yl]-2-oxoethyl]carbamoyl}ethyl]-N-methylcarbamate (10a) (56 mg, 70%). LCMS: C₃₆H₅₂N₄O₇S requires: 685, found: m/z=708 [M+Na]⁺.

### Step 2: Synthesis of tert-butyl N-[(1S)-1-{[(1S)-1-cyclohexyl-2-oxo-2-[(2S)-2-(4-{3-[(5-oxopentyl)oxy]benzoyl}-1,3-thiazol-2-yl)pyrrolidin-1-yl]ethyl]carbamoyl}ethyl]-N-methylcarbamate (10b)

To a solution of tert-butyl N-[(1S)-1-{[(1S)-1-cyclohexyl-2-[(2S)-2-(4-{3-[(5-hydroxypentyl)oxy]benzoyl}-1,3-thiazol-2-yl)pyrrolidin-1-yl]-2-oxoethyl]carbamoyl}ethyl]-N-methylcarbamate (10a) (30.00 mg, 0.04 mmol) in DCM was added Dess-Martin reagent 1,1-bis(acetyloxy)-3-oxo-3H-1lambda5,2-benziodaoxol-1-yl acetate (18.58 mg, 0.04 mmol) and the reaction was stirred for one hour. LCMS showed no starting material left after one hour. The reaction mixture was then quenched with Na₂S₂O₃ (0.5 N solution, 0.5 mL) and NaHCO₃ aqueous solution (0.5 N, 0.5 mL) and stirred for fifteen minutes. The reaction mixture was then extracted with DCM, dried over Na₂SO₄, filtered, and concentrated. The crude product was used in the next step without purification. LCMS: C₃₆H₅₀N₄O₇S requires: 683, found: m/z=684 [M+H]⁺.

### Step 3: Synthesis of tert-butyl N-[(1S)-1-{[(1S)-2-[(2S)-2-(4-{3-[(5-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}pentyl)oxy]benzoyl}-1,3-thiazol-2-yl)pyrrolidin-1-yl]-1-cyclohexyl-2-oxoethyl]carbamoyl}ethyl]-N-methylcarbamate (10c)

To a solution of tert-butyl N-[(1S)-1-{[(1S)-1-cyclohexyl-2-oxo-2-[(2S)-2-(4-{3-[(5-oxopentyl)oxy]benzoyl}-1,3-thiazol-2-yl)pyrrolidin-1-yl]ethyl]carbamoyl}ethyl]-N-methylcarbamate (10b) (25.00 mg, 0.04 mmol) and (4aS,5aR)—N-{1-[(S)-(azetidin-3-yl)(phenyl)methyl]-1H-pyrazol-4-yl}-5,5-difluoro-5a-methyl-1H,4H,4aH,5H,5aH,6H-cyclopropa]indazole-3-carboxamide (16.05 mg, 0.04 mmol) in DCM (1 mL) was added acetic acid (3.3 mg, 0.05 mmol) and sodium triacetoxyborohydride (11.64 mg, 0.05 mmol) and the reaction was stirred for one hour. Prep-HPLC purification provided tert-butyl N-[(1S)-1-{[(1S)-2-[(2S)-2-(4-{3-[(5-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}pentyl)oxy]benzoyl}-1,3-thiazol-2-yl)pyrrolidin-1-yl]-1-cyclohexyl-2-oxoethyl]carbamoyl}ethyl]-N-methylcarbamate (10c) (10 mg, 24.7%). LCMS: C₅₉H₇₄F₂N₁₀O₇S requires: 1105, found: m/z=1106 [M+H]⁺.

### Step 4: Synthesis of (4aS,5aR)—N-{1-[(S)-{1-[5-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)pentyl]azetidin-3-yl}(phenyl)methyl]pyrazol-4-yl}-5,5-difluoro-5a-methyl-1H,4H,4aH,6H-cyclopropa]indazole-3-carboxamide (10)

tert-butyl N-[(1S)-1-{[(1S)-2-[(2S)-2-(4-{3-[(5-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}pentyl)oxy]benzoyl}-1,3-thiazol-2-yl)pyrrolidin-1-yl]-1-cyclohexyl-2-oxoethyl]carbamoyl}ethyl]-N-methylcarbamate (10c) (10 mg, 0.01 mmol) was dissolved in DCM (1 mL) and trifluoroacetic acid (1 mL). After twenty minutes, trifluoroacetic acid and DCM were evaporated. The crude product was purified by HPLC eluting with acetonitrile and water to afford (4aS,5aR)—N-{1-[(S)-{1-[5-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)pentyl]azetidin-3-yl}(phenyl)methyl]pyrazol-4-yl}-5,5-difluoro-5a-methyl-1H,4H,4aH,6H-cyclopropa]indazole-3-carboxamide (10) as a white solid (6 mg, 66%). ¹H NMR (500 MHz, Methanol-d₄) δ 8.30 (s, 1H), 7.80-7.65 (m, 2H), 7.59 (d, J=2.1 Hz, 1H), 7.47-7.29 (m, 6H), 7.18 (dd, J=8.2, 2.5 Hz, 1H), 5.66 (d, J=9.0 Hz, 1H), 5.46 (dd, J=7.9, 3.4 Hz, 1H), 4.56 (d, J=7.2 Hz, 1H), 4.23 (d, J=159.3 Hz, 5H), 4.02-3.81 (m, 4H), 3.19-3.01 (m, 6H), 2.78 (d, J=17.0 Hz, 1H), 2.66 (s, 3H), 2.41-2.18 (m, 3H), 2.12 (s, 1H), 1.89-1.52 (m, 14H), 1.47 (d, J=7.0 Hz, 3H), 1.38 (s, 4H), 1.31-1.01 (m, 7H). LCMS: C₅₄H₆₆F₂N₁₀O₅S requires: 1005, found: m/z=1006 [M+H]⁺.

### Example 58. (4aS,5aR)—N-(1-((S)-(1-(2-(2-(3-(2-((S)-1-((S)-2-cyclohexyl-2-((S)-2-(methylamino)propanamido)acetyl)pyrrolidin-2-yl)thiazole-4-carbonyl)phenoxy)ethoxy)ethyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (11)

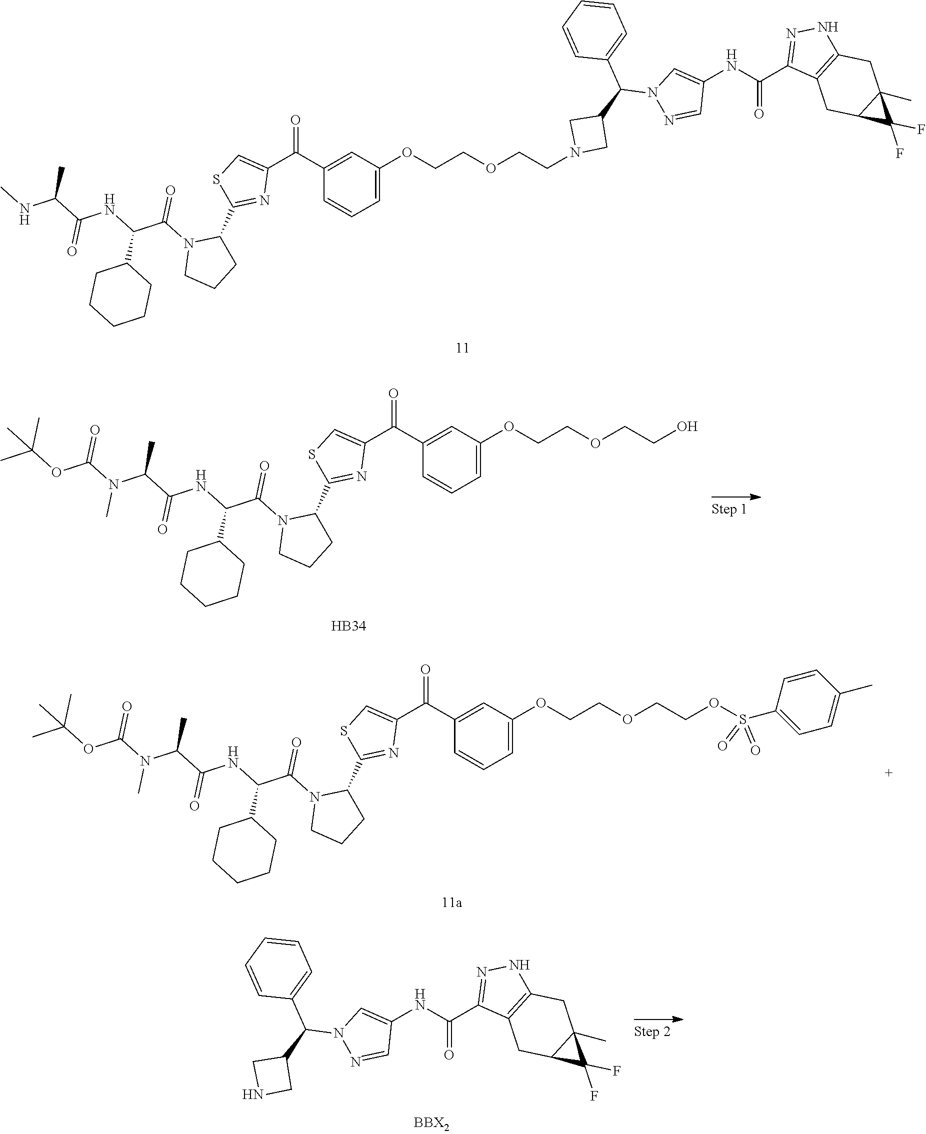

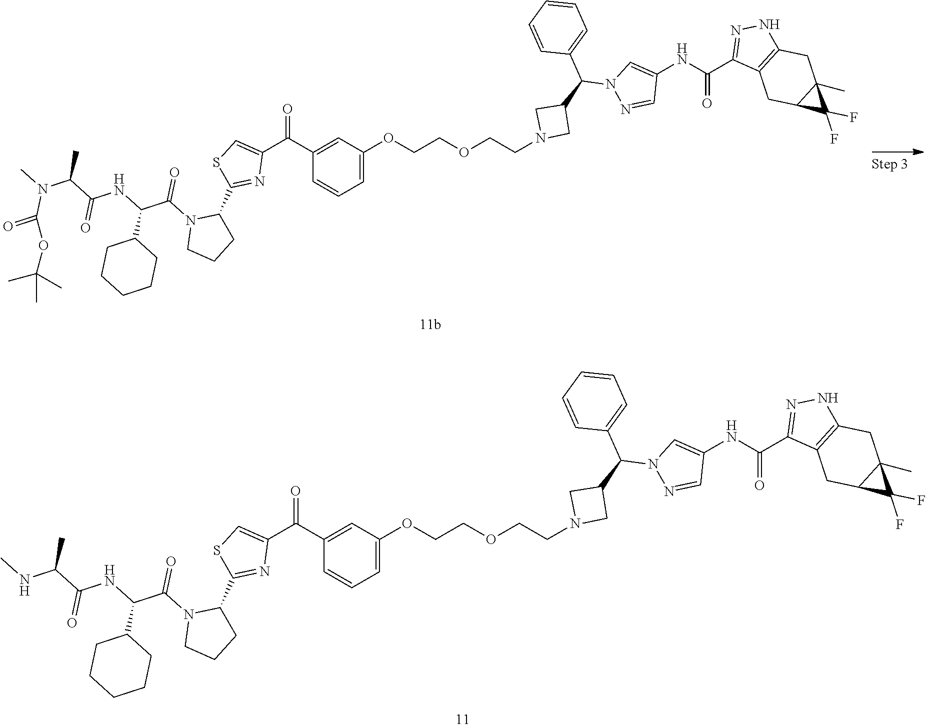 [SMILES: C[C@@H](C(=O)N[C@H](C(=O)N1CCC[C@H]1c1nc(C(=O)c2cccc(OCCOCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1)N(C)C(=O)OC(C)(C)C ; CN[C@@H](C)C(=O)N[C@H](C(=O)N1CCC[C@H]1c1nc(C(=O)c2cccc(OCCOCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1]

### Step 1: Synthesis of tert-butyl N-[(1S)-1-{[(1S)-1-cyclohexyl-2-[(2S)-2-{4-[3-(2-{2-[(4-methylbenzenesulfonyl)oxy]ethoxy}ethoxy)benzoyl]-1,3-thiazol-2-yl}pyrrolidin-1-yl]-2-oxoethyl]carbamoyl}ethyl]-N-methylcarbamate (11a)

To a solution of tert-butyl N-[(1S)-1-{[(1S)-1-cyclohexyl-2-[(2S)-2-(4-{3-[2-(2-hydroxyethoxy)ethoxy]benzoyl}-1,3-thiazol-2-yl)pyrrolidin-1-yl]-2-oxoethyl]carbamoyl}ethyl]-N-methylcarbamate (200.00 mg, 0.29 mmol), 4-dimethylaminopyridine (3.60 mg, 0.03 mmol), triethylamine (81.17 μL, 0.58 mmol) in DCM (9 mL) was added p-toluenesulfonyl chloride (66.61 mg, 0.35 mmol) in DCM (2 mL) and the reaction was stirred at room temperature overnight. The reaction was terminated with a 1 N HCl solution and the organics were washed with brine, dried, filtered, and concentrated. The crude product was purified by ISCO silica gel column chromatography eluting with EtOAc in Hexanes (10%-100%) and provided tert-butyl N-[(1S)-1-{[(1S)-1-cyclohexyl-2-[(2S)-2-{4-[3-(2-{2-[(4-methylbenzenesulfonyl)oxy]ethoxy}ethoxy)benzoyl]-1,3-thiazol-2-yl}pyrrolidin-1-yl]-2-oxoethyl]carbamoyl}ethyl]-N-methylcarbamate (143 mg, 58.4%). LCMS: C₄₂H₅₆N₄O₁₀S₂ requires: 841, found: m/z=842 [M+H]⁺.

### Step 2: Synthesis of tert-butyl N-[(1S)-1-{[(1S)-2-[(2S)-2-(4-{3-[2-(2-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}ethoxy)ethoxy]benzoyl}-1,3-thiazol-2-yl)pyrrolidin-1-yl]-1-cyclohexyl-2-oxoethyl]carbamoyl}ethyl]-N-methylcarbamate (11b)

(4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (52.00 mg, 0.12 mmol), tert-butyl N-[(1S)-1-{[(1S)-1-cyclohexyl-2-[(2S)-2-{4-[3-(2-{2-[(4-methylbenzenesulfonyl)oxy]ethoxy}ethoxy)benzoyl]-1,3-thiazol-2-yl}pyrrolidin-1-yl]-2-oxoethyl]carbamoyl}ethyl]-N-methylcarbamate (99.74 mg, 0.12 mmol), and N,N-diisopropylethylamine (61.97 μL, 0.36 mmol) in DMF (2 mL) was stirred at 70° C. for six hours. LCMS indicated the completion of reaction. The reaction solution was diluted with EtOAc (30 mL), washed with water twice, dried, filtered, and concentrated. The crude product was purified by ISCO silica gel column chromatography eluting with MeOH in EtOAc (0-30%) and provided tert-butyl N-[(1S)-1-{[(1S)-2-[(2S)-2-(4-{3-[2-(2-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}ethoxy)ethoxy]benzoyl}-1,3-thiazol-2-yl)pyrrolidin-1-yl]-1-cyclohexyl-2-oxoethyl]carbamoyl}ethyl]-N-methylcarbamate (38.8 mg, 29.5%). LCMS: C₅₈H₇₂F₂N₁₀O₈S requires: 1107, found: m/z=1108 [M+H]⁺.

### Step 3: Synthesis of (4aS,5aR)—N-{1-[(S)-(1-{2-[2-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)ethoxy]ethyl}azetidin-3-yl)(phenyl)methyl]pyrazol-4-yl}-5,5-difluoro-5a-methyl-1H,4H,4aH,6H-cyclopropa[indazole-3-carboxamide (11)

tert-butyl N-[(1S)-1-{[(1S)-2-[(2S)-2-(4-{3-[2-(2-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}ethoxy)ethoxy]benzoyl}-1,3-thiazol-2-yl)pyrrolidin-1-yl]-1-cyclohexyl-2-oxoethyl]carbamoyl}ethyl]-N-methylcarbamate (11b) (18.60 mg, 0.1 mmol) was dissolved in TFA/DCM (1:1, 2 mL) and the mixture was stirred for twenty minutes. Thereafter, TFA and DCM were evaporated. The crude was redissolved in DCM (20 mL) and washed with ammonium hydroxide (10% solution, 1 mL), followed by brine (1 mL), and the DCM was evaporated. The residue was then lyophilized to dryness. The crude was purified using a C18 column eluting with acetonitrile in water to provide (4aS,5aR)—N-{1-[(S)-(1-{2-[2-(3-{2-[(2S)-1-[(2S)-2-cyclohexyl-2-[(2S)-2-(methylamino)propanamido]acetyl]pyrrolidin-2-yl]-1,3-thiazole-4-carbonyl}phenoxy)ethoxy]ethyl}azetidin-3-yl)(phenyl)methyl]pyrazol-4-yl}-5,5-difluoro-5a-methyl-1H,4H,4aH,6H-cyclopropa[indazole-3-carboxamide (6.6 mg, 65.5%). LCMS: C₅₃H₆₄F₂N₁₀O₆S requires: 1007, found: m/z=1008 [M+H]⁺. ¹H NMR (500 MHz, DMSO-d₆) δ 12.94 (s, 1H), 10.13 (s, 1H), 8.49 (s, 1H), 8.06 (s, 2H), 7.74-7.55 (m, 3H), 7.42 (t, J=8.0 Hz, 1H), 7.40-7.22 (m, 6H), 7.16 (dd, J=8.1, 2.6 Hz, 1H), 5.63-5.51 (m, 1H), 5.38 (dd, J=8.0, 3.1 Hz, 1H), 4.49 (dd, J=8.7, 6.8 Hz, 1H), 4.13 (t, J=4.6 Hz, 2H), 3.79 (s, 2H), 3.71 (s, 3H), 3.07-2.89 (m, 7H), 2.81 (dd, J=13.6, 2.9 Hz, 2H), 2.22 (d, J=36.3 Hz, 6H), 2.03 (s, 3H), 1.80-1.42 (m, 10H), 1.42-0.65 (m, 21H).

### Example 59. (4aS,5aR)-5,5-difluoro-N-(1-((S)-(1-((S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl)azetidin-3-yl)(phenyl)methyl)-1H-pyrazol-4-yl)-5a-methyl-1,4,4a,5,5a,6-hexahydrocyclopropa[f]indazole-3-carboxamide (12)

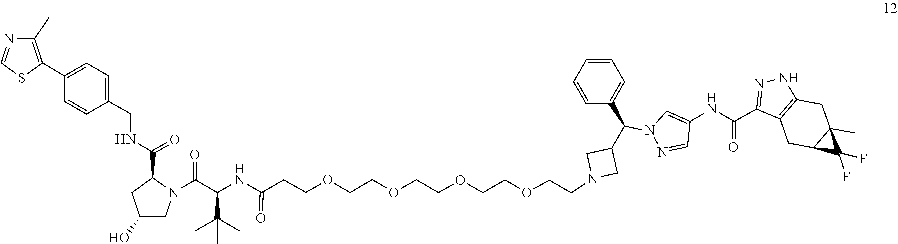 [SMILES: Cc1ncsc1-c1ccc(CNC(=O)[C@@H]2C[C@@H](O)CN2C(=O)[C@@H](NC(=O)CCOCCOCCOCCOCCN2CC([C@@H](c3ccccc3)n3cc(NC(=O)c4n[nH]c5c4C[C@@H]4C(F)(F)[C@]4(C)C5)cn3)C2)C(C)(C)C)cc1]

Using General Procedure 8, (S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl 4-methylbenzenesulfonate (HB8) (38 mg, 0.046 mmol) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) (20 mg, 0.046 mmol) to afford (4aS,5aR)-5,5-difluoro-N-(1-((S)-(1-((S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl)azetidin-3-yl)(phenyl)methyl)-1H-pyrazol-4-yl)-5a-methyl-1,4,4a,5,5a,6-hexahydrocyclopropa[f]indazole-3-carboxamide (12). LCMS: C56H₇₂F₂N₁₀O₉S requires: 1099, found: m/z=1100 [M+H]⁺.

### Example 60. (4aS,5aR)-5,5-difluoro-N-(1-((S)-(1-((S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoyl)azetidin-3-yl)(phenyl)methyl)-1H-pyrazol-4-yl)-5a-methyl-1,4,4a,5,5a,6-hexahydrocyclopropa[f]indazole-3-carboxamide (13)

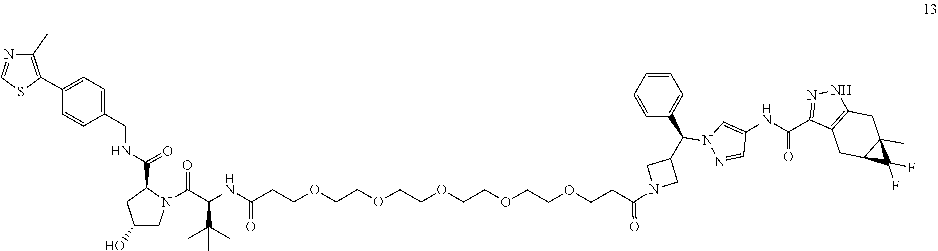 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

Using General Procedure 7, (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (HB31) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) to afford (4aS,5aR)-5,5-difluoro-N-(1-((S)-(1-((S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoyl)azetidin-3-yl)(phenyl)methyl)-1H-pyrazol-4-yl)-5a-methyl-1,4,4a,5,5a,6-hexahydrocyclopropa[f]indazole-3-carboxamide (13). ¹H NMR (500 MHz, MeOD) δ 8.86 (s, 1H), 8.05 (d, J=6.4 Hz, 1H), 7.68 (d, J=1.5 Hz, 1H), 7.48-7.29 (m, 9H), 5.57 (dd, J=10.8, 3.7 Hz, 1H), 4.65 (s, 1H), 4.57 (dd, J=9.0, 7.6 Hz, 1H), 4.55-4.46 (m, 2H), 4.40-4.25 (m, 2H), 4.13-4.02 (m, 1H), 4.02-3.95 (m, 1H), 3.89 (d, J=11.0 Hz, 1H), 3.82-3.77 (m, 1H), 3.77-3.65 (m, 4H), 3.65-3.53 (m, 17H), 3.20-3.02 (m, 3H), 2.78 (dd, J=17.0, 3.1 Hz, 1H), 2.54 (dddd, J=13.4, 11.0, 6.9, 4.3 Hz, 1H), 2.46 (s, 3H), 2.49-2.40 (m, 1H), 2.35 (qd, J=6.3, 4.8 Hz, 2H), 2.26-2.16 (m, 1H), 2.08 (ddd, J=13.3, 9.1, 4.5 Hz, 1H), 1.69-1.58 (m, 1H), 1.38 (d, J=2.3 Hz, 3H), 1.35-1.32 (m, 3H), 1.29 (s, 3H), 1.06-1.02 (m, 8H), 1.00 (d, J=8.3 Hz, 1H), 0.93-0.83 (m, 2H).

### Example 61. (4aS,5aR)-5,5-difluoro-N-(1-((S)-(1-((S)-24-((2S,4R)-4-hydroxy-2-((4-(4-methylthiazol-5-yl)benzyl)carbamoyl)pyrrolidine-1-carbonyl)-25,25-dimethyl-22-oxo-4,7,10,13,16,19-hexaoxa-23-azahexacosanoyl)azetidin-3-yl)(phenyl)methyl)-1H-pyrazol-4-yl)-5a-methyl-1,4,4a,5,5a,6-hexahydrocyclopropa[f]indazole-3-carboxamide (14)

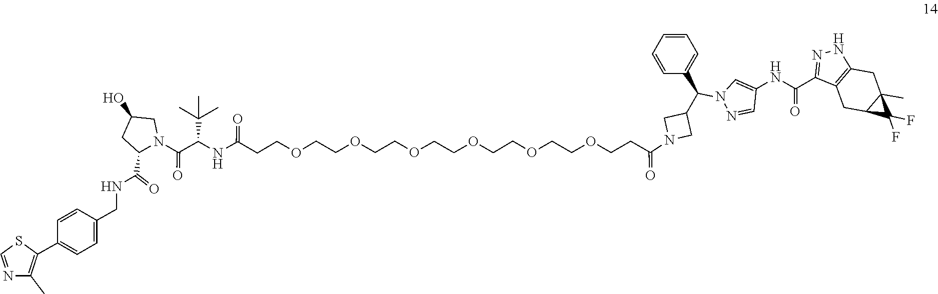 [SMILES: Cc1ncsc1-c1ccc(CNC(=O)[C@@H]2C[C@@H](O)CN2C(=O)[C@@H](NC(=O)CCOCCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

Using General Procedure 7, (S)-24-((2S,4R)-4-hydroxy-2-((4-(4-methylthiazol-5-yl)benzyl)carbamoyl)pyrrolidine-1-carbonyl)-25,25-dimethyl-22-oxo-4,7,10,13,16,19-hexaoxa-23-azahexacosanoic acid (HB30) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) to afford (4aS,5aR)-5,5-difluoro-N-(1-((S)-(1-((S)-24-((2S,4R)-4-hydroxy-2-((4-(4-methylthiazol-5-yl)benzyl)carbamoyl)pyrrolidine-1-carbonyl)-25,25-dimethyl-22-oxo-4,7,10,13,16,19-hexaoxa-23-azahexacosanoyl)azetidin-3-yl)(phenyl)methyl)-1H-pyrazol-4-yl)-5a-methyl-1,4,4a,5,5a,6-hexahydrocyclopropa[f]indazole-3-carboxamide (14). ¹H NMR (500 MHz, (CD₃)₂SO) δ 12.95 (s, 1H), 10.16 (d, J=3.8 Hz, 1H), 8.98 (s, 1H), 8.56 (t, J=6.1 Hz, 1H), 8.06 (s, 1H), 7.91 (d, J=9.3 Hz, 1H), 7.65 (s, 1H), 7.46-7.28 (m, 8H), 5.64 (dd, J=11.0, 5.9 Hz, 1H), 5.12 (d, J=3.6 Hz, 1H), 4.55 (d, J=9.3 Hz, 1H), 4.47-4.39 (m, 2H), 4.35 (s, 1H), 4.21 (dt, J=16.5, 6.9 Hz, 2H), 3.85 (t, J=8.0 Hz, 1H), 3.79 (t, J=7.9 Hz, 1H), 3.71-3.53 (m, 6H), 3.51-3.44 (m, 22H), 3.04 (s, 2H), 3.00 (s, 1H), 2.81 (d, J=17.3 Hz, 1H), 2.58-2.52 (m, 1H), 2.44 (s, 3H), 2.30-2.20 (m, 2H), 2.03 (t, J=10.2 Hz, 1H), 1.90 (ddd, J=12.8, 8.6, 4.6 Hz, 1H), 1.80-1.73 (m, 1H), 1.34 (s, 3H), 1.23 (s, 1H), 0.93 (s, 10H). LCMS: C₆₁H₈₀F₂N₁₀O₁₂S requires: 1215, found: m/z=1216 [M+H]⁺.

### Example 62. (4aS,5aR)-5,5-difluoro-N-(1-((S)-(1-((S)-15-((2S,4R)-4-hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoyl)azetidin-3-yl)(phenyl)methyl)-1H-pyrazol-4-yl)-5a-methyl-1,4,4a,5,5a,6-hexahydrocyclopropa[f]indazole-3-carboxamide (15)

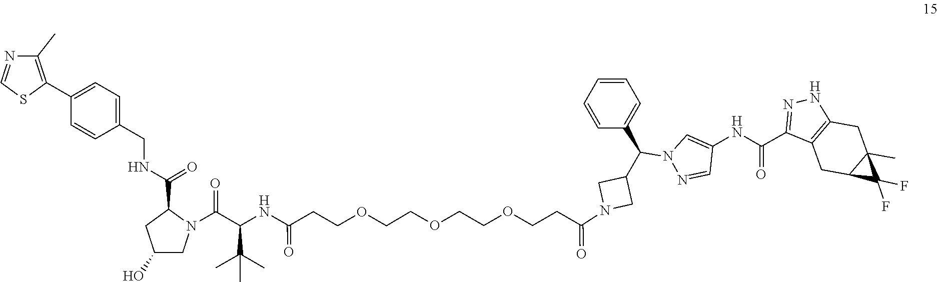 [SMILES: Cc1ncsc1-c1ccc(CNC(=O)[C@@H]2C[C@@H](O)CN2C(=O)[C@@H](NC(=O)CCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

Using General Procedure 7, 3-[2-[2-[3-[[(1S)-1-[(2S,4R)-4-hydroxy-2-[[4-(4-methylthiazol-5-yl)phenyl]methylcarbamoyl]pyrrolidine-1carbonyl]-2,2-dimethyl-propyl]amino]-3-oxo-propoxy]ethoxy]ethoxy]propanoic acid (HB13) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) to afford (4aS,5aR)-5,5-difluoro-N-(1-((S)-(1-((S)-15-((2S,4R)-4-hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoyl)azetidin-3-yl)(phenyl)methyl)-1H-pyrazol-4-yl)-5a-methyl-1,4,4a,5,5a,6-hexahydrocyclopropa[f]indazole-3-carboxamide (15). ¹H NMR (500 MHz, (CD₃)₂SO) δ 12.95 (s, 1H), 10.16 (d, J=3.7 Hz, 1H), 8.98 (s, 1H), 8.56 (t, J=6.1 Hz, 1H), 8.06 (d, J=1.5 Hz, 1H), 7.91 (dd, J=9.3, 2.5 Hz, 1H), 7.65 (d, J=1.3 Hz, 1H), 7.44-7.27 (m, 9H), 5.64 (dd, J=11.0, 6.0 Hz, 1H), 5.12 (d, J=3.5 Hz, 1H), 4.55 (dd, J=9.4, 1.7 Hz, 1H), 4.47-4.39 (m, 2H), 4.35 (s, 1H), 4.25-4.08 (m, 2H), 3.85 (t, J=8.0 Hz, 1H), 3.79 (t, J=7.9 Hz, 1H), 3.72-3.53 (m, 6H), 3.53-3.41 (m, 8H), 3.10-2.97 (m, 3H), 2.80 (d, J=17.0 Hz, 1H), 2.57-2.50 (m, 1H), 2.44 (s, 2H), 2.35 (dt, J=14.6, 6.2 Hz, 1H), 2.25 (dd, J=9.1, 5.6 Hz, 2H), 2.03 (t, J=10.4 Hz, 1H), 1.90 (ddd, J=12.9, 8.6, 4.6 Hz, 1H), 1.80-1.72 (m, 1H), 1.34 (s, 3H), 1.23 (s, 1H), 1.00-0.88 (m, 9H). LCMS: C₅₅H₆₈F₂N₁₀O₉S requires: 1082, found: m/z=1083 [M+H]⁺.

### Example 63. (4aS,5aR)-5,5-difluoro-N-(1-((S)-(1-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (16)

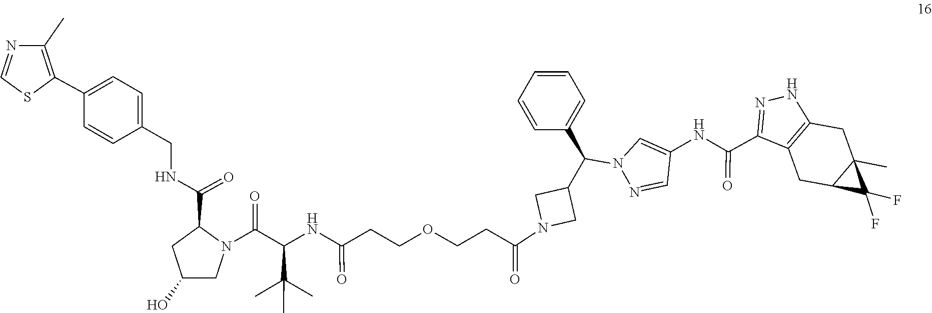 [SMILES: Cc1ncsc1-c1ccc(CNC(=O)[C@@H]2C[C@@H](O)CN2C(=O)[C@@H](NC(=O)CCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

Using General Procedure 7, 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (HB32) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)-5,5-difluoro-N-(1-((S)-(1-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoyl)azetidin-3-yl)(phenyl)methyl)-1H-pyrazol-4-yl)-5a-methyl-1,4,4a,5,5a,6-hexahydrocyclopropafindazole-3-carboxamide (16). ¹H NMR (500 MHz, (CD₃)₂SO) δ 12.95 (d, J=2.1 Hz, 1H), 10.16 (d, J=2.2 Hz, 1H), 8.98 (d, J=1.5 Hz, 1H), 8.55 (t, J=6.0 Hz, 1H), 8.06 (d, J=1.4 Hz, 1H), 7.91 (dd, J=9.5, 3.8 Hz, 1H), 7.65 (d, J=1.1 Hz, 1H), 7.44-7.27 (m, 9H), 5.66 (dd, J=11.0, 7.9 Hz, 1H), 5.12 (dd, J=3.6, 2.3 Hz, 1H), 4.55 (dd, J=9.5, 5.8 Hz, 1H), 4.46-4.37 (m, 2H), 4.34 (s, 1H), 4.29-4.14 (m, 2H), 4.10 (t, J=8.5 Hz, 1H), 3.88-3.75 (m, 2H), 3.68-3.59 (m, 1H), 3.61-3.46 (m, 4H), 3.04 (s, 2H), 3.00 (s, 1H), 2.80 (d, J=17.1 Hz, 1H), 2.44 (d, J=1.8 Hz, 3H), 2.33 (dd, J=14.1, 6.5 Hz, 1H), 2.29-2.18 (m, 2H), 2.03 (t, J=10.3 Hz, 1H), 1.90 (d, J=9.9 Hz, 1H), 1.76 (d, J=13.1 Hz, 1H), 1.34 (s, 3H), 1.23 (s, 1H), 0.92 (s, 10H). LCMS: C₅₁H₆₀F₂N₁₀O₇S requires: 994, found: m/z=995 [M+H]⁺.

### Example 64. (4aS,5aR)-5,5-difluoro-N-(1-((S)-(1-(3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (17)

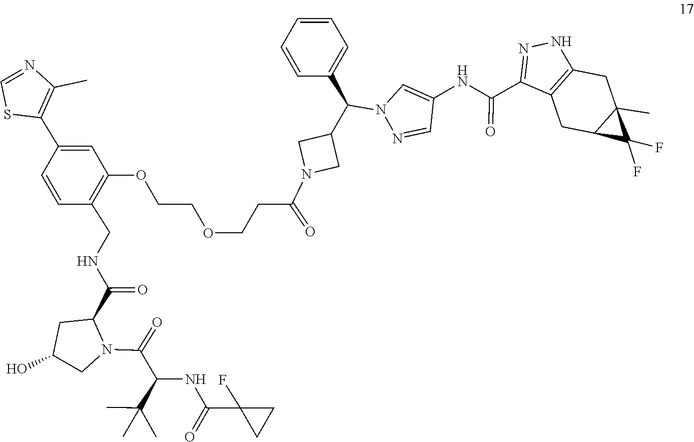 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCC(=O)N2CC([C@@H](c3ccccc3)n3cc(NC(=O)c4n[nH]c5c4C[C@@H]4C(F)(F)[C@]4(C)C5)cn3)C2)c1]

Using General Procedure 6, 3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)propanoic acid was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)-5,5-difluoro-N-(1-((S)-(1-(3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)propanoyl)azetidin-3-yl)(phenyl)methyl)-1H-pyrazol-4-yl)-5a-methyl-1,4,4a,5,5a,6-hexahydrocyclopropafindazole-3-carboxamide (17). ¹H NMR (500 MHz, CD₃CN) δ 9.21 (d, J=10.3 Hz, 1H), 8.91 (d, J=11.1 Hz, 1H), 7.95 (d, J=5.3 Hz, 1H), 7.61 (d, J=1.9 Hz, 1H), 7.48 (dt, J=8.5, 4.4 Hz, 1H), 7.41-7.27 (m, 6H), 7.02 (dd, J=14.5, 7.8 Hz, 3H), 5.45 (t, J=10.8 Hz, 1H), 4.64 (d, J=9.2 Hz, 1H), 4.53 (td, J=8.4, 3.1 Hz, 1H), 4.50-4.38 (m, 2H), 4.33 (ddd, J=16.0, 12.1, 5.5 Hz, 1H), 4.24-4.09 (m, 3H), 4.00-3.89 (m, 1H), 3.87-3.70 (m, 4H), 3.67 (dd, J=10.8, 4.3 Hz, 1H), 3.15 (d, J=17.9 Hz, 1H), 3.06 (dd, J=17.6, 8.9 Hz, 2H), 2.77 (d, J=17.2 Hz, 1H), 2.49 (d, J=3.0 Hz, 3H), 2.32 (dt, J=11.9, 6.2 Hz, 2H), 2.15-2.07 (m, 1H), 2.06-1.98 (m, 1H), 1.66 (dd, J=15.1, 6.9 Hz, 1H), 1.36 (s, 3H), 1.34-1.15 (m, 3H), 0.97 (d, J=4.7 Hz, 9H). LCMS: C₅₄H₆₃F₃N₁₀O₈S requires: 1068, found: m/z=1069 [M+H]⁺.

### Example 65. (4aS,5aR)-5,5-difluoro-N-(1-((S)-(1-(6-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)hexanoyl)azetidin-3-yl)(phenyl)methyl)-1H-pyrazol-4-yl)-5a-methyl-1,4,4a,5,5a,6-hexahydrocyclopropa[f]indazole-3-carboxamide (18)

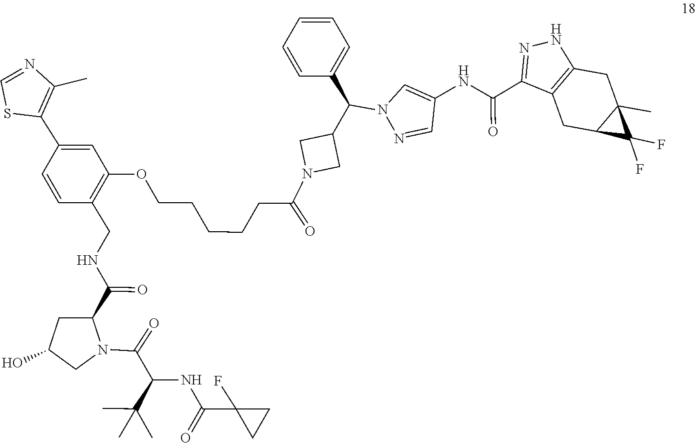 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)N2CC([C@@H](c3ccccc3)n3cc(NC(=O)c4n[nH]c5c4C[C@@H]4C(F)(F)[C@]4(C)C5)cn3)C2)c1]

Using General Procedure 6, 3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (HB32) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)-5,5-difluoro-N-(1-((S)-(1-(6-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)hexanoyl)azetidin-3-yl)(phenyl)methyl)-1H-pyrazol-4-yl)-5a-methyl-1,4,4a,5,5a,6-hexahydrocyclopropafindazole-3-carboxamide (18). ¹H NMR (500 MHz, DMSO-d₆) δ 12.95 (s, 1H), 10.15 (s, 1H), 8.97 (d, J=3.5 Hz, 1H), 8.49 (t, J=6.0 Hz, 1H), 8.06 (s, 1H), 7.65 (s, 1H), 7.43-7.24 (m, 6H), 6.99 (s, 1H), 6.94 (d, J=7.8 Hz, 1H), 5.66 (dd, J=11.0, 6.7 Hz, 1H), 4.59 (d, J=9.2 Hz, 1H), 4.51 (t, J=8.2 Hz, 1H), 4.34 (s, 1H), 4.29 (dd, J=16.6, 6.2 Hz, 1H), 4.23-4.13 (m, 1H), 4.12-3.97 (m, 3H), 3.85 (t, J=8.9 Hz, 1H), 3.81-3.74 (m, 1H), 3.72-3.44 (m, 5H), 3.03 (dd, J=15.3, 9.4 Hz, 3H), 2.80 (d, J=17.4 Hz, 1H), 2.45 (s, 3H), 2.05 (dq, J=15.0, 7.9 Hz, 3H), 1.91 (ddt, J=13.3, 9.4, 5.1 Hz, 1H), 1.75 (h, J=6.8 Hz, 3H), 1.57-1.30 (m, 9H), 1.22 (dd, J=8.4, 2.9 Hz, 2H), 0.97-0.91 (m, 9H). LCMS: C₅₅H₆₅F₃N₁₀O₇S requires: 1067, found: m/z=1068 [M+H]⁺.

### Example 66. (4aS,5aR)-5,5-difluoro-N-(1-((S)-(1-(3-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (19)

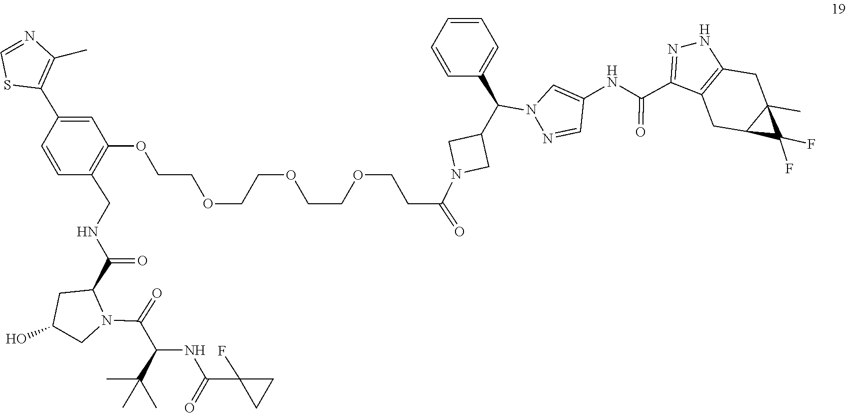 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCC(=O)N2CC([C@@H](c3ccccc3)n3cc(NC(=O)c4n[nH]c5c4C[C@@H]4C(F)(F)[C@]4(C)C5)cn3)C2)c1]

Using General Procedure 6, 3-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)propanoic acid (HB20) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropa[f]indazole-3-carboxamide (BBX₂) to afford (4aS,5aR)-5,5-difluoro-N-(1-((S)-(1-(3-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (19). ¹H NMR (500 MHz, DMSO-d₆) δ 13.19 (s, 1H), 10.89 (s, 1H), 10.15 (d, J=4.1 Hz, 1H), 8.98 (s, 1H), 8.76 (d, J=11.9 Hz, 1H), 8.49 (q, J=6.1 Hz, 1H), 8.19 (d, J=13.8 Hz, 1H), 7.55-7.25 (m, 6H), 7.03 (t, J=2.1 Hz, 1H), 6.96 (t, J=6.4 Hz, 1H), 5.67-5.59 (m, 1H), 4.59 (d, J=9.3 Hz, 1H), 4.51 (t, J=8.2 Hz, 1H), 4.47-4.07 (m, 5H), 3.77 (q, J=3.7 Hz, 2H), 3.70-3.33 (m, 9H), 3.03 (s, 3H), 2.86-2.77 (m, 1H), 2.46 (d, J=3.5 Hz, 2H), 2.24 (td, J=8.8, 5.9 Hz, 2H), 2.09 (dd, J=12.9, 7.8 Hz, 1H), 1.91 (ddd, J=13.2, 9.4, 4.4 Hz, 1H), 1.77 (s, 1H), 1.41-1.19 (m, 6H), 0.95 (s, 8H). LCMS: C₅₈H₇₁F₃N₁₀O₁₀S requires: 1157, found: m/z=1158 [M+H]⁺.

### Example 67. (4aS,5aR)-5,5-difluoro-N-(1-((S)-(1-(8-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)octanoyl)azetidin-3-yl)(phenyl)methyl)-1H-pyrazol-4-yl)-5a-methyl-1,4,4a,5,5a,6-hexahydrocyclopropa[f]indazole-3-carboxamide (20)

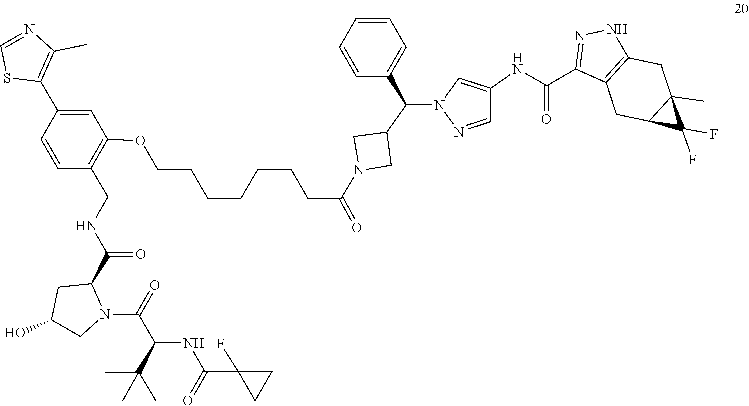 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)N2CC([C@@H](c3ccccc3)n3cc(NC(=O)c4n[nH]c5c4C[C@@H]4C(F)(F)[C@]4(C)C5)cn3)C2)c1]

Using General Procedure 6, 8-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)octanoic acid (HB18) was treated with (4aS,5aR)—N-(1-((S)-azetidin-3-yl(phenyl)methyl)-1H-pyrazol-4-yl)-5,5-difluoro-5a-methyl-1,4,4a,5,5a,6-hexahydrocyclopropafindazole-3-carboxamide (BBX₂) to afford (4aS,5aR)-5,5-difluoro-N-(1-((S)-(1-(8-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)octanoyl)azetidin-3-yl)(phenyl)methyl)-1H-pyrazol-4-yl)-5a-methyl-1,4,4a,5,5a,6-hexahydrocyclopropafindazole-3-carboxamide (20). ¹H NMR (500 MHz, DMSO-d₆) δ 12.95 (s, 1H), 10.15 (d, J=2.3 Hz, 1H), 8.98 (d, J=3.5 Hz, 1H), 8.48 (t, J=6.1 Hz, 1H), 8.06 (s, 1H), 7.65 (s, 1H), 7.51-7.25 (m, 6H), 6.99 (d, J=5.7 Hz, 1H), 6.94 (td, J=5.9, 3.0 Hz, 1H), 5.66 (dd, J=10.9, 6.8 Hz, 1H), 4.59 (d, J=9.2 Hz, 1H), 4.51 (t, J=8.2 Hz, 1H), 4.35 (s, 1H), 4.28 (dd, J=16.5, 6.2 Hz, 1H), 4.23-4.12 (m, 2H), 4.12-3.99 (m, 3H), 3.88-3.72 (m, 2H), 3.72-3.45 (m, 4H), 3.10-2.98 (m, 3H), 2.80 (dd, J=17.1, 3.2 Hz, 1H), 2.45 (d, J=1.9 Hz, 3H), 2.08 (dd, J=12.9, 8.1 Hz, 1H), 2.00 (q, J=7.7 Hz, 2H), 1.92 (ddd, J=13.0, 8.6, 4.5 Hz, 1H), 1.74 (h, J=7.0 Hz, 3H), 1.49-1.17 (m, 16H), 0.95 (s, 9H). LCMS: C₅₇H₆₉F₃N₁₀O₇S requires: 1095, found: m/z=1096 [M+H]⁺.

### Example 68. (4aS,5aR)-5,5-difluoro-N-(1-((S)-(1-(1-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyrimidin-5-yl)piperidine-4-carbonyl)azetidin-3-yl)(phenyl)methyl)-1H-pyrazol-4-yl)-5a-methyl-1,4,4a,5,5a,6-hexahydrocyclopropa[f]indazole-3-carboxamide (21)

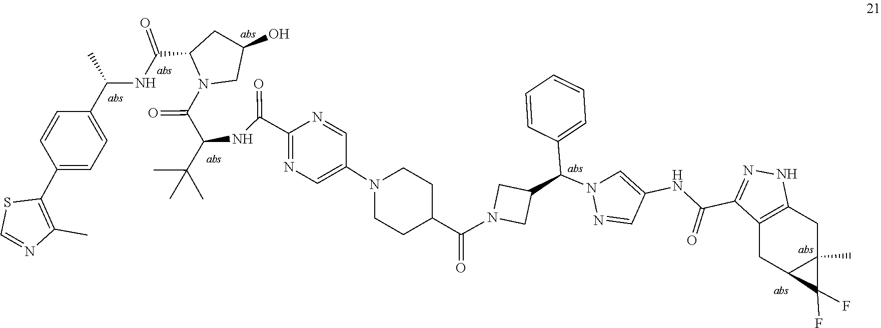 [SMILES: Cc1ncsc1-c1ccc([C@H](C)NC(=O)[C@@H]2C[C@@H](O)CN2C(=O)[C@@H](NC(=O)c2ncc(N3CCC(C(=O)N4CC([C@@H](c5ccccc5)n5cc(NC(=O)c6n[nH]c7c6C[C@@H]6C(F)(F)[C@]6(C)C7)cn5)C4)CC3)cn2)C(C)(C)C)cc1]

A mixture of (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (15.00 mg, 0.03 mmol), PyBOP (23.1 mg, 0.04 mmol), 1-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}pyrimidin-5-yl)piperidine-4-carboxylic acid (23.2 mg, 0.03 mmol), and N,N-diisopropylethylamine (HB15) (0.04 mL, 0.21 mmol) in DMF (0.3 mL) was allowed to stir at rt for one hour. The mixture was purified by HPLC (5-95% MeCN in H₂O with 0.1% TFA) to afford 5-(4-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidine-1-carbonyl}piperidin-1-yl)-N-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]pyrimidine-2-carboxamide (0.0161 g, 37.7%). LCMS: C₅₇H₆₅F₂N₁₃O₆S requires: 1097, found: m/z=1098 [M+H]⁺.

### Example 69. (4aS,5aR)-5,5-difluoro-N-(1-((S)-(1-(5-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperidin-1-yl)picolinoyl)azetidin-3-yl)(phenyl)methyl)-1H-pyrazol-4-yl)-5a-methyl-1,4,4a,5,5a,6-hexahydrocyclopropa[f]indazole-3-carboxamide (22)

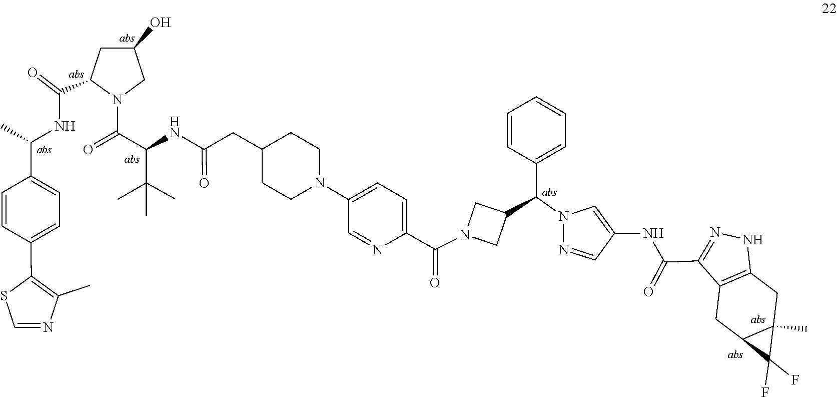 [SMILES: Cc1ncsc1-c1ccc([C@H](C)NC(=O)[C@@H]2C[C@@H](O)CN2C(=O)[C@@H](NC(=O)CC2CCN(c3ccc(C(=O)N4CC([C@@H](c5ccccc5)n5cc(NC(=O)c6n[nH]c7c6C[C@@H]6C(F)(F)[C@]6(C)C7)cn5)C4)nc3)CC2)C(C)(C)C)cc1]

A mixture of 5-[4-({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperidin-1-yl]pyridine-2-carboxylic acid (HB14) (23.6 mg, 0.03 mmol), PyBOP (23.1 mg, 0.04 mmol), (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (15.0 mg, 0.03 mmol), and N,N-diisopropylethylamine (0.04 mL, 0.21 mmol) in DMF (0.3 mL) was allowed to stir at rt for one hour. The mixture was purified by HPLC (5-95% MeCN in H₂O with 0.1% TFA) to afford (2S,4R)-1-[(2S)-2-{2-[1-(6-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidine-1-carbonyl}pyridin-3-yl)piperidin-4-yl]acetamido}-3,3-dimethylbutanoyl]-4-hydroxy-N-[(1S)-1-[4-(4-methyl-1,3-thiazol-5-yl)phenyl]ethyl]pyrrolidine-2-carboxamide (0.0161 g, 39.4%). LCMS: C₅₉H₆₈F₂N₁₂O₆S requires: 1110, found: m/z=1111 [M+H]⁺.

### Example 70. (4aS,5aR)-5,5-difluoro-N-(1-((S)-(1-(2-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidin-4-yl)acetyl)azetidin-3-yl)(phenyl)methyl)-1H-pyrazol-4-yl)-5a-methyl-1,4,4a,5,5a,6-hexahydrocyclopropa[f]indazole-3-carboxamide (23)

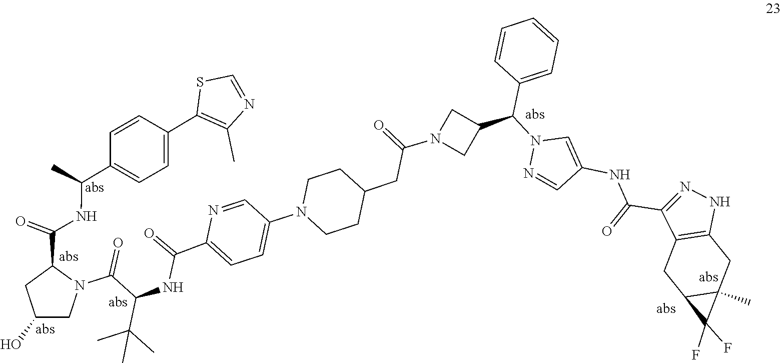 [SMILES: Cc1ncsc1-c1ccc([C@H](C)NC(=O)[C@@H]2C[C@@H](O)CN2C(=O)[C@@H](NC(=O)c2ccc(N3CCC(CC(=O)N4CC([C@@H](c5ccccc5)n5cc(NC(=O)c6n[nH]c7c6C[C@@H]6C(F)(F)[C@]6(C)C7)cn5)C4)CC3)cn2)C(C)(C)C)cc1]

A mixture of [1-(6-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}pyridin-3-yl)piperidin-4-yl]acetic acid (HB12) (23.63 mg, 0.03 mmol), PyBOP (23.1 mg, 0.04 mmol), (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (15.0 mg, 0.03 mmol), and N,N-diisopropylethylamine (0.04 mL, 0.21 mmol) in DMF (0.3 mL) was allowed to stir at rt for one hour. The mixture was purified by HPLC (5-95% MeCN in H₂O with 0.1% TFA) to afford 5-[4-(2-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}-2-oxoethyl)piperidin-1-yl]-N-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]pyridine-2-carboxamide (0.0117 g, 26.5%). LCMS: C₅₉H₆₈F₂N₁₂O₆S requires: 1110, found: m/z=1111 [M+H]⁺.

### Example 71. (4aS,5aR)-5,5-difluoro-N-(1-((S)-(1-(2-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)piperazin-1-yl)acetyl)azetidin-3-yl)(phenyl)methyl)-1H-pyrazol-4-yl)-5a-methyl-1,4,4a,5,5a,6-hexahydrocyclopropa[f]indazole-3-carboxamide (24)

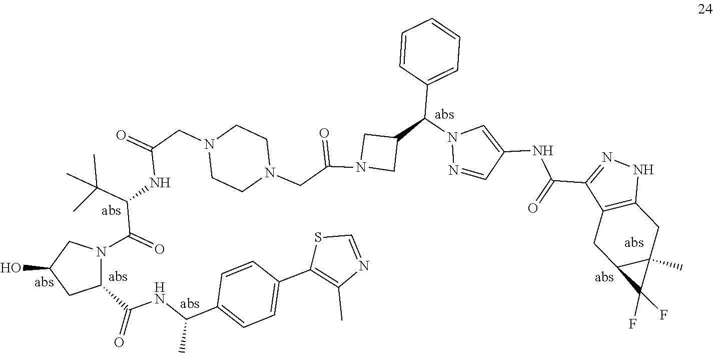 [SMILES: Cc1ncsc1-c1ccc([C@H](C)NC(=O)[C@@H]2C[C@@H](O)CN2C(=O)[C@@H](NC(=O)CN2CCN(CC(=O)N3CC([C@@H](c4ccccc4)n4cc(NC(=O)c5n[nH]c6c5C[C@@H]5C(F)(F)[C@]5(C)C6)cn4)C3)CC2)C(C)(C)C)cc1]

A mixture of (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (11.00 mg, 0.03 mmol), PyBOP (17 mg, 0.03 mmol), [4-({[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}methyl)piperazin-1-yl]acetic acid (HB35) (15.8 mg, 0.03 mmol), and N,N-diisopropylethylamine (0.03 mL, 0.15 mmol) in DMF (0.3 mL) was allowed to stir at rt for one hour. The mixture was purified by HPLC (5-95% MeCN in H₂O with 0.1% TFA) to afford (2S,4R)-1-[(2S)-2-{2-[4-(2-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}-2-oxoethyl)piperazin-1-yl]acetamido}-3,3-dimethylbutanoyl]-4-hydroxy-N-[(1S)-1-[4-(4-methyl-1,3-thiazol-5-yl)phenyl]ethyl]pyrrolidine-2-carboxamide (0.0112 g, 38.3%). LCMS: C₅₄H₆₆F₂N₁₂O₆S requires: 1048, found: m/z=1049 [M+H]⁺.

### Example 72. (4aS,5aR)-5,5-difluoro-N-(1-((S)-(1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (25)

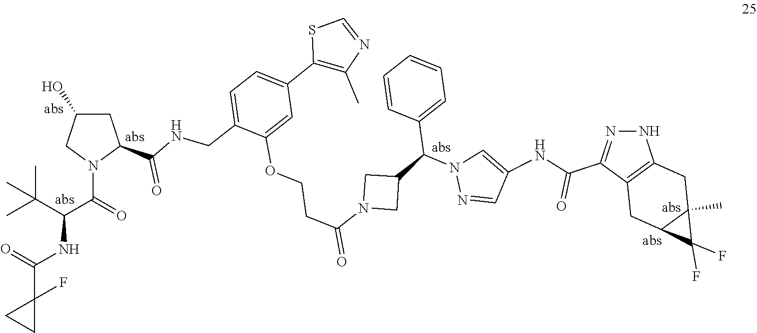 [SMILES: Cc1ncsc1-c1ccc(CNC(=O)[C@@H]2C[C@@H](O)CN2C(=O)[C@@H](NC(=O)C2(F)CC2)C(C)(C)C)c(OCCC(=O)N2CC([C@@H](c3ccccc3)n3cc(NC(=O)c4n[nH]c5c4C[C@@H]4C(F)(F)[C@]4(C)C5)cn3)C2)c1]

A mixture of 3-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]propanoic acid (HB36) (15.2 mg, 0.03 mmol), (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (11.0 mg, 0.03 mmol), PyBOP (13.1 mg, 0.03 mmol), and N,N-diisopropylethylamine (0.02 mL, 0.10 mmol) in DMF (0.3 mL) was allowed to stir at rt for one hour. The mixture was purified by HPLC (5-95% MeCN in H₂O with 0.1% TFA) to afford (2S,4R)—N-{[2-(3-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}-3-oxopropoxy)-4-(4-methyl-1,3-thiazol-5-yl)phenyl]methyl}-1-[(2S)-2-[(1-fluorocyclopropyl)formamido]-3,3-dimethylbutanoyl]-4-hydroxypyrrolidine-2-carboxamide (0.0061 g, 23.7%). LCMS: C₅₂H₅₉F₃N₁₀O₇S requires: 1024, found: m/z=1025 [M+H]⁺.

### Example 73. (4aS,5aR)-5,5-difluoro-N-(1-((S)-(1-(3-(1-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)piperidin-4-yl)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (26)

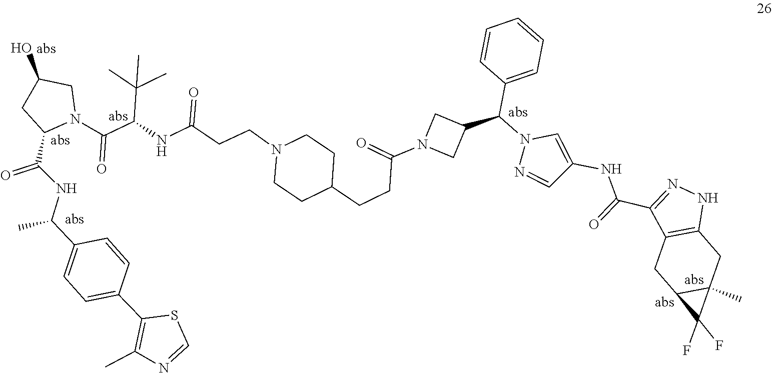 [SMILES: Cc1ncsc1-c1ccc([C@H](C)NC(=O)[C@@H]2C[C@@H](O)CN2C(=O)[C@@H](NC(=O)CCN2CCC(CCC(=O)N3CC([C@@H](c4ccccc4)n4cc(NC(=O)c5n[nH]c6c5C[C@@H]5C(F)(F)[C@]5(C)C6)cn4)C3)CC2)C(C)(C)C)cc1]

A mixture of (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (10.0 mg, 0.02 mmol), PyBOP (15.4 mg, 0.03 mmol), 3-[1-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)piperidin-4-yl]propanoic acid (HB38) (15.0 mg, 0.02 mmol), and N,N-diisopropylethylamine (0.02 mL, 0.14 mmol) in DMF (0.3 mL) was allowed to stir at rt for one hour. The mixture was purified by HPLC (5-95% MeCN in H₂O with 0.1% TFA) to afford (2S,4R)-1-[(2S)-2-{3-[4-(3-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}-3-oxopropyl)piperidin-1-yl]propanamido}-3,3-dimethylbutanoyl]-4-hydroxy-N-[(1S)-1-[4-(4-methyl-1,3-thiazol-5-yl)phenyl]ethyl]pyrrolidine-2-carboxamide (0.0156 g, 45.1%). LCMS: C₅₇H₇₁F₂N₁₁O₆S requires: 1075, found: m/z=1076 [M+H]⁺.

### Example 74. (4aS,5aR)-5,5-difluoro-N-(1-((S)-(1-(11-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-11-oxoundecanoyl)azetidin-3-yl)(phenyl)methyl)-1H-pyrazol-4-yl)-5a-methyl-1,4,4a,5,5a,6-hexahydrocyclopropa[f]indazole-3-carboxamide (27)

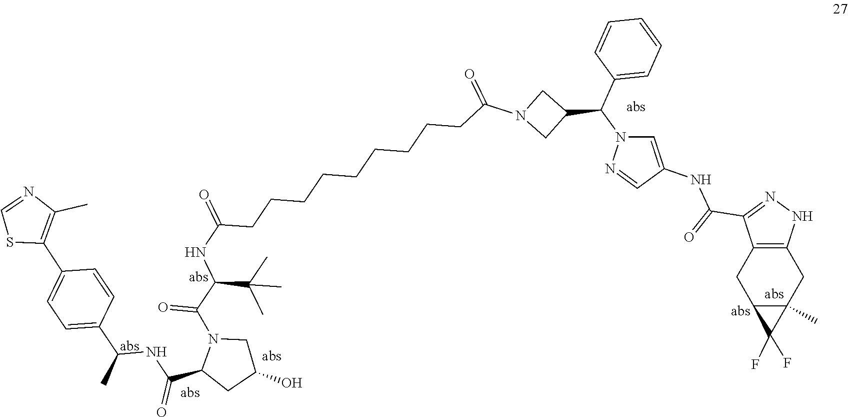 [SMILES: Cc1ncsc1-c1ccc([C@H](C)NC(=O)[C@@H]2C[C@@H](O)CN2C(=O)[C@@H](NC(=O)CCCCCCCCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

A mixture of 10-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}decanoic acid (HB1l) (16.1 mg, 0.03 mmol), PyBOP (17.0 mg, 0.03 mmol), (4aS,5aR)—N-{1-[(S)-azetidin-3-yl(phenyl)methyl]pyrazol-4-yl}-5,5-difluoro-5a-methyl-1H,4H,4aH,6H-cyclopropa]indazole-3-carboxamide (BBX₂) (11.0 mg, 0.03 mmol) and N,N-diisopropylethylamine (0.03 mL, 0.15 mmol) in DMF (0.3 mL) was allowed to stir at rt for one hour. The mixture was purified by HPLC (5-95% MeCN in H₂O with 0.1% TFA) to afford (2S,4R)-1-[(2S)-2-(11-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}-11-oxoundecanamido)-3,3-dimethylbutanoyl]-4-hydroxy-N-[(1S)-1-[4-(4-methyl-1,3-thiazol-5-yl)phenyl]ethyl]pyrrolidine-2-carboxamide (0.0098 g, 32.0%). LCMS: C₅₇H₇₂F₂N₁₀O₆S requires: 1062, found: m/z=1063 [M+H]⁺.

### Example 75. (4aS,5aR)-5,5-difluoro-N-(1-((S)-(1-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)azetidin-3-yl)(phenyl)methyl)-1H-pyrazol-4-yl)-5a-methyl-1,4,4a,5,5a,6-hexahydrocyclopropa[f]indazole-3-carboxamide (28)

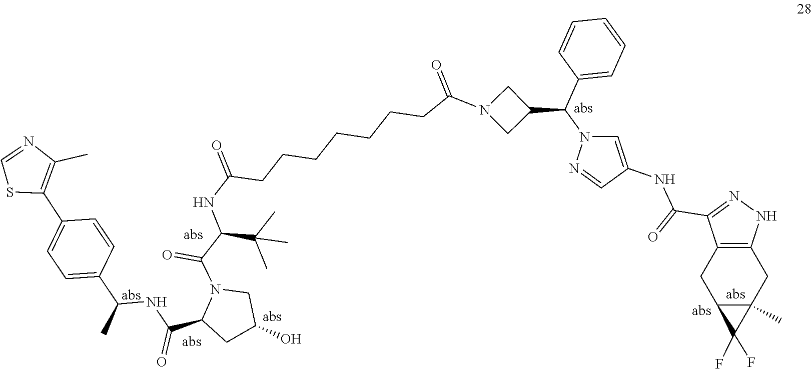 [SMILES: Cc1ncsc1-c1ccc([C@H](C)NC(=O)[C@@H]2C[C@@H](O)CN2C(=O)[C@@H](NC(=O)CCCCCCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

A mixture of (4aS,5aR)—N-{1-[(S)-azetidin-3-yl(phenyl)methyl]pyrazol-4-yl}-5,5-difluoro-5a-methyl-1H,4H,4aH,6H-cyclopropa[f]indazole-3-carboxamide (BBX₂) (11.0 mg, 0.03 mmol), PyBOP (17.0 mg, 0.03 mmol), 8-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}octanoic acid (HB10) (15.4 mg, 0.03 mmol) and N,N-diisopropylethylamine (0.03 mL, 0.15 mmol) in DMF (0.3 mL) was allowed to stir at rt for one hour. The mixture was purified by HPLC (5-95% MeCN in H₂O with 0.1% TFA) to afford (2S,4R)-1-[(2S)-2-(9-{3-[(S)-{4-[(4aS,5aR)-5,5-difluoro-5a-methyl-1H,4H,4aH,6H-cyclopropa[f]indazole-3-amido]pyrazol-1-yl}(phenyl)methyl]azetidin-1-yl}-9-oxononanamido)-3,3-dimethylbutanoyl]-4-hydroxy-N-[(1S)-1-[4-(4-methyl-1,3-thiazol-5-yl)phenyl]ethyl]pyrrolidine-2-carboxamide (0.0033 g, 10.9%). LCMS: C₅₅H₆₈F₂N₁₀O₆S requires: 1034, found: m/z=1035 [M+H]⁺.

### Example 76. (4aS,5aR)-5,5-difluoro-N-(1-((S)-(1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)azetidin-3-yl)(phenyl)methyl)-1H-pyrazol-4-yl)-5a-methyl-1,4,4a,5,5a,6-hexahydrocyclopropa[f]indazole-3-carboxamide (29)

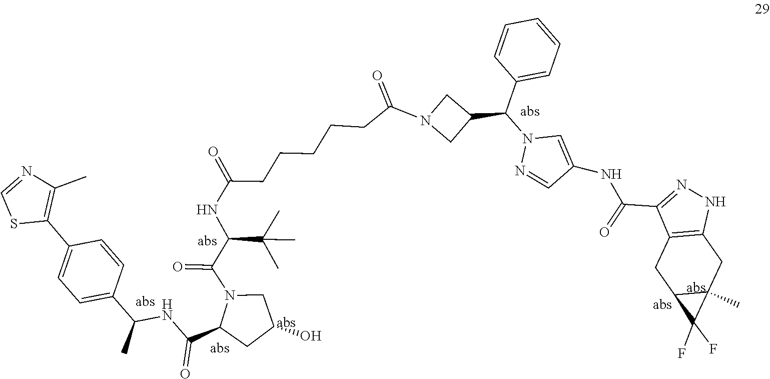 [SMILES: Cc1ncsc1-c1ccc([C@H](C)NC(=O)[C@@H]2C[C@@H](O)CN2C(=O)[C@@H](NC(=O)CCCCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

A mixture of 6-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}hexanoic acid (HB13) (14.7 mg, 0.03 mmol), PyBOP (17.0 mg, 0.03 mmol), (4aS,5aR)—N-{1-[(S)-azetidin-3-yl(phenyl)methyl]pyrazol-4-yl}-5,5-difluoro-5a-methyl-1H,4H,4aH,6H-cyclopropa]indazole-3-carboxamide (BBX₂) (11.0 mg, 0.03 mmol), and N,N-diisopropylethylamine (0.03 mL, 0.15 mmol) in DMF (0.3 mL) was allowed to stir at rt for one hour. The mixture was purified by HPLC (5-95% MeCN in H₂O with 0.1% TFA) to afford (2S,4R)-1-[(2S)-2-(7-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}-7-oxoheptanamido)-3,3-dimethylbutanoyl]-4-hydroxy-N-[(1S)-1-[4-(4-methyl-1,3-thiazol-5-yl)phenyl]ethyl]pyrrolidine-2-carboxamide (0.0008 g, 2.8%). LCMS: C₅₃H₆₄F₂N₁₀O₆S requires: 1006, found: m/z=1007 [M+H]⁺.

### Example 77. (4aS,5aR)-5,5-difluoro-N-(1-((S)-(1-(3-(2-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)ethoxy)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (30)

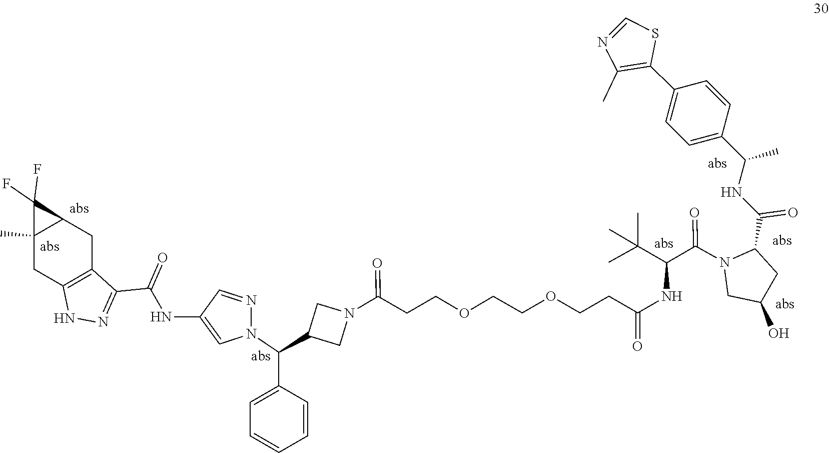 [SMILES: Cc1ncsc1-c1ccc([C@H](C)NC(=O)[C@@H]2C[C@@H](O)CN2C(=O)[C@@H](NC(=O)CCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1]

A mixture of 3-[2-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethoxy)ethoxy]propanoic acid (HB39) (27.4 mg, 0.04 mmol), (4aS,5aR)—N-{1-[(S)-azetidin-3-yl(phenyl)methyl]pyrazol-4-yl}-5,5-difluoro-5a-methyl-1H,4H,4aH,6H-cyclopropa]indazole-3-carboxamide (BBX₂) (19.0 mg, 0.04 mmol), HATU (17.3 mg, 0.05 mmol), and N,N-diisopropylethylamine (0.03 mL, 0.17 mmol) in DMF (0.3 mL) was allowed to stir at rt for one hour. The mixture was purified by HPLC (5-95% MeCN in H₂O with 0.1% TFA) to afford (2S,4R)-1-[(2S)-2-{3-[2-(3-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}-3-oxopropoxy)ethoxy]propanamido}-3,3-dimethylbutanoyl]-4-hydroxy-N-[(1S)-1-[4-(4-methyl-1,3-thiazol-5-yl)phenyl]ethyl]pyrrolidine-2-carboxamide (0.0108 g, 18.5%). LCMS: C₅₄H₆₆F₂N₁₀O₈S requires: 1052, found: m/z=1053 [M+H]⁺.

### Example 78. (4aS,5aR)-5,5-difluoro-N-(1-((S)-(1-(1-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)pyridin-3-yl)piperidine-4-carbonyl)azetidin-3-yl)(phenyl)methyl)-1H-pyrazol-4-yl)-5a-methyl-1,4,4a,5,5a,6-hexahydrocyclopropa[f]indazole-3-carboxamide (31)

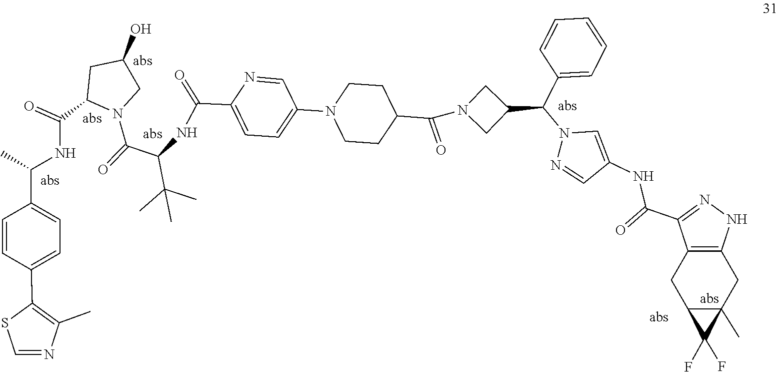 [SMILES: Cc1ncsc1-c1ccc([C@H](C)NC(=O)[C@@H]2C[C@@H](O)CN2C(=O)[C@@H](NC(=O)c2ccc(N3CCC(C(=O)N4CC([C@@H](c5ccccc5)n5cc(NC(=O)c6n[nH]c7c6C[C@@H]6C(F)(F)[C@]6(C)C7)cn5)C4)CC3)cn2)C(C)(C)C)cc1]

A mixture of 1-(6-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}pyridin-3-yl)piperidine-4-carboxylic acid (HB37) (34.0 mg, 0.05 mmol), (4aS,5aR)—N-{1-[(S)-azetidin-3-yl(phenyl)methyl]pyrazol-4-yl}-5,5-difluoro-5a-methyl-1H,4H,4aH,6H-cyclopropa]indazole-3-carboxamide (BBX₂) (20.0 mg, 0.05 mmol), HATU (26.0 mg, 0.07 mmol), and N,N-diisopropylethylamine (0.03 mL, 0.18 mmol) in DMF (0.3 mL) was allowed to stir at rt for one hour. The mixture was purified by HPLC (5-95% MeCN in H₂O with 0.1% TFA) to afford 5-(4-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidine-1-carbonyl}piperidin-1-yl)-N-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]pyridine-2-carboxamide (0.0088 g, 16.7%). LCMS: C₅₈H₆₆F₂N₁₂O₆S requires: 1096, found: m/z=1097 [M+H]⁺.

### Example 79. (4aS,5aR)-5,5-difluoro-N-(1-((S)-(1-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetyl)azetidin-3-yl)(phenyl)methyl)-1H-pyrazol-4-yl)-5a-methyl-1,4,4a,5,5a,6-hexahydrocyclopropa[f]indazole-3-carboxamide (32)

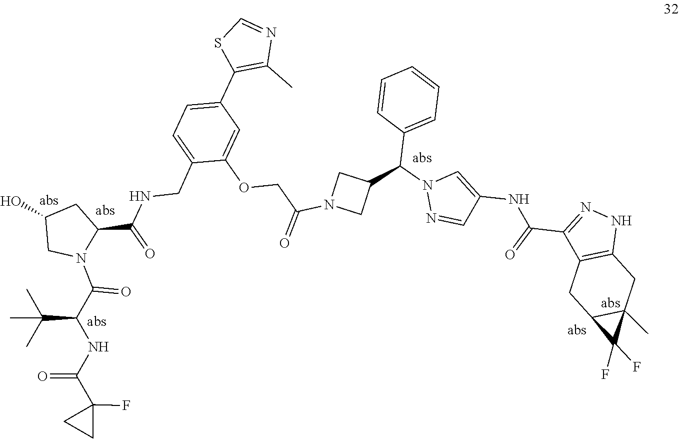 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N2CC([C@@H](c3ccccc3)n3cc(NC(=O)c4n[nH]c5c4C[C@@H]4C(F)(F)[C@]4(C)C5)cn3)C2)c1]

A mixture of 2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxyacetic acid (HB21) (40.4 mg, 0.07 mmol), (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (25.0 mg, 0.06 mmol), [(dimethylamino)({[1,2,3]triazolo[4,5-b]pyridin-3-yloxy})methylidene]dimethylazanium hexafluoro-lambda5-phosphanuide (32.52 mg, 0.09 mmol), and N,N-diisopropylethylamine (0.04 mL, 0.23 mmol) in DMF (0.3 mL) was allowed to stir at rt for one hour. The mixture was purified by HPLC (5-95% MeCN in H₂O with 0.15% TFA) to afford (2S,4R)—N-{[2-(2-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}-2-oxoethoxy)-4-(4-methyl-1,3-thiazol-5-yl)phenyl]methyl}-1-[(2S)-2-[(1-fluorocyclopropyl)formamido]-3,3-dimethylbutanoyl]-4-hydroxypyrrolidine-2-carboxamide (0.0218 g, 33.7%). LCMS: C₅₁H₅₇F₃N₁₀O₇S requires: 1010, found: m/z=1011 [M+H]⁺.

### Example 80. (4aS,5aR)-5,5-difluoro-N-(1-((S)-(1-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)butanoyl)azetidin-3-yl)(phenyl)methyl)-1H-pyrazol-4-yl)-5a-methyl-1,4,4a,5,5a,6-hexahydrocyclopropa[f]indazole-3-carboxamide (33)

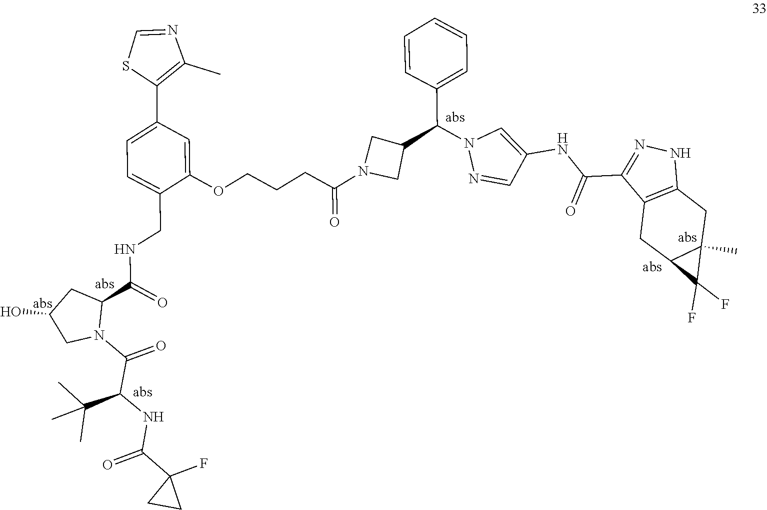

A mixture of (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (24.10 mg, 0.05 mmol), 4-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]butanoic acid (HB23) (34.00 mg, 0.05 mmol), [(dimethylamino)({[1,2,3]triazolo[4,5-b]pyridin-3-yloxy})methylidene]dimethylazanium hexafluoro-lambda5-phosphanuide (25.86 mg, 0.07 mmol), and N,N-diisopropylethylamine (0.04 mL, 0.03 g, 0.22 mmol) in DMF (1.00 mL) was allowed to stir at rt for 1.5 h. The reaction was then diluted with ethyl acetate and water. The aqueous phase was extracted with ethyl acetate. The combined organic phases were washed with brine, dried over Na₂SO₄, filtered, and concentrated. The resulting yellow oil was purified by reverse phase-HPLC, concentrated, and lyophilized to provide (2S,4R)—N-{[2-(4-{3-[(S)-{4-[(4aS,5aR)-5,5-difluoro-5a-methyl-1H,4H,4aH,6H-cyclopropa[indazole-3-amido]pyrazol-1-yl}(phenyl)methyl]azetidin-1-yl}-4-oxobutoxy)-4-(4-methyl-1,3-thiazol-5-yl)phenyl]methyl}-1-[(2S)-2-[(1-fluorocyclopropyl)formamido]-3,3-dimethylbutanoyl]-4-hydroxypyrrolidine-2-carboxamide (0.0195 g, 34.1%) as a white solid. ¹H NMR (500 MHz, DMSO-d₆) δ 12.95 (s, 1H), 10.15 (s, 1H), 8.98 (d, J=4.1 Hz, 1H), 8.48 (t, J=6.1 Hz, 1H), 8.04 (d, J=4.1 Hz, 1H), 7.65 (d, J=2.3 Hz, 1H), 7.40 (dd, J=7.9, 2.7 Hz, 1H), 7.35 (t, J=6.0 Hz, 4H), 7.33-7.26 (m, 2H), 6.99 (d, J=3.9 Hz, 1H), 6.95 (dd, J=7.9, 4.0 Hz, 1H), 5.66 (dd, J=11.0, 3.7 Hz, 1H), 4.59 (d, J=9.2 Hz, 1H), 4.51 (t, J=8.2 Hz, 1H), 4.34 (s, 1H), 4.32-4.00 (m, 5H), 3.02 (d, J=19.5 Hz, 3H), 2.81 (d, J=17.2 Hz, 1H), 2.45 (d, J=1.7 Hz, 3H), 2.23 (q, J=8.0 Hz, 2H), 2.07 (dd, J=13.0, 7.8 Hz, 1H), 2.01-1.85 (m, 3H), 1.76 (d, J=10.5 Hz, 1H), 1.43-1.29 (m, 5H), 1.28-1.13 (m, 2H), 0.95 (d, J=2.5 Hz, 9H). LCMS: C₅₃H₆₁F₃N₁₀O₇S requires: 1038.4, found: m/z=1040.2 [M+H]⁺.

### Example 81. (4aS,5aR)-5,5-difluoro-N-(1-((S)-(1-(5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentanoyl)azetidin-3-yl)(phenyl)methyl)-1H-pyrazol-4-yl)-5a-methyl-1,4,4a,5,5a,6-hexahydrocyclopropa[f]indazole-3-carboxamide (34)

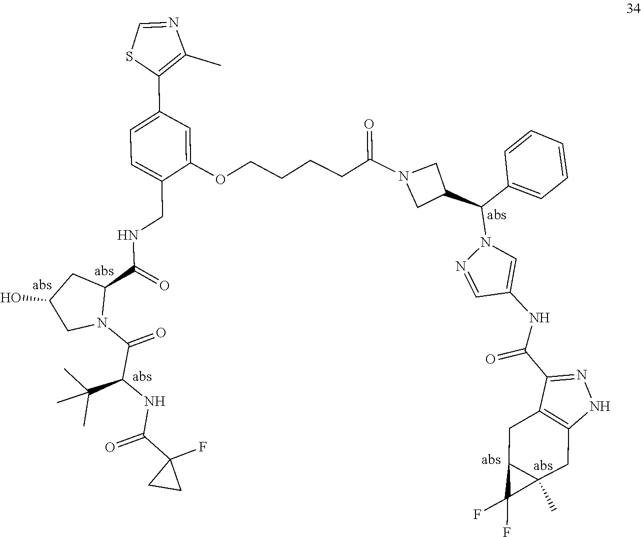 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCC(=O)N2CC([C@@H](c3ccccc3)n3cc(NC(=O)c4n[nH]c5c4C[C@@H]4C(F)(F)[C@]4(C)C5)cn3)C2)c1]

A mixture of (4aS,5aR)—N-{1-[(S)-azetidin-3-yl(phenyl)methyl]pyrazol-4-yl}-5,5-difluoro-5a-methyl-1H,4H,4aH,6H-cyclopropa[indazole-3-carboxamide (BBX₂) (35.80 mg, 0.08 mmol), 5-[2-({[(2S,4R)-1-[(2S)-2-[(1-fluorocyclopropyl)formamido]-3,3-dimethylbutanoyl]-4-hydroxypyrrolidin-2-yl]formamido}methyl)-5-(4-methyl-1,3-thiazol-5-yl)phenoxy]pentanoic acid (HB25) (51.66 mg, 0.08 mmol), [(dimethylamino)({[1,2,3]triazolo[4,5-b]pyridin-3-yloxy})methylidene]dimethylazanium hexafluoro-lambda5-phosphanuide (42.30 mg, 0.11 mmol), and N,N-diisopropylethylamine (0.06 mL, 0.04 g, 0.33 mmol) in dimethylformamide (1.50 mL) was allowed to stir at rt for 3.5 h. The reaction was then diluted with ethyl acetate and water. The aqueous phase was extracted with ethyl acetate. The combined organic phases were washed with brine, dried over Na₂SO₄, filtered, and concentrated. The resulting yellow oil was purified by reverse phase-HPLC, concentrated, and lyophilized to provide (2S,4R)—N-({2-[(5-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}-5-oxopentyl)oxy]-4-(4-methyl-1,3-thiazol-5-yl)phenyl}methyl)-1-[(2S)-2-[(1-fluorocyclopropyl)formamido]-3,3-dimethylbutanoyl]-4-hydroxypyrrolidine-2-carboxamide (0.0106 g, 12.3%) as an off-white solid. ¹H NMR (500 MHz, DMSO-d₆) δ 12.95 (s, 1H), 10.15 (s, 1H), 8.97 (d, J=4.9 Hz, 1H), 8.49 (d, J=5.4 Hz, 1H), 8.06 (s, 1H), 7.65 (s, 1H), 7.44-7.26 (m, 7H), 6.99 (s, 1H), 6.94 (d, J=7.9 Hz, 1H), 5.67 (dd, J=11.0, 7.9 Hz, 1H), 4.59 (d, J=9.2 Hz, 1H), 4.51 (t, J=8.3 Hz, 1H), 4.37-4.07 (m, 4H), 4.03 (t, J=6.3 Hz, 2H), 3.89-3.74 (m, 2H), 3.02 (d, J=19.5 Hz, 3H), 2.80 (d, J=17.1 Hz, 1H), 2.45 (s, 3H), 2.17-2.02 (m, 4H), 1.92 (ddd, J=12.9, 8.6, 4.3 Hz, 1H), 1.79-1.72 (m, 2H), 1.64 (t, J=7.6 Hz, 2H), 1.36 (d, J=14.6 Hz, 5H), 1.22 (d, J=8.3 Hz, 2H), 0.95 (s, 9H). LCMS: C₅₄H₆₃F₃N₁₀O₇S requires: 1052.5, found: m/z=1053.7 [M+H]⁺.

### Example 82. (4aS,5aR)-5,5-difluoro-N-(1-((S)-(1-(4-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)azetidin-1-yl)pyridin-2-yl)azetidin-3-yl)(phenyl)methyl)-1H-pyrazol-4-yl)-5a-methyl-1,4,4a,5,5a,6-hexahydrocyclopropa[f]indazole-3-carboxamide (35)

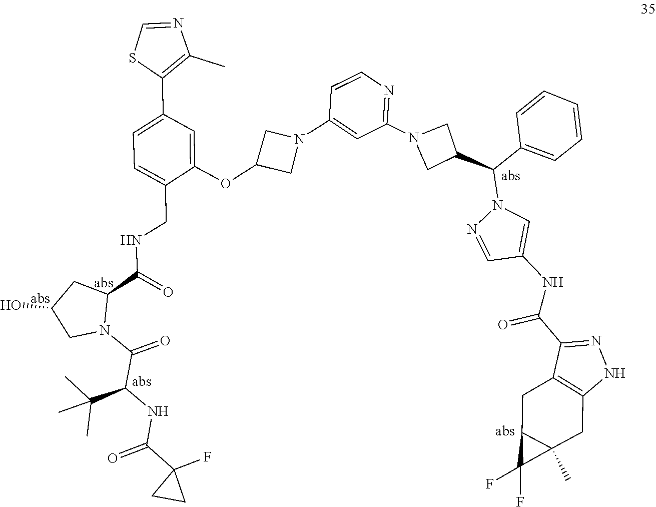 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CN(c3ccnc(N4CC([C@@H](c5ccccc5)n5cc(NC(=O)c6n[nH]c7c6C[C@@H]6C(F)(F)[C@]6(C)C7)cn5)C4)c3)C2)c1]

(2S,4R)-1-[(2S)-2-[(1-fluorocyclopropyl)formamido]-3,3-dimethylbutanoyl]-N-[(2-{[1-(2-fluoropyridin-4-yl)azetidin-3-yl]oxy}-4-(4-methyl-1,3-thiazol-5-yl)phenyl)methyl]-4-hydroxypyrrolidine-2-carboxamide (HB27) (113.00 mg, 0.17 mmol) and (4aS,5aR)—N-{1-[(S)-azetidin-3-yl(phenyl)methyl]pyrazol-4-yl}-5,5-difluoro-5a-methyl-1H,4H,4aH,6H-cyclopropa]indazole-3-carboxamide (BBX₂) (128.00 mg, 0.29 mmol) were dissolved in dimethyl sulfoxide (1.00 mL) and triethylamine (95.00 μL, 0.69 mmol) was added dropwise. The reaction was transferred to a 0.5-2 mL microwave vial and heated to 120° C. for 12 h. The reaction was then diluted with dichloromethane and washed with water. The organic phase was concentrated. The resulting yellow oil was purified by reverse phase-HPLC, concentrated, and lyophilized to provide (2S,4R)—N-[(2-{[1-(2-{3-[(S)-{4-[(4aS,5aR)-5,5-difluoro-5a-methyl-1H,4H,4aH,6H-cyclopropa]indazole-3-amido]pyrazol-1-yl}(phenyl)methyl]azetidin-1-yl}pyridin-4-yl)azetidin-3-yl]oxy}-4-(4-methyl-1,3-thiazol-5-yl)phenyl)methyl]-1-[(2S)-2-[(1-fluorocyclopropyl)formamido]-3,3-dimethylbutanoyl]-4-hydroxypyrrolidine-2-carboxamide (0.0125 g, 6.2%) as a white solid. ¹H NMR (500 MHz, CD₃CN) δ 10.72 (s, 1H), 8.89 (s, 1H), 8.76 (s, 1H), 8.01 (s, 1H), 7.63 (s, 1H), 7.45 (d, J=7.7 Hz, 2H), 7.43-7.34 (m, 5H), 7.13-7.05 (m, 2H), 7.05 (s, 2H), 6.71 (d, J=1.7 Hz, 1H), 6.01 (dd, J=7.4, 2.2 Hz, 1H), 5.60 (d, J=10.2 Hz, 1H), 5.26-5.19 (m, 1H), 4.63 (d, J=9.3 Hz, 1H), 4.55 (s, 1H), 4.51-4.44 (m, 1H), 4.38 (t, J=6.2 Hz, 2H), 4.25 (d, J=9.1 Hz, 1H), 4.12 (t, J=8.2 Hz, 1H), 3.96 (d, J=6.1 Hz, 2H), 3.83 (s, 1H), 3.72 (d, J=11.1 Hz, 1H), 3.64 (dd, J=11.0, 3.9 Hz, 1H), 3.15 (d, J=17.7 Hz, 2H), 3.06 (d, J=17.9 Hz, 3H), 2.76 (d, J=17.0 Hz, 2H), 2.48 (s, 3H), 1.71-1.63 (m, 1H), 1.36 (s, 5H), 1.34-1.19 (m, 7H), 0.95 (s, 9H). LCMS: C₅₇H₆₃F₃N₁₂O₆S requires: 1100.5, found: m/z=1101.9 [M+H]⁺.

### Example 83. (4aR,5aS)—N-(1-((1R)-(1-((3S)-3-(4-bromophenyl)-3-((2S,4R)-4-hydroxy-1-(3-methyl-2-(3-methylisoxazol-5-yl)butanoyl)pyrrolidine-2-carboxamido)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (36)

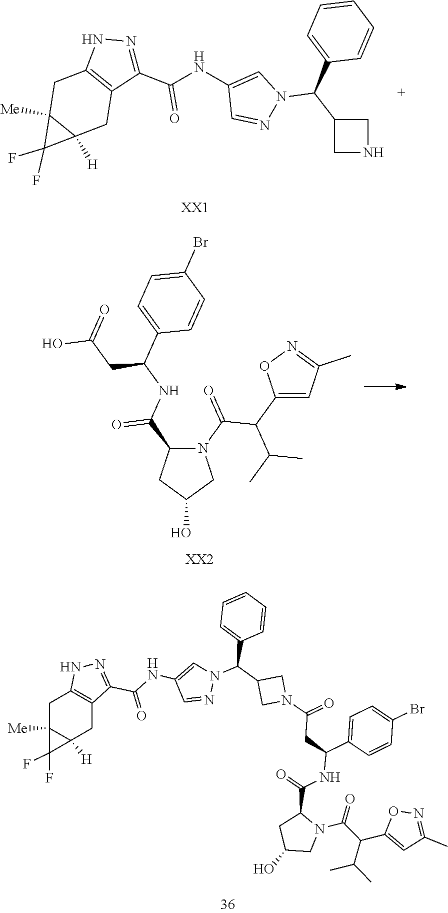

XX1 (14.0 mg, 0.03 mmol), XX2 (16.7 mg, 0.03 mmol), and HATU (12.1 mg, 0.03 mmol) were combined and suspended in DMF (1 mL). N,N-diisopropylethylamine (0.01 mL, 0.06 mmol) was added and the reaction was stirred overnight at room temperature. The reaction was purified by preparatory HPLC (5-95% MeCN in H₂O with 0.1% TFA) to afford (4aR,5aS)—N-(1-((1R)-(1-((3S)-3-(4-bromophenyl)-3-((2S,4R)-4-hydroxy-1-(3-methyl-2-(3-methylisoxazol-5-yl)butanoyl)pyrrolidine-2-carboxamido)propanoyl)azetidin-3-yl)(phenyl)methyl)-1H-pyrazol-4-yl)-5,5-difluoro-5a-methyl-1,4,4a,5,5a,6-hexahydrocyclopropa]indazole-3-carboxamide (11 mg, 36%). LCMS: C₄₆H₅₀BrF₂N₉O₆ requires: 941.3, found: m/z=942.3 [M+H]⁺. ¹H NMR (500 MHz, DMSO-d₆) δ 10.17 (d, J=7.5 Hz, 2H), 8.74 (d, J=25.3 Hz, 1H), 8.40 (s, 1H), 8.23 (t, J=9.0 Hz, 1H), 8.05 (d, J=15.2 Hz, 2H), 7.66 (d, J=7.9 Hz, 1H), 7.53 (dd, J=19.7, 8.2 Hz, 2H), 7.43-7.32 (m, 4H), 7.32-7.10 (m, 3H), 6.36-6.16 (m, 2H), 5.66 (dd, J=20.9, 10.9 Hz, 1H), 5.40 (d, J=10.6 Hz, 1H), 5.09 (s, 3H), 4.34 (d, J=54.9 Hz, 4H), 3.75 (dd, J=24.2, 13.4 Hz, 4H), 3.62 (d, J=6.0 Hz, 4H), 3.40 (d, J=11.7 Hz, 5H), 3.03 (d, J=19.0 Hz, 4H), 2.82 (d, J=17.2 Hz, 2H), 2.59 (d, J=8.7 Hz, 1H), 2.39 (d, J=10.3 Hz, 2H), 2.34-2.14 (m, 4H), 2.09 (d, J=2.5 Hz, 3H), 1.97 (d, J=13.2 Hz, 2H), 1.90-1.65 (m, 5H), 1.36 (s, 3H), 0.95 (dt, J=12.3, 6.9 Hz, 3H), 0.89-0.66 (m, 4H).

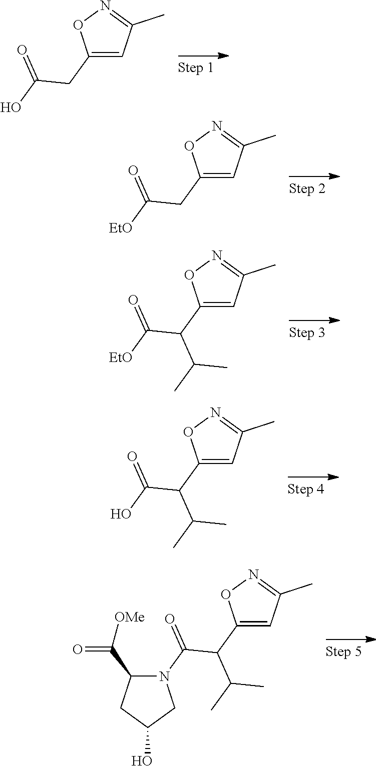

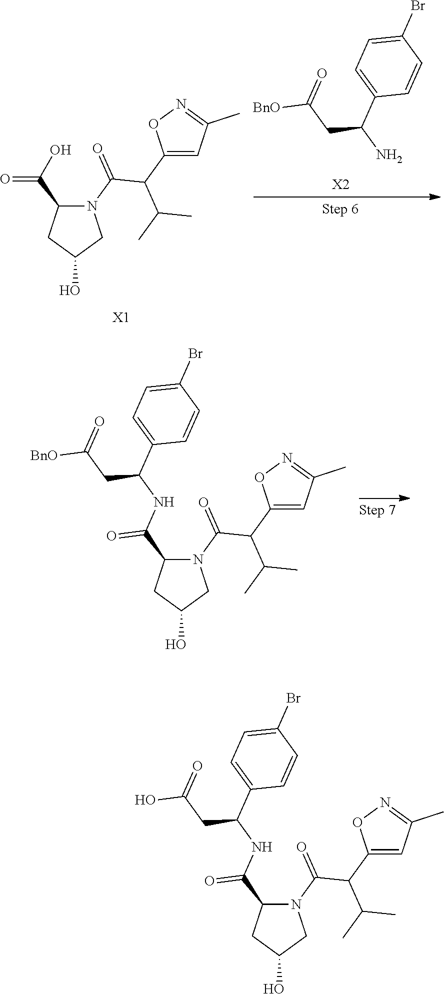

### Step 1: Synthesis of ethyl 2-(3-methylisoxazol-5-yl)acetate

Dissolved (3-methyl-1,2-oxazol-5-yl)acetic acid (4 g, 28.3435 mmol) in ethanol (30 mL) and added sulfuric acid (0.05 mL). The reaction was stirred over two days at room temperature. The reaction was then concentrated and purified by SiO₂ column chromatography eluting with 0-50% ethyl acetate:hexanes. Isolated ethyl 2-(3-methylisoxazol-5-yl)acetate as a clear oil (4.08 g, 85%). LCMS: C₈H₁₁NO₃ requires: 169.18, found: m/z=170.3 [M+H]⁺. ¹H NMR (500 MHz, Chloroform-d) δ 7.29 (s, 1H), 6.13 (s, 1H), 4.24 (q, J=7.1 Hz, 2H), 3.80 (s, 2H), 2.32 (s, 3H), 1.31 (t, J=7.1 Hz, 3H).

### Step 2: Synthesis of ethyl 3-methyl-2-(3-methylisoxazol-5-yl)butanoate

Dissolved ethyl 2-(3-methylisoxazol-5-yl)acetate (4.08 g, 24.1163 mmol) in THE (30 mL) and added potassium tert-butoxide (4.06 g, 36.1745 mmol). The reaction was stirred at 0° C. for 20 min, and then 2-iodopropane (3.13 mL, 31.3512 mmol) was added. The resulting reaction mixture was stirred overnight at room temperature. The reaction was then quenched with ice water and extracted with ethyl acetate. The organic layer was washed with brine, dried over sodium sulfate, filtered, and concentrated. Purification was via column chromatography using 0-50% ethyl acetate:heptane on SiO₂. Ethyl 3-methyl-2-(3-methylisoxazol-5-yl)butanoate was isolated as a clear oil (2.37 g, 47%). LCMS: C₁₁H₁₇NO₃ requires: 211.26, found: m/z=212.5 [M+H]⁺. ¹H NMR (500 MHz, Chloroform-d) δ 6.11 (s, 1H), 4.31-4.11 (m, 2H), 3.59 (d, J=8.7 Hz, 1H), 2.38 (dt, J=8.7, 6.7 Hz, 1H), 2.30 (s, 3H), 1.28 (t, J=7.2 Hz, 3H), 1.02 (d, J=6.7 Hz, 3H), 0.92 (d, J=6.7 Hz, 3H).

### Step 3: Synthesis of 3-methyl-2-(3-methylisoxazol-5-yl)butanoic acid

Dissolved ethyl 3-methyl-2-(3-methylisoxazol-5-yl)butanoate (2.37 g, 11.2373 mmol) in THE (15 mL) and added lithium hydroxide monohydrate (471.52 mg, 11.2373 mmol) and water (1 mL). The reaction was then stirred overnight at room temperature. The reaction was then concentrated and re-dissolved in 1:1 MeCN:H₂O (with 0.1% TFA additive). The solution was frozen in a −78° C. bath and lyophilized to a white solid. Isolated 3-methyl-2-(3-methylisoxazol-5-yl)butanoic acid as a white solid (1.49 g, 72%).

### Step 4: Synthesis of methyl (2S,4R)-4-hydroxy-1-(3-methyl-2-(3-methylisoxazol-5-yl)butanoyl)pyrrolidine-2-carboxylate

Combined 3-methyl-2-(3-methylisoxazol-5-yl)butanoic acid (300 mg, 1.6375 mmol), methyl (2S,4R)-4-hydroxypyrrolidine-2-carboxylate hydrochloride (297.4 mg, 1.6375 mmol), and HATU (622.63 mg, 1.6375 mmol) in DCM (10 mL) as a suspension. Added N,N-diisopropylethylamine (0.88 mL, 4.9125 mmol) and the reaction was stirred overnight at room temperature. The reaction was then concentrated and taken into the next step without purification. LCMS: C₁₅H₂₂N₂O₅ requires: 310.35, found: m/z=311.4 [M+H]⁺.

### Step 5: Synthesis of (2S,4R)-4-hydroxy-1-(3-methyl-2-(3-methylisoxazol-5-yl)butanoyl)pyrrolidine-2-carboxylic acid

Dissolved methyl (2S,4R)-4-hydroxy-1-(3-methyl-2-(3-methylisoxazol-5-yl)butanoyl)pyrrolidine-2-carboxylate (450 mg, 1.45 mmol) in THE (10 mL) and then added lithium hydroxide monohydrate (73.01 mg, 1.74 mmol) and water (1 mL). The reaction was stirred overnight at room temperature. The reaction was then concentrated and re-dissolved in MeCN:H₂O (with 0.1% TFA additive). Lyophilization provided a white solid. This material taken forward without further purification. LCMS: C₁₄H₂₀N₂O₅ requires: 296.32, found: m/z=297.4 [M+H]⁺.

### Step 6: Synthesis of benzyl (3S)-3-(4-bromophenyl)-3-((2S,4R)-4-hydroxy-1-(3-methyl-2-(3-methylisoxazol-5-yl)butanoyl)pyrrolidine-2-carboxamido)propanoate

Compound X2 (300 mg, 0.9 mmol) and Compound X1 (266 mg, 0.9 mmol) were suspended in DCM (8 mL) and HATU (340 mg, 0.9 mmol) and N,N-diisopropylethylamine (0.48 mL, 2.69 mmol) was added. The reaction was stirred overnight at room temperature. The reaction was then concentrated and directly purified by SiO₂ column chromatography, eluting with 0-100% ethyl acetate:hexanes. Isolated benzyl (3S)-3-(4-bromophenyl)-3-((2S,4R)-4-hydroxy-1-(3-methyl-2-(3-methylisoxazol-5-yl)butanoyl)pyrrolidine-2-carboxamido)propanoate as a clear oil (415 mg, 75%). LCMS: C₃₀H₃₄BrN₃O₆ requires: 612.52, found: m/z=614.4 [M+H]⁺.

### Step 7: Synthesis of (3S)-3-(4-bromophenyl)-3-((2S,4R)-4-hydroxy-1-(3-methyl-2-(3-methylisoxazol-5-yl)butanoyl)pyrrolidine-2-carboxamido)propanoic acid

Benzyl (3S)-3-(4-bromophenyl)-3-((2S,4R)-4-hydroxy-1-(3-methyl-2-(3-methylisoxazol-5-yl)butanoyl)pyrrolidine-2-carboxamido)propanoate (415 mg, 0.68 mmol) was dissolved in THE (5 mL) and then lithium hydroxide hydrate (28.4 mg, 0.68 mmol) and water (0.5 mL) was added. The reaction was stirred overnight at room temperature. The reaction was then concentrated and the crude was taken into the next step without purification. LCMS: C₂₃H₂₈BrN₃O₆ requires: 521.1, found: m/z=523.2 [M+H]⁺.

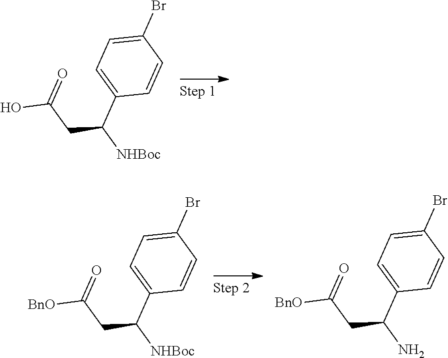 [SMILES: CC(C)(C)OC(=O)N[C@@H](CC(=O)O)c1ccc(Br)cc1 ; CC(C)(C)OC(=O)N[C@@H](CC(=O)OCc1ccccc1)c1ccc(Br)cc1 ; N[C@@H](CC(=O)OCc1ccccc1)c1ccc(Br)cc1]

### Step 1: Synthesis of benzyl (S)-3-(4-bromophenyl)-3-((tert-butoxycarbonyl)amino)propanoate

Combined (3S)-3-(4-bromophenyl)-3-[(tert-butoxycarbonyl)amino]propanoic acid (750 mg, 2.18 mmol) and HATU (828.51 mg, 2.18 mmol) in DCM (8 mL) as a suspension. Benzyl alcohol (0.25 mL, 2.4 mmol) and N,N-diisopropylethylamine (0.78 mL, 4.36 mmol) were then added and the reaction was stirred overnight at room temperature. Concentration and purification by SiO₂ column chromatography, eluting with 0-100% ethyl acetate:hexanes, provided benzyl (3S)-3-(4-bromophenyl)-3-[(tert-butoxycarbonyl)amino]propanoate as a white solid (745 mg, 79%). LCMS: C₁₆H₁₆BrNO₂ requires: 434.33, found: m/z=458.1.0 [M+Na]⁺. Step 2: Synthesis of benzyl (S)-3-amino-3-(4-bromophenyl)propanoate

Dissolved benzyl (3S)-3-(4-bromophenyl)-3-[(tert-butoxycarbonyl)amino]propanoate (745 mg, 1.72 mmol) in DCM (6 mL) and then added HCl in dioxane (4M, 0.60 mL). The reaction was then stirred overnight at room temperature and concentrated to a white solid. The crude material was taken into the next step without purification. LCMS: C₁₆H₁₆BrNO₂ requires: 334.21, found: m/z=336.0 [M+H]⁺.

### Example 84. (4aR,5aS)—N-(1-((1R)-(1-((3S)-3-((2S,4R)-1-(2-(3-cyano-1H-pyrazol-1-yl)-3-methylbutanoyl)-4-hydroxypyrrolidine-2-carboxamido)-3-(4-(4-methylthiazol-5-yl)phenyl)propanoyl)azetidin-3-yl)(phenyl)methyl)-1H-pyrazol-4-yl)-5,5-difluoro-5a-methyl-1,4,4a,5,5a,6-hexahydrocyclopropa[f]indazole-3-carboxamide (37)

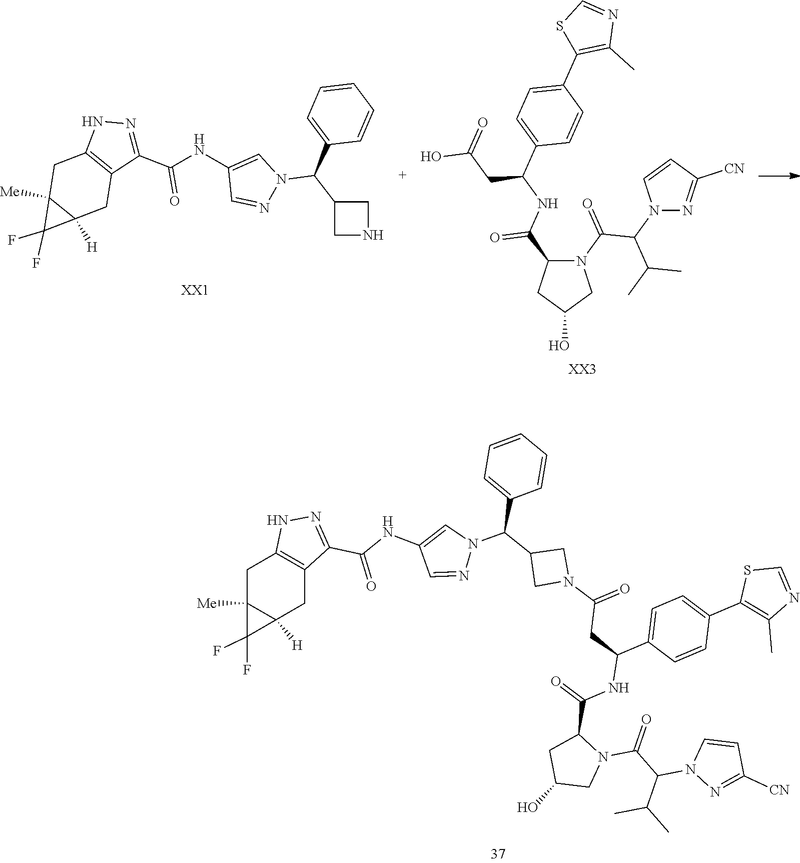

Combined XX3 (15 mg, 0.0272 mmol), XX1 (11.94 mg, 0.0272 mmol), and HATU (10.36 mg, 0.0272 mmol) in DMF (1 mL) as a suspension. N,N-diisopropylethylamine (0.01 mL, 0.0817 mmol) was then added and the reaction was stirred overnight at room temperature. The reaction was then filtered through a syringe filter and purified by preparatory HPLC (5-95% MeCN:H₂O with 0.1% TFA modifier) to provide (4aR,5aS)—N-(1-((1R)-(1-((3S)-3-((2S,4R)-1-(2-(3-cyano-1H-pyrazol-1-yl)-3-methylbutanoyl)-4-hydroxypyrrolidine-2-carboxamido)-3-(4-(4-methylthiazol-5-yl)phenyl)propanoyl)azetidin-3-yl)(phenyl)methyl)-1H-pyrazol-4-yl)-5,5-difluoro-5a-methyl-1,4,4a,5,5a,6-hexahydrocyclopropa[indazole-3-carboxamide (7 mg, 25%) as a white solid after lyophilization. C₅₀H₅₂F₂N₁₂O₅S requires: 970.4, found: m/z=971.2 [M+H]⁺.

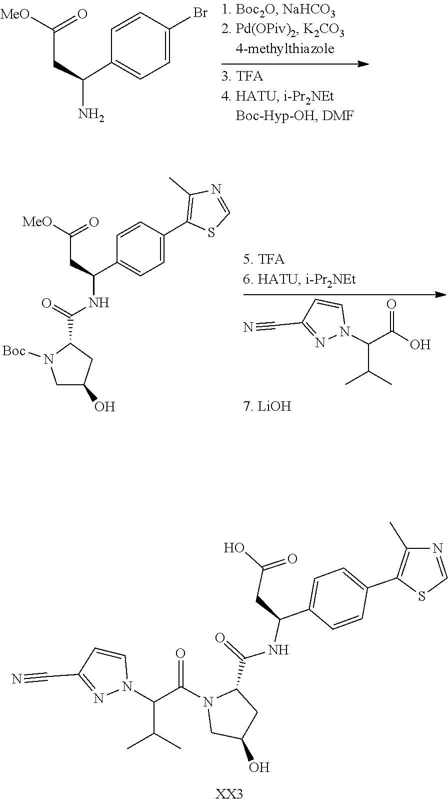

### Step 1: Synthesis of methyl (S)-3-(4-bromophenyl)-3-((tert-butoxycarbonyl)amino)propanoate

To a solution of methyl (S)-3-amino-3-(4-bromophenyl)propanoate (5.29 g, 20.5 mmol) in EtOAc (60 mL) and 10% wt. NaHCO₃ (60 mL) at 0° C. was added di-tert-butyl dicarbonate (4.70 g, 21.5 mmol, 1.05 equiv) portionwise over 15 min. After 4 h, the solution was separated, and the water layer was extracted with EtOAc. The combined organic layers were washed with brine, dried over Na₂SO₄, filtered, and concentrated to afford a white solid, which was used in the next step without purification. LCMS (ESI) m/z 359.06 [M+H]⁺ and m/z 302.0 [M−C₄H₉+H]⁺.

### Step 2: Synthesis of methyl (S)-3-((tert-butoxycarbonyl)amino)-3-(4-(4-methylthiazol-5-yl)phenyl)propanoate

An oven dried 100 mL round-bottomed flask equipped with a stir bar was charged with methyl (S)-3-(4-bromophenyl)-3-((tert-butoxycarbonyl)amino)propanoate (7.34 g, 20.5 mmol, 1 equiv), anhydrous K₂CO₃ (5.67 g, 41.0 mmol, 2 equiv), 4-methylthiazole (3.76 mL, 41 mmol, 2 equiv), and Pd(OPiv)₂ (0.126 g, 0.41 mmol, 0.02 equiv) and the mixture was dissolved in anhydrous DMA (25 mL). While flushing the flask with N₂, the solution was heated to 130° C. and the reaction was maintained at that temperature under a N₂ atmosphere for 8 h. At reaction completion, the mixture was cooled to rt, diluted with H₂O (100 mL), and extracted with EtOAc (3×100 mL). The combined organic layers were washed with brine (100 mL), dried over Na₂SO₄, filtered, and concentrated in vacuo. The crude product was purified by silica preparatory chromatography eluting with 10-50% EtOAc:hexanes to afford the desired product (10.7 g, 52% yield) as a light-yellow oil that solidified to a pale-yellow solid. ¹H NMR (500 MHz, DMSO-d₆) 9.00 (s, 1H), 7.59-7.52 (m, 1H), 7.47-7.44 (m, 2H), 7.42-7.39 (m, 2H), 5.01-4.92 (m, 1H), 3.57 (s, 3H), 2.78-2.73 (m, 2H), 2.45 (s, 3H), 1.36 (s, 9H). LCMS (ESI) m/z 377.3 [M+H]⁺.

### Step 3: Synthesis of methyl (S)-3-amino-3-(4-(4-methylthiazol-5-yl)phenyl)propanoate

Methyl (S)-3-((tert-butoxycarbonyl)amino)-3-(4-(4-methylthiazol-5-yl)phenyl)propanoate (1.88 g, 5 mmol, 1 equiv) was dissolved in CH₂Cl₂ (25 mL) and water (1 mL) at rt and then TFA (25 mL) was added. The mixture was stirred at rt for 2 h. After concentration under reduced pressure, the oily residue was further dried by adding toluene and concentrating (2×) and was finally placed under high vacuum. The resulting thick amber oil was used as is in the following coupling step.

### Step 4: Synthesis of tert-butyl (2S,4R)-4-hydroxy-2-(((S)-3-methoxy-1-(4-(4-methylthiazol-5-yl)phenyl)-3-oxopropyl)carbamoyl)pyrrolidine-1-carboxylate

To a solution of trans-N-(tert-butoxycarbonyl)-4-hydroxy-L-proline (1.16 g, 5 mmol, 1 equiv) and anhydrous i-Pr₂NEt (3.48 mL, 20 mmol, 4 equiv) in anhydrous DMF (25 mL) at rt under N₂ was added HATU (2.09 g, 5.5 mmol, 1.1 equiv). After 15 min, methyl (S)-3-amino-3-(4-(4-methylthiazol-5-yl)phenyl)propanoate TFA (1.95 g, 5 mmol, 1 equiv) dissolved in anhydrous DMF (5 mL) was added and the reaction was stirred for another 2 h or until complete as confirmed by LCMS. At completion, the solution was diluted with EtOAc and washed with water (2×). The combined water layers were back extracted with EtOAc. The combined organic layers were washed with brine, dried over anhydrous Na₂SO₄, filtered, and concentrated under reduced pressure. The crude product was purified by silica preparatory chromatography eluting with 0-12% MeOH:CH₂Cl₂ to afford (1.56 g, 64% yield) as a light-yellow thick oil. ¹H NMR (500 MHz, DMSO-d₆) 9.01 (s, 1H), 7.60-7.52 (m, 1H), 7.51-7.44 (m, 2H), 7.42-7.39 (m, 2H), 5.01-4.92 (m, 1H), 3.57 (s, 3H), 3.51 (dd, J=11.0, 4.7 Hz, 2H), 2.78-2.73 (m, 2H), 2.61-2.48 (m, 4H), 2.45 (s, 3H), 2.03 (d, J=7.4 Hz, 2H), 1.42 (s, 9H). LCMS (ESI) m/z 490.2 [M+H]⁺ and m/z 433.2 [M−C₄H₉+H]⁺.

### Step 5: Synthesis of methyl (S)-3-((2S,4R)-4-hydroxypyrrolidine-2-carboxamido)-3-(4-(4-methylthiazol-5-yl)phenyl)propanoate

Tert-butyl (2S,4R)-4-hydroxy-2-(((S)-3-methoxy-1-(4-(4-methylthiazol-5-yl)phenyl)-3-oxopropyl)carbamoyl)pyrrolidine-1-carboxylate (1.22 g, 2.5 mmol, 1 equiv) was dissolved in CH₂Cl₂ (12.5 mL) and water (0.5 mL) at rt and then TFA (12.5 mL) was added. The mixture was stirred at rt for 2 h. After concentration under reduced pressure, the oily residue was further dried by adding toluene and concentrating (2×) and was finally placed under high vacuum. The resulting thick amber oil was used as is in the following coupling step.

### Step 6: Synthesis of methyl (S)-3-((2S,4R)-1-((S)-2-(3-cyano-1H-pyrazol-1-yl)-3-methylbutanoyl)-4-hydroxypyrrolidine-2-carboxamido)-3-(4-(4-methylthiazol-5-yl)phenyl)propanoate

To a solution of 2-(3-cyano-1H-pyrazol-1-yl)-3-methylbutanoic acid (0.483 g, 2.5 mmol, 1 equiv) and anhydrous i-Pr₂NEt (1.74 mL, 10 mmol, 4 equiv) in anhydrous DMF (12.5 mL) at rt under N₂ was added HATU (1.05 g, 2.75 mmol, 1.1 equiv). After 15 min, methyl (S)-3-((2S,4R)-4-hydroxypyrrolidine-2-carboxamido)-3-(4-(4-methylthiazol-5-yl)phenyl)propanoate TFA (1.26 g, 2.5 mmol, 1 equiv) dissolved in anhydrous DMF (3 mL) was added and the reaction was stirred for another 2 h or until completion as confirmed by LCMS. At completion, the solution was diluted with EtOAc and washed with water (2×). The combined water layers were back extracted with EtOAc. The combined organic layers were washed with brine, dried over anhydrous Na₂SO₄, filtered, and concentrated under reduced pressure. The crude product was purified by silica preparatory chromatography eluting with 0-12% MeOH:CH₂Cl₂ to afford (0.931 g, 66% yield) as a light-yellow thick oil. LCMS (ESI) m/z 565.2 [M+H]⁺.

### Step 7: Synthesis of (S)-3-((2S,4R)-1-((S)-2-(3-cyano-1H-pyrazol-1-yl)-3-methylbutanoyl)-4-hydroxypyrrolidine-2-carboxamido)-3-(4-(4-methylthiazol-5-yl)phenyl)propanoic acid

To a solution of methyl (S)-3-((2S,4R)-1-((S)-2-(3-cyano-1H-pyrazol-1-yl)-3-methylbutanoyl)-4-hydroxypyrrolidine-2-carboxamido)-3-(4-(4-methylthiazol-5-yl)phenyl)propanoate (0.706 g, 1.25 mmol, 1 equiv) in THE (5 mL) at 0° C. was added a solution of LiOH monohydrate (0.105 g, 2.5 mmol, 2 equiv) in water (5 mL). After completion at approximately 4 h, the solution was diluted with MTBE and separated. The water layer was made acidic with 4 M HCl and extracted into CH₂Cl₂ (3×). The combined organic layers were dried over Na₂SO₄, filtered, and concentrated to afford the crude product. LCMS (ESI) m/z 551.2 [M+H⁺.

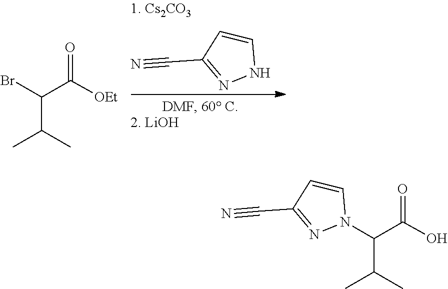

### Step 1: Synthesis of ethyl 2-(3-cyano-1H-pyrazol-1-yl)-3-methylbutanoate

To a suspension of 1H-pyrazole-3-carbonitrile (0.931 g, 10 mmol, 1 equiv), Cs₂CO₃ (4.07 g, 12.5 mmol, 1.25 equiv) in anhydrous DMF (6 mL) was added ethyl 2-bromo-3-methylbutanoate (1.8 mL, 11 mmol, 1.1 equiv) dropwise. The reaction was stirred at room temperature for approximately 5 h or until complete by LCMS. At completion, the reaction was diluted with EtOAc, washed with water, and the layers were separated. The organic layer was back extracted with EtOAc. The combined organic layers were washed with water, dried over Na₂SO₄, filtered, and concentrated. The crude product was purified by silica flash chromatography eluting with a gradient of 10-50% EtOAc in hexanes to afford a colorless oil (1.64 g, 74% yield). ¹H NMR (500 MHz, DMSO-d₆) δ 8.08 (m, 1H), 6.98 (m, 1H), 4.87 (d, J=8.5 Hz, 2H), 4.18 (m, 2H), 2.93 (s, 1H), 1.11 (m, 3H), 0.96 (dt, J=10.8, 5.4 Hz, 3H), 0.72 (d, J=6.6 Hz, 3H). LCMS (ESI) m/z 222.12 [M+H]⁺.

### Step 2: Synthesis of 2-(3-Cyano-1H-pyrazol-1-yl)-3-methylbutanoic acid

To a solution of ethyl 2-(3-cyano-1H-pyrazol-1-yl)-3-methylbutanoate (1.11 g, 5 mmol) in THE (10 mL) at 0° C. was added a solution of LiOH (0.420 g, 10 mmol, 2 equiv) in water (10 mL). After completion at approximately 4 h, the solution was diluted with MTBE and separated. The water layer was made acidic with 4 M HCl and extracted into CH₂Cl₂ (3×). The combined organic layers were dried over Na₂SO₄, filtered, and concentrated to afford the crude product. The crude product was purified by silica preparatory chromatography eluting with 0-12% MeOH:CH₂Cl₂ with 0.1% TFA to afford an off-white solid (0.792 g, 82%). ¹H NMR (500 MHz, DMSO-d₆) δ 12.01 (s, 1H), 8.09 (m, 1H), 6.99 (m, 1H), 4.87 (d, J=8.5 Hz, 2H), 2.92 (s, 1H), 0.94 (dt, J=10.8, 5.4 Hz, 3H), 0.74 (d, J=6.6 Hz, 3H). LCMS (ESI) m/z 194.09 [M+H]⁺.

### Example 85. (4aR,5aS)-5,5-difluoro-N-(1-((1R)-(1-((3S)-3-((2S,4R)-4-hydroxy-1-(3-methyl-2-(3-methylisoxazol-5-yl)butanoyl)pyrrolidine-2-carboxamido)-3-(4-(4-methylthiazol-5-yl)phenyl)propanoyl)azetidin-3-yl)(phenyl)methyl)-1H-pyrazol-4-yl)-5a-methyl-1,4,4a,5,5a,6-hexahydrocyclopropa[f]indazole-3-carboxamide (38)

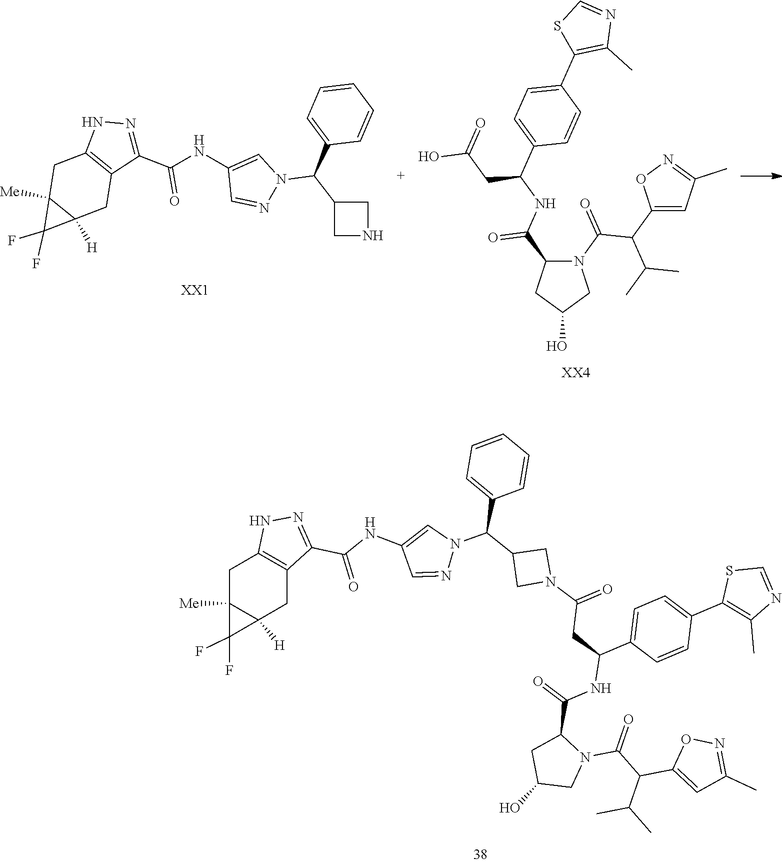

Combined XX4 (10 mg, 0.0185 mmol), XX1 (8.11 mg, 0.0185 mmol), and HATU (7.03 mg, 0.0185 mmol) in DMF (1 mL) as a suspension. N,N-diisopropylethylamine (0.01 mL, 0.0555 mmol) was then added and the reaction was stirred overnight at room temperature. The reaction was then filtered through a syringe filter and purified by preparatory HPLC (5-95% MeCN:H₂O with 0.1% TFA modifier) to provide (4aR,5aS)-5,5-difluoro-N-(1-((1R)-(1-((3S)-3-((2S,4R)-4-hydroxy-1-(3-methyl-2-(3-methylisoxazol-5-yl)butanoyl)pyrrolidine-2-carboxamido)-3-(4-(4-methylthiazol-5-yl)phenyl)propanoyl)azetidin-3-yl)(phenyl)methyl)-1H-pyrazol-4-yl)-5a-methyl-1,4,4a,5,5a,6-hexahydrocyclopropafindazole-3-carboxamide as a white solid (7 mg, 37%). C₅₀H₅₄F₂N₁₀O₆S requires: 960.4, found: m/z=961.4 [M+H]⁺.

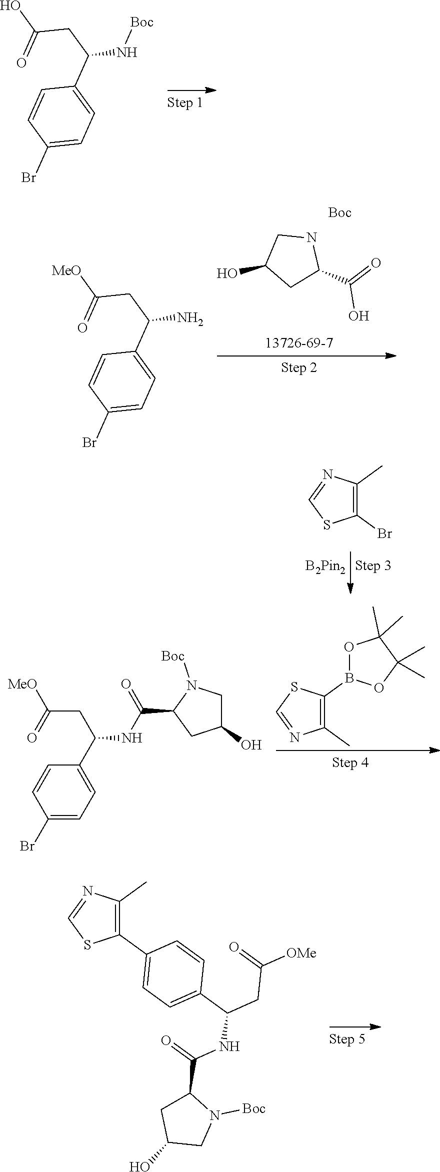

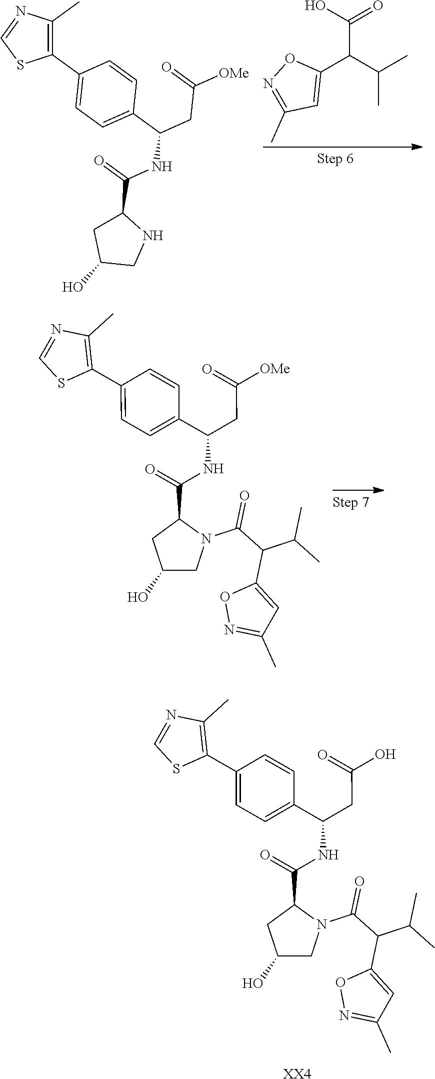

### Step 1: Synthesis of methyl (3S)-3-amino-3-(4-bromophenyl)propanoate

To a solution of (3S)-3-(4-bromophenyl)-3-{[(tert-butoxy)carbonyl]amino}propanoic acid (8 g, 0.023 mmol) in methanol (100 mL, 0.01 M) at 0° C. was slowly added a cooled solution of HCl (3 M in MeOH, 160 mL, 0.01 M). The mixture was stirred at rt for 16 h. The crude reaction was concentrated in vacuo at 30° C. and then a solution of HCl in Et₂O (3 M, 40 mL) was added, followed by concentration in vacuo to provide the title compound as a foamy white solid (5.71 g, 95%). The product was isolated as an HCl salt and was introduced in the next step without additional purification. ESI(+) [M+H]⁺=258.00. ¹H NMR (300 MHz, Methanol-d₄) δ 7.63 (d, J=8.2 Hz, 2H), 7.40 (d, J=8.2 Hz, 2H), 4.73 (t, J=7.1 Hz, 1H), 3.70 (s, 3H), 3.19-2.97 (m, 2H).

### Step 2: Synthesis of tert-butyl (2S,4R)-2-{[(1S)-1-(4-bromophenyl)-3-methoxy-3-oxopropyl]carbamo-yl}-4-hydroxypyrrolidine-1-carboxylate

To a solution of (2S,4R)-1-[(tert-butoxy)carbonyl]-4-hydroxypyrrolidine-2-carboxylic acid (5.71 g, 24.7 mmol) in DMF (45 mL, 0.5 M) at 0° C. was added DIPEA (6 mL). Then, a solution of HATU (8.53 g, 22.5 mmol) in DMF (45 mL, 0.5 M) was added slowly at 0° C. The reaction mixture was stirred at room temperature for 0.5 h and was then slowly added at −30° C. to a cooled solution of methyl (3S)-3-amino-3-(4-bromophenyl)propanoate (6.7 g, 21.5 mmol) in DMF (35 mL, 0.6 M) pre-treated with DIPEA (20 mL). The mixture was stirred at -30° C. and slowly warmed to rt in 2 h. The crude reaction was then poured on crushed ice and extracted with DCM (6×500 mL). The combined organic layers were dried over Na₂SO₄ and concentrated in vacuo. The residue was purified by silica gel flash column chromatography eluting with DCM:MeOH (9:1) to provide the title compound as a foamy white solid (10.54 g, quant.). ESI(+) [M+H]⁺=471.10. ¹H NMR (300 MHz, DMSO-d₆) δ 7.47 (t, J=7.9 Hz, 2H), 7.36-7.20 (m, 2H), 5.32 (t, J=7.4 Hz, 1H), 4.29 (dd, J=15.4, 6.9 Hz, 2H), 3.62 (s, 3H), 3.60-3.40 (m, 1H), 3.03-2.73 (m, 2H), 2.34-2.08 (m, 1H), 2.03-1.76 (m, 1H), 1.47 (s, 3H), 1.40-1.29 (s, 6H).

### Step 3: Synthesis of methyl-5-(4,4,5,5-tetramethyl-1,3,2-dioxaborolan-2-yl)-1,3-thiazole

A suspension of 5-bromo-4-methyl-1,3-thiazole (7.5 g, 42.1 mmol), KOAc (12.4 g, 126.4 mmol), 4,4,5,5-tetramethyl-2-(4,4,5,5-tetramethyl-1,3,2-dioxaborolan-2-yl)-1,3,2-dioxaborolane (21.4 g, 1.85 mmol), and Pd(PPh₃)₄ (10 g, 20 mol %) in dioxane (375 mL, 0.1 M) was purged with argon for 10 min and then stirred at 95° C. for 16 h. The mixture was then cooled to rt, filtered through a pad of Celite, and concentrated in vacuo. The residue was purified by short manual column chromatography eluting with hexane:EtOAc (1:1) to provide the title product as an off-white solid (10.25 g, 52% yield, contaminated with pinacol derivatives 50% wt). ¹H NMR (300 MHz, Chloroform-d) δ 8.92 (s, 1H), 2.70 (s, 3H), 1.34 (s, 12H).

### Step 4: (3S)-3-{[(2S,4R)-1-[(tert-butoxy)carbonyl]-4-hydroxypyrrolidin-2-yl]formami-do}-3-[4-(4-methyl-1,3-thiazol-5-yl)phenyl]propanoic acid methyl ester

A mixture of tert-butyl (2S,4R)-2-{[(1S)-1-(4-bromophenyl)-3-methoxy-3-oxopropyl]carbamoyl}-4-hydroxypyrrolidine-1-carboxylate (9 g, 19.09 mmol, 1 equiv), 4-methyl-5-(4,4,5,5-tetramethyl-1,3,2-dioxaborolan-2-yl)-1,3-thiazole (9.91 g, 21 mmol, 1.2 equiv), K₂CO₃ (13.2 g, 95.5 mmol), and Pd(dppf)Cl₂·DCM (1.6 g, 10 mol %) in dioxane:H₂O (5:1, 380 mL, 0.05 M) was purged with argon for 20 min and stirred at 110° C. for 2 h. The mixture was then cooled to room temperature, filtered through a pad of Celite, and concentrated in vacuo. The residue was purified by flash column chromatography eluting with DCM:MeOH:AcOH (8:2:0.2 to 6:4:0.2) and triturated with diethyl ether to provide the title compound as a grey solid (6.6 g, 76%). ESI(+) [M+H]⁺=476.07. ¹H NMR (300 MHz, Methanol-d₄) δ 8.88 (s, 1H), 7.47 (m, 4H), 5.54-5.28 (m, 1H), 4.33 (d, J=9.9 Hz, 2H), 3.68-3.40 (m, 2H), 3.60 (s, 3H), 2.88 (m, 2H), 2.48 (s, 3H), 2.31-2.14 (m, 1H), 1.99 (s, 1H), 1.48 (s, 3H), 1.33 (s, 6H).

### Step 5: Synthesis of methyl (3S)-3-{[(2S,4R)-4-hydroxypyrrolidin-2-yl]formamido}-3-[4-(4-methyl-1,3-thiazol-5-yl)phenyl]propanoate

A mixture of (3S)-3-{[(2S,4R)-1-[(tert-butoxy)carbonyl]-4-hydroxypyrrolidin-2-yl]formamido}-3-[4-(4-methyl-1,3-thiazol-5-yl)phenyl]propanoic acid methyl ester (0.3 g, 0.61 mmol) and HCl in MeOH (2 N, 10 equiv) was stirred at rt for 2 h. The volatiles were removed in vacuo and the resulting solid was triturated with anhydrous diethyl ether to provide the title compound as a viscous brown oil (0.22 g, 83%). ESI(+) [M+H]⁺=390.45. ¹H NMR (300 MHz, DMSO-d₆) δ 9.89 (s, 1H), 9.32 (d, J=7.9 Hz, 1H), 9.03 (s, 1H), 8.65 (s, 1H), 7.51-7.39 (m, 4H), 4.33 (s, 2H), 3.61 (s, 3H), 3.51 (s, 1H), 3.41 (s, 2H), 3.07 (d, J=4.7 Hz, 1H), 2.88 (d, J=7.5 Hz, 2H), 2.33 (s, 1H), 1.78 (m, 1H).

### Step 6: Synthesis of (3S)-3-{[(2S,4R)-4-hydroxy-1-[3-methyl-2-(3-methyl-1,2-oxazol-5-yl)butanoyl]-pyrrolidin-2-yl]formamido}-3-[4-(4-methyl-1,3-thiazol-5-yl)phenyl]propanoic acid

To a solution of methyl (3S)-3-{[(2S,4R)-4-hydroxypyrrolidin-2-yl]formamido}-3-[4-(4-methyl-1,3-thiazol-5-yl)phenyl]propanoate (0.23 g, 0.56 mmol) and 3-methyl-2-(3-methyl-1,2-oxazol-5-yl)butanoic acid (0.11 g, 0.62 mmol) in DCM (6 mL, 0.1 M) was added DIPEA (0.22 mL, 1.7 mmol) and HATU (0.32 g, 0.84 mmol). The mixture was stirred at 25° C. overnight. The reaction mixture was then quenched with water and extracted with ethyl acetate. The combined organic layers were washed with acidic water, brine, dried over Na₂SO₄, and concentrated to provide a crude product, which was then purified via silica flash chromatography, eluting with 10% MeOH:DCM. ESI(+) [M+H]⁺=556.04. ¹H NMR (300 MHz, DMSO-d₆) δ 8.72 (s, 1H), 7.47-7.35 (br m, 4H), 6.10 (m, 1H), 5.35 (m, 1H), 4.60 (m, 2H), 3.72 (m, 4H), 3.60 (s, 3H), 2.77 (m, 2H), 2.50 (m, 4H), 2.25 (m, 4H), 2.06 (m, 1H), 1.07 (m, 3H), 0.89 (m, 3H).

### Step 7: Synthesis of (3S)-3-{[(2S,4R)-4-hydroxy-1-[3-methyl-2-(3-methyl-1,2-oxazol-5-yl)butanoyl]-pyrrolidin-2-yl]formamido}-3-[4-(4-methyl-1,3-thiazol-5-yl)phenyl]propanoic acid

To a solution of (3S)-3-{[(2S,4R)-4-hydroxy-1-[3-methyl-2-(3-methyl-1,2-oxazol-5-yl)butanoyl]-pyrrolidin-2-yl]formamido}-3-[4-(4-methyl-1,3-thiazol-5-yl)phenyl]propanoic acid (0.28 g, 0.54 mmol) in methanol:water (3:1; 0.14 M) was added and sodium hydroxide (0.03 g, 0.75 mmol) and the resulting mixture was stirred at rt until the reaction was completed. The organic solvent was evaporated under reduced pressure and the water residue was acidified with 1 N HCl to pH=4. The resulting solution was purified with reversed-phase flash chromatography (eluting with 5 to 30% acetonitrile in water) to provide a white solid (0.1 g, 37%). LCMS: ESI(+) [M+H]=542.66, method: LCMS-019-10-70-95-6-1-25-UV, Rₜ=2.443 min, 98.32% purity (254 nm). ¹H NMR (300 MHz, DMSO-d₆) δ 8.90 (s, 1H), 7.47 (m, 4H), 6.25 (d, J=5.9 Hz, 1H), 5.38 (m, 1H), 4.62-4.37 (m, 2H), 3.94-3.41 (m, 4H), 3.10-2.78 (m, 2H), 2.50 (m, 4H), 2.25 (m, 4H), 1.98 (m, 1H), 1.07 (d, J=7.6 Hz, 3H), 0.94-0.82 (m, 3H).

### Example 86. (2S,4R)—N-[(2-{[1-(4-{3-[(S)-{4-[(4aS,5aR)-5,5-difluoro-5a-methyl-1H,4H,4aH,5H,5aH,6H-cyclopropa[f]indazole-3-amido]-1H-pyrazol-1-yl}(phenyl)methyl]azetidin-1-yl}-4-oxobutanoyl)piperidin-4-yl]oxy}-4-bromophenyl)methyl]-4-hydroxy-1-[(2RS)-3-methyl-2-(3-methyl-1,2-oxazol-5-yl)butanoyl]pyrrolidine-2-carboxamide (39)

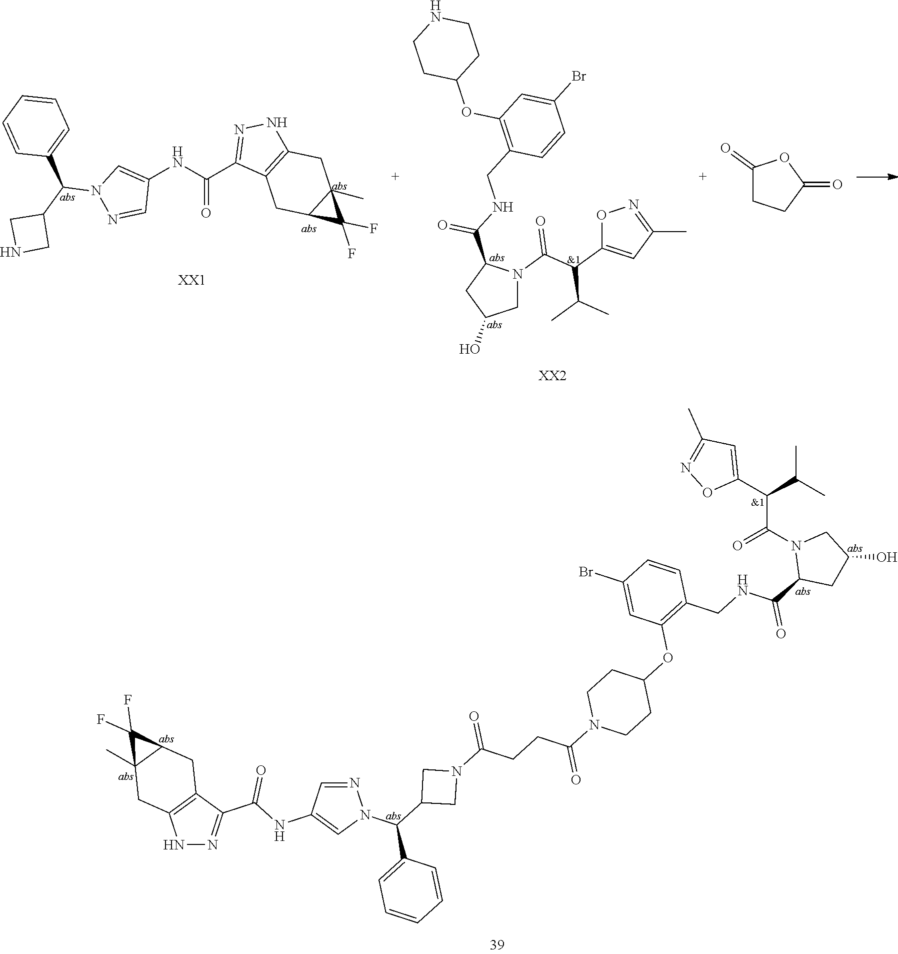

To a one dram vial, succinic anhydride (2.28 mg, 0.0228 mmol) and XX2 (14.28 mg, 0.0228 mmol) were added followed by DMIF (0.25 mL). The mixture was stirred at rt for one hour before XX1 (10 mg, 0.0228 mmol), [(dimethylamino)({[1,2,3]triazolo[4,5-b]pyridin-3-yloxy})methylidene]dimethylazanium hexafluoro-lambda5-phosphanuide (8.67 mg, 0.0228 mmol), and DIPEA (19.92 μL, 0.114 mmol) were added. The reaction was then stirred for another hour before purification by preparatory HIPLC (5-95% MeCN in H₂O with 0.1% TFA) to afford (2S,4R)—N-[(2-{[1-(4-{3-[(S)-{4-[(4aS,5aR)-5,5-difluoro-5a-methyl-1H,4H,4aH,5H,5aH,6H-cyclopropa[f]indazole-3-amido]-1H-pyrazol-1-yl}(phenyl)methyl]azetidin-1-yl}-4-oxobutanoyl)piperidin-4-yl]oxy}-4-bromophenyl)methyl]-4-hydroxy-1-[(2RS)-3-methyl-2-(3-methyl-1,2-oxazol-5-yl)butanoyl]pyrrolidine-2-carboxamide (7.5 mg, 27.6%). LCMS: C₅₃H₆₁BrF₂N₁₀O₈ requires: 1082.4, found: m/z=1083.3 [M+H]⁺. ¹H NMR (500 MHz, DMSO-d₆) δ 12.98 (s, 2H), 10.18 (d, J=5.4 Hz, 2H), 8.34 (d, J=6.1 Hz, 1H), 8.27 (s, 1H), 8.10 (d, J=7.8 Hz, 2H), 7.68 (d, J=4.0 Hz, 2H), 7.52 (s, 1H), 7.45-7.30 (m, 9H), 7.29-7.23 (m, 2H), 7.15 (d, J=7.6 Hz, 1H), 7.08 (q, J=9.5 Hz, 2H), 6.24 (d, J=5.8 Hz, 1H), 5.68 (dd, J=11.0, 5.5 Hz, 2H), 4.76 (s, 2H), 4.46 (s, 1H), 4.36 (d, J=8.3 Hz, 2H), 4.24-4.16 (m, 7H), 3.92 (s, 1H), 3.86 (t, J=8.2 Hz, 2H), 3.83-3.72 (m, 1H), 3.69 (s, 17H), 3.58 (s, 1H), 3.07 (s, 5H), 3.03 (s, 1H), 2.83 (d, J=17.2 Hz, 2H), 2.57 (s, 4H), 2.22 (ddt, J=27.2, 18.1, 10.0 Hz, 11H), 2.02 (s, 2H), 1.69 (s, 2H), 1.59 (s, 2H), 1.37 (d, J=5.7 Hz, 6H), 0.96 (dd, J=18.6, 6.4 Hz, 5H), 0.80 (dd, J=18.1, 6.6 Hz, 5H), 0.68 (s, 1H), 0.57 (s, 1H).

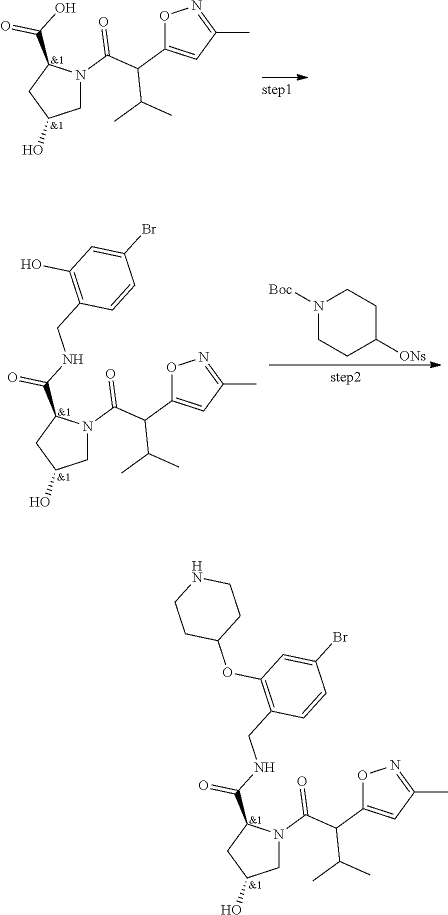

### Step 1: Synthesis of (2S,4R)—N-(4-bromo-2-hydroxybenzyl)-4-hydroxy-1-(3-methyl-2-(3-methylisoxazol-5-yl)butanoyl)pyrrolidine-2-carboxamide

Combined 2-(aminomethyl)-5-bromophenol (200 mg, 0.9898 mmol), (2S,4R)-4-hydroxy-1-(3-methyl-2-(3-methylisoxazol-5-yl)butanoyl)pyrrolidine-2-carboxylic acid (293.32 mg, 0.9898 mmol) in DCM (6 mL) and then added N,N-diisopropylethylamine (0.53 mL, 2.9695 mmol). The reaction was stirred until a solution formed and then HATU (376.38 mg, 0.9898 mmol) was added. The reaction was then stirred at room temperature for four hours. The reaction was concentrated and directly purified by SiO₂ column chromatography eluting with 0-10% MeOH:DCM. Isolated (2S,4R)—N-(4-bromo-2-hydroxybenzyl)-4-hydroxy-1-(3-methyl-2-(3-methylisoxazol-5-yl)butanoyl)pyrrolidine-2-carboxamide as a yellow oil (470 mg, 98%). LCMS: C₂₁H₂₆BrN₃O₅ requires: 480.36, found: m/z=480.4 [M+H]⁺.

### Step 2: Synthesis of (2S,4R)—N-{[4-bromo-2-(piperidin-4-yloxy)phenyl]methyl}-4-hydroxy-1-[3-methyl-2-(3-methyl-1,2-oxazol-5-yl)butanoyl]pyrrolidine-2-carboxamide

To a four dram vial was added (2S,4R)—N-[(4-bromo-2-hydroxyphenyl)methyl]-4-hydroxy-1-[3-methyl-2-(3-methyl-1,2-oxazol-5-yl)butanoyl]pyrrolidine-2-carboxamide (201.8 mg, 0.4201 mmol), tert-butyl 4-[(4-nitrobenzenesulfonyl)oxy]piperidine-1-carboxylate (194.8 mg, 0.5041 mmol), and caesium carbonate (342.19 mg, 1.0503 mmol) followed by DMF (4 mL). The reaction was then heated to 100° C. for 3 h before concentration. HCl in dioxane (2M, 4 mL) was then added to the mixture. The crude was stirred for another two hours before concentration and purification via reverse phase column chromatography (5-95% MeCN in H₂O with 0.1% TFA). LCMS: C₂₆H₃₅BrN₄O₅ requires 562.18 found: m/z=563.46 [M+H]⁺.

### Biological Example 1—In Vitro ITK Degradation after Oral Administration

### ITK Degradation HiBiT Assay

Compounds provided herein were assayed in vitro with ITK HiBit cell lines. Compound dilution series (11-point, 5-fold dilutions in DMSO, columns 2-12 with replicate in rows A/B, C/D, E/F, G, and H at 2000× the final required concentrations were prepared in 96-well plate (Falcon, cat. no. 353077). Column 1, rows A-H were control DMSO. The 2000× solutions ranged from 2 mM to 1.024 nM (final assay concentration range 1 μM to 0.512 pM). The 2000×solutions were added to cells in 10 μL volume, for a final DMSO concentration of 0.5% and final assay compound concentration of 1×. For the cells, C-terminal HiBiT-tagged Molt4 cells (ATCC CRL-1552, monoclonal cell line clone 1C10) were plated at 1×10⁶ cells/mL, 100 μL/well (100×10⁴ cells/well) in complete RPMI (10% FBS, 1% L-glutamine). The cells were incubated with compounds 1-25, 31, and 31 for 4 hrs at 32° C./6% CO₂.

Following incubation, 100 μL of complete Nano-Glo HiBiT Lytic Detection Reagent (Nano-Glo HiBiT Lytic Buffer with 1:50 Nano-Glo HiBiT Lytic Substrate and 1:100 LgBiT Protein; Promega cat. no. N₃O₄₀) was added. Cells were further incubated for 10 min at room temperature. Luminescence units (LU) were read on an EnVision plate reader (Perkin Elmer, 0.1 sec per well). Percent ITK remaining per sample was calculated as follows:

$$\% BTK remaining = 100 - [ \frac{Control LU - Sample LU}{Control LU} ] \times 100$$

Using Graphpad Prism, % ITK remaining values were plotted as a function of compound concentration. To determine DC₅₀ and Dₘₐₓ values, resulting curves were fit to the Prism curve-fitting equation “log(inhibitor) vs response−Variable slope (four parameters)” (reported best fit value IC₅₀ used as DC₅₀). ITK was measured with antibody-based MSD (Meso-Scale Discovery) assays shown below.

### ITK Degradation MSD (Meso Scale Discovery) Assay

Compound dilution series (7-point, 5-fold dilutions in DMSO, rows B—H with replicate in Column 1/2, 3/4, 5/6, 7, and 8 at 2000× the final required concentrations were prepared in 96-well plate (Falcon, cat. no. 353077). Row A, Column 1-8 were control DMSO. The 2000× solutions ranged from 2 mM to 128 nM (final assay concentration range 1 μM to 64 μM). The 2000×solutions were added to cells in 10 μL volume, for a final DMSO concentration of 0.5% and final assay compound concentration of 1×. For the cells, either human Jurkat (Clone E6-1 ATCC TIB-152) or Motl4 (ATCC CRL-1552), were plated at 1×10⁶ cells/mL, 100 μL/well (100×10⁴ cells/well) in complete RPMI (10% FBS, 1% L-glutamine). The cells were incubated with compounds 26-29 for 4 or 6 hrs at 32° C./6% CO₂.

Following incubation, plates were centrifuged at 1200 rpm for 5 min. Supernatant was removed and 50 μL of cell lysis buffer (MSD Tris lysis buffer (R60TX), complete Mini EDTA-free protease inhibitor (Sigma 11836170001), Protease Inhibitor Cocktail (Sigma, P2714), Phosphatase Inhibitor Cocktail 2 and 3 (Sigma, P5726 and P0044), Benzonase (Sigma, E1014)) was added to each well. Plate was sealed and shook at 4° C. for 30 min. Plate was centrifuged and 45 μL was removed for plate assay.

Assay plate preparation: Meso Scale Discovery (MSD) multi-array sm spot 96-well plates (Goat anti-Rabbit L⁴⁵-RA), were blocked with 3% BSA blocking buffer (3% Bovine Serum Albumin (Sigma A3059)+TBS 0.2% Tween-20) for one hour with gentle rocking at room temperature. Plate was then washed with 200 μL of 1×TBST (TBS 0.2% Tween-20) three times. After the last wash, all liquid was removed and 50 μL per well of capture antibody (abcam ITK Y402-ab32507) was added to plate at a 1:1000 dilution in blocking buffer (see above). Plate was sealed and rocked at room temperature for 2 hrs. Plate was then washed three times with 200 μL of 1×TBST. After the last wash, all liquid was removed and 45 μL of cell lysate (from above) was added to plate. Plate was sealed and rocked overnight at 4° C. The next day, cell lysates were removed, and assay plate was washed three times with 200 μL of 1×TBST. After the last wash, liquid was removed and detection antibody (CST ITK (2F12) #2380) was added at 50 μL per well at a dilution of 1:1000 in blocking buffer. Plate was sealed and rocked at room temperature for 2 hrs. Plate was washed three times with 200 μL of 1×TBST. After the last wash, all liquid was removed, MSD Mouse anti-Rabbit sulfo tag (R32AC-1) was diluted to 1:1000 in blocking buffer. 50 μL was added to each well on the plate. Plate was sealed and incubated at room temperature for one hour. Plate was washed three times with 200 μL of 1×TBST. After last wash, all liquid was removed and 150 μL of 1×MSD Read Buffer T (R92PC) was then added to each well for ECL read out.

For reading ECL signal, plate was read on a Meso Scale Discovery (MSD) MESO Sector S 600 plate reader. Percent ITK was then calculated as described below.

Percent ITK remaining per sample was calculated as follows:

$$\% ITK remaining = 100 - [ \frac{Control ECL - Sample ECL}{Control ECL} ] \times 100$$

Using Graphpad Prism, % ITK remaining values were plotted as a function of compound concentration. To determine DC₅₀ and Dₘₐₓ values, resulting curves were fit to the Prism curve-fitting equation “log(inhibitor) vs response−Variable slope (four parameters)” (reported best fit value IC₅₀ used as DC₅₀).

### Biological Example 2—In vivo Degradation After Oral Administration

### Western Assay for ITK Degradation in Mouse Splenocytes

Compounds 17 and 18 were administered to mice orally. After six hours, splenocyte cells were harvested. ITK was evaluated by Western blotting. Media was removed and cell pellets were lysed in 100 μL lysis buffer (RIPA buffer (Fisher, PI89901), complete Mini EDTA-free protease inhibitor (Sigma 11836170001), Protease Inhibitor Cocktail (Sigma, P2714), Phosphatase Inhibitor Cocktail 2 and 3 (Sigma, P5726 and P0044), Benzonase (Sigma, E1014)). Cells were lysed overnight at −20° C. Following thaw, cells were centrifuged for 10 min at 13000 rpm, then transferred to a new tube. Protein levels were determined by BCA Assay performed according to manufacturer's protocol (EMD Millipore, cat. no. 71285-3). Samples were combined with (4×) LDS Sample Buffer and (10×) Reducing Agent and H₂O to equally load 20 μg protein per lane of a 26-well NuPAGE 4-12% Bis-Tris protein gel (1.0 mm, Thermo cat. no. NP0326). Samples were separated by running gels at constant 150 V in NuPAGE MES SDS Running Buffer. Following electrophoresis, proteins were transferred to nitrocellulose membranes using an iBlot Gel Transfer Device and iBlot Gel Transfer Stacks (Thermo cat. no. IB21001 and IB301001) and transfer method P3 (20 V for 7 min). Membranes were blocked for one hour in 5% milk solution (TBS (0.2% Tween-20)). Following blocking, membranes were incubated with primary antibody (1:1000 CST ITK (2F12) #2380) overnight at 4° C. with gentle shaking. Blots were washed 2× in TBS (0.2% Tween-20), 30 min per wash. Following washes, blots were incubated in secondary HRP-conjugated antibody (Promega anti-Mouse IgG (H+L) HRP cat. no. W4021), 1:5000 in 5% milk solution (TBS (0.2% Tween-20)), for one hour at room temperature with gentle shaking. Blots were washed 2× in TBS (0.2% Tween-20), 30 min per wash. Blots were incubated with 1:1 mix of ECL reagents 1 & 2 (Amersham ECL Western Blotting Detection Reagent, cat. no. RPN2106) for 2 min at room temperature. Bands were visualized using a Protein Simple imager. Blots were then re-probed with a combination of anti-actin antibody (Sigma Monoclonal Mouse Anti-β-Actin (clone AC-15), cat. no. A5441) and secondary HRP-conjugated antibody (Promega anti-Mouse IgG (H+L) HRP, cat. no. W4021) and similar steps were taken for incubation, wash, detection, and visualization steps as above. The data was analyzed using Alpha View software. The densitometric reading for each sample band was normalized to that of the corresponding actin band per lane. Approximate % ITK remaining per sample was calculated as follows:

$$\% ITK normalized to actin = [ \frac{BTK band - blot background}{actin band - blot background} ]$$

$$\% ITK remaining = [ \frac{Normalized BTK Sample}{Mean normalized BTK Control} ] \times 100$$

Once % ITK remaining had been calculated for each sample, groups were averaged together to show a mean % ITK normalized to actin and relative to control.

### Biological Example 3—In Vivo Degradation after Oral Administration

Mouse PK Analysis Compounds 17 and 18 were administered to mice orally. After six hours or twenty-four hours, splenocyte cells were harvested. ITK was evaluated by Western blotting. Plasma concentrations were determined by LC/MS/MS. Plasma samples were protein precipitated by addition of 100 μl of acetonitrile containing 50 ng/ml of internal standard. The resulting mixture was vortexed and centrifuged at 4000 rpm for five minutes. An aliquot of the resultant supernatant (75 μl) was added to 75 μl of 0.1% formic acid in water to constitute the final sample for injection. Samples were injected on a Shimadzu Exion LC Binary Gradient AD Pump HLPC system connected to a Sciex QTRAP 6500+ mass spectrometer. 5 μL of sample was injected onto a Waters Acquity UPLC BEH C18 column 130A (2.1×30 mm, 1.7 m) at 40° C. utilizing a flow rate of 700 μL/minute.

Mobile Phase A was 0.1% formic acid in water and mobile phase B was 0.1% formic acid in acetonitrile. A linear gradient of 15-95% B over 1.0 minute was used. The mass spectrometer was operated in positive ion electrospray mode with multiple reaction monitoring for maximum sensitivity. Sciex Analyst software (version 1.6.3) was used for LC/MS/MS instrument control and acquisition. Compound concentration was determined against a standard curve of internal standard versus compound peak area ratio. Noncompartmental PK parameters were determined using Phoenix 32 software (version 8.2.0.4383) from Certara. Plasma concentrations from the LC/MS/MS analyses, dose, route of administration, and desired units were utilized for PK parameter calculations.

TABLE 2

| Compound | Timepoint (h) | Concentration (μM) |
|---|---|---|
| 17 | 2 | 0.0018 |
| 17 | 6 | 0.0045 |
| 18 | 2 | 0.0061 |
| 18 | 6 | 0.0162 |

### Noncompartmental PK Parameters

Cmax, Tmax, and AUClast were all calculated using a WinNonLin Phoenix 64 v 8.2.0.4383. Using non-compartmental analysis, Tmax, Cmax, and AUClast were determined as follows:

Tmax—Time of maximum observed concentration.

Cmax—Maximum observed concentration, occurring at time Tmax, as defined above.

AUClast—Area under the curve from the time of dosing to the time of the last measurable (positive) concentration (Tlast).

Results of biological assays are reported in Table 3A and 3B.

TABLE 3A

| | HiBiT: | HiBiT: |
|---|---|---|
| Compound No. | DC₅₀ (nM) | Dₘₐₓ (%) |
| 26 | 6.611 | NC |
| 27 | NC | 35 |
| 28 | 1.414 | 91 |
| 29 | 19.72 | 84 |
| 35 | NC | 35 |

TABLE 3B

| | MSD: | MSD: |
|---|---|---|
| Compound No. | DC₅₀ (nM) | Dₘₐₓ (%) |
| 1 | 251 | NA |
| 2 | 79.7 | NA |
| 4 | 13.6 | NA |
| 5 | 14.6 | NA |
| 6 | 14 | NA |
| 7 | 4.77 | NA |
| 8 | 49 | NA |
| 9 | 52.9 | NA |
| 10 | 22.2 | NA |
| 11 | 10.65 | NA |
| 12 | 114 | NA |
| 14 | 134 | NA |
| 15 | 54.7 | NA |
| 16 | 479 | NA |
| 17 | 4.509 | 95.72 |
| 18 | 1.472 | 95.082 |
| 19 | 2.604 | 95.856 |
| 20 | 1.275 | 94.564 |
| 21 | 64.73 | 95 |
| 22 | 3.782 | 88 |
| 23 | 13.27 | 91 |
| 24 | 92.7 | 93 |
| 30 | 38.78 | 86.27 |
| 31 | 30.13 | 88.76 |
| 32 | 20.15 | 63.41 |
| 33 | 27.89 | 94.497 |
| 34 | 10.81 | 94 |

### OTHER EMBODIMENTS

It is to be understood that the foregoing description is intended to illustrate and not limit the scope of this disclosure, which is defined by the scope of the appended claims. Other aspects, advantages, and modifications are within the scope of the following claims.

What is claimed is:

1. A compound of Formula (I) 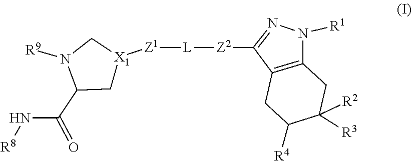
wherein
X¹ is C—H or nitrogen;
Z¹ is a bond, —CH₂, —C(O)—, —C(O)—N(R)—, or —N(R)—, wherein R is hydrogen or CH₃;
L is a linker according to -L¹-L²-L³-L⁴-L⁵-L⁶-L⁷- or -L⁷-L⁶-L⁵-L⁴-L³-L²-L¹-, wherein
L¹- is absent, —N(R¹⁰)—, —C(R¹¹)₂—, —C(O)—, —C₁₋₈ alkylene-, —C₂₋₈ alkynylene-, —C₆-C₁₀ aryl-, —C₆-C₁₀ heteroaryl-, -Q¹-, or -Q²-;
each -L²-, -L³-, -L⁴-, and -L⁵- is independently, absent, —N(R¹⁰)—, —C(R¹¹)₂—, —C(O)—, —O—, —(CH₂—CH₂—O)₁₋₈—, —C₁₋₈ alkylene-, —C₂₋₈ alkynylene-, —C₆-C₁₀ aryl-, —C₆-C₁₀ heteroaryl-, -Q¹-, -Q²-, or -Q³-;
each -L⁶- and -L⁷- is independently, absent, —N(R¹⁰)—, —C(R¹⁰)₂—, —C(O)—, —C(O)—N(R¹⁰)—, —N(R¹⁰)—C(O)—, or —C(R¹¹)₂—C(O)—N(R¹¹)—;
each -Q¹- is a three- to seven-membered heterocycloalkylene comprising at least one nitrogen;
each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring;
each -Q³- is a three- to six-membered cycloalkylene;
Z² is 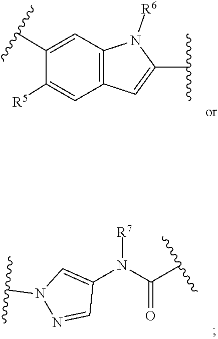
R¹ is hydrogen or methyl;
R² is methyl;
R³ is methyl or methylene bound to R⁴ to form a substituted cyclopropyl;
R⁴ is hydrogen or methylene bound to R³ to form a substituted cyclopropyl;
wherein when R³ and R⁴ form the substituted cyclopropyl, then the cyclopropyl is substituted with difluoro;
R⁵ is hydrogen or halogen;
each R⁶ is hydrogen or methyl;
each R⁷ is hydrogen or methyl;
R⁸ is alkyl, alkenyl, alkylene, alkynyl, aryl, cycloalkyl, heterocycloalkyl, heteroaryl, —S(O)(R^{q}), or —S(O)₂(R^{q}); wherein Ra is hydrogen, —OH, alkyl, alkenyl, alkynyl, aryl, heterocycle or heterocyclyl, or heteroaryl, wherein each alkyl, alkenyl, alkylene, alkynyl, aryl, cycloalkyl, heterocycloalkyl, and heteroaryl is optionally substituted;
R⁹ is hydrogen, optionally substituted C₁₋₈ alkyl, or -AA¹-AA²-R¹⁵, wherein each AA¹ and AA² is, independently, an amino acid residue or a 2-amino-2-cyclohexylacetic acid residue,
R¹⁵ is hydrogen or methyl;
each R¹⁰ is independently, hydrogen or methyl; and
each R¹¹ is, independently, hydrogen, methyl, aryl, or heteroaryl; or
a stereoisomer and/or pharmaceutical salt thereof.

2. The compound of claim 1, wherein X¹ is C—H.

3. The compound of claim 1 or 2, wherein Z² is

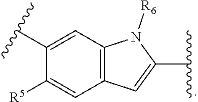

4. The compound of claim 1 or 2, wherein Z² is

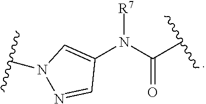

5. The compound of any one of the previous claims, wherein R² is methyl; R³ is methyl; and R⁴ is hydrogen.

6. The compound of any one of the previous claims, wherein R² is hydrogen; and R³ and R⁴ form difluorocyclopropane.

7. The compound of any one of the previous claims, wherein R⁸ is tetrahydronaphthyl.

8. The compound of claim 7, wherein R⁸ has the following structure

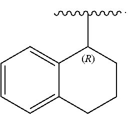

9. The compound of any one of the previous claims, wherein R⁹ is -AA¹-AA²-R¹⁵.

10. The compound of claim 9, wherein AA¹ is a phenylalanine or 2-amino-2-cyclohexylacetic acid residue and AA² is an alanine residue.

11. The compound of claim 9, wherein R⁹ has the following structure

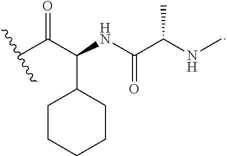 [SMILES: *C(=O)[C@@H](NC(=O)[C@H](C)NC)C1CCCCC1]

12. The compound of any one of the previous claims, wherein Z¹ is —N—H.

13. The compound of any one of the previous claims, wherein Z¹ is a bond.

14. A compound of Formula (II) 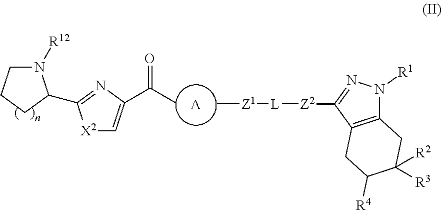
wherein
X² is —CH₂—, —N—R, oxygen, or sulfur, wherein R is hydrogen or CH₃;
A is phenyl or C₅₋₆ heteroaryl;
Z¹ is a bond, —CH₂—, —C(O)—, —C(O)—N(R)—, —N(R)—, or —O—, wherein R is hydrogen or CH₃;
L is a linker according to -L¹-L²-L³-L⁴-L⁵-L⁶-L⁷- or -L⁷-L⁶-L⁵-L⁴-L³-L²-L¹-, wherein
L¹- is absent, —N(R¹⁰)—, —C(R¹¹)₂—, —C(O)—, —C₁₋₈ alkylene-, —C₂₋₈ alkynylene-, —C₆-C₁₀ aryl-, —C₆-C₁₀ heteroaryl-, -Q¹-, or -Q²-;
each -L²-, -L³-, -L⁴-, and -L⁵- is independently, absent, —N(R¹⁰)—, —C(R¹¹)₂—, —C(O)—, —O—, —(CH₂—CH₂—O)₁₋₈—, —C₁₋₈ alkylene-, —C₂₋₈ alkynylene-, —C₆-C₁₀ aryl-, —C₆-C₁₀ heteroaryl-, -Q¹-, -Q²-, or -Q³-;
each -L⁶- and -L⁷- is independently, absent, —N(R¹⁰)—, —C(R¹⁰)₂—, —C(O)—, —C(O)—N(R¹⁰)—, —N(R¹⁰)—C(O)—, or —C(R¹¹)₂—C(O)—N(R¹⁰)—;
each -Q¹- is a three- to seven-membered heterocycloalkylene comprising at least one nitrogen;
each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring;
each -Q³- is a three- to six-membered cycloalkylene;
Z² is 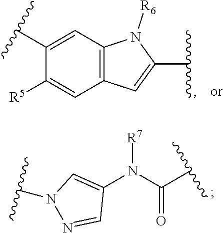
R¹ is hydrogen or methyl;
R² is methyl;
R³ is methyl or methylene bound to R⁴ to form a substituted cyclopropyl;
R⁴ is hydrogen or methylene bound to R³ to form a substituted cyclopropyl;
wherein when R³ and R⁴ form the substituted cyclopropyl, then the cyclopropyl is substituted with difluoro;
R⁵ is hydrogen or halogen;
each R⁶ is hydrogen or methyl;
each R⁷ is hydrogen or methyl;
each R¹⁰ is, independently, hydrogen or methyl; and
each R¹¹ is, independently, hydrogen, methyl, aryl, or heteroaryl;
R¹² is optionally substituted C₁₋₈ alkyl, or -AA¹-AA²-R⁵, wherein each AA¹ and AA² is, independently, an amino acid residue or a 2-amino-2-cyclohexylacetic acid residue;
R¹⁵ is hydrogen or methyl; and
n is zero or one; or
a stereoisomer and/or pharmaceutical salt thereof.

15. The compound of claim 14, wherein X² is sulfur.

16. The compound of claim 14 or 15, wherein Z² is

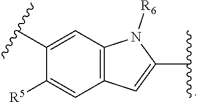

17. The compound of claim 14 or 15, wherein Z² is

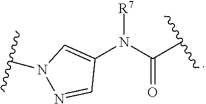

18. The compound of any one of claims 14-17, wherein R² is methyl; R³ is methyl; and R⁴ is hydrogen.

19. The compound of any one of claims 14-17, wherein R² is hydrogen; and R³ and R⁴ form difluorocyclopropane.

20. The compound of any one of claims 14-19, wherein A is phenyl.

21. The compound of any one of claims 14-20, wherein R¹² is -AA¹-AA²-R⁵.

22. The compound of claim 21, wherein AA¹ is a phenylalanine or 2-amino-2-cyclohexylacetic acid residue and AA² is an alanine residue.

23. The compound of claim 21, wherein R¹² has the following structure

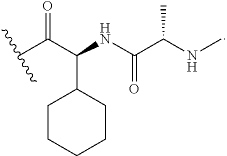 [SMILES: *C(=O)[C@@H](NC(=O)[C@H](C)NC)C1CCCCC1]

24. The compound of any one of claims 14-23, wherein Z¹ is oxygen.

25. A compound of Formula (III) 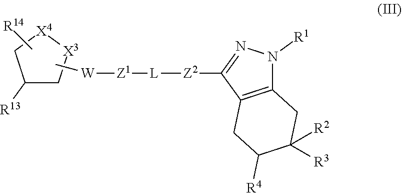
wherein
X³ is nitrogen and X⁴ is C—H;
Z¹ is a bond, —CH₂—, —C(O)—, —C(O)—N(R)—, or —N(R)—, wherein R is hydrogen or CH₃;
L is a linker according to -L¹-L²-L³-L⁴-L⁵-L⁶-L⁷- or -L⁷-L⁶-L⁵-L⁴-L³-L²-L¹-, wherein
L¹- is absent, —N(R¹⁰)—, —C(R¹¹)₂—, —C(O)—, —C₁₋₈ alkylene-, —C₂₋₈ alkynylene-, —C₆-C₁₀ aryl-, —C₆-C₁₀ heteroaryl-, -Q¹-, or -Q²-;
each -L²-, -L³-, -L⁴-, and -L⁵- is independently, absent, —N(R¹⁰)—, —C(R¹¹)₂—, —C(O)—, —O—, —(CH₂—CH₂—O)₁₋₈—, —C₁₋₈ alkylene-, —C₂₋₈ alkynylene-, —C₆-C₁₀ aryl-, substituted —C₆-C₁₀ aryl-, —C₄-C₁₀ heteroaryl-, -Q¹-, -Q²-, or -Q³-;
each -L⁶- and -L⁷- is independently, absent, —N(R¹⁰)—, —C(R¹⁰)₂—, —C(O)—, —C(O)—N(R¹⁰)—, —N(R¹⁰)—C(O)—, —C₄-C₁₀ heteroaryl-, 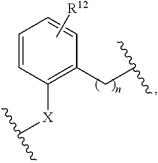
or —C(R¹¹)₂—C(O)—N—(R¹⁰)—;
each -Q¹- is a three- to seven-membered heterocycloalkylene comprising at least one nitrogen;
each -Q²- is a five to thirteen-membered bicyclic heterocycloalkylene comprising at least one nitrogen, wherein the five- to thirteen-membered bicyclic heterocycloalkylene is optionally a spiro bicyclic heterocycloalkylene ring;
each -Q³- is a three- to six-membered cycloalkylene;
W is 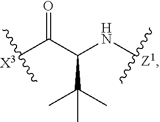
—C(O)—, or 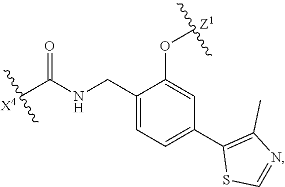
wherein 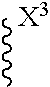
designates attachment to X³, wherein 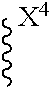
designates attachment to X⁴, and wherein 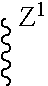
designates attachment to Z¹;
Z² is 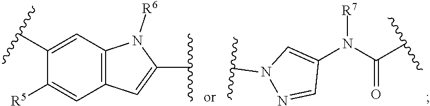
R¹ is hydrogen or methyl;
R² is methyl;
R³ is methyl or methylene bound to R⁴ to form a substituted cyclopropyl;
R⁴ is hydrogen or methylene bound to R³ to form a substituted cyclopropyl;
wherein when R³ and R⁴ form the substituted cyclopropyl, then the cyclopropyl is substituted with difluoro;
R⁵ is hydrogen or halogen;
each R⁶ is hydrogen or methyl;
each R⁷ is hydrogen or methyl;
each R¹⁰ is, independently, hydrogen or methyl; and
each R¹¹ is, independently, hydrogen, methyl, aryl, substituted aryl, or heteroaryl;
R¹² is halo;
R¹³ is hydrogen, —OH, halogen, —NH₂, —C₁-C₃ alkyl, —C₂-C₆ alkenyl, —C₂-C₆ alkynyl, —C₁-C₃ alkoxy, —C₁-C₃ thioalkyl, —C₁-C₃ alkylamine, —C₆-C₁₀ aryl, cycloalkyl, heterocycloalkyl, or heteroaryl;
R¹⁴ is —C(O)—N(H)—C₆-C₁₀ aralkyl, 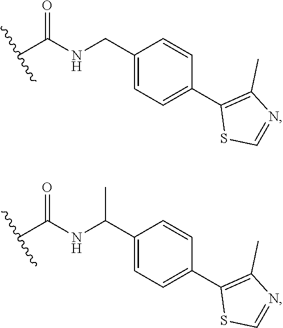 [SMILES: *C(=O)NCc1ccc(-c2scnc2C)cc1 ; *C(=O)NC(C)c1ccc(-c2scnc2C)cc1]
C(O)—CH(t-butyl)-N(H)—C₃-C₆ cycloalkyl, —C(O)—CH(t-butyl)-N(H)C(O)—C₃-C₆ cycloalkyl, 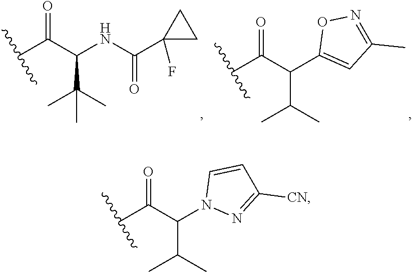
or -AA¹-AA²-R¹⁵, wherein each AA¹ and AA² is, independently, an amino acid residue or a 2-amino-2-cyclohexylacetic acid residue, and
R¹⁵ is hydrogen or methyl;
X is oxygen or sulfur;
n is an integer from one to eight; or
a stereoisomer and/or pharmaceutical salt thereof.

26. The compound of claim 25, wherein X³ is attached to W, and X⁴ is attached to R¹⁴.

27. The compound of claim 25, wherein X⁴ is attached to W, and X³ is attached to R¹⁴.

28. The compound of any one of claims 25-27, wherein Z² is

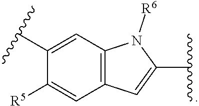

29. The compound of any one of claims 25-27, wherein Z² is

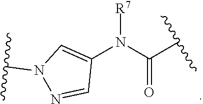

30. The compound of any one of claims 25, 26, 28, or 29, wherein W is 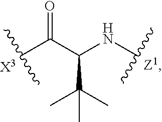
and R¹⁴ is S

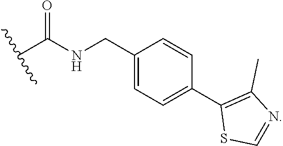 [SMILES: *C(=O)NCc1ccc(-c2scnc2C)cc1]

31. The compound of any one of claims 25 or 27-29, wherein W is 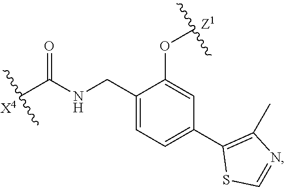
and R¹⁴ is

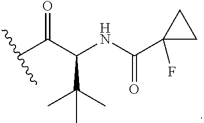 [SMILES: *C(=O)[C@@H](NC(=O)C1(F)CC1)C(C)(C)C]

32. The compound of any one of claims 25-31, wherein R² is methyl; R³ is methyl; and R⁴ is hydrogen.

33. The compound of any one of claims 25-31, wherein R² is hydrogen; and R³ and R⁴ form difluorocyclopropane.

34. The compound of any one of claims 25-33, wherein R¹³ is —OH.

35. The compound of any one of claims 25-34 wherein Z¹ is a bond.

36. The compound of claim 25, having the following Formula (III-I) 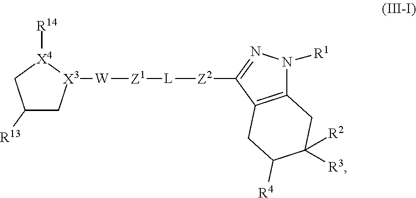
or
a stereoisomer and/or pharmaceutical salt thereof.

37. The compound of claim 25, having the following Formula (III-II) 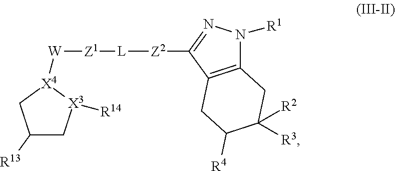
or
a stereoisomer and/or pharmaceutical salt thereof.

38. The compound of claim 1 Formula (I), claim 14 Formula (II), claim 25 Formula (III), claim 36 Formula (III-I), or claim 37 Formula (III-II), wherein L comprises at least one -Q¹- according to 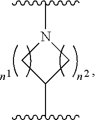
further wherein n¹ is one or two, and n² is one or two.

39. The compound of claim 1 Formula (I), claim 14 Formula (II), claim 25 Formula (III), claim 36 Formula (III-I), or claim 37 Formula (III-II), wherein L is selected from
a. -Q¹-N(Me)-CH₂-Q¹-C(O)—;
b. —N(Me)-Q¹-CH₂-Q¹-C(O)—;
c. -Q²-CH₂-Q¹-C(O)—;
d. -Q¹-CH₂-Q¹-C(O)—;
e. -Q¹-Q¹-C(O)—;
f. -Q¹-CH₂—N(Me)-Q¹-C(O)—;
g. -Q¹-CH₂-Q¹-CH₂—C(O)—N(Me)-;
h. -Q¹-CH₂-Q¹-;
i. -Q¹-CH₂-Q²-;
j. -Q¹-CH₂—CH₂-Q¹-;
k. -Q¹-CH₂—CH₂-Q²-;
l. -Q¹-C(O)-Q¹-;
m. -Q¹-C(O)-Q₂-;
n. -Q¹-CH₂-Q¹-N(Me)-C(O)—;
o. —CH₂—CH₂—CH₂—CH₂-Q¹-C(O)—;
p. -Q¹-C(O)-;
q. -Q¹-C(O)-Q¹-CH(C₆H₅)—;
r. —C≡CCH₂-Q¹-C(O)—;
s. -Q¹-CH₂-Q¹-NH—C(O)—;
t. —CH₂—CH₂—CH₂-Q¹-C(O)—;
u. -Q¹-CH₂-Q¹-C(Me)-C(O)—N(Me)-;
v. —CH₂-Q¹-;
w. -Q¹-C(O)-Q¹-CH₂—;
x. —N(H)—(CH₂)₅—C(O)-Q¹-CH(C₆H₅)—;
y. —N(H)—(CH₂)₂—O—(CH₂)₂—C(O)-Q¹-CH(C₆H₅)—;
z. -Q¹-(CH₂)₃—C(O)-Q¹-CH(C₆H₅)—;
aa. -Q²-C(O)-Q¹-CH(C₆H₅)—;
bb. -Q²-CH₂—C(O)-Q¹-CH(C₆H₅)—;
cc. -Q²-(CH₂)₃—C(O)-Q¹-CH(C₆H₅)—;
dd. -Q²-(CH₂)₂—C(O)-Q¹-CH(C₆H₅)—;
ee. —(CH₂)₆-Q¹-CH(C₆H₅)—;
ff. -Q¹-Q¹-C(O)-Q¹-CH(C₆H₅)—;
gg. -Q¹-CH₂—C(O)-Q¹-CH(C₆H₅)—;
hh. -Q¹-(CH₂)₂—C(O)-Q¹-CH(C₆H₅)—;
ii. —(CH₂)₃—C(O)-Q¹-CH(C₆H₅)—;
jj. —(CH₂)₄—C(O)-Q¹-CH(C₆H₅)—;
kk. —(CH₂)₅—C(O)-Q¹-CH(C₆H₅)—;
ll. —(CH₂)₆—C(O)-Q¹-CH(C₆H₅)—;
mm. —(CH₂)₃-Q¹-CH₂—C(O)-Q¹-CH(C₆H₅)—;
nn. —(CH₂)₃—O-Q³-C(O)-Q¹-CH(C₆H₅)—;
oo. —(CH₂)₃—O—(CH₂)₂—C(O)-Q¹-CH(C₆H₅)—;
pp. —(CH₂)₃—O—(CH₂)₂—C(O)-Q¹-CH(pyrid-2-yl)-;
qq. —(CH₂)₄-Q¹-CH(C₆H₅)—;
rr. —(CH₂)₅-Q¹-CH(C₆H₅)—;
ss. —(CH₂)₆-Q¹-CH(pyrid-2-yl)-;
tt. —(CH₂)₇-Q¹-CH(C₆H₅)—;
uu. —(CH₂)₇-Q¹-CH(Me)-C(O)—N(Me)-;
vv. —N(H)—(CH₂)₂—O—(CH₂)₂-Q¹-CH(Me)-C(O)—N(Me)-;
ww. —(CH₂)₃—O—(CH₂)₂—C(O)-Q¹-CH(Me)-C(O)—N(Me)-;
xx. —N(H)—(CH₂)₂—O—(CH₂)₂-Q¹-CH(C₆H₅)—;
yy. —N(H)—(CH₂)₂—[O—(CH₂)₂]₂—C(O)-Q¹-CH(C₆H₅)—;
zz. —N(H)—(CH₂)₂—[O—(CH₂)₂]₃—C(O)-Q¹-CH(C₆H₅)—;
aaa. —N(H)—(CH₂)₂—[O—(CH₂)₂]₄—C(O)-Q¹-CH(C₆H₅)—;
bbb. —N(H)—(CH₂)₂—[O—(CH₂)₂]₅—C(O)-Q¹-CH(C₆H₅)—;
ccc. —N(H)—(CH₂)₂—[O—(CH₂)₂]₆—C(O)-Q¹-CH(C₆H₅)—;
ddd. —N(H)—(CH₂)₂—[O—(CH₂)₂]₇—C(O)-Q¹-CH(C₆H₅)—;
eee. —N(H)—(CH₂)₂—[O—(CH₂)₂]s-C(O)-Q¹-CH(C₆H₅)—;
ooo. —N(H)-Q³-O—(CH₂)₂—CH₂—;
ppp. —N(H)—(CH₂)₃-Q¹-(CH₂)₂—;
qqq. —C(O)—N(H)—[(CH₂)₃—O]₃—(CH₂)₂—NH—;
rrr. —C(O)—N(H)—[(CH₂)₃—O]₃—(CH₂)₂—;
sss. -Q¹-C(O)—[(CH₂)₂—O]₃—(CH₂)₂—NH—;
ttt. -Q¹-(CH₂)₃—O—CH₂—;
uuu. -Q¹-C(O)—(C₆H₆)—CH₂—;
vvv. -Q¹-(2-pyridyl)-O—CH₂— or 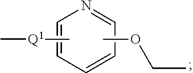
www. —N(H)-Q³-X-(2-pyridyl)-O— or 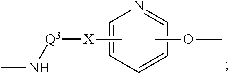
xxx. —N(H)-Q³-X-(4-pyridyl)- or 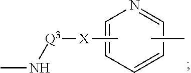
yyy. —N(H)—(CH₂)₂-Q³-X-(2-pyridyl)-O—CH₂— or 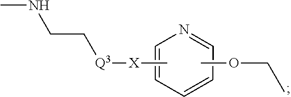
zzz. —C≡C—(CH₂)₂-Q¹-;
aaaa. -Q¹-;
bbbb. —C(O)—(CH₂—CH₂—O)—(CH₂)₂—C(O)-Q¹-CH(C₆H₅)—;
cccc. —N(H)—C(O)—(CH₂—CH₂—O)₄—(CH₂)₂—C(O)-Q¹-CH(C₆H₅)—;
dddd. —N(H)—C(O)—(CH₂—CH₂—O)₅—(CH₂)₂—C(O)-Q¹-CH(C₆H₅)—;
eeee. —(CH₂—CH₂—O)₅—(CH₂)₂—C(O)-Q¹-CH(C₆H₅)—;
ffff. —(CH₂—CH₂—O)—(CH₂)₂—C(O)-Q¹-CH(C₆H₅)—;
gggg. —(CH₂—CH₂—O)₄—(CH₂)₂—C(O)-Q¹-CH(C₆H₅)—;
hhhh. —(CH₂)₄-Q¹-CH(C₆H₅)—;
iiii. —(CH₂)₃-Q¹-CH(C₆H₅)—;
jjjj. —(CH₂)₆-Q¹-CH(C₆H₅)—;
kkkk. —(CH₂)₅-Q¹-CH(C₆H₅)—;
llll. —(CH₂—CH₂—O)—(CH₂)₂-Q¹-CH(C₆H₅)—;
mmmm. —C(O)—(CH₂—CH₂—O)₄—(CH₂)₂-Q¹-CH(C₆H₅)—;
nnnn. —C(O)—(CH₂—CH₂—O)₅—(CH₂)₂—C(O)-Q¹-CH(C₆H₅)—;
oooo. —C(O)—(CH₂—CH₂—O)₆—(CH₂)₂—C(O)-Q¹-CH(C₆H₅)—;
pppp. —C(O)—(CH₂—CH₂—O)₃—(CH₂)₂—C(O)-Q¹-CH(C₆H₅)—;
ssss. —(CH₂)₅—C(O)-Q¹-CH(C₆H₅)—;
tttt. —(CH₂—CH₂—O)₃—(CH₂)₂—C(O)-Q¹-CH(C₆H₅)—;
uuuu. —(CH₂)₇—C(O)-Q¹-CH(C₆H₅)—;
vvvv. —C(O)-pyrimidine-Q¹-C(O)-Q¹-CH(C₆H₅)— or 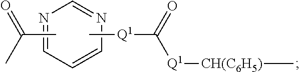
wwww. —C(O)—(CH₂)-Q¹-3-pyridyl-C(O)-Q¹-CH(C₆H₅)— or 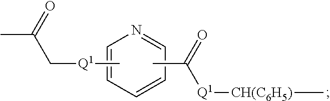
xxxx. —C(O)-2-pyridyl-Q¹-CH₂—C(O)-Q¹-CH(C₆H₅)— or 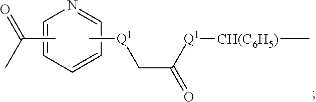
yyyy. —C(O)—(CH₂)-Q¹-CH₂—C(O)-Q¹-CH(C₆H₅)—;
zzzz. —(CH₂)₂—C(O)-Q¹-CH(C₆H₅)—;
aaaaa. —C(O)—(CH₂)₂-Q¹-(CH₂)₂—C(O)-Q¹-CH(C₆H₅)—;
bbbbb. —C(O)—(CH₂)₉—C(O)-Q¹-CH(C₆H₅)—;
ccccc. —C(O)—(CH₂)₇—C(O)-Q¹-CH(C₆H₅)—;
ddddd. —C(O)—(CH₂)₅—C(O)-Q¹-CH(C₆H₅)—;
eeeee. —C(O)—(CH₂—CH₂—O)₂—(CH₂)₂—C(O)-Q¹-CH(C₆H₅)—;
fffff. —C(O)-2-pyridyl-Q¹-C(O)-Q¹-CH(C₆H₅)— or 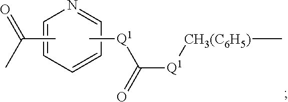
ggggg. —CH₂—C(O)-Q¹-CH(C₆H₅)—;
hhhhh. —(CH₂)₃—C(O)-Q¹-CH(C₆H₅)—;
iiiii. —(CH₂)₄—C(O)-Q¹-CH(C₆H₅)—; and
jjjjj. -Q¹-4-pyridyl-Q¹-CH(C₆H₅)— or 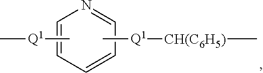
wherein X is oxygen or sulfur.

40. The compound of claim 1 Formula (I), claim 14 Formula (II), claim 25 Formula (III), claim 36 Formula (III-I), or claim 37 Formula (III-II), wherein L comprises at least one -Q¹- selected from the group consisting of

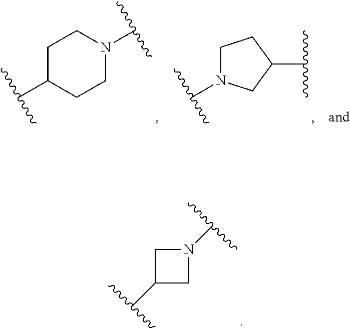 [SMILES: *C1CCN(*)CC1 ; *C1CCN(*)C1 ; *C1CN(*)C1]

41. The compound of claim 1 Formula (I), claim 14 Formula (II), claim 25 Formula (III), claim 36 Formula (III-I), or claim 37 Formula (III-II), wherein L comprises at least one -Q²- according to 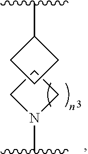
wherein n³ is one or two.

42. The compound of claim 1 Formula (I), claim 14 Formula (II), claim 25 Formula (III), claim 36 Formula (III-I), or claim 37 Formula (III-II), wherein L comprises at least one -Q²- according to

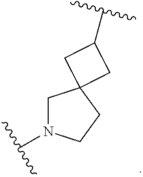 [SMILES: *C1CC2(CCN(*)C2)C1]

43. The compound of claim 1 Formula (I), claim 14 Formula (II), claim 25 Formula (III), claim 36 Formula (III-I). or claim 37 Formula (III-II). wherein L comprises at least one -Q²- according to 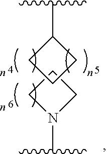
wherein n⁴ is one or two, n⁵ is one or two, and n⁶ is one or two.

44. The compound of claim 1 Formula (I), claim 14 Formula (II), claim 25 Formula (III), claim 36 Formula (III-I), or claim 37 Formula (III-II), wherein L comprises at least one -Q²- according to

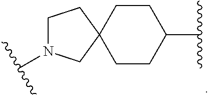 [SMILES: *C1CCC2(CC1)CCN(*)C2]

45. The compound of claim 1 Formula (I), claim 14 Formula (II), claim 25 Formula (III), claim 36 Formula (III-I), or claim 37 Formula (III-II), wherein L comprises at least one -Q²- according to 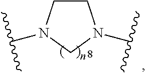
wherein n⁸ is one or two.

46. The compound of claim 1 Formula (I), claim 14 Formula (II), claim 25 Formula (III), claim 36 Formula (III-I), or claim 37 Formula (III-II), wherein L comprises at least one -Q²- according to

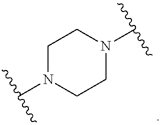 [SMILES: *N1CCN(*)CC1]

47. The compound of claim 1 Formula (I), claim 14 Formula (II), claim 25 Formula (III), claim 36 Formula (III-I), or claim 37 Formula (III-II), wherein L comprises at least one -Q²- according to 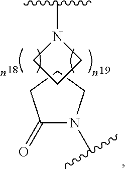
wherein n¹⁸ and n¹⁹ is two, or n¹⁸ is two and n¹⁹ is three, or n¹⁸ is three and n¹⁹ is two.

48. The compound of claim 1 Formula (I), claim 14 Formula (II), claim 25 Formula (III), claim 36 Formula (III-I), or claim 37 Formula (III-II), wherein L comprises at least one -Q²- according to

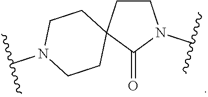 [SMILES: *N1CCC2(CC1)CCN(*)C2=O]

49. The compound of claim 1 Formula (I), claim 14 Formula (II), claim 25 Formula (III), claim 36 Formula (III-I), or claim 37 Formula (III-II), wherein L comprises at least one -Q²- according to 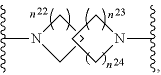
wherein n²² is zero to two, n²³ is zero to two, and n²⁴ is one or two.

50. The compound of claim 1 Formula (I), claim 14 Formula (II), claim 25 Formula (III), claim 36 Formula (III-I), or claim 37 Formula (III-II), wherein n²² is two and each n²³ and n²⁴ is one; or
n²² is two and each n²³ and n²⁴ is two.

51. The compound of claim 1 Formula (I), claim 14 Formula (II), claim 25 Formula (III), claim 36 Formula (III-I), or claim 37 Formula (III-II), wherein L comprises at least one -Q²- according to

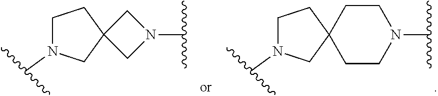 [SMILES: *N1CCC2(C1)CN(*)C2 ; *N1CCC2(CC1)CCN(*)C2]

52. The compound of claim 1 Formula (I), claim 14 Formula (II), claim 25 Formula (III), claim 36 Formula (III-I) or claim 37 Formula (III-II), wherein L comprises at least one -Q²- according to

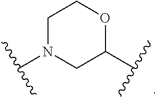 [SMILES: *C1CN(*)CCO1]

53. The compound of claim 1 Formula (I), claim 14 Formula (II), claim 25 Formula (III), claim 36 Formula (III-I) or claim 37 Formula (III-II), wherein L comprises at least one -Q³- according to 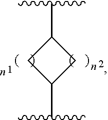
wherein n¹ is one or two, and n² is one or two.

54. The compound of claim 1 Formula (I), claim 14 Formula (II), claim 25 Formula (III), claim 36 Formula (III-I). or claim 37 Formula (III-II). wherein L comprises at least one -Q³- selected from the group consisting of

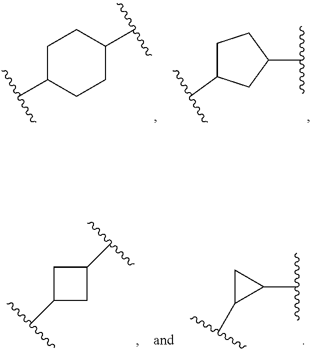 [SMILES: *C1CCC(*)CC1 ; *C1CCC(*)C1 ; *C1CC(*)C1 ; *C1CC1*]

55. The compound of claim 1 Formula (I), claim 14 Formula (II), claim 25 Formula (III), claim 36 Formula (III-I), or claim 37 Formula (III-II), wherein the linker L is selected from 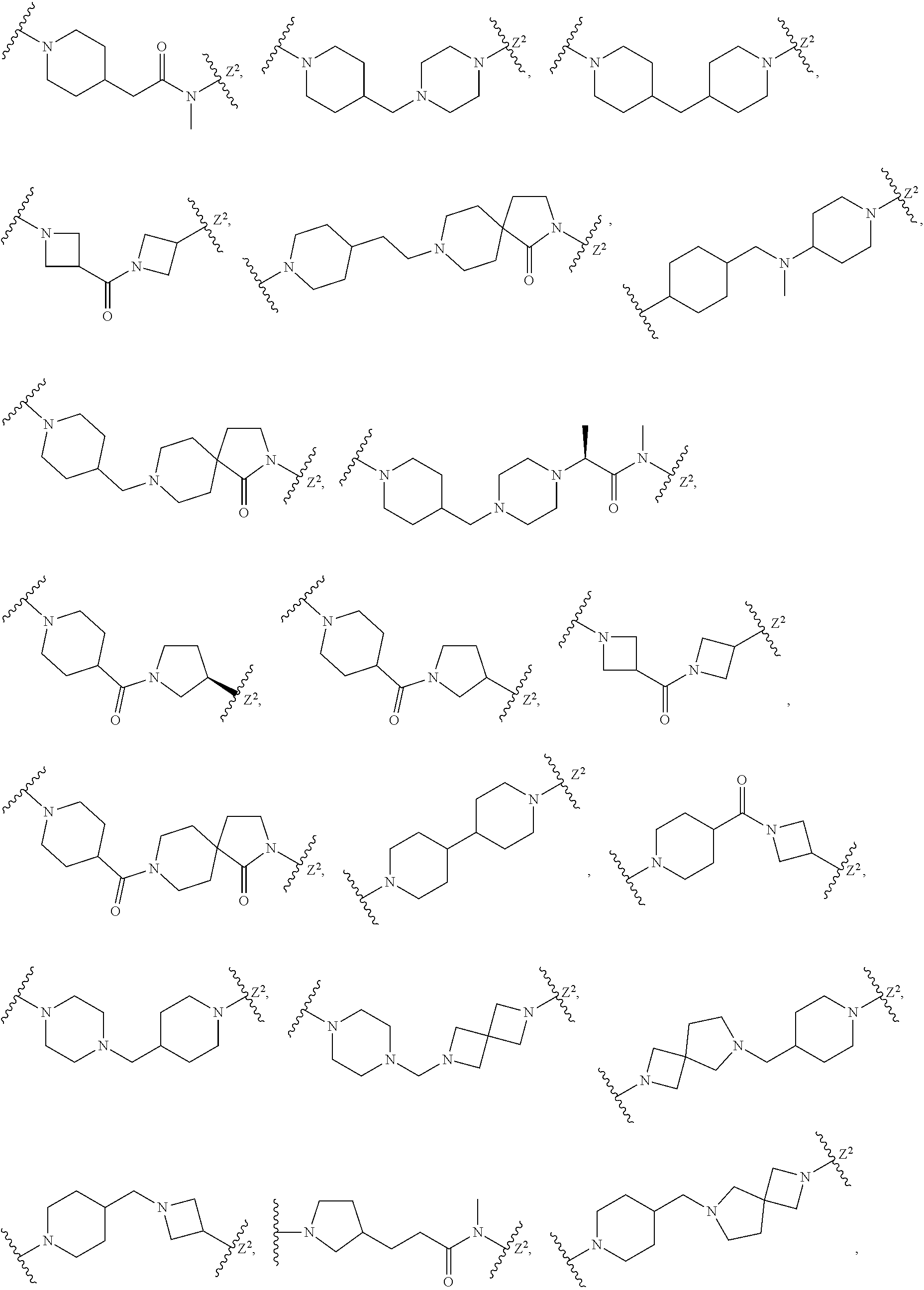 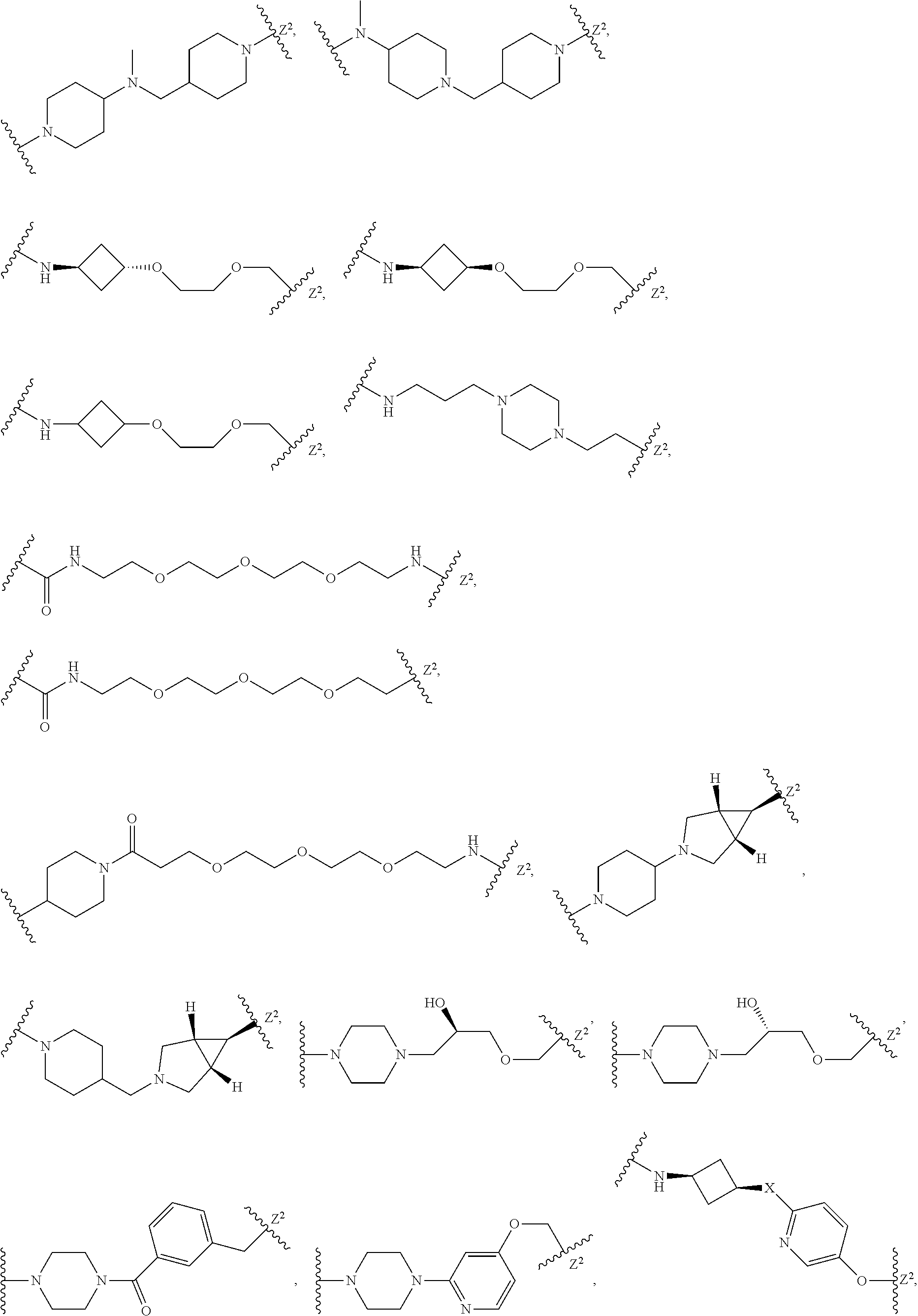 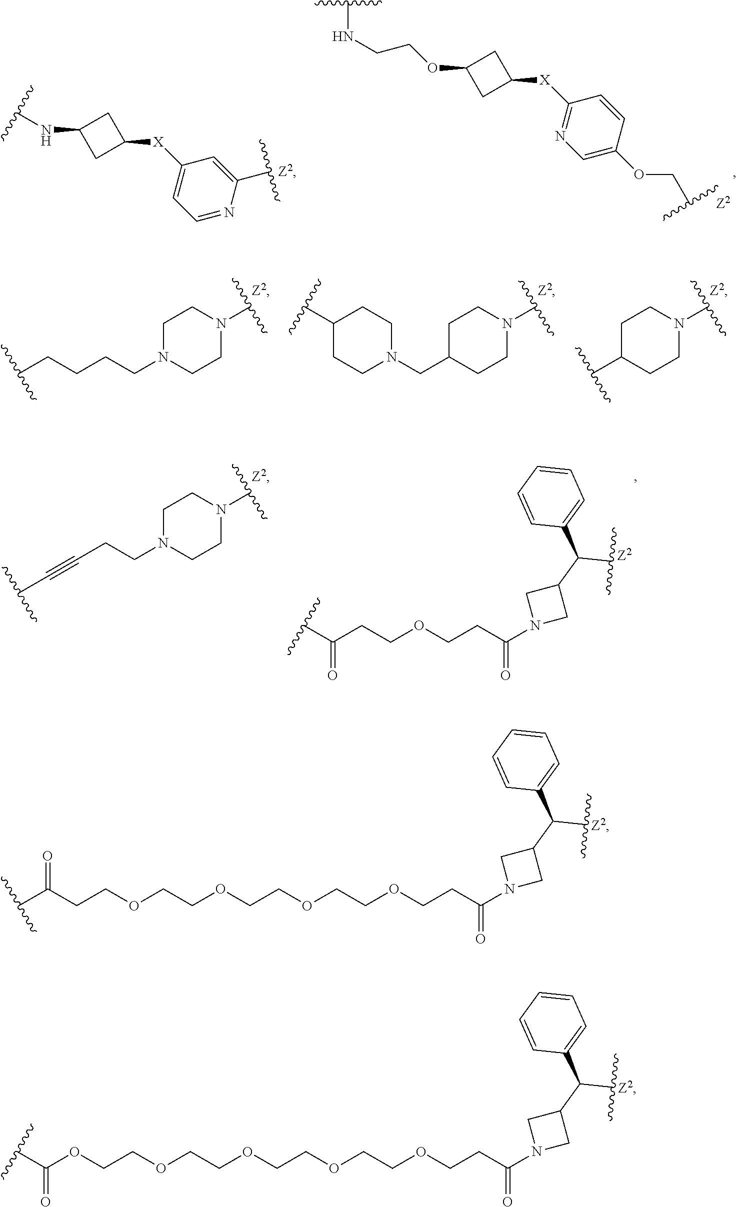 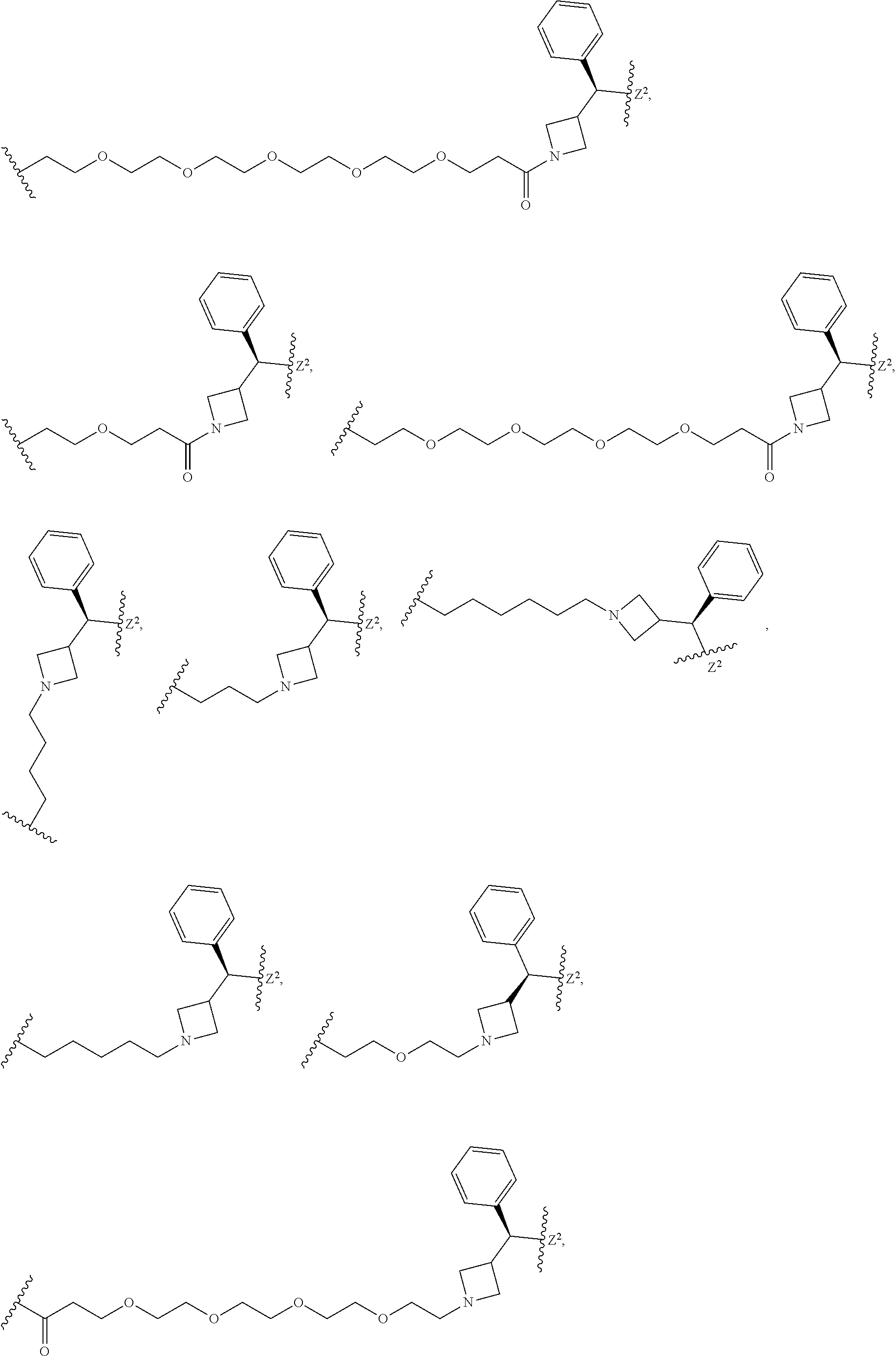 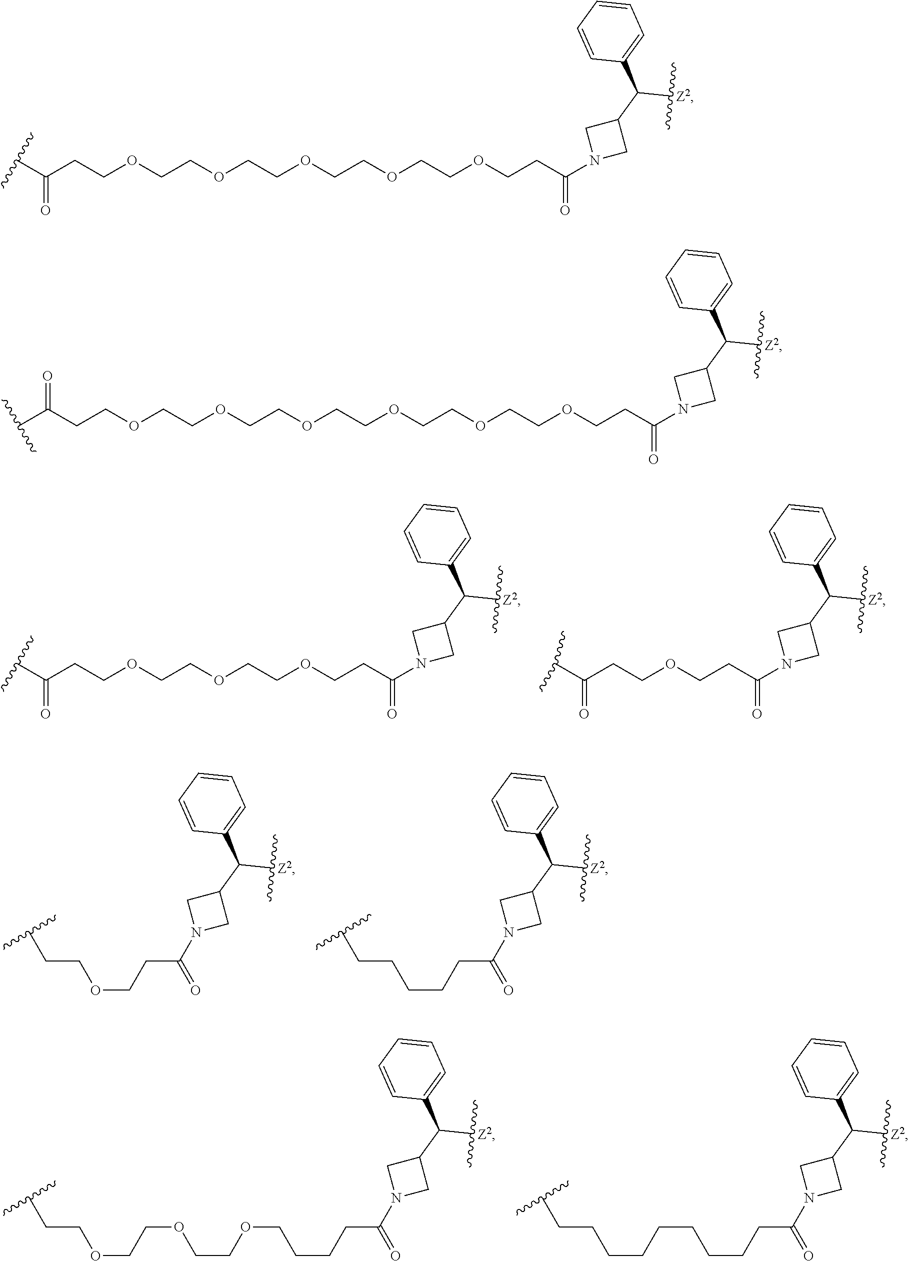 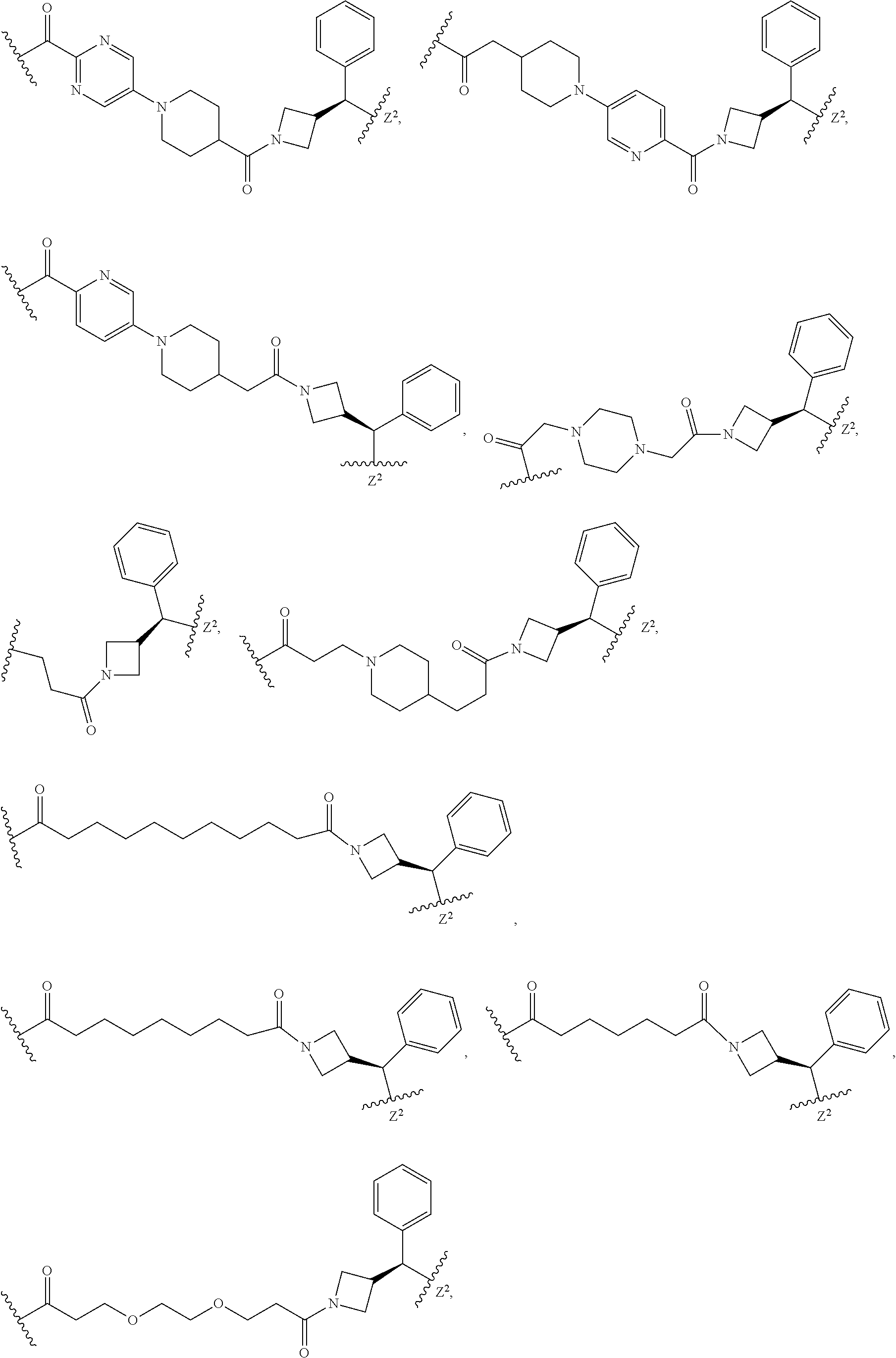 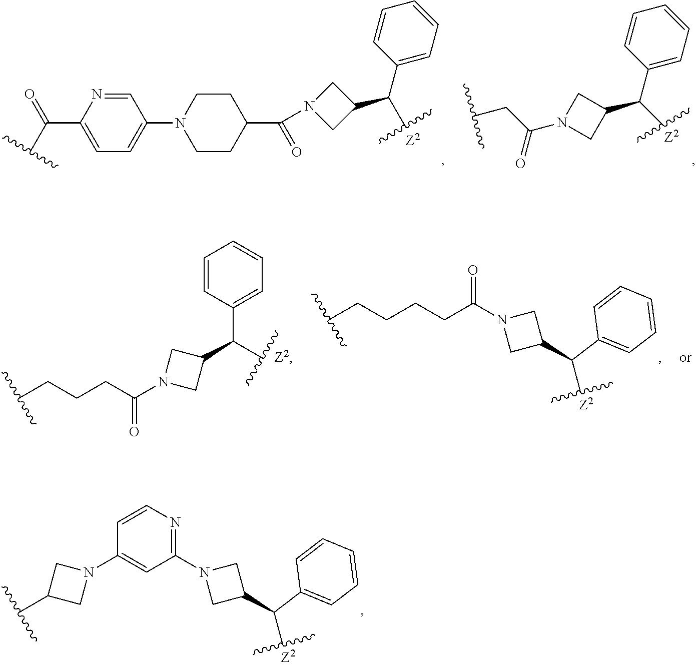 [SMILES: *C(=O)c1ccc(N2CCC(C(=O)N3CC([C@H](*)c4ccccc4)C3)CC2)cn1 ; *CC(=O)N1CC([C@H](*)c2ccccc2)C1 ; *CCCC(=O)N1CC([C@H](*)c2ccccc2)C1 ; *CCCCC(=O)N1CC([C@H](*)c2ccccc2)C1 ; *C1CN(c2ccnc(N3CC([C@H](*)c4ccccc4)C3)c2)C1]
wherein 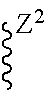
designates attachment to Z² and wherein X is oxygen or sulfur.

56. The compound of claim 1, having the following Formula (Ia) 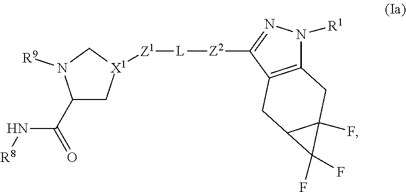
or
a stereoisomer and/or pharmaceutical salt thereof.

57. The compound of claim 56, having the following Formula (Ib) 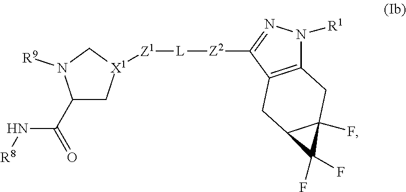
or
a stereoisomer and/or pharmaceutical salt thereof.

58. The compound of claim 14, having the following Formula (IIa) 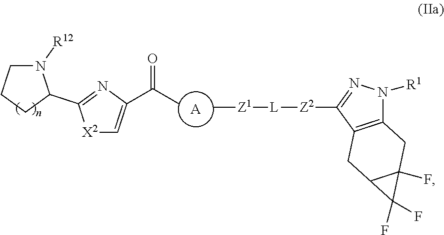
or
a stereoisomer and/or pharmaceutical salt thereof.

59. The compound of claim 58, having the following Formula (IIb) 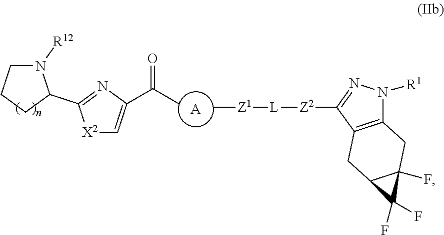
or
a stereoisomer and/or pharmaceutical salt thereof.

60. The compound of claim 36, having the following Formula (III-Ia) 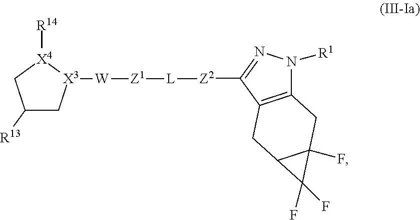
or
a stereoisomer and/or pharmaceutical salt thereof.

61. The compound of claim 60, having the following Formula (III-Ib) 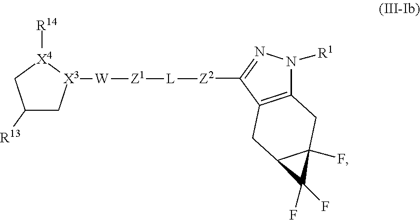
or
a stereoisomer and/or pharmaceutical salt thereof.

62. The compound of claim 37, having the following Formula (III-IIa) 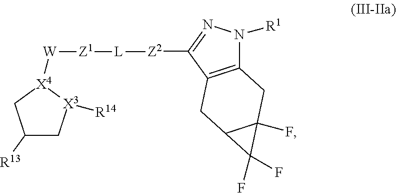
or
a stereoisomer and/or pharmaceutical salt thereof.

63. The compound of claim 62, having the following Formula (III-IIb) 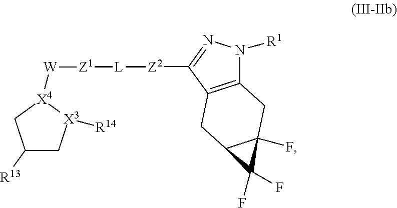
or
a stereoisomer, and/or pharmaceutical salt thereof.

64. The compound of any one of the previous claims, wherein the compound is selected from

| Compound | |
|---|---|
| No. | Structure |
| 1 | 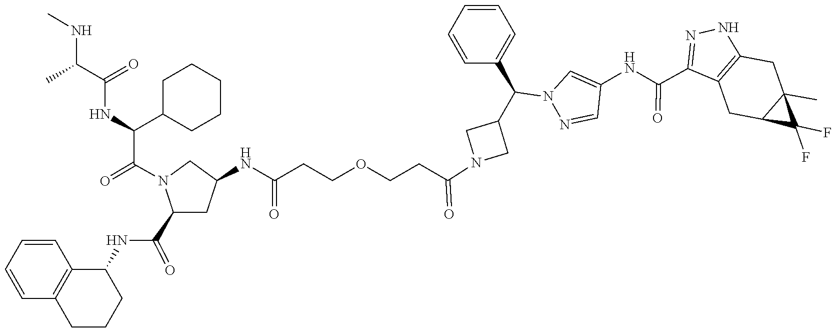 [SMILES: CN[C@@H](C)C(=O)N[C@H](C(=O)N1C[C@@H](NC(=O)CCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1] |
| 2 | 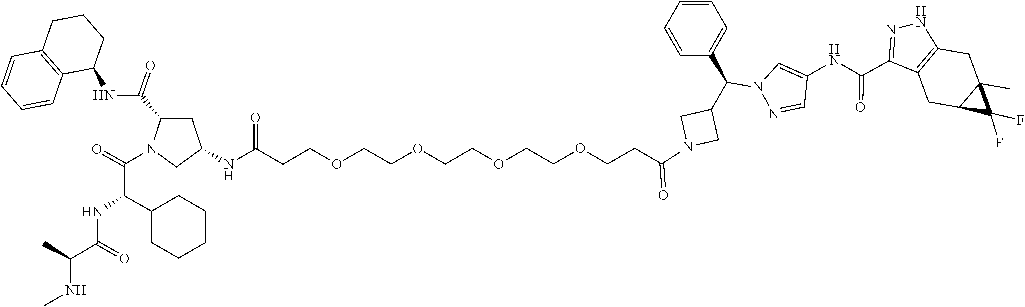 [SMILES: CN[C@@H](C)C(=O)N[C@H](C(=O)N1C[C@@H](NC(=O)CCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1] |
| 3 | 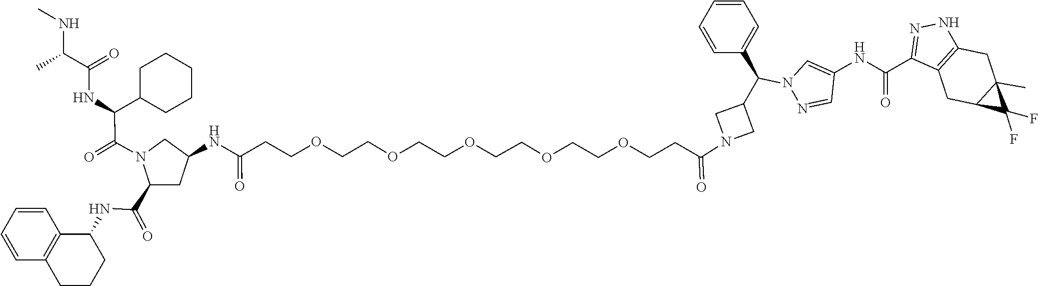 [SMILES: CN[C@@H](C)C(=O)N[C@H](C(=O)N1C[C@@H](NC(=O)CCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C[C@H]1C(=O)N[C@@H]1CCCc2ccccc21)C1CCCCC1] |
| 4 | 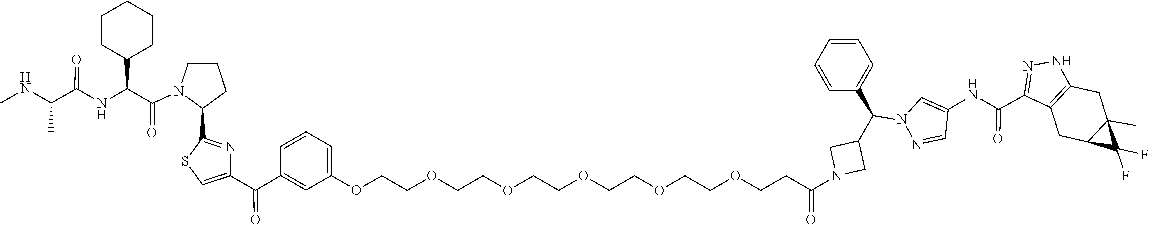 [SMILES: CN[C@@H](C)C(=O)N[C@H](C(=O)N1CCC[C@H]1c1nc(C(=O)c2cccc(OCCOCCOCCOCCOCCOCCC(=O)N3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 5 | 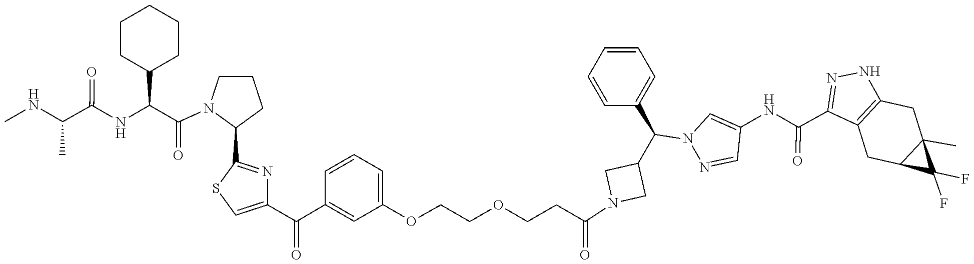 [SMILES: CN[C@@H](C)C(=O)N[C@H](C(=O)N1CCC[C@H]1c1nc(C(=O)c2cccc(OCCOCCC(=O)N3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 6 | 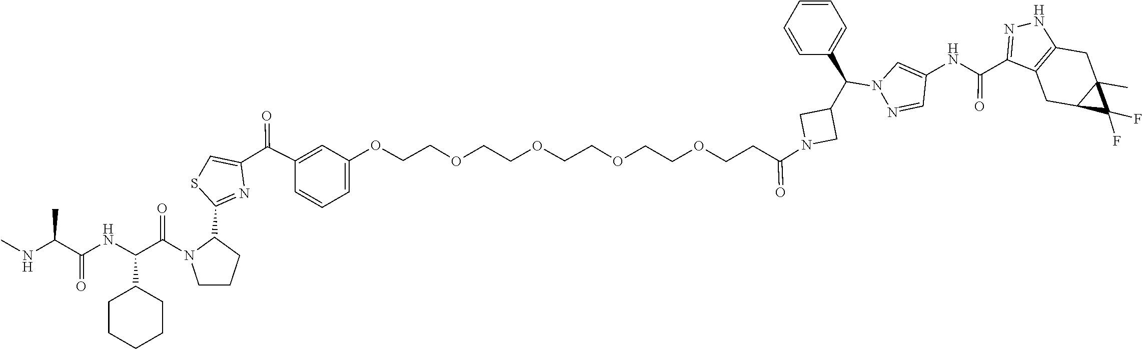 |
| 7 | 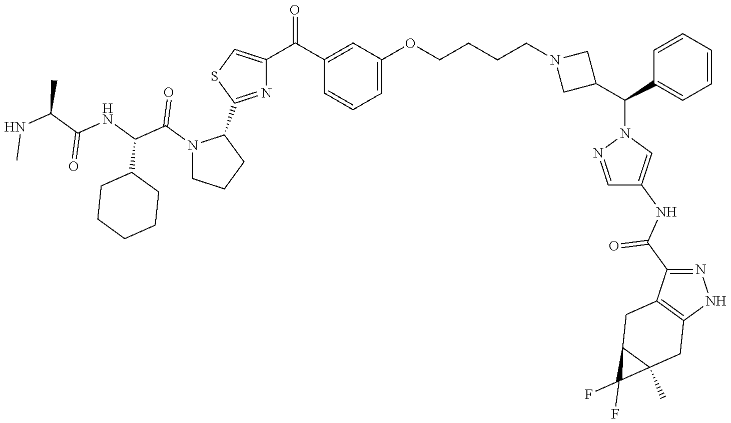 [SMILES: CN[C@@H](C)C(=O)N[C@H](C(=O)N1CCC[C@H]1c1nc(C(=O)c2cccc(OCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 8 | 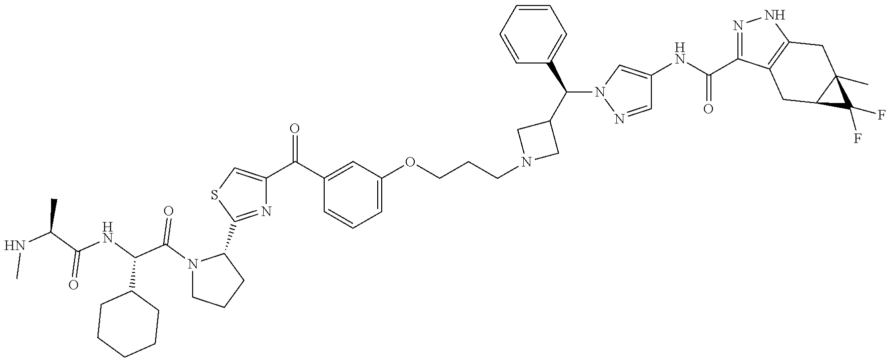 [SMILES: CN[C@@H](C)C(=O)N[C@H](C(=O)N1CCC[C@H]1c1nc(C(=O)c2cccc(OCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 9 | 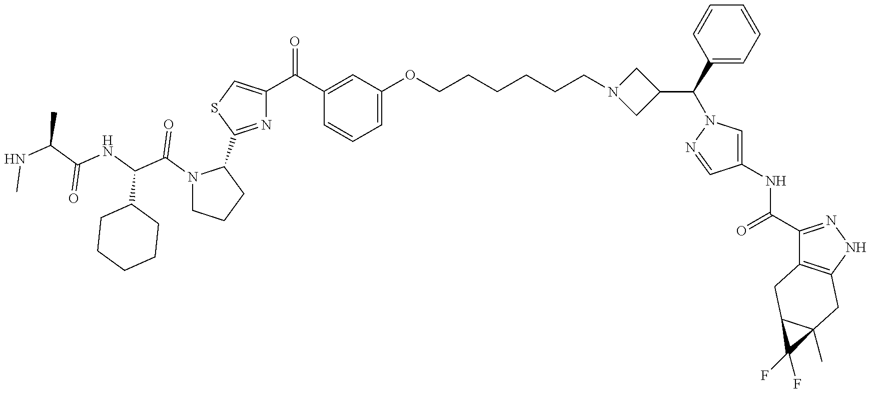 [SMILES: CN[C@@H](C)C(=O)N[C@H](C(=O)N1CCC[C@H]1c1nc(C(=O)c2cccc(OCCCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 10 | 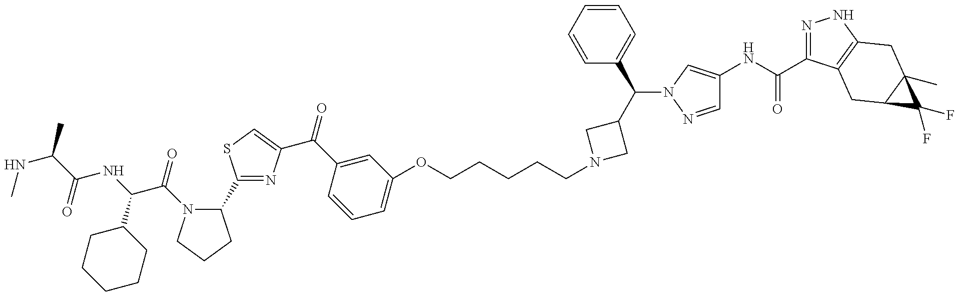 [SMILES: CN[C@@H](C)C(=O)N[C@H](C(=O)N1CCC[C@H]1c1nc(C(=O)c2cccc(OCCCCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 11 | 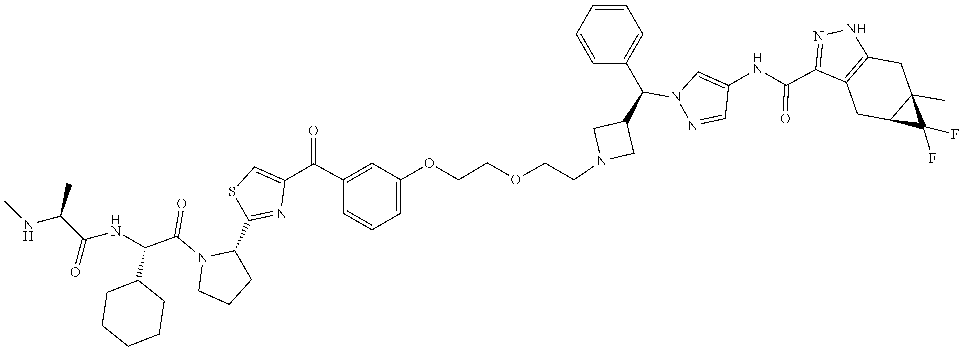 [SMILES: CN[C@@H](C)C(=O)N[C@H](C(=O)N1CCC[C@H]1c1nc(C(=O)c2cccc(OCCOCCN3CC([C@@H](c4ccccc4)n4cc(NC(=O)c5n[nH]c6c5C[C@@H]5C(F)(F)[C@]5(C)C6)cn4)C3)c2)cs1)C1CCCCC1] |
| 12 | 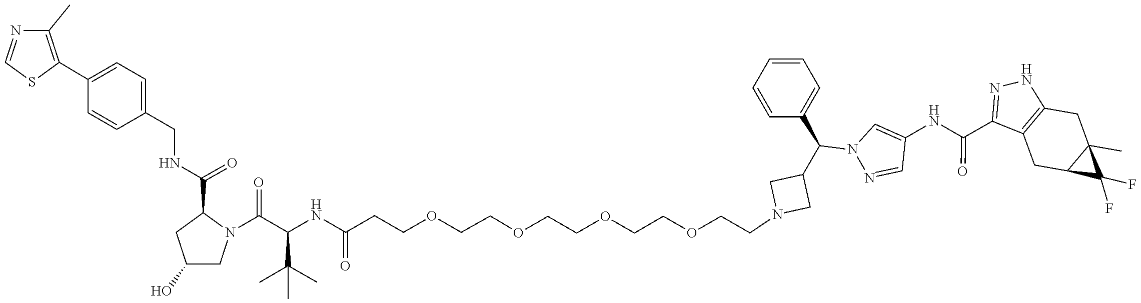 [SMILES: Cc1ncsc1-c1ccc(CNC(=O)[C@@H]2C[C@@H](O)CN2C(=O)[C@@H](NC(=O)CCOCCOCCOCCOCCN2CC([C@@H](c3ccccc3)n3cc(NC(=O)c4n[nH]c5c4C[C@@H]4C(F)(F)[C@]4(C)C5)cn3)C2)C(C)(C)C)cc1] |
| 13 | 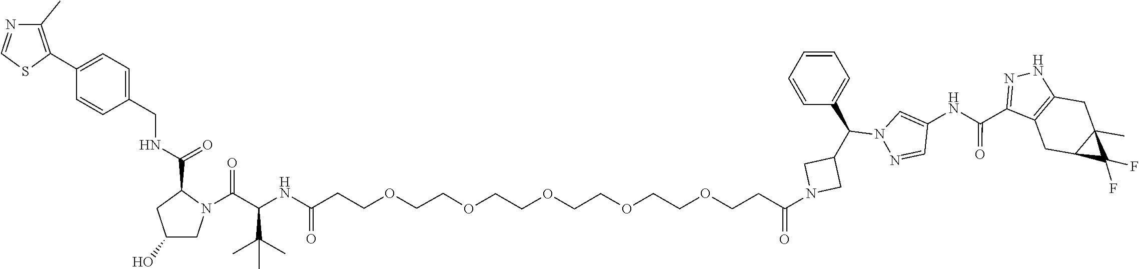 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 14 | 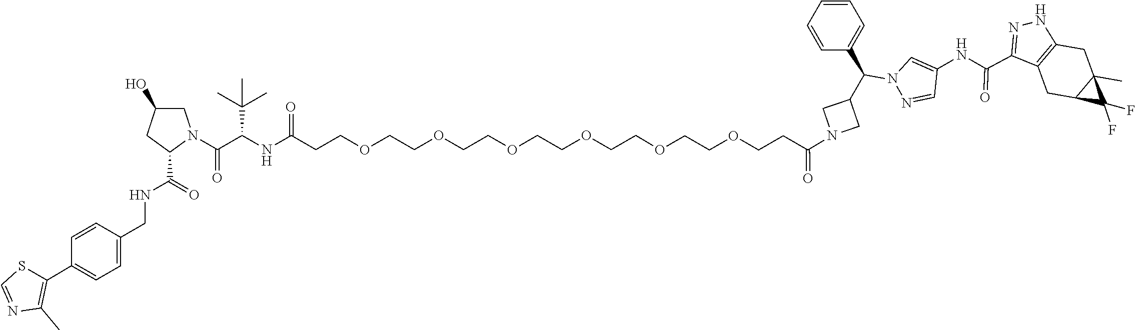 [SMILES: Cc1ncsc1-c1ccc(CNC(=O)[C@@H]2C[C@@H](O)CN2C(=O)[C@@H](NC(=O)CCOCCOCCOCCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 15 | 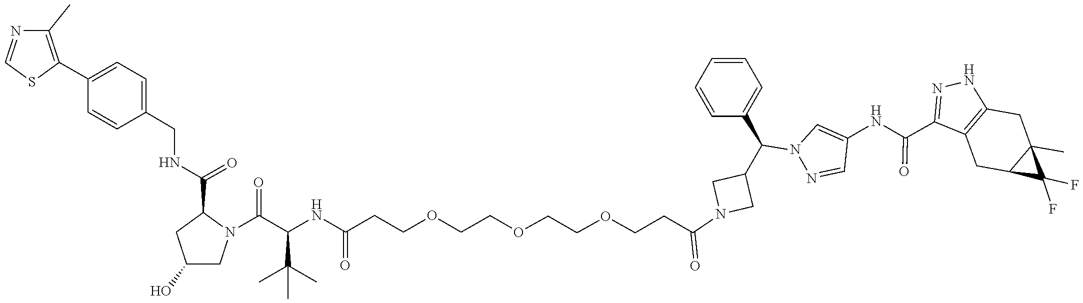 [SMILES: Cc1ncsc1-c1ccc(CNC(=O)[C@@H]2C[C@@H](O)CN2C(=O)[C@@H](NC(=O)CCOCCOCCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 16 | 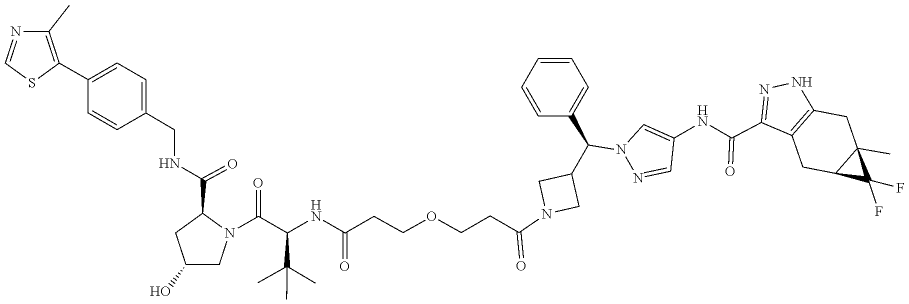 [SMILES: Cc1ncsc1-c1ccc(CNC(=O)[C@@H]2C[C@@H](O)CN2C(=O)[C@@H](NC(=O)CCOCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 17 | 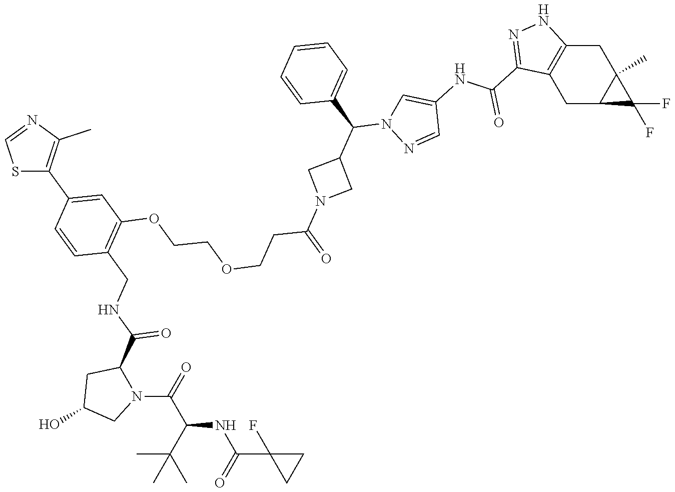 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCC(=O)N2CC([C@@H](c3ccccc3)n3cc(NC(=O)c4n[nH]c5c4C[C@@H]4C(F)(F)[C@]4(C)C5)cn3)C2)c1] |
| 18 | 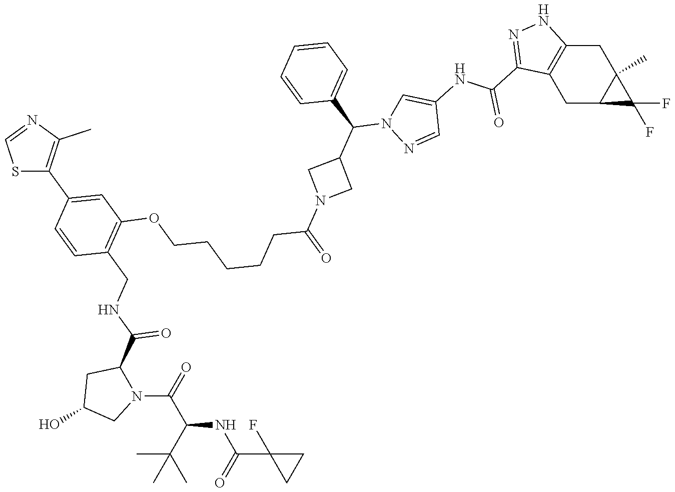 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)N2CC([C@@H](c3ccccc3)n3cc(NC(=O)c4n[nH]c5c4C[C@@H]4C(F)(F)[C@]4(C)C5)cn3)C2)c1] |
| 19 | 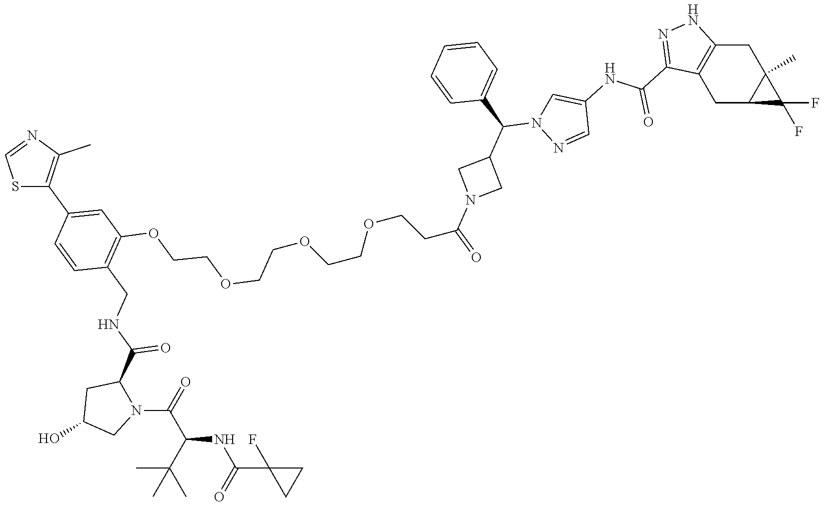 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCC(=O)N2CC([C@@H](c3ccccc3)n3cc(NC(=O)c4n[nH]c5c4C[C@@H]4C(F)(F)[C@]4(C)C5)cn3)C2)c1] |
| 20 | 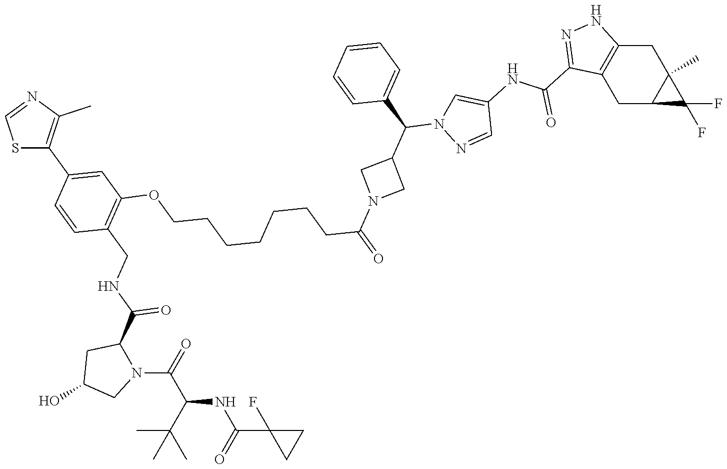 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)N2CC([C@@H](c3ccccc3)n3cc(NC(=O)c4n[nH]c5c4C[C@@H]4C(F)(F)[C@]4(C)C5)cn3)C2)c1] |
| 21 | 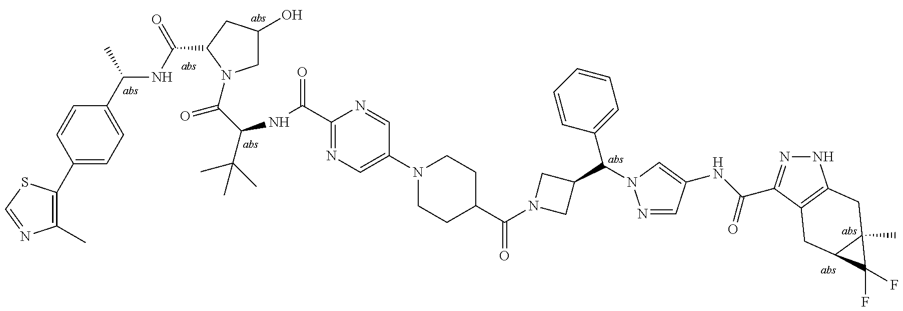 |
| 22 | 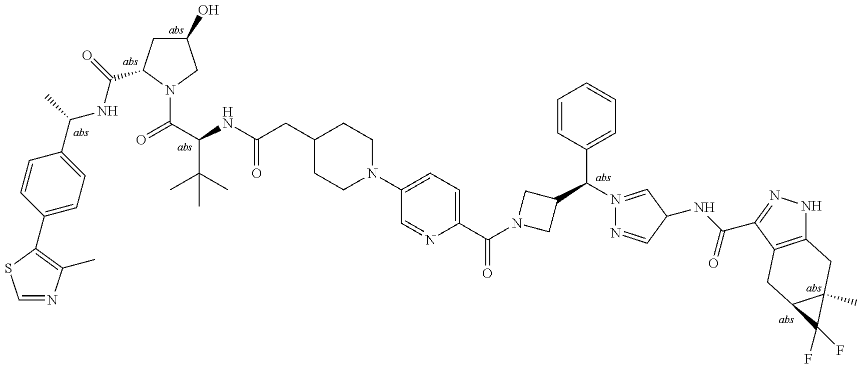 |
| 23 | 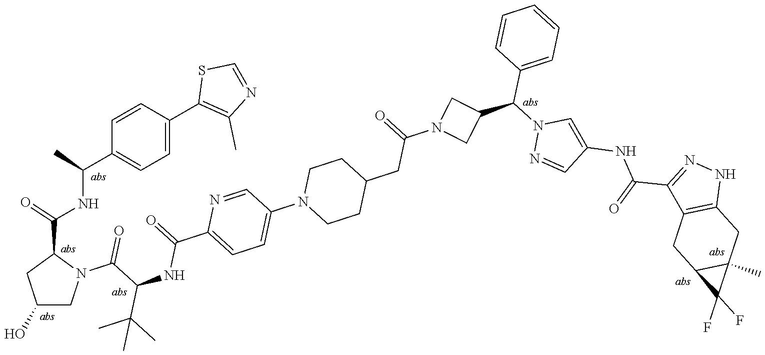 [SMILES: Cc1ncsc1-c1ccc([C@H](C)NC(=O)[C@@H]2C[C@@H](O)CN2C(=O)[C@@H](NC(=O)c2ccc(N3CCC(CC(=O)N4CC([C@@H](c5ccccc5)n5cc(NC(=O)c6n[nH]c7c6C[C@@H]6C(F)(F)[C@]6(C)C7)cn5)C4)CC3)cn2)C(C)(C)C)cc1] |
| 24 | 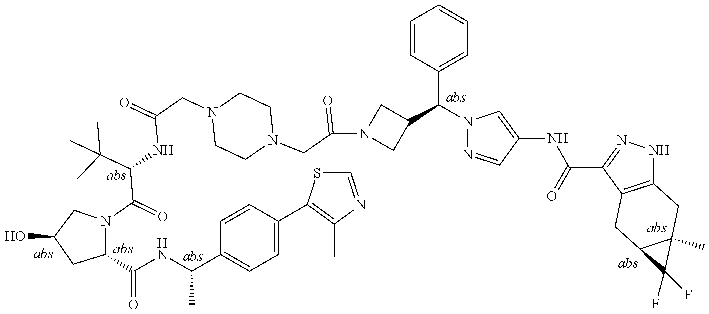 [SMILES: Cc1ncsc1-c1ccc([C@H](C)NC(=O)[C@@H]2C[C@@H](O)CN2C(=O)[C@@H](NC(=O)CN2CCN(CC(=O)N3CC([C@@H](c4ccccc4)n4cc(NC(=O)c5n[nH]c6c5C[C@@H]5C(F)(F)[C@]5(C)C6)cn4)C3)CC2)C(C)(C)C)cc1] |
| 25 | 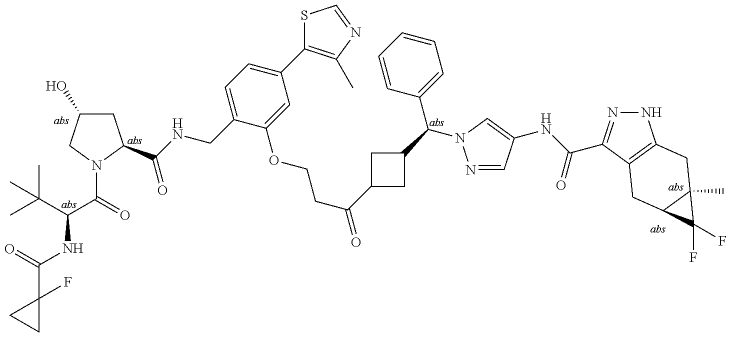 [SMILES: Cc1ncsc1-c1ccc(CNC(=O)[C@@H]2C[C@@H](O)CN2C(=O)[C@@H](NC(=O)C2(F)CC2)C(C)(C)C)c(OCCC(=O)C2CC([C@@H](c3ccccc3)n3cc(NC(=O)c4n[nH]c5c4C[C@@H]4C(F)(F)[C@]4(C)C5)cn3)C2)c1] |
| 26 | 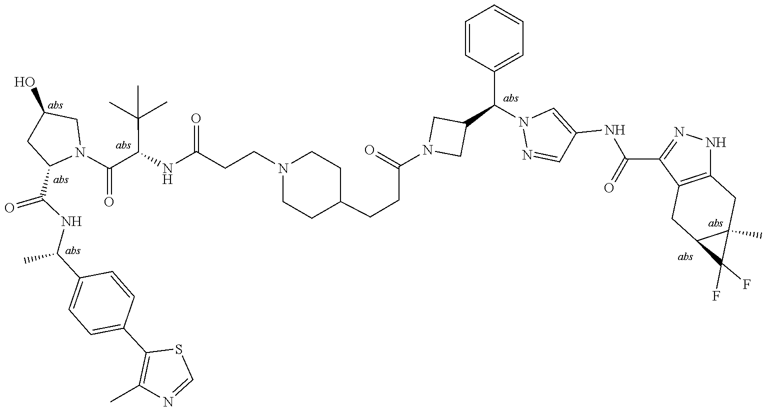 [SMILES: Cc1ncsc1-c1ccc([C@H](C)NC(=O)[C@@H]2C[C@@H](O)CN2C(=O)[C@@H](NC(=O)CCN2CCC(CCC(=O)N3CC([C@@H](c4ccccc4)n4cc(NC(=O)c5n[nH]c6c5C[C@@H]5C(F)(F)[C@]5(C)C6)cn4)C3)CC2)C(C)(C)C)cc1] |
| 27 | 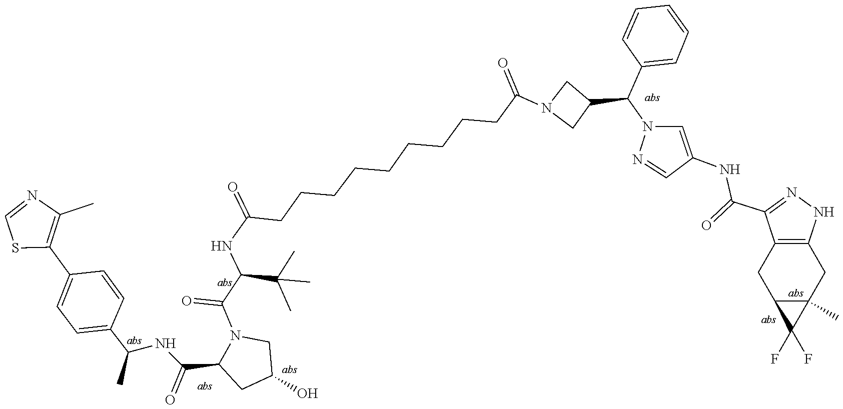 [SMILES: Cc1ncsc1-c1ccc([C@H](C)NC(=O)[C@@H]2C[C@@H](O)CN2C(=O)[C@@H](NC(=O)CCCCCCCCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 28 | 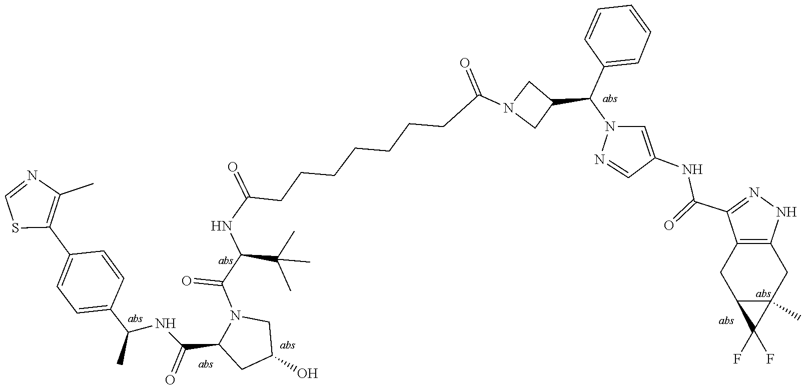 [SMILES: Cc1ncsc1-c1ccc([C@H](C)NC(=O)[C@@H]2C[C@@H](O)CN2C(=O)[C@@H](NC(=O)CCCCCCCC(=O)N2CC([C@@H](c3ccccc3)n3cc(NC(=O)c4n[nH]c5c4C[C@@H]4C(F)(F)[C@]4(C)C5)cn3)C2)C(C)(C)C)cc1] |
| 29 | 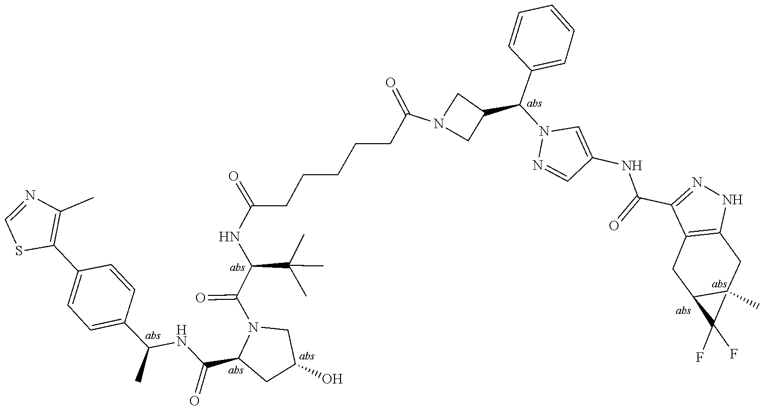 [SMILES: Cc1ncsc1-c1ccc([C@H](C)NC(=O)[C@@H]2C[C@@H](O)CN2C(=O)[C@@H](NC(=O)CCCCCC(=O)N2CC([C@@H](c3ccccc3)n3cc(NC(=O)c4n[nH]c5c4C[C@H]4[C@@H](C)C(C5)C4(F)F)cn3)C2)C(C)(C)C)cc1] |
| 30 | 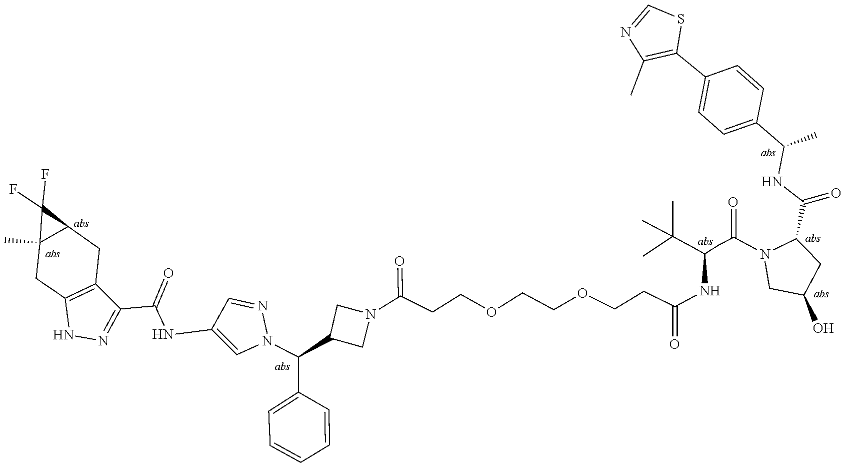 [SMILES: Cc1ncsc1-c1ccc([C@H](C)NC(=O)[C@@H]2C[C@@H](O)CN2C(=O)C[C@H](CC(C)(C)C)NC(=O)CCOCCOCCC(=O)N2CC([C@@H](c3ccccc3)n3cc(NC(=O)c4n[nH]c5c4C[C@@H]4C(F)(F)[C@]4(C)C5)cn3)C2)cc1] |
| 31 | 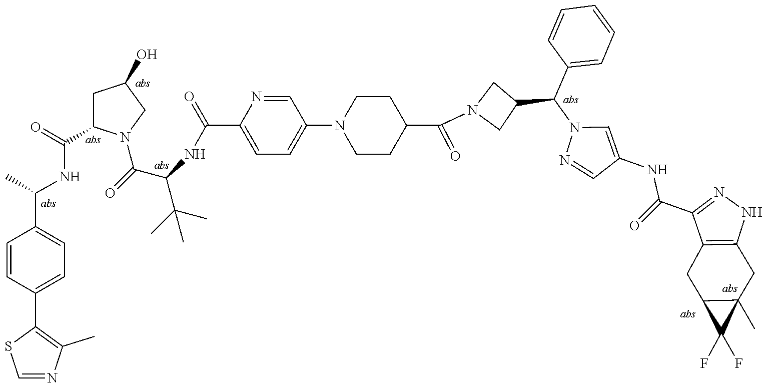 [SMILES: Cc1ncsc1-c1ccc([C@H](C)NC(=O)[C@@H]2C[C@@H](O)CN2C(=O)[C@@H](NC(=O)c2ccc(N3CCC(C(=O)N4CC([C@@H](c5ccccc5)n5cc(NC(=O)c6n[nH]c7c6C[C@@H]6C(F)(F)[C@]6(C)C7)cn5)C4)CC3)cn2)C(C)(C)C)cc1] |
| 32 | 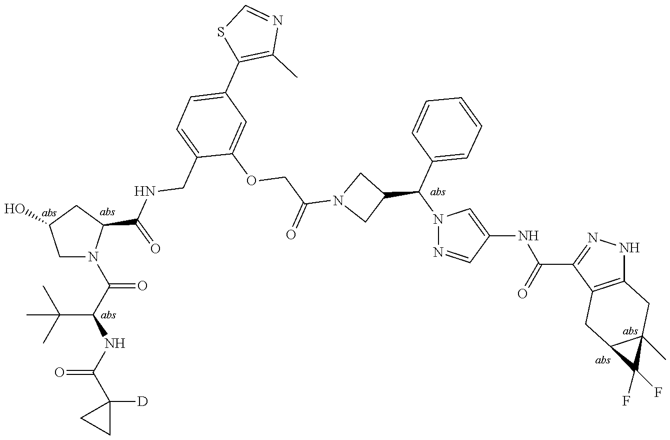 [SMILES: [2H]C1(C(=O)N[C@H](C(=O)N2C[C@H](O)C[C@H]2C(=O)NCc2ccc(-c3scnc3C)cc2OCC(=O)N2CC([C@@H](c3ccccc3)n3cc(NC(=O)c4n[nH]c5c4C[C@@H]4C(F)(F)[C@]4(C)C5)cn3)C2)C(C)(C)C)CC1] |
| 33 | 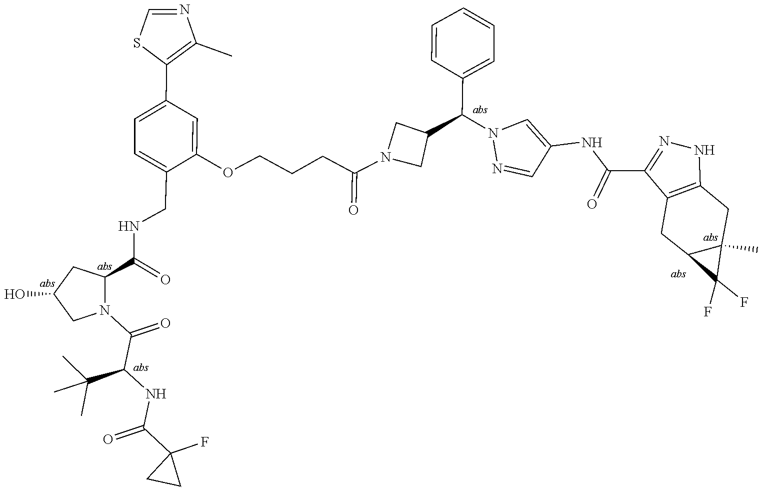 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N2CC([C@@H](c3ccccc3)n3cc(NC(=O)c4n[nH]c5c4C[C@@H]4C(F)(F)[C@]4(C)C5)cn3)C2)c1] |
| 34 | 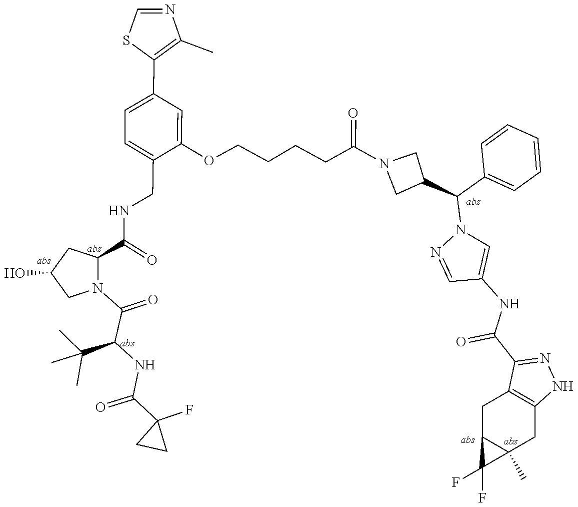 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCC(=O)N2CC([C@@H](c3ccccc3)n3cc(NC(=O)c4n[nH]c5c4C[C@@H]4C(F)(F)[C@]4(C)C5)cn3)C2)c1] |
| 35 | 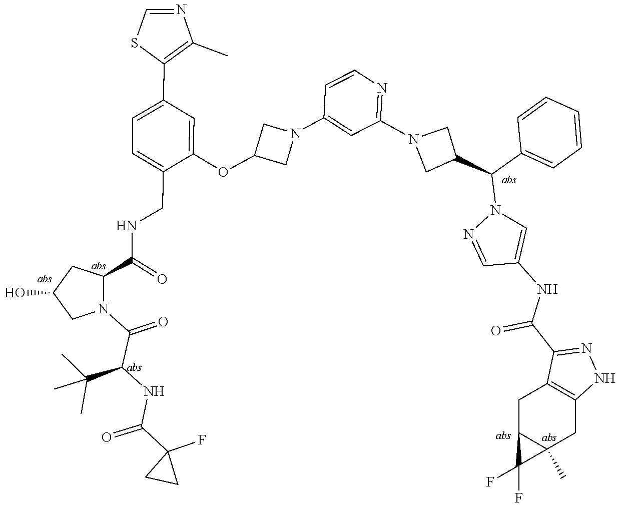 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CN(c3ccnc(N4CC([C@@H](c5ccccc5)n5cc(NC(=O)c6n[nH]c7c6C[C@@H]6C(F)(F)[C@]6(C)C7)cn5)C4)c3)C2)c1] |
| 36 | 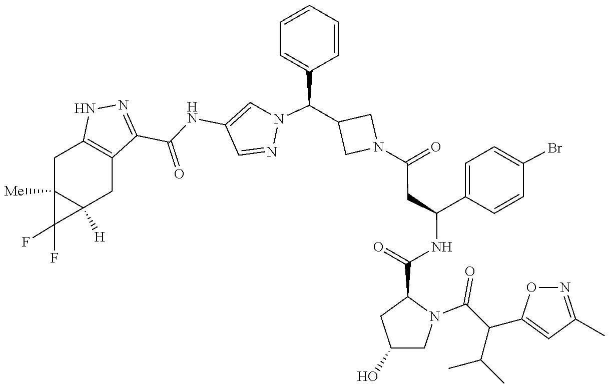 [SMILES: Cc1cc(C(C(=O)N2C[C@H](O)C[C@H]2C(=O)N[C@@H](CC(=O)N2CC([C@H](c3ccccc3)n3cc(NC(=O)c4n[nH]c5c4C[C@H]4C(F)(F)[C@@]4(C)C5)cn3)C2)c2ccc(Br)cc2)C(C)C)on1] |
| 37 | 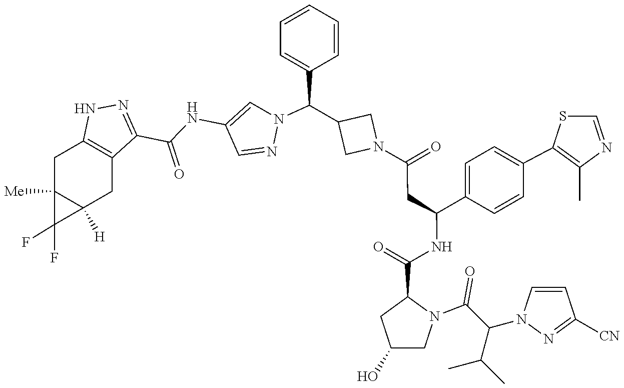 [SMILES: Cc1ncsc1-c1ccc([C@H](CC(=O)N2CC([C@H](c3ccccc3)n3cc(NC(=O)c4n[nH]c5c4C[C@H]4C(F)(F)[C@@]4(C)C5)cn3)C2)NC(=O)[C@@H]2C[C@@H](O)CN2C(=O)C(C(C)C)n2ccc(C#N)n2)cc1] |
| 38 | 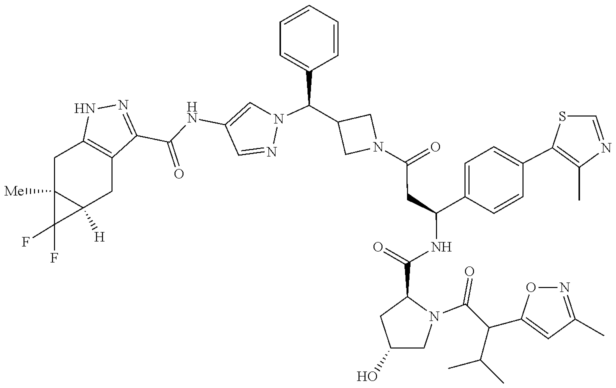 [SMILES: Cc1cc(C(C(=O)N2C[C@H](O)C[C@H]2C(=O)N[C@@H](CC(=O)N2CC([C@H](c3ccccc3)n3cc(NC(=O)c4n[nH]c5c4C[C@H]4C(F)(F)[C@@]4(C)C5)cn3)C2)c2ccc(-c3scnc3C)cc2)C(C)C)on1] |
| 39 | 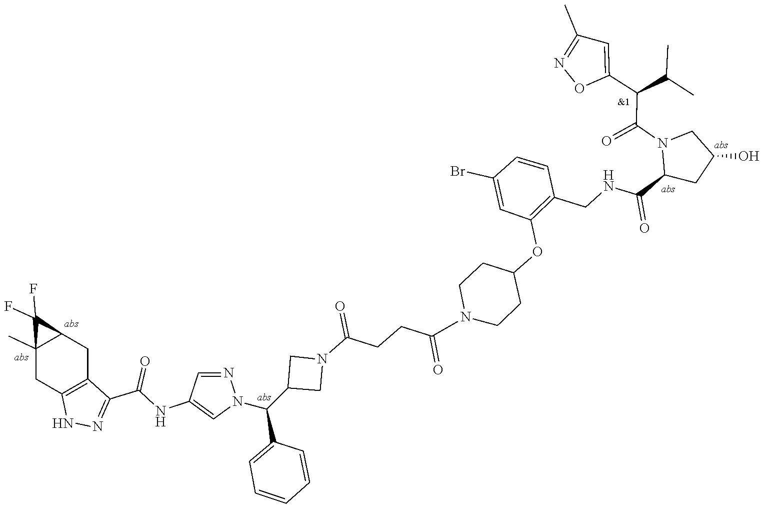 [SMILES: Cc1cc([C@H](C(=O)N2C[C@H](O)C[C@H]2C(=O)NCc2ccc(Br)cc2OC2CCN(C(=O)CCC(=O)N3CC([C@@H](c4ccccc4)n4cc(NC(=O)c5n[nH]c6c5C[C@@H]5C(F)(F)[C@]5(C)C6)cn4)C3)CC2)C(C)C)on1] |

or a stereoisomer and/or pharmaceutical salt thereof.

65. A pharmaceutical composition comprising the compound of any one of the previous claims and a pharmaceutically acceptable carrier, excipient, and/or diluent.

66. A method of treating a disease or disorder in a subject in need thereof comprising administering to the subject a therapeutically effective amount of the compound or composition of any one of the previous claims.

67. The method of claim 66, wherein the disease or disorder is cancer.

68. The compound or composition of any one of the previous claims for use in therapy.

69. The compound or composition of any one of the previous claims for use in the treatment of cancer.
